SEQUENCE LISTING (301 sequences)

SEQ ID NO: 1 - protein, 133 aa, Homo sapiens [sequence omitted]

SEQ ID NO: 2 - protein, 133 aa, Artificial Sequence
  Synthetic: IL-2v
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 3 - protein, 133 aa, Artificial Sequence
  Synthetic: IL-2-P65R
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFCQSIISTLT

SEQ ID NO: 4 - protein, 133 aa, Artificial Sequence
  Synthetic: IL-2-H16A
APTSSSTKKTQLQLEALLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 5 - protein, 133 aa, Artificial Sequence
  Synthetic: IL-2-D84S
APTSSSTKKTQLQLEHLLLDLQMILNGINNYKNPKLTRMLTFKFYMPKKATELKHLQCLEEELKPLEEVLNLAQSKNFHLRPRSLISNINVIVLELKGSETTFMCEYADETATIVEFLNRWITFCQSIISTLT

SEQ ID NO: 6 - protein, 133 aa, Artificial Sequence
  Synthetic: IL-2-E15S
APTSSSTKKTQLQLSHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 133 aa, Artificial Sequence
  Synthetic: IL-2-M23A
APTSSSTKKTQLQLEHLLLDLQAILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 133 aa, Artificial Sequence
  Synthetic: IL-2-E95Q
APTSSSTKKTQLQLEHLLLDLQMILNGINNYKNPKLTRMLTFKFYMPKKATELKHLQCLEEELKPLEEVLNLAQSKNFHLRPRDLISNINVIVLQLKGSETTFMCEYADETATIVEFLNRWITFCQSIISTLT

SEQ ID NO: 9 - protein, 133 aa, Artificial Sequence
  Synthetic: IL-2-P65E
APTSSSTKKTQLQLEHLLLDLQMILNGINNYKNPKLTRMLTFKFYMPKKATELKHLQCLEEELKELEEVLNLAQSKNFHLRPRDLISNINVIVLELKGSETTFMCEYADETATIVEFLNRWITFCQSIISTLT

SEQ ID NO: 10 - protein, 133 aa, Artificial Sequence
  Synthetic: IL-2-F42K
APTSSSTKKTQLQLEHLLLDLQMILNGINNYKNPKLTRMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 11 - protein, 133 aa, Artificial Sequence
  Synthetic: IL-2-H16A-F42K
APTSSSTKKTQLQLEALLLDLQMILNGINNYKNPKLTRMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 133 aa, Artificial Sequence
  Synthetic: IL-2-D84S-F42K
APTSSSTKKTQLQLEHLLLDLQMILNGINNYKNPKLTRMLTKKFYMPKKATELKHLQCLEEELKPLEEVLNLAQSKNFHLRPRSLISNINVIVLELKGSETTFMCEYADETATIVEFLNRWITFCQSIISTLT

SEQ ID NO: 13 - protein, 133 aa, Artificial Sequence
  Synthetic: IL-2-E15S-F42K
APTSSSTKKTQLQLSHLLLDLQMILNGINNYKNPKLTRMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 14 - protein, 133 aa, Artificial Sequence
  Synthetic: IL-2-M23A-F42K
APTSSSTKKTQLQLEHLLLDLQAILNGINNYKNPKLTRMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 15 - protein, 133 aa, Artificial Sequence
  Synthetic: IL-2-E95Q-F42K
APTSSSTKKTQLQLEHLLLDLQMILNGINNYKNPKLTRMLTKKFYMPKKATELKHLQCLEEELKPLEEVLNLAQSKNFHLRPRDLISNINVIVLQLKGSETTFMCEYADETATIVEFLNRWITFCQSIISTLT

SEQ ID NO: 16 - protein, 133 aa, Artificial Sequence
  Synthetic: IL-2-P65R-H16A
APTSSSTKKTQLQLEALLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFCQSIISTLT

SEQ ID NO: 17 - protein, 133 aa, Artificial Sequence
  Synthetic: IL-2-P65R-D84S
APTSSSTKKTQLQLEHLLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFCQSIISTLT

SEQ ID NO: 18 - protein, 133 aa, Artificial Sequence
  Synthetic: IL-2-P65R-E15S
APTSSSTKKTQLQLSHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFCQSIISTLT

SEQ ID NO: 19 - protein, 133 aa, Artificial Sequence
  Synthetic: IL-2-P65R-M23A
APTSSSTKKTQLQLEHLLLDLQAILNGINNYKNPKLTRMLTFKFYMPKKATELKHLQCLEEELKRLEEVLNLAQSKNFHLRPRDLISNINVIVLELKGSETTFMCEYADETATIVEFLNRWITFCQSIISTLT

SEQ ID NO: 20 - protein, 133 aa, Artificial Sequence
  Synthetic: IL-2-P65R-E95Q
APTSSSTKKTQLQLEHLLLDLQMILNGINNYKNPKLTRMLTFKFYMPKKATELKHLQCLEEELKRLEEVLNLAQSKNFHLRPRDLISNINVIVLQLKGSETTFMCEYADETATIVEFLNRWITFCQSIISTLT

SEQ ID NO: 21 - protein, 133 aa, Artificial Sequence
  Synthetic: IL-2-P65R-H16A-D84S (IL-2-RAS)
APTSSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFCQSIISTLT

SEQ ID NO: 22 - protein, 133 aa, Artificial Sequence
  Synthetic: IL-2-T3A-C125S
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 23 - protein, 133 aa, Artificial Sequence
  Synthetic: IL-2-T3A-P65R-C125S
APASSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFSQSIISTLT

SEQ ID NO: 24 - protein, 133 aa, Artificial Sequence
  Synthetic: IL-2-T3A-H16A-C125S
APASSSTKKTQLQLEALLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 25 - protein, 133 aa, Artificial Sequence
  Synthetic: IL-2-T3A-D84S-C125S
APASSSTKKTQLQLEHLLLDLQMILNGINNYKNPKLTRMLTFKFYMPKKATELKHLQCLEEELKPLEEVLNLAQSKNFHLRPRSLISNINVIVLELKGSETTFMCEYADETATIVEFLNRWITFSQSIISTLT

SEQ ID NO: 26 - protein, 133 aa, Artificial Sequence
  Synthetic: IL-2-T3A-H16A-P65R-C125S
APASSSTKKTQLQLEALLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFSQSIISTLT

SEQ ID NO: 27 - protein, 133 aa, Artificial Sequence
  Synthetic: IL-2-T3A-P65R-D84S-C125S
APASSSTKKTQLQLEHLLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 28 - protein, 133 aa, Artificial Sequence
  Synthetic: IL-2-T3A-H16A-P65R-D84S-C125S
APASSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 29 - protein, 124 aa, Artificial Sequence
  Synthetic: IL-2-no9-H16A-P65R-C125S
TQLQLEALLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFSQSIISTLT

SEQ ID NO: 30 - protein, 124 aa, Artificial Sequence
  Synthetic: IL-2-no9-P65R-D84S-C125S
TQLQLEHLLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 31 - protein, 124 aa, Artificial Sequence
  Synthetic: IL-2-no9-H16A-P65R-D84S-C125S
TQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 32 - protein, 362 aa, Artificial Sequence
  Synthetic: Wild type IL-2-xELL knob Fc
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 362 aa, Artificial Sequence
  Synthetic: IL-2-F42K-xELL knob Fc
APTSSSTKKTQLQLEHLLLDLQMILNGINNYKNPKLTRMLTKKFYMPKKATELKHLQCLEEELKPLEEVLNLAQSKNFHLRPRDLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 362 aa, Artificial Sequence
  Synthetic: IL-2-P65E-xELL knob Fc
APTSSSTKKTQLQLEHLLLDLQMILNGINNYKNPKLTRMLTFKFYMPKKATELKHLQCLEEELKELEEVLNLAQSKNFHLRPRDLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 362 aa, Artificial Sequence
  Synthetic: IL-2-P65R-xELL knob Fc
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 362 aa, Artificial Sequence
  Synthetic: IL-2-F42K-D84S-xELL knob Fc
APTSSSTKKTQLQLEHLLLDLQMILNGINNYKNPKLTRMLTKKFYMPKKATELKHLQCLEEELKPLEEVLNLAQSKNFHLRPRSLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 362 aa, Artificial Sequence
  Synthetic: IL-2-F42K-E95Q-xELL knob Fc
APTSSSTKKTQLQLEHLLLDLQMILNGINNYKNPKLTRMLTKKFYMPKKATELKHLQCLEEELKPLEEVLNLAQSKNFHLRPRDLISNINVIVLQLKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 362 aa, Artificial Sequence
  Synthetic: IL-2-F42K-M23A-xELL knob Fc
APTSSSTKKTQLQLEHLLLDLQAILNGINNYKNPKLTRMLTKKFYMPKKATELKHLQCLEEELKPLEEVLNLAQSKNFHLRPRDLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 362 aa, Artificial Sequence
  Synthetic: IL-2-H16A-F42K- xELL knob Fc
APTSSSTKKTQLQLEALLLDLQMILNGINNYKNPKLTRMLTKKFYMPKKATELKHLQCLEEELKPLEEVLNLAQSKNFHLRPRDLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 362 aa, Artificial Sequence
  Synthetic: IL-2-H16A-xELL knob Fc
APTSSSTKKTQLQLEALLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 362 aa, Artificial Sequence
  Synthetic: IL-2-P65R-H16A-xELL knob Fc
APTSSSTKKTQLQLEALLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 362 aa, Artificial Sequence
  Synthetic: IL-2-P65R-D84S-xELL knob Fc
APTSSSTKKTQLQLEHLLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 362 aa, Artificial Sequence
  Synthetic: IL-2-RAS-xELL knob Fc
APTSSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 230 aa, Artificial Sequence
  Synthetic: Linker-EVN xELL hole Fc
KPGGGGDKTHTCPPCPAPGGPSVFLFPPKPKDTLMRSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 361 aa, Artificial Sequence
  Synthetic: xELL knob Fc-IL-2-T3G-C125S
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPGSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 46 - protein, 361 aa, Artificial Sequence
  Synthetic: xELL knob Fc-IL-2-RAS-T3G-C125S
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPGSSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 47 - protein, 226 aa, Homo sapiens
  SITE (1)..(226): Fc region 1 (human wild type IgG1)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 223 aa, Homo sapiens
  SITE (1)..(223): Fc region 2 (human IgG1 xELL knob)
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 223 aa, Homo sapiens
  SITE (1)..(223): Fc region 3 (human IgG1 EVN xELL hole I253R)
DKTHTCPPCPAPGGPSVFLFPPKPKDTLMRSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 50 - protein, 226 aa, Artificial Sequence; identical to SEQ ID NO: 47
  Synthetic: Fc region

SEQ ID NO: 51 - protein, 223 aa, Artificial Sequence
  Synthetic: Fc region xELL
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 222 aa, Artificial Sequence
  Synthetic: Fc region xELL H435R
DKTHTCPPCPAPGGPSVFLFPPKPKDTLMISRTPETCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 53 - protein, 223 aa, Artificial Sequence
  Synthetic: Fc region xELL M252Y and M428V (YV)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNHYTQKSLSLSPG

SEQ ID NO: 54 - protein, 223 aa, Artificial Sequence
  Synthetic: Fc region xELL M252Y and M428L (YL)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 55 - protein, 223 aa, Artificial Sequence
  Synthetic: Fc region xELL M252Y, M428L, H435R (YLR)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNRYTQKSLSLSPG

SEQ ID NO: 56 - protein, 223 aa, Artificial Sequence
  Synthetic: Fc region xELL M252Y, M428V, H435R (YVR)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNRYTQKSLSLSPG

SEQ ID NO: 57 - protein, 223 aa, Artificial Sequence; identical to SEQ ID NO: 48
  Synthetic: Fc region xELL S354C T366W knob

SEQ ID NO: 58 - protein, 223 aa, Artificial Sequence
  Synthetic: Fc region xELL H435R S354C T366W knob
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRYTQKSLSLSPG

SEQ ID NO: 59 - protein, 223 aa, Artificial Sequence
  Synthetic: Fc region xELL M252Y and M428V (YV) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPG

SEQ ID NO: 60 - protein, 223 aa, Artificial Sequence
  Synthetic: Fc region xELL M252Y and M428L (YL) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPG

SEQ ID NO: 61 - protein, 223 aa, Artificial Sequence
  Synthetic: Fc region xELL M252Y, M428L, H435R (YLR) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNRYTQKSLSLSPG

SEQ ID NO: 62 - protein, 223 aa, Artificial Sequence
  Synthetic: Fc region xELL M252Y, M428V, H435R (YVR) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNRYTQKSLSLSPG

SEQ ID NO: 63 - protein, 223 aa, Artificial Sequence
  Synthetic: Fc region xELL T366S, L368A, Y407V hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 64 - protein, 223 aa, Artificial Sequence
  Synthetic: Fc region xELL H435R, T366S, L368A, Y407V hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 65 - protein, 223 aa, Artificial Sequence
  Synthetic: Fc region xELL M252Y and M428V (YV) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNHYTQKSLSLSPG

SEQ ID NO: 66 - protein, 223 aa, Artificial Sequence
  Synthetic: Fc region xELL M252Y and M428L (YL) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPG

SEQ ID NO: 67 - protein, 223 aa, Artificial Sequence
  Synthetic: Fc region xELL M252Y, M428L, H435R (YLR) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRYTQKSLSLSPG

SEQ ID NO: 68 - protein, 223 aa, Artificial Sequence
  Synthetic: Fc region xELL M252Y, M428V, H435R (YVR) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNRYTQKSLSLSPG

SEQ ID NO: 69 - protein, 226 aa, Artificial Sequence
  Synthetic: Fc region H435R
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 70 - protein, 226 aa, Artificial Sequence
  Synthetic: Fc region M252Y and M428V (YV)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNHYTQKSLSLSPG

SEQ ID NO: 71 - protein, 226 aa, Artificial Sequence
  Synthetic: Fc region M252Y and M428L (YL)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 72 - protein, 226 aa, Artificial Sequence
  Synthetic: Fc region M252Y, M428L, H435R (YLR)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNRYTQKSLSLSPG

SEQ ID NO: 73 - protein, 226 aa, Artificial Sequence
  Synthetic: Fc region M252Y, M428V, H435R (YVR)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNRYTQKSLSLSPG

SEQ ID NO: 74 - protein, 226 aa, Artificial Sequence
  Synthetic: Fc region S354C T366W knob
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 226 aa, Artificial Sequence
  Synthetic: Fc region H435R S354C T366W knob
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRYTQKSLSLSPG

SEQ ID NO: 76 - protein, 226 aa, Artificial Sequence
  Synthetic: Fc region M252Y and M428L (YL) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPG

SEQ ID NO: 77 - protein, 226 aa, Artificial Sequence
  Synthetic: Fc region M252Y, M428L, H435R (YLR) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNRYTQKSLSLSPG

SEQ ID NO: 78 - protein, 226 aa, Artificial Sequence
  Synthetic: Fc region M252Y, M428V, H435R (YVR) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNRYTQKSLSLSPG

SEQ ID NO: 79 - protein, 226 aa, Artificial Sequence
  Synthetic: Fc region T366S, L368A, Y407V hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 80 - protein, 226 aa, Artificial Sequence
  Synthetic: Fc region H435R, T366S, L368A, Y407V hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 81 - protein, 226 aa, Artificial Sequence
  Synthetic: Fc region M252Y and M428V (YV) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNHYTQKSLSLSPG

SEQ ID NO: 82 - protein, 226 aa, Artificial Sequence
  Synthetic: Fc region M252Y and M428L (YL) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPG

SEQ ID NO: 83 - protein, 226 aa, Artificial Sequence
  Synthetic: Fc region M252Y, M428L, H435R (YLR) T366S, L368A,Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRYTQKSLSLSPG

SEQ ID NO: 84 - protein, 119 aa, Homo sapiens
  SITE (1)..(119): Truncated wild-type human IL-2
QLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSII

SEQ ID NO: 85 - protein, 362 aa, Artificial Sequence
  Synthetic: IL-2-F42K-E15S-xELL knob Fc
APTSSSTKKTQLQLSHLLLDLQMILNGINNYKNPKLTRMLTKKFYMPKKATELKHLQCLEEELKPLEEVLNLAQSKNFHLRPRDLISNINVIVLELKGSETTFMCEYADETATIVEFLNRWITFCQSIISTLTPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 361 aa, Artificial Sequence
  Synthetic: xELL-Knob Fc-IL-2-T3A, C125S
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 87 - protein, 361 aa, Artificial Sequence
  Synthetic: xELL-Knob Fc-IL-2-RAS-T3A, C125S
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 88 - protein, 584 aa, Artificial Sequence
  Synthetic: Pembrolizumab analog Knob Fc-IL-2-RAS-T3A, C125S
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYDTTPPVLDSDGSFFLYSDLTVDKSRWQQGNVFSCSVMHEALHNRYTQKSLSLSPGGSGGSAPASSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 89 - protein, 447 aa, Artificial Sequence
  Synthetic: Pembrolizumab analog Hole Fc
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDKLTKNQVSLSCAVKGFYPSDIAVEWKSNGQPENNYKTTPPVLDSKGSFFLVSKLTVDKSRWQQGNVFSCSVMHEALHNHYTQKSLSLSPGK

SEQ ID NO: 90 - protein, 218 aa, Artificial Sequence
  Synthetic: Pembrolizumab Light Chain analog
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 584 aa, Artificial Sequence
  Synthetic: Pembrolizumab analog IL-2-RAS-T3G, C125S
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGSGGSAPGSSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 92 - protein, 611 aa, Artificial Sequence
  Synthetic: NKp46-scFv xELL-Knob Fc-IL-2-RAS-T3A, C125S
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSVEGGSGGSGGSGGSGGVDDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYDTTPPVLDSDGSFFLYSDLTVDKSRWQQGNVFSCSVMHEALHNRYTQKSLSLSPGGSGGSAPASSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 93 - protein, 474 aa, Artificial Sequence
  Synthetic: NKp46-scFv xELL-Hole Fc
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSVEGGSGGSGGSGGSGGVDDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDKLTKNQVSLSCAVKGFYPSDIAVEWKSNGQPENNYKTTPPVLDSKGSFFLVSKLTVDKSRWQQGNVFSCSVMHEALHNHYTQKSLSLSPGK

SEQ ID NO: 94 - protein, 477 aa, Artificial Sequence
  Synthetic: NKp46-scFv xELL-Fc
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSVEGGSGGSGGSGGSGGVDDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKPGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 587 aa, Artificial Sequence
  Synthetic: LAG3-MAb xELL-Knob Fc-IL-2-RAS-TGCS
QVQLVQSGAEVKKPGASVKVSCKASGYTFTGYYMHWVRQAPGQGLEWMGWINANSGGTNYAQKFQGRVTMTRDTSISTAYMELSRLRSDDTAVYYCARDIYDSSDQLNVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPGSSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 96 - protein, 447 aa, Artificial Sequence
  Synthetic: LAG3-MAb xELL-Hole Fc
QVQLVQSGAEVKKPGASVKVSCKASGYTFTGYYMHWVRQAPGQGLEWMGWINANSGGTNYAQKFQGRVTMTRDTSISTAYMELSRLRSDDTAVYYCARDIYDSSDQLNVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPGGPSVFLFPPKPKDTLMRSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 97 - protein, 214 aa, Artificial Sequence
  Synthetic: LAG3-MAb Light Chain
EIVLTQSPATLSLSPGERATLSCRASQSVSSYLAWYQQKPGQAPRLLIYDASNRATGIPARFSGSGSGTDFTLTISSLEPEDFAVYYCQQASIWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 450 aa, Artificial Sequence
  Synthetic: LAG3-MAb IgG1
QVQLVQSGAEVKKPGASVKVSCKASGYTFTGYYMHWVRQAPGQGLEWMGWINANSGGTNYAQKFQGRVTMTRDTSISTAYMELSRLRSDDTAVYYCARDIYDSSDQLNVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 492 aa, Artificial Sequence
  Synthetic: LAG3-VHH xELL-Knob Fc-IL-2-RAS-TGCS
EVQLVESGGGWQPGGSLRLSCAASGRTFSDYVMGWFRQAPGKEREFVAAISESGGRTHYADSVKGRFTISRDNSKNTLYLQMNSLRPEDTALYYCATTLLWWTSEYAPIKANDYDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPGSSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 100 - protein, 355 aa, Artificial Sequence
  Synthetic: LAG3-VHH xELL-Hole T366S, L368A, Y407V, H435R Fc
EVQLVESGGGWQPGGSLRLSCAASGRTFSDYVMGWFRQAPGKEREFVAAISESGGRTHYADSVKGRFTISRDNSKNTLYLQMNSLRPEDTALYYCATTLLWWTSEYAPIKANDYDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 101 - protein, 355 aa, Artificial Sequence
  Synthetic: LAG3-VHH xELL-Knob Fc
EVQLVESGGGWQPGGSLRLSCAASGRTFSDYVMGWFRQAPGKEREFVAAISESGGRTHYADSVKGRFTISRDNSKNTLYLQMNSLRPEDTALYYCATTLLWWTSEYAPIKANDYDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 361 aa, Artificial Sequence
  Synthetic: xELL-Knob Fc-IL-2-RAS-M23A-T3A, C125S
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPGGSGGSAPASSSTKKTQLQLEALLLDLQAILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 103 - protein, 361 aa, Artificial Sequence
  Synthetic: xELL-Knob Fc-IL-2-RAS-E95Q-T3A, C125S
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPGGSGGSAPASSSTKKTQLQLEALLLDLQMILNGINNYKNPKLTRMLTFKFYMPKKATELKHLQCLEEELKRLEEVLNLAQSKNFHLRPRSLISNINVIVLQLKGSETTFMCEYADETATIVEFLNRWITFSQSIISTLT

SEQ ID NO: 104 - protein, 361 aa, Artificial Sequence
  Synthetic: xELL-Knob Fc-IL-2-RAS-M23A-E95Q-T3A, C125S
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPGGSGGSAPASSSTKKTQLQLEALLLDLQAILNGINNYKNPKLTRMLTFKFYMPKKATELKHLQCLEEELKRLEEVLNLAQSKNFHLRPRSLISNINVIVLQLKGSETTFMCEYADETATIVEFLNRWITFSQSIISTLT

SEQ ID NO: 105 - protein, 133 aa, Artificial Sequence
  Synthetic: IL-2-T3G-C125S
APGSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 106 - protein, 133 aa, Artificial Sequence
  Synthetic: IL-2-T3A-E61R-P65R-C125S
APASSSTKKTQLQLEHLLLDLQMILNGINNYKNPKLTRMLTFKFYMPKKATELKHLQCLERELKRLEEVLNLAQSKNFHLRPRDLISNINVIVLELKGSETTFMCEYADETATIVEFLNRWITFSQSIISTLT

SEQ ID NO: 107 - protein, 133 aa, Artificial Sequence
  Synthetic: IL-2-T3A-R38A-P65R-C125S
APASSSTKKTQLQLEHLLLDLQMILNGINNYKNPKLTAMLTFKFYMPKKATELKHLQCLEEELKRLEEVLNLAQSKNFHLRPRDLISNINVIVLELKGSETTFMCEYADETATIVEFLNRWITFSQSIISTLT

SEQ ID NO: 108 - protein, 133 aa, Artificial Sequence
  Synthetic: IL-2-T3A-H16A-E61R-P65R-D84S-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 109 - protein, 133 aa, Artificial Sequence
  Synthetic: IL-2-T3A-H16A-R38A-P65R-D84S-C125S
APASSSTKKTQLQLEALLLDLQMILNGINNYKNPKLTAMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 110 - protein, 133 aa, Artificial Sequence
  Synthetic: IL-2-T3A-H16A-L19A-P65R-D84S-C125S
APASSSTKKTQLQLEALLLALQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 111 - protein, 133 aa, Artificial Sequence
  Synthetic: IL-2-T3A-H16A-P65R-D84S-N88S-C125S
APASSSTKKTQLQLEALLLDLQMILNGINNYKNPKLTRMLTFKFYMPKKATELKHLQCLEEELKRLEEVLNLAQSKNFHLRPRSLISSINVIVLELKGSETTFMCEYADETATIVEFLNRWITFSQSIISTLT

SEQ ID NO: 112 - protein, 133 aa, Artificial Sequence
  Synthetic: IL-2-T3A-H16A-P65R-D84S-E95Q-C125S
APASSSTKKTQLQLEALLLDLQMILNGINNYKNPKLTRMLTFKFYMPKKATELKHLQCLEEELKRLEEVLNLAQSKNFHLRPRSLISNINVIVLQLKGSETTFMCEYADETATIVEFLNRWITFSQSIISTLT

SEQ ID NO: 113 - protein, 133 aa, Artificial Sequence
  Synthetic: IL-2-T3A-H16A-M23A-P65R-D84S-C125S
APASSSTKKTQLQLEALLLDLQAILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 114 - protein, 133 aa, Artificial Sequence
  Synthetic: IL-2-T3A-H16A-M23A-P65R-D84S-E95Q-C125S
APASSSTKKTQLQLEALLLDLQAILNGINNYKNPKLTRMLTFKFYMPKKATELKHLQCLEEELKRLEEVLNLAQSKNFHLRPRSLISNINVIVLQLKGSETTFMCEYADETATIVEFLNRWITFSQSIISTLT

SEQ ID NO: 115 - protein, 133 aa, Artificial Sequence
  Synthetic: IL-2-T3A-H16A-L19A-E61R-P65R-D84S-C125S
APASSSTKKTQLQLEALLADLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 116 - protein, 133 aa, Artificial Sequence
  Synthetic: IL-2-T3A-H16A-M23A-E61R-P65R-D84S-E95Q-C125S
APASSSTKKTQLQLEALLLDLQAILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 117 - protein, 133 aa, Artificial Sequence
  Synthetic: IL-2-T3A-H16A-M23A-E61R-P65R-D84S-N88S-C125S
APASSSTKKTQLQLEALLLDLQAILNGINNYKNPKLTRMLTFKFYMPKKATELKHLQCLERELKRLEEVLNLAQSKNFHLRPRSLISSINVIVLELKGSETTFMCEYADETATIVEFLNRWITFSQSIISTLT

SEQ ID NO: 118 - protein, 133 aa, Artificial Sequence
  Synthetic: IL-2-T3A-H16A-E61R-P65R-D84S-N88S-E95Q-C125S
APASSSTKKTQLQLEALLLDLQMILNGINNYKNPKLTRMLTFKFYMPKKATELKHLQCLERELKRLEEVLNLAQSKNFHLRPRSLISSINVIVLQLKGSETTFMCEYADETATIVEFLNRWITFSQSIISTLT

SEQ ID NO: 119 - protein, 133 aa, Artificial Sequence
  Synthetic: IL-2-T3A-H16A-M23A-E61R-P65R-D84S-N88S-E95Q-C125S
APASSSTKKTQLQLEALLLDLQAILNGINNYKNPKLTRMLTFKFYMPKKATELKHLQCLERELKRLEEVLNLAQSKNFHLRPRSLISSINVIVLQLKGSETTFMCEYADETATIVEFLNRWITFSQSIISTLT

SEQ ID NO: 120 - protein, 133 aa, Artificial Sequence
  Synthetic: IL-2-T3A-H16A-L19A-R38A-P65R-D84S-C125S
APASSSTKKTQLQLEALLADLQMILNGINNYKNPKLTAMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 121 - protein, 133 aa, Artificial Sequence
  Synthetic: IL-2-T3A-H16A-M23A-R38A-P65R-D84S-E95Q-C125S
APASSSTKKTQLQLEALLLDLQAILNGINNYKNPKLTAMLTFKFYMPKKATELKHLQCLEEELKRLEEVLNLAQSKNFHLRPRSLISNINVIVLQLKGSETTFMCEYADETATIVEFLNRWITFSQSIISTLT

SEQ ID NO: 122 - protein, 133 aa, Artificial Sequence
  Synthetic: IL-2-T3A-H16A-M23A-R38A-P65R-D84S-N88S-C125S
APASSSTKKTQLQLEALLLDLQAILNGINNYKNPKLTAMLTFKFYMPKKATELKHLQCLEEELKRLEEVLNLAQSKNFHLRPRSLISSINVIVLELKGSETTFMCEYADETATIVEFLNRWITFSQSIISTLT

SEQ ID NO: 123 - protein, 133 aa, Artificial Sequence
  Synthetic: IL-2-T3A-H16A-R38A-P65R-D84S-N88S-E95Q-C125S
APASSSTKKTQLQLEALLLDLQMILNGINNYKNPKLTAMLTFKFYMPKKATELKHLQCLEEELKRLEEVLNLAQSKNFHLRPRSLISSINVIVLQLKGSETTFMCEYADETATIVEFLNRWITFSQSIISTLT

SEQ ID NO: 124 - protein, 133 aa, Artificial Sequence
  Synthetic: IL-2-T3A-H16A-M23A-R38A-P65R-IL-2-D84S-N88S-E95Q-C125S
APASSSTKKTQLQLEALLLDLQAILNGINNYKNPKLTAMLTFKFYMPKKATELKHLQCLEEELKRLEEVLNLAQSKNFHLRPRSLISSINVIVLQLKGSETTFMCEYADETATIVEFLNRWITFSQSIISTLT

SEQ ID NO: 125 - protein, 133 aa, Artificial Sequence
  Synthetic: IL-2-T3A-H16A-L19N-P65R-D84S-C125S
APASSSTKKTQLQLEALLNDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 126 - protein, 133 aa, Artificial Sequence
  Synthetic: IL-2-T3A-H16A-L19S-P65R-D84S-C125S
APASSSTKKTQLQLEALLSDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 127 - protein, 133 aa, Artificial Sequence
  Synthetic: IL-2-T3A-H16A-L19T-P65R-D84S-C125S
APASSSTKKTQLQLEALLTDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 128 - protein, 133 aa, Artificial Sequence
  Synthetic: IL-2-T3A-H16A-L19V-P65R-D84S-C125S
APASSSTKKTQLQLEALLVDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 129 - protein, 133 aa, Artificial Sequence
  Synthetic: IL-2-T3A-H16A-L19N-M23A-E61R-P65R-D84S-E95Q-C125S
APASSSTKKTQLQLEALLNDLQAILNGINNYKNPKLTRMLTFKFYMPKKATELKHLQCLERELKRLEEVLNLAQSKNFHLRPRSLISNINVIVLELQGSETTFMCEYADETATIVEFLNRWITFSQSIISTLT

SEQ ID NO: 130 - protein, 133 aa, Artificial Sequence
  Synthetic: IL-2-T3A-H16A-L19N-M23A-R38A-P65R-D84S-E95Q-C125S
APASSSTKKTQLQLEALLNDLQAILNGINNYKNPKLTAMLTFKFYMPKKATELKHLQCLEEELKRLEEVLNLAQSKNFHLRPRSLISNINVIVLQLKGSETTFMCEYADETATIVEFLNRWITFSQSIISTLT

SEQ ID NO: 131 - protein, 133 aa, Artificial Sequence
  Synthetic: IL-2-T3A-H16A-L19N-M23A-R38A-P65R-D84S-C125S
APASSSTKKTQLQLEALLNDLQAILNGINNYKNPKLTAMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 132 - protein, 133 aa, Artificial Sequence
  Synthetic: IL-2-T3A-H16A-L19N-R38A-P65R-D84S-E95Q-C125S
APASSSTKKTQLQLEALLNDLQMILNGINNYKNPKLTAMLTFKFYMPKKATELKHLQCLEEELKRLEEVLNLAQSKNFHLRPRSLISNINVIVLQLKGSETTFMCEYADETATIVEFLNRWITFSQSIISTLT

SEQ ID NO: 133 - protein, 133 aa, Artificial Sequence
  Synthetic: IL-2-T3A-H16A-L19N-R38A-P65R-D84S-C125S
APASSSTKKTQLQLEALLNDLQMILNGINNYKNPKLTAMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 134 - protein, 133 aa, Artificial Sequence
  Synthetic: IL-2-T3A-H16A-L19N-E61R-P65R-D84S-C125S
APASSSTKKTQLQLEALLNDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 135 - protein, 133 aa, Artificial Sequence
  Synthetic: IL-2-T3A-H16A-L19N-E61R-P65R-D84S-E95Q-C125S
APASSSTKKTQLQLEALLNDLQMILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 136 - protein, 133 aa, Artificial Sequence
  Synthetic: IL-2-T3A-H16A-L19N-M23A-E61R-P65R-D84S-C125S
APASSSTKKTQLQLEALLNDLQAILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 137 - protein, 133 aa, Artificial Sequence
  Synthetic: IL-2-T3A-H16A-L19N-Q22A-E61R-P65R-D84S-C125S
APASSSTKKTQLQLEALLNDLAMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 138 - protein, 133 aa, Artificial Sequence
  Synthetic: IL-2-T3A-H16A-L19N-E61R-P65R-D84S-R120A-C125S
APASSSTKKTQLQLEALLNDLQMILNGINNYKNPKLTRMLTFKFYMPKKATELKHLQCLERELKRLEEVLNLAQSKNFHLRPRSLISNINVIVLELKGSETTFMCEYADETATIVEFLNAWITFSQSIISTLT

SEQ ID NO: 139 - protein, 133 aa, Artificial Sequence
  Synthetic: IL-2-T3A-H16A-L19N-E61R-P65R-D84S-C125S-Q126A
APASSSTKKTQLQLEALLNDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSASIISTLT

SEQ ID NO: 140 - protein, 133 aa, Artificial Sequence
  Synthetic: IL-2-T3A-H16S-L19N-E61R-P65R-D84S-C125S
APASSSTKKTQLQLESLLNDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 141 - protein, 133 aa, Artificial Sequence
  Synthetic: IL-2-T3A-H16T-L19N-E61R-P65R-D84S-C125S
APASSSTKKTQLQLETLLNDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 142 - protein, 133 aa, Artificial Sequence
  Synthetic: IL-2-T3A-H16V-L19N-E61R-P65R-D84S-C125S
APASSSTKKTQLQLEVLLNDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 143 - protein, 133 aa, Artificial Sequence
  Synthetic: IL-2-T3A-H16N-L19N-E61R-P65R-D84S-C125S
APASSSTKKTQLQLENLLNDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 144 - protein, 133 aa, Artificial Sequence
  Synthetic: IL-2-T3A-H16A-L19N-E61R-P65R-D84T-C125S
APASSSTKKTQLQLEALLNDLQMILNGINNYKNPKLTRMLTFKFYMPKKATELKHLQCLERELKRLEEVLNLAQSKNFHLRPRTLISNINVIVLELKGSETTFMCEYADETATIVEFLNRWITFSQSIISTLT

SEQ ID NO: 145 - protein, 133 aa, Artificial Sequence
  Synthetic: IL-2-T3A-H16A-L19N-E61R-P65R-D84V-C125S
APASSSTKKTQLQLEALLNDLQMILNGINNYKNPKLTRMLTFKFYMPKKATELKHLQCLERELKRLEEVLNLAQSKNFHLRPRVLISNINVIVLELKGSETTFMCEYADETATIVEFLNRWITFSQSIISTLT

SEQ ID NO: 146 - protein, 133 aa, Artificial Sequence
  Synthetic: IL-2-T3A-H16A-D20S-E61R-P65R-D84S-C125S
APASSSTKKTQLQLEALLLSLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 147 - protein, 133 aa, Artificial Sequence
  Synthetic: IL-2-T3A-H16A-D20T-E61R-P65R-D84S-C125S
APASSSTKKTQLQLEALLLTLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 148 - protein, 133 aa, Artificial Sequence
  Synthetic: IL-2-T3A-H16A-D20N-E61R-P65R-D84S-C125S
APASSSTKKTQLQLEALLLNLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 149 - protein, 133 aa, Artificial Sequence
  Synthetic: IL-2-T3A-H16A-D20A-E61R-P65R-D84S-C125S
APASSSTKKTQLQLEALLLALQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 150 - protein, 133 aa, Artificial Sequence
  Synthetic: IL-2-T3A-H16A-L19N-E61R-P65R-D84N-C125S
APASSSTKKTQLQLEALLNDLQMILNGINNYKNPKLTRMLTFKFYMPKKATELKHLQCLERELKRLEEVLNLAQSKNFHLRPRNLISNINVIVLELKGSETTFMCEYADETATIVEFLNRWITFSQSIISTLT

SEQ ID NO: 151 - protein, 133 aa, Artificial Sequence
  Synthetic: IL-2-T3A-H16A-E61R-P65R-D84S-N88T-C125S
APASSSTKKTQLQLEALLLDLQMILNGINNYKNPKLTRMLTFKFYMPKKATELKHLQCLERELKRLEEVLNLAQSKNFHLRPRSLISTINVIVLELKGSETTFMCEYADETATIVEFLNRWITFSQSIISTLT

SEQ ID NO: 152 - protein, 133 aa, Artificial Sequence
  Synthetic: IL-2-T3A-H16A-E61R-P65R-D84S-N88A-C125S
APASSSTKKTQLQLEALLLDLQMILNGINNYKNPKLTRMLTFKFYMPKKATELKHLQCLERELKRLEEVLNLAQSKNFHLRPRSLISAINVIVLELKGSETTFMCEYADETATIVEFLNRWITFSQSIISTLT

SEQ ID NO: 153 - protein, 133 aa, Artificial Sequence
  Synthetic: IL-2-T3A-H16P-L19A-E61R-P65R-D84S-C125S
APASSSTKKTQLQLEPLLADLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 154 - protein, 133 aa, Artificial Sequence
  Synthetic: IL-2-T3A-H16A-L19H-E61R-P65R-D84S-C125S
APASSSTKKTQLQLEALLHDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 155 - protein, 133 aa, Artificial Sequence
  Synthetic: IL-2-T3A-H16A-L19P-E61R-P65R-D84S-C125S
APASSSTKKTQLQLEALLPDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 156 - protein, 133 aa, Artificial Sequence
  Synthetic: IL-2-T3A-H16A-L19Q-E61R-P65R-D84S-C125S
APASSSTKKTQLQLEALLQDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 157 - protein, 133 aa, Artificial Sequence
  Synthetic: IL-2-T3A-H16A-L19Y-E61R-P65R-D84S-C125S
APASSSTKKTQLQLEALLYDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 158 - protein, 133 aa, Artificial Sequence
  Synthetic: IL-2-T3A-H16A-L19A-E61R-P65R-D84P-C125S
APASSSTKKTQLQLEALLADLQMILNGINNYKNPKLTRMLTFKFYMPKKATELKHLQCLERELKRLEEVLNLAQSKNFHLRPRPLISNINVIVLELKGSETTFMCEYADETATIVEFLNRWITFSQSIISTLT

SEQ ID NO: 159 - protein, 133 aa, Artificial Sequence
  Synthetic: IL-2-T3A-H16A-L19A-E61R-P65R-D84V-C125S
APASSSTKKTQLQLEALLADLQMILNGINNYKNPKLTRMLTFKFYMPKKATELKHLQCLERELKRLEEVLNLAQSKNFHLRPRVLISNINVIVLELKGSETTFMCEYADETATIVEFLNRWITFSQSIISTLT

SEQ ID NO: 160 - protein, 133 aa, Artificial Sequence
  Synthetic: IL-2-T3A-H16A-L19A-E61R-P65R-D84Y-C125S
APASSSTKKTQLQLEALLADLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 161 - protein, 133 aa, Artificial Sequence
  Synthetic: IL-2-T3A-H16A-M23A-E61R-P65R-D84S-E95H-C125S
APASSSTKKTQLQLEALLLDLQAILNGINNYKNPKLTRMLTFKFYMPKKATELKHLQCLERELKRLEEVLNLAQSKNFHLRPRSLISNINVIVLHLKGSETTFMCEYADETATIVEFLNRWITFSQSIISTLT

SEQ ID NO: 162 - protein, 133 aa, Artificial Sequence
  Synthetic: IL-2-T3A-H16A-M23A-E61R-P65R-D84S-E95N-C125S
APASSSTKKTQLQLEALLLDLQAILNGINNYKNPKLTRMLTFKFYMPKKATELKHLQCLERELKRLEEVLNLAQSKNFHLRPRSLISNINVIVLNLKGSETTFMCEYADETATIVEFLNRWITFSQSIISTLT

SEQ ID NO: 163 - protein, 133 aa, Artificial Sequence
  Synthetic: IL-2-T3A-H16A-M23A-E61R-P65R-D84S-E95P-C125S
APASSSTKKTQLQLEALLLDLQAILNGINNYKNPKLTRMLTFKFYMPKKATELKHLQCLERELKRLEEVLNLAQSKNFHLRPRSLISNINVIVLPLKGSETTFMCEYADETATIVEFLNRWITFSQSIISTLT

SEQ ID NO: 164 - protein, 133 aa, Artificial Sequence
  Synthetic: IL-2-T3A-H16A-M23A-E61R-P65R-D84S-E95S-C125S
APASSSTKKTQLQLEALLLDLQAILNGINNYKNPKLTRMLTFKFYMPKKATELKHLQCLERELKRLEEVLNLAQSKNFHLRPRSLISNINVIVLSLKGSETTFMCEYADETATIVEFLNRWITFSQSIISTLT

SEQ ID NO: 165 - protein, 133 aa, Artificial Sequence
  Synthetic: IL-2-T3A-H16A-M23A-E61R-P65R-D84S-E95T-C125S
APASSSTKKTQLQLEALLLDLQAILNGINNYKNPKLTRMLTFKFYMPKKATELKHLQCLERELKRLEEVLNLAQSKNFHLRPRSLISNINVIVLTLKGSETTFMCEYADETATIVEFLNRWITFSQSIISTLT

SEQ ID NO: 166 - protein, 133 aa, Artificial Sequence
  Synthetic: IL-2-T3A-H16A-M23A-E61R-P65R-D84S-E95V-C125S
APASSSTKKTQLQLEALLLDLQAILNGINNYKNPKLTRMLTFKFYMPKKATELKHLQCLERELKRLEEVLNLAQSKNFHLRPRSLISNINVIVLVLKGSETTFMCEYADETATIVEFLNRWITFSQSIISTLT

SEQ ID NO: 167 - protein, 133 aa, Artificial Sequence
  Synthetic: IL-2-T3A-H16A-M23A-E61R-P65R-D84S-E95Y-C125S
APASSSTKKTQLQLEALLLDLQAILNGINNYKNPKLTRMLTFKFYMPKKATELKHLQCLERELKRLEEVLNLAQSKNFHLRPRSLISNINVIVLYLKGSETTFMCEYADETATIVEFLNRWITFSQSIISTLT

SEQ ID NO: 168 - protein, 133 aa, Artificial Sequence
  Synthetic: IL-2-T3A-H16A-L19A-E61A-P65R-D84S-C125S
APASSSTKKTQLQLEALLADLQMILNGINNYKNPKLTRMLTFKFYMPKKATELKHLQCLEAELKRLEEVLNLAQSKNFHLRPRSLISNINVIVLELKGSETTFMCEYADETATIVEFLNRWITFSQSIISTLT

SEQ ID NO: 169 - protein, 133 aa, Artificial Sequence
  Synthetic: IL-2-T3A-H16A-L19A-E61G-P65R-D84S-C125S
APASSSTKKTQLQLEALLADLQMILNGINNYKNPKLTRMLTFKFYMPKKATELKHLQCLEGELKRLEEVLNLAQSKNFHLRPRSLISNINVIVLELKGSETTFMCEYADETATIVEFLNRWITFSQSIISTLT

SEQ ID NO: 170 - protein, 133 aa, Artificial Sequence
  Synthetic: IL-2-T3A-H16A-L19A-E61H-P65R-D84S-C125S
APASSSTKKTQLQLEALLADLQMILNGINNYKNPKLTRMLTFKFYMPKKATELKHLQCLEHELKRLEEVLNLAQSKNFHLRPRSLISNINVIVLELKGSETTFMCEYADETATIVEFLNRWITFSQSIISTLT

SEQ ID NO: 171 - protein, 133 aa, Artificial Sequence
  Synthetic: IL-2-T3A-H16A-L19A-E61N-P65R-D84S-C125S
APASSSTKKTQLQLEALLADLQMILNGINNYKNPKLTRMLTFKFYMPKKATELKHLQCLENELKRLEEVLNLAQSKNFHLRPRSLISNINVIVLELKGSETTFMCEYADETATIVEFLNRWITFSQSIISTLT

SEQ ID NO: 172 - protein, 133 aa, Artificial Sequence
  Synthetic: IL-2-T3A-H16A-L19A-E61P-P65R-D84S-C125S
APASSSTKKTQLQLEALLADLQMILNGINNYKNPKLTRMLTFKFYMPKKATELKHLQCLEPELKRLEEVLNLAQSKNFHLRPRSLISNINVIVLELKGSETTFMCEYADETATIVEFLNRWITFSQSIISTLT

SEQ ID NO: 173 - protein, 133 aa, Artificial Sequence
  Synthetic: IL-2-T3A-H16A-L19A-E61Q-P65R-D84S-C125S
APASSSTKKTQLQLEALLADLQMILNGINNYKNPKLTRMLTFKFYMPKKATELKHLQCLEQELKRLEEVLNLAQSKNFHLRPRSLISNINVIVLELKGSETTFMCEYADETATIVEFLNRWITFSQSIISTLT

SEQ ID NO: 174 - protein, 133 aa, Artificial Sequence
  Synthetic: IL-2-T3A-H16A-L19A-E61S-P65R-D84S-C125S
APASSSTKKTQLQLEALLADLQMILNGINNYKNPKLTRMLTFKFYMPKKATELKHLQCLESELKRLEEVLNLAQSKNFHLRPRSLISNINVIVLELKGSETTFMCEYADETATIVEFLNRWITFSQSIISTLT

SEQ ID NO: 175 - protein, 133 aa, Artificial Sequence
  Synthetic: IL-2-T3A-H16A-L19A-E61T-P65R-D84S-C125S
APASSSTKKTQLQLEALLADLQMILNGINNYKNPKLTRMLTFKFYMPKKATELKHLQCLETELKRLEEVLNLAQSKNFHLRPRSLISNINVIVLELKGSETTFMCEYADETATIVEFLNRWITFSQSIISTLT

SEQ ID NO: 176 - protein, 133 aa, Artificial Sequence
  Synthetic: IL-2-T3A-H16A-L19A-E61V-P65R-D84S-C125S
APASSSTKKTQLQLEALLADLQMILNGINNYKNPKLTRMLTFKFYMPKKATELKHLQCLEVELKRLEEVLNLAQSKNFHLRPRSLISNINVIVLELKGSETTFMCEYADETATIVEFLNRWITFSQSIISTLT

SEQ ID NO: 177 - protein, 133 aa, Artificial Sequence
  Synthetic: IL-2-T3A-H16A-L19A-E61Y-P65R-D84S-C125S
APASSSTKKTQLQLEALLADLQMILNGINNYKNPKLTRMLTFKFYMPKKATELKHLQCLEYELKRLEEVLNLAQSKNFHLRPRSLISNINVIVLELKGSETTFMCEYADETATIVEFLNRWITFSQSIISTLT

SEQ ID NO: 178 - protein, 133 aa, Artificial Sequence
  Synthetic: IL-2-T3A-H16A-M23D-E61R-P65R-D84S-E95Q-C125S
APASSSTKKTQLQLEALLLDLQDILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 179 - protein, 133 aa, Artificial Sequence
  Synthetic: IL-2-T3A-H16A-M23E-E61R-P65R-D84S-E95Q-C125S
APASSSTKKTQLQLEALLLDLQEILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 180 - protein, 133 aa, Artificial Sequence
  Synthetic: IL-2-T3A-H16A-M23G-E61R-P65R-D84S-E95Q-C125S
APASSSTKKTQLQLEALLLDLQGILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 181 - protein, 133 aa, Artificial Sequence
  Synthetic: IL-2-T3A-H16A-M23I-E61R-P65R-D84S-E95Q-C125S
APASSSTKKTQLQLEALLLDLQIILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 182 - protein, 133 aa, Artificial Sequence
  Synthetic: IL-2-T3A-H16A-M23K-E61R-P65R-D84S-E95Q-C125S
APASSSTKKTQLQLEALLLDLQKILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 183 - protein, 133 aa, Artificial Sequence
  Synthetic: IL-2-T3A-H16A-M23L-E61R-P65R-D84S-E95Q-C125S
APASSSTKKTQLQLEALLLDLQLILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 184 - protein, 133 aa, Artificial Sequence
  Synthetic: IL-2-T3A-H16A-M23N-E61R-P65R-D84S-E95Q-C125S
APASSSTKKTQLQLEALLLDLQNILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 185 - protein, 133 aa, Artificial Sequence
  Synthetic: IL-2-T3A-H16A-M23P-E61R-P65R-D84S-E95Q-C125S
APASSSTKKTQLQLEALLLDLQPILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 186 - protein, 133 aa, Artificial Sequence
  Synthetic: IL-2-T3A-H16A-M23Q-E61R-P65R-D84S-E95Q-C125S
APASSSTKKTQLQLEALLLDLQQILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 187 - protein, 133 aa, Artificial Sequence
  Synthetic: IL-2-T3A-H16A-M23R-E61R-P65R-D84S-E95Q-C125S
APASSSTKKTQLQLEALLLDLQRILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 188 - protein, 133 aa, Artificial Sequence
  Synthetic: IL-2-T3A-H16A-M23T-E61R-P65R-D84S-E95Q-C125S
APASSSTKKTQLQLEALLLDLQTILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 189 - protein, 133 aa, Artificial Sequence
  Synthetic: IL-2-T3A-H16A-M23V-E61R-P65R-D84S-E95Q-C125S
APASSSTKKTQLQLEALLLDLQVILNGINNYKNPKLTRMLTFKFYMPKKATELKHLQCLERELKRLEEVLNLAQSKNFHLRPRSLISNINVIVLQLKGSETTFMCEYADETATIVEFLNRWITFSQSIISTLT

SEQ ID NO: 190 - protein, 133 aa, Artificial Sequence
  Synthetic: IL-2-T3A-H16A-Q22A-E61R-P65R-D84S-C125S
APASSSTKKTQLQLEALLLDLAMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 191 - protein, 133 aa, Artificial Sequence
  Synthetic: IL-2-T3A-H16A-Q22D-E61R-P65R-D84S-C125S
APASSSTKKTQLQLEALLLDLDMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 192 - protein, 133 aa, Artificial Sequence
  Synthetic: IL-2-T3A-H16A-Q22E-E61R-P65R-D84S-C125S
APASSSTKKTQLQLEALLLDLEMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 193 - protein, 133 aa, Artificial Sequence
  Synthetic: IL-2-T3A-H16A-Q22G-E61R-P65R-D84S-C125S
APASSSTKKTQLQLEALLLDLGMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 194 - protein, 133 aa, Artificial Sequence
  Synthetic: IL-2-T3A-H16A-Q22H-E61R-P65R-D84S-C125S
APASSSTKKTQLQLEALLLDLHMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 195 - protein, 133 aa, Artificial Sequence
  Synthetic: IL-2-T3A-H16A-Q22K-E61R-P65R-D84S-C125S
APASSSTKKTQLQLEALLLDLKMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 196 - protein, 133 aa, Artificial Sequence
  Synthetic: IL-2-T3A-H16A-Q22N-E61R-P65R-D84S-C125S
APASSSTKKTQLQLEALLLDLNMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 197 - protein, 133 aa, Artificial Sequence
  Synthetic: IL-2-T3A-H16A-Q22P-E61R-P65R-D84S-C125S
APASSSTKKTQLQLEALLLDLPMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 198 - protein, 133 aa, Artificial Sequence
  Synthetic: IL-2-T3A-H16A-Q22R-E61R-P65R-D84S-C125S
APASSSTKKTQLQLEALLLDLRMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 199 - protein, 133 aa, Artificial Sequence
  Synthetic: IL-2-T3A-H16A-Q22S-E61R-P65R-D84S-C125S
APASSSTKKTQLQLEALLLDLSMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 200 - protein, 133 aa, Artificial Sequence
  Synthetic: IL-2-T3A-H16A-Q22T-E61R-P65R-D84S-C125S
APASSSTKKTQLQLEALLLDLTMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 201 - protein, 133 aa, Artificial Sequence
  Synthetic: IL-2-T3A-H16A-Q22V-E61R-P65R-D84S-C125S
APASSSTKKTQLQLEALLLDLVMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 202 - protein, 133 aa, Artificial Sequence
  Synthetic: IL-2-T3A-H16A-Q22Y-E61R-P65R-D84S-C125S
APASSSTKKTQLQLEALLLDLYMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIISTLT

SEQ ID NO: 203 - protein, 133 aa, Artificial Sequence
  Synthetic: IL-2-T3A-H16A-E61R-P65R-D84S-R120A-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNAWITFSQSIISTLT

SEQ ID NO: 204 - protein, 133 aa, Artificial Sequence
  Synthetic: IL-2-T3A-H16A-E61R-P65R-D84S-R120D-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNDWITFSQSIISTLT

SEQ ID NO: 205 - protein, 133 aa, Artificial Sequence
  Synthetic: IL-2-T3A-H16A-E61R-P65R-D84S-R120E-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNEWITFSQSIISTLT

SEQ ID NO: 206 - protein, 133 aa, Artificial Sequence
  Synthetic: IL-2-T3A-H16A-E61R-P65R-D84S-R120F-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNFWITFSQSIISTLT

SEQ ID NO: 207 - protein, 133 aa, Artificial Sequence
  Synthetic: IL-2-T3A-H16A-E61R-P65R-D84S-R120G-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNGWITFSQSIISTLT

SEQ ID NO: 208 - protein, 133 aa, Artificial Sequence
  Synthetic: IL-2-T3A-H16A-E61R-P65R-D84S-R120H-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNHWITFSQSIISTLT

SEQ ID NO: 209 - protein, 133 aa, Artificial Sequence
  Synthetic: IL-2-T3A-H16A-E61R-P65R-D84S-R120K-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNKWITFSQSIISTLT

SEQ ID NO: 210 - protein, 133 aa, Artificial Sequence
  Synthetic: IL-2-T3A-H16A-E61R-P65R-D84S-R120N-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNNWITFSQSIISTLT

SEQ ID NO: 211 - protein, 133 aa, Artificial Sequence
  Synthetic: IL-2-T3A-H16A-E61R-P65R-D84S-R120P-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNPWITFSQSIISTLT

SEQ ID NO: 212 - protein, 133 aa, Artificial Sequence
  Synthetic: IL-2-T3A-H16A-E61R-P65R-D84S-R120Q-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNQWITFSQSIISTLT

SEQ ID NO: 213 - protein, 133 aa, Artificial Sequence
  Synthetic: IL-2-T3A-H16A-E61R-P65R-D84S-R120S-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNSWITFSQSIISTLT

SEQ ID NO: 214 - protein, 133 aa, Artificial Sequence
  Synthetic: IL-2-T3A-H16A-E61R-P65R-D84S-R120T-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNTWITFSQSIISTLT

SEQ ID NO: 215 - protein, 133 aa, Artificial Sequence
  Synthetic: IL-2-T3A-H16A-E61R-P65R-D84S-R120V-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNVWITFSQSIISTLT

SEQ ID NO: 216 - protein, 133 aa, Artificial Sequence
  Synthetic: IL-2-T3A-H16A-E61R-P65R-D84S-R120Y-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNYWITFSQSIISTLT

SEQ ID NO: 217 - protein, 133 aa, Artificial Sequence
  Synthetic: IL-2-T3A-H16A-E61R-P65R-D84S-T123D-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWIDFSQSIISTLT

SEQ ID NO: 218 - protein, 133 aa, Artificial Sequence
  Synthetic: IL-2-T3A-H16A-E61R-P65R-D84S-T123E-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWIEFSQSIISTLT

SEQ ID NO: 219 - protein, 133 aa, Artificial Sequence
  Synthetic: IL-2-T3A-H16A-E61R-P65R-D84S-T123H-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWIHFSQSIISTLT

SEQ ID NO: 220 - protein, 133 aa, Artificial Sequence
  Synthetic: IL-2-T3A-H16A-E61R-P65R-D84S-T123K-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWIKFSQSIISTLT

SEQ ID NO: 221 - protein, 133 aa, Artificial Sequence
  Synthetic: IL-2-T3A-H16A-E61R-P65R-D84S-T123N-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWINFSQSIISTLT

SEQ ID NO: 222 - protein, 133 aa, Artificial Sequence
  Synthetic: IL-2-T3A-H16A-E61R-P65R-D84S-T123Q-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWIQFSQSIISTLT

SEQ ID NO: 223 - protein, 133 aa, Artificial Sequence
  Synthetic: IL-2-T3A-H16A-E61R-P65R-D84S-T123R-C125S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWIRFSQSIISTLT

SEQ ID NO: 224 - protein, 133 aa, Artificial Sequence
  Synthetic: IL-2-T3A-H16A-E61R-P65R-D84S-C125S-S130D
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIIDTLT

SEQ ID NO: 225 - protein, 133 aa, Artificial Sequence
  Synthetic: IL-2-T3A-H16A-E61R-P65R-D84S-C125S-S130E
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIIETLT

SEQ ID NO: 226 - protein, 133 aa, Artificial Sequence
  Synthetic: IL-2-T3A-H16A-E61R-P65R-D84S-C125S-S130K
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIIKTLT

SEQ ID NO: 227 - protein, 133 aa, Artificial Sequence
  Synthetic: IL-2-T3A-H16A-E61R-P65R-D84S-C125S-S130N
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIINTLT

SEQ ID NO: 228 - protein, 133 aa, Artificial Sequence
  Synthetic: IL-2-T3A-H16A-E61R-P65R-D84S-C125S-S130Q
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIIQTLT

SEQ ID NO: 229 - protein, 133 aa, Artificial Sequence
  Synthetic: IL-2-T3A-H16A-E61R-P65R-D84S-C125S-S130R
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIIRTLT

SEQ ID NO: 230 - protein, 133 aa, Artificial Sequence
  Synthetic: IL-2-T3A-H16A-E61R-P65R-D84S-C125S-S127D
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQDIISTLT

SEQ ID NO: 231 - protein, 133 aa, Artificial Sequence
  Synthetic: IL-2-T3A-H16A-E61R-P65R-D84S-C125S-S127E
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQEIISTLT

SEQ ID NO: 232 - protein, 133 aa, Artificial Sequence
  Synthetic: IL-2-T3A-H16A-E61R-P65R-D84S-C125S-S127H
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQHIISTLT

SEQ ID NO: 233 - protein, 133 aa, Artificial Sequence
  Synthetic: IL-2-T3A-H16A-E61R-P65R-D84S-C125S-S127P
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQPIISTLT

SEQ ID NO: 234 - protein, 133 aa, Artificial Sequence
  Synthetic: IL-2-T3A-H16A-E61R-P65R-D84S-C125S-S127Q
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQQIISTLT

SEQ ID NO: 235 - protein, 133 aa, Artificial Sequence
  Synthetic: IL-2-T3A-H16A-E61R-P65R-D84S-C125S-S127R
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQRIISTLT

SEQ ID NO: 236 - protein, 133 aa, Artificial Sequence
  Synthetic: IL-2-T3A-H16A-L19A-Q22A-R38A-P65R-D84S-C125S
APASSSTKKTQLQLEALLADLAMILNGINNYKNPKLTAMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 237 - protein, 133 aa, Artificial Sequence
  Synthetic: IL-2-T3A-H16A-L19A-R38A-P65R-D84S-R120A-C125S
APASSSTKKTQLQLEALLADLQMILNGINNYKNPKLTAMLTFKFYMPKKATELKHLQCLEEELKRLEEVLNLAQSKNFHLRPRSLISNINVIVLELKGSETTFMCEYADETATIVEFLNAWITFSQSIISTLT

SEQ ID NO: 238 - protein, 133 aa, Artificial Sequence
  Synthetic: IL-2-T3A-H16A-L19A-Q22A-R38A-P65R-D84S-R120A-C125S
APASSSTKKTQLQLEALLADLAMILNGINNYKNPKLTAMLTFKFYMPKKATELKHLQCLEEELKRLEEVLNLAQSKNFHLRPRSLISNINVIVLELKGSETTFMCEYADETATIVEFLNAWITFSQSIISTLT

SEQ ID NO: 239 - protein, 133 aa, Artificial Sequence
  Synthetic: IL-2-T3A-H16A-Q22A-M23A-R38A-P65R-D84S-E95Q-C125S
APASSSTKKTQLQLEALLLDLAAILNGINNYKNPKLTAMLTFKFYMPKKATELKHLQCLEEELKRLEEVLNLAQSKNFHLRPRSLISNINVIVLQLKGSETTFMCEYADETATIVEFLNRWITFSQSIISTLT

SEQ ID NO: 240 - protein, 133 aa, Artificial Sequence
  Synthetic: IL-2-T3A-H16A-M23A-R38A-P65R-D84S-E95Q-R120A-C125S
APASSSTKKTQLQLEALLLDLQAILNGINNYKNPKLTAMLTFKFYMPKKATELKHLQCLEEELKRLEEVLNLAQSKNFHLRPRSLISNINVIVLQLKGSETTFMCEYADETATIVEFLNAWITFSQSIISTLT

SEQ ID NO: 241 - protein, 133 aa, Artificial Sequence
  Synthetic: IL-2-T3A-H16A-Q22A-M23A-R38A-P65R-D84S-E95Q-R120A-C125S
APASSSTKKTQLQLEALLLDLAAILNGINNYKNPKLTAMLTFKFYMPKKATELKHLQCLEEELKRLEEVLNLAQSKNFHLRPRSLISNINVIVLQLKGSETTFMCEYADETATIVEFLNAWITFSQSIISTLT

SEQ ID NO: 242 - protein, 133 aa, Artificial Sequence
  Synthetic: IL-2-T3A-H16A-L19N-Q22A-R38A-P65R-D84S-C125S
APASSSTKKTQLQLEALLNDLAMILNGINNYKNPKLTAMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 243 - protein, 133 aa, Artificial Sequence
  Synthetic: IL-2-T3A-H16A-L19N-R38A-P65R-D84S-R120A-C125S
APASSSTKKTQLQLEALLNDLQMILNGINNYKNPKLTAMLTFKFYMPKKATELKHLQCLEEELKRLEEVLNLAQSKNFHLRPRSLISNINVIVLELKGSETTFMCEYADETATIVEFLNAWITFSQSIISTLT

SEQ ID NO: 244 - protein, 133 aa, Artificial Sequence
  Synthetic: IL-2-T3A-H16A-L19N-Q22A-R38A-P65R-D84S-R120A-C125S
APASSSTKKTQLQLEALLNDLAMILNGINNYKNPKLTAMLTFKFYMPKKATELKHLQCLEEELKRLEEVLNLAQSKNFHLRPRSLISNINVIVLELKGSETTFMCEYADETATIVEFLNAWITFSQSIISTLT

SEQ ID NO: 245 - protein, 133 aa, Artificial Sequence
  Synthetic: IL-2-A1G-P2G-T3S-S4G-S5G-H16A-L19A-R38A-P65R-D84S-C125S
GGSGGSTKKTQLQLEALLADLQMILNGINNYKNPKLTAMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 246 - protein, 128 aa, Artificial Sequence
  Synthetic: IL-2-del1-5-H16A-L19A-R38A-P65R-D84S-C125S
STKKTQLQLEALLADLQMILNGINNYKNPKLTAMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 247 - protein, 128 aa, Artificial Sequence
  Synthetic: IL-2-del1-5-H16A-L19A-R38A-P65R-D84S-C125V
STKKTQLQLEALLADLQMILNGINNYKNPKLTAMLTFKFYMPKKATELKHLQCLEEELKRLEEVLNLAQSKNFHLRPRSLISNINVIVLELKGSETTFMCEYADETATIVEFLNRWITFVQSIISTLT

SEQ ID NO: 248 - protein, 133 aa, Artificial Sequence
  Synthetic: IL-2-T3A-L19N-R38A-P65R-D84S-C125S
APASSSTKKTQLQLEHLLNDLQMILNGINNYKNPKLTAMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 249 - protein, 133 aa, Artificial Sequence
  Synthetic: IL-2-T3A-H16A-L19N-R38A-P65R-C125S
APASSSTKKTQLQLEALLNDLQMILNGINNYKNPKLTAMLTFKFYMPKKATELKHLQCLEEELKRLEEVLNLAQSKNFHLRPRDLISNINVIVLELKGSETTFMCEYADETATIVEFLNRWITFSQSIISTLT

SEQ ID NO: 250 - protein, 133 aa, Artificial Sequence
  Synthetic: IL-2-T3A-L19N-Q22A-R38A-P65R-D84S-C125S
APASSSTKKTQLQLEHLLNDLAMILNGINNYKNPKLTAMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 251 - protein, 133 aa, Artificial Sequence
  Synthetic: IL-2-T3A-H16A-L19N-Q22A-R38A-P65R-C125S
APASSSTKKTQLQLEALLNDLAMILNGINNYKNPKLTAMLTFKFYMPKKATELKHLQCLEEELKRLEEVLNLAQSKNFHLRPRDLISNINVIVLELKGSETTFMCEYADETATIVEFLNRWITFSQSIISTLT

SEQ ID NO: 252 - protein, 133 aa, Artificial Sequence
  Synthetic: IL-2-T3A-L19N-Q22A-R38A-P65R-D84S-R120A-C125S
APASSSTKKTQLQLEHLLNDLAMILNGINNYKNPKLTAMLTFKFYMPKKATELKHLQCLEEELKRLEEVLNLAQSKNFHLRPRSLISNINVIVLELKGSETTFMCEYADETATIVEFLNAWITFSQSIISTLT

SEQ ID NO: 253 - protein, 133 aa, Artificial Sequence
  Synthetic: IL-2-T3A-H16A-L19N-Q22A-R38A-P65R-R120A-C125S
APASSSTKKTQLQLEALLNDLAMILNGINNYKNPKLTAMLTFKFYMPKKATELKHLQCLEEELKRLEEVLNLAQSKNFHLRPRDLISNINVIVLELKGSETTFMCEYADETATIVEFLNAWITFSQSIISTLT

SEQ ID NO: 254 - protein, 133 aa, Artificial Sequence
  Synthetic: IL-2-T3A-Q22A-M23A-R38A-P65R-D84S-E95Q-C125S
APASSSTKKTQLQLEHLLLDLAAILNGINNYKNPKLTAMLTFKFYMPKKATELKHLQCLEEELKRLEEVLNLAQSKNFHLRPRSLISNINVIVLQLKGSETTFMCEYADETATIVEFLNRWITFSQSIISTLT

SEQ ID NO: 255 - protein, 133 aa, Artificial Sequence
  Synthetic: IL-2-T3A-Q22A-M23A-R38A-P65R-E95Q-C125S
APASSSTKKTQLQLEALLLDLAAILNGINNYKNPKLTAMLTFKFYMPKKATELKHLQCLEEELKRLEEVLNLAQSKNFHLRPRDLISNINVIVLQLKGSETTFMCEYADETATIVEFLNRWITFSQSIISTLT

SEQ ID NO: 256 - protein, 133 aa, Artificial Sequence
  Synthetic: IL-2-T3A-Q22A-M23A-R38A-P65R-D84S-E95Q-R120A-C125S
APASSSTKKTQLQLEHLLLDLAAILNGINNYKNPKLTAMLTFKFYMPKKATELKHLQCLEEELKRLEEVLNLAQSKNFHLRPRSLISNINVIVLQLKGSETTFMCEYADETATIVEFLNAWITFSQSIISTLT

SEQ ID NO: 257 - protein, 133 aa, Artificial Sequence
  Synthetic: IL-2-T3A-H16A-Q22A-M23A-R38A-P65R-E95Q-R120A-C125S
APASSSTKKTQLQLEALLLDLAAILNGINNYKNPKLTAMLTFKFYMPKKATELKHLQCLEEELKRLEEVLNLAQSKNFHLRPRDLISNINVIVLQLKGSETTFMCEYADETATIVEFLNAWITFSQSIISTLT

SEQ ID NO: 258 - protein, 133 aa, Artificial Sequence
  Synthetic: IL-2-T3A-L19A-Q22A-R38A-P65R-D84S-C125S
APASSSTKKTQLQLEHLLADLAMILNGINNYKNPKLTAMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 259 - protein, 133 aa, Artificial Sequence
  Synthetic: IL-2-T3A-H16A-L19A-Q22A-R38A-P65R-C125S
APASSSTKKTQLQLEALLADLAMILNGINNYKNPKLTAMLTFKFYMPKKATELKHLQCLEEELKRLEEVLNLAQSKNFHLRPRDLISNINVIVLELKGSETTFMCEYADETATIVEFLNRWITFSQSIISTLT

SEQ ID NO: 260 - protein, 133 aa, Artificial Sequence
  Synthetic: IL-2-T3A-L19A-Q22A-R38A-P65R-D84S-R120A-C125S
APASSSTKKTQLQLEHLLADLAMILNGINNYKNPKLTAMLTFKFYMPKKATELKHLQCLEEELKRLEEVLNLAQSKNFHLRPRSLISNINVIVLELKGSETTFMCEYADETATIVEFLNAWITFSQSIISTLT

SEQ ID NO: 261 - protein, 133 aa, Artificial Sequence
  Synthetic: IL-2-T3A-H16A-L19A-Q22A-R38A-P65R-R120A-C125S
APASSSTKKTQLQLEALLADLAMILNGINNYKNPKLTAMLTFKFYMPKKATELKHLQCLEEELKRLEEVLNLAQSKNFHLRPRDLISNINVIVLELKGSETTFMCEYADETATIVEFLNAWITFSQSIISTLT

SEQ ID NO: 262 - protein, 133 aa, Artificial Sequence
  Synthetic: IL-2-T3A-H16A-E61R-P65R-D84S-C125S-I129A
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIASTLT

SEQ ID NO: 263 - protein, 133 aa, Artificial Sequence
  Synthetic: IL-2-T3A-H16A-E61R-P65R-D84S-C125S-I129H
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIHSTLT

SEQ ID NO: 264 - protein, 133 aa, Artificial Sequence
  Synthetic: IL-2-T3A-H16A-E61R-P65R-D84S-C125S-I129R
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIRSTLT

SEQ ID NO: 265 - protein, 133 aa, Artificial Sequence
  Synthetic: IL-2-T3A-H16A-E61R-P65R-D84S-C125S-I129S
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSISSTLT

SEQ ID NO: 266 - protein, 133 aa, Artificial Sequence
  Synthetic: IL-2-T3A-H16A-E61R-P65R-D84S-C125C-Q126N
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSNSIISTLT

SEQ ID NO: 267 - protein, 133 aa, Artificial Sequence
  Synthetic: IL-2-T3A-H16A-E61R-P65R-D84S-C125C-Q126Y
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSYSIISTLT

SEQ ID NO: 268 - protein, 133 aa, Artificial Sequence
  Synthetic: IL-2-T3A-H16A-E61R-P65R-D84S-C125C-S127N
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQNIISTLT

SEQ ID NO: 269 - protein, 133 aa, Artificial Sequence
  Synthetic: IL-2-T3A-H16A-E61R-P65R-D84S-C125C-S130P
APASSSTKKTQLQLEALLLDLQMILNGINNYKNPKLTRMLTFKFYMPKKATELKHLQCLERELKRLEEVLNLAQSKNFHLRPRSLISNINVIVLELKGSETTFMCEYADETATIVEFLNRWITFSQSIIPTLT

SEQ ID NO: 270 - protein, 133 aa, Artificial Sequence
  Synthetic: IL-2-T3A-H16A-E61R-P65R-D84Y-C125S
APASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 271 - protein, 133 aa, Artificial Sequence
  Synthetic: IL-2-T3A-H16A-M23T-E61R-P65R-D84Y-E95Q-C125S
APASSSTKKTQLQLEALLLDLQTILNGINNYKNPKLTRMLTFKFYMPKKATELKHLQCLERELKRLEEVLNLAQSKNFHLRPRYLISNINVIVLQLKGSETTFMCEYADETATIVEFLNRWITFSQSIISTLT

SEQ ID NO: 272 - protein, 133 aa, Artificial Sequence
  Synthetic: IL-2-T3A-H16A-L19N-E61R-P65R-D84Y-C125S
APASSSTKKTQLQLEALLNDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 273 - protein, 133 aa, Artificial Sequence
  Synthetic: IL-2-T3A-H16A-L19N-M23T-E61R-P65R-D84Y-E95Q-C125S
APASSSTKKTQLQLEALLNDLQTILNGINNYKNPKLTRMLTFKFYMPKKATELKHLQCLERELKRLEEVLNLAQSKNFHLRPRYLISNINVIVLQLKGSETTFMCEYADETATIVEFLNRWITFSQSIISTLT

SEQ ID NO: 274 - protein, 133 aa, Artificial Sequence
  Synthetic: IL-2-T3A-H16A-E61R-P65R-D84Y-C125S-S127D
APASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQDIISTLT

SEQ ID NO: 275 - protein, 133 aa, Artificial Sequence
  Synthetic: IL-2-T3A-H16A-M23T-E61R-P65R-D84Y-E95Q-C125S-S127D
APASSSTKKTQLQLEALLLDLQTILNGINNYKNPKLTRMLTFKFYMPKKATELKHLQCLERELKRLEEVLNLAQSKNFHLRPRYLISNINVIVLQLKGSETTFMCEYADETATIVEFLNRWITFSQDIISTLT

SEQ ID NO: 276 - protein, 133 aa, Artificial Sequence
  Synthetic: IL-2-T3A-H16A-L19N-E61R-P65R-D84Y-C125S-S127D
APASSSTKKTQLQLEALLNDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQDIISTLT

SEQ ID NO: 277 - protein, 133 aa, Artificial Sequence
  Synthetic: IL-2-T3A-H16A-L19N-M23T-E61R-P65R-D84Y-E95Q-C125S-S127D
APASSSTKKTQLQLEALLNDLQTILNGINNYKNPKLTRMLTFKFYMPKKATELKHLQCLERELKRLEEVLNLAQSKNFHLRPRYLISNINVIVLQLKGSETTFMCEYADETATIVEFLNRWITFSQDIISTLT

SEQ ID NO: 278 - protein, 132 aa, Artificial Sequence
  Synthetic: IL-2-T3A-H16A-delta-F42-P65R-D84S-C125S
APASSSTKKTQLQLEALLLDLQMILNGINNYKNPKLTRMLTKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 279 - protein, 133 aa, Artificial Sequence
  Synthetic: IL-2-T3A-H16A-K43E-Y45R-P65R-D84S-C125S
APASSSTKKTQLQLEALLLDLQMILNGINNYKNPKLTRMLTFEFRMPKKATELKHLQCLEEELKRLEEVLNLAQSKNFHLRPRSLISNINVIVLELKGSETTFMCEYADETATIVEFLNRWITFSQSIISTLT

SEQ ID NO: 280 - protein, 133 aa, Artificial Sequence
  Synthetic: IL-2-T3A-H16A-K43E-P65R-D84S-C125S
APASSSTKKTQLQLEALLLDLQMILNGINNYKNPKLTRMLTFEFYMPKKATELKHLQCLEEELKRLEEVLNLAQSKNFHLRPRSLISNINVIVLELKGSETTFMCEYADETATIVEFLNRWITFSQSIISTLT

SEQ ID NO: 281 - protein, 133 aa, Artificial Sequence
  Synthetic: IL-2-T3A-H16A-P65R-D84S-I114F-C125S
APASSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATFVEFLNRWITFSQSIISTLT

SEQ ID NO: 282 - protein, 133 aa, Artificial Sequence
  Synthetic: IL-2-T3A-H16A-F42R-P65R-D84S-C125S
APASSSTKKTQLQLEALLLDLQMILNGINNYKNPKLTRMLTRKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 283 - protein, 133 aa, Artificial Sequence
  Synthetic: IL-2-T3A-K43E-Y45R-C125S
APASSSTKKTQLQLEHLLLDLQMILNGINNYKNPKLTRMLTFEFRMPKKATELKHLQCLEEELKPLEEVLNLAQSKNFHLRPRDLISNINVIVLELKGSETTFMCEYADETATIVEFLNRWITFSQSIISTLT

SEQ ID NO: 284 - protein, 133 aa, Artificial Sequence
  Synthetic: IL-2-T3A-E61R-C125S
APASSSTKKTQLQLEHLLLDLQMILNGINNYKNPKLTRMLTFKFYMPKKATELKHLQCLERELKPLEEVLNLAQSKNFHLRPRDLISNINVIVLELKGSETTFMCEYADETATIVEFLNRWITFSQSIISTLT

SEQ ID NO: 285 - protein, 133 aa, Artificial Sequence
  Synthetic: IL-2-T3A-I114F-C125S
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATFVEFLNRWITFSQSIISTLT

SEQ ID NO: 286 - protein, 133 aa, Artificial Sequence
  Synthetic: IL-2-T3A-K43E-C125S
APASSSTKKTQLQLEHLLLDLQMILNGINNYKNPKLTRMLTFEFYMPKKATELKHLQCLEEELKPLEEVLNLAQSKNFHLRPRDLISNINVIVLELKGSETTFMCEYADETATIVEFLNRWITFSQSIISTLT

SEQ ID NO: 287 - protein, 133 aa, Artificial Sequence
  Synthetic: IL-2-T3A-P65R-I114F-C125S
APASSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATFVEFLNRWITFSQSIISTLT

SEQ ID NO: 288 - protein, 133 aa, Artificial Sequence
  Synthetic: IL-2-T3A-K43E-P65R-C125S
APASSSTKKTQLQLEHLLLDLQMILNGINNYKNPKLTRMLTFEFYMPKKATELKHLQCLEEELKRLEEVLNLAQSKNFHLRPRDLISNINVIVLELKGSETTFMCEYADETATIVEFLNRWITFSQSIISTLT

SEQ ID NO: 289 - protein, 133 aa, Artificial Sequence
  Synthetic: IL-2-T3A-K43E-Y45R-P65R-C125S
APASSSTKKTQLQLEHLLLDLQMILNGINNYKNPKLTRMLTFEFRMPKKATELKHLQCLEEELKRLEEVLNLAQSKNFHLRPRDLISNINVIVLELKGSETTFMCEYADETATIVEFLNRWITFSQSIISTLT

SEQ ID NO: 290 - protein, 133 aa, Artificial Sequence
  Synthetic: IL-2-T3A-R38A-C125S
APASSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 291 - protein, 115 aa, Artificial Sequence
  Synthetic: IL-15 WT
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 292 - protein, 223 aa, Artificial Sequence
  Synthetic: Fc region xELL P329G knob
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 293 - protein, 223 aa, Artificial Sequence
  Synthetic: Fc region xELL P329G, H435R hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 294 - protein, 361 aa, Artificial Sequence; identical to SEQ ID NO: 46
  Synthetic: xELL-knob Fc  IL-2-RAS-T3G, C125S

SEQ ID NO: 295 - protein, 358 aa, Artificial Sequence
  Synthetic: gdTCR 5C8 VHH-xELL- hole T366S, L368A, Y407V, H435R Fc
EVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 296 - protein, 495 aa, Artificial Sequence
  Synthetic: gdTCR 5C8 VHH-xELL-knob Fc-IL-2-T3A-H16A- E61R-P65R-D84Y-C125S
EVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 297 - skipped

SEQ ID NO: 298 - protein, 488 aa, Artificial Sequence
  Synthetic: gdTCR 6C4 VHH-xELL-knob Fc-IL-2- T3A-H16A- E61R-P65R-D84Y-C125S
EVQLVESGGGLVQAGGSLRLSCAVSVRTFSDYRMGWFRQAPGKEREFVSTISWSGGLTYYADSVKGRFTISRDNSKNTLYLQMNSLKPEDTAVYYCAAGGGYAGGTYYHPEEWGQGTQVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 299 - protein, 351 aa, Artificial Sequence
  Synthetic: gdTCR 6C4 VHH-xELL-hole T366S, L368A, Y407V H435R Fc
EVQLVESGGGLVQAGGSLRLSCAVSVRTFSDYRMGWFRQAPGKEREFVSTISWSGGLTYYADSVKGRFTISRDNSKNTLYLQMNSLKPEDTAVYYCAAGGGYAGGTYYHPEEWGQGTQVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 300 - protein, 345 aa, Artificial Sequence
  Synthetic: 410A9v1 (untargeted) VHH-xELL-hole T366S, L368A, Y407V H435R Fc
EVQLVESGGGEVQPGGSLRLSCAASGSINSINVMEWYRQAPGKERDLVAGITSDGDTNYAESVKGRFTISRDNAKNTLYLQMSSLRAEDTAVYYCRARDWGSLTDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 301 - protein, 482 aa, Artificial Sequence
  Synthetic: 410A9v1 (untargeted) VHH- xELL-knob Fc- IL-2-T3A-H16A- E61R-P65R-D84Y-C125S
EVQLVESGGGEVQPGGSLRLSCAASGSINSINVMEWYRQAPGKERDLVAGITSDGDTNYAESVKGRFTISRDNAKNTLYLQMSSLRAEDTAVYYCRARDWGSLTDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

(19) United States
(12) Patent Application Publication
(10) Pub. No.: US 2023/0235005 A1
(43) Pub. Date: Jul. 27, 2023

(54) Polypeptides Comprising Modified IL-2 Polypeptides and Uses Thereof

(71) Applicant: Inhibrx, Inc., La Jolla, CA (US)

(72) Inventors: John C. Timmer, San Diego, CA (US); Florian Sulzmaier, San Diego, CA (US); Katelyn M. Willis, San Diego, CA (US); Bryan R. Becklund, San Diego, CA (US); Brendan P. Eckelman, Encinitas, CA (US)

(73) Assignee: Inhibrx, Inc., La Jolla, CA (US)

(21) Appl. No.: 18/002,613

(22) PCT Filed: Jul. 1, 2021
(86) PCT No.: PCT/US2021/040061

§ 371 Date: Dec. 20, 2022

Related U.S. Application Data
(60) Provisional application No. 63/047,681, filed on Jul. 2, 2020.

(51) Int. Cl. C07K 14/55; A61K 45/06; A61K 38/20

(52) U.S. Cl. CPC C07K 14/55; A61K 45/06; A61K 38/2013; C07K 2319/30

(57) ABSTRACT

Provided herein are polypeptide comprising a modified IL-2, wherein the modified IL-2 has reduced affinity for the IL-2 receptor relative to wild type IL-2. In some embodiments, polypeptides comprising a modified IL-2 that bind and agonize activated T cells are provided. Uses of the polypeptides comprising a modified IL-2 are also provided.

### CROSS-REFERENCE TO RELATED APPLICATIONS

This application claims the benefit of priority of U.S. Provisional Application No. 63/047,681 filed Jul. 2, 2020, which is incorporated by reference herein in its entirety for any purpose.

### FIELD

The present invention relates to modified IL-2 with reduced affinity to CD25 and CD122 and such modified IL-2 fused to targeting moieties. The invention also relates to methods of using modified IL-2 and polypeptides comprising modified IL-2, including, but not limited to, methods of treating cancer.

### BACKGROUND

IL-2 is a potent cytokine that stimulates T and NK cell proliferation through either a heterotrimeric IL-2 receptor (IL-2R) composed of CD25, CD122 and CD132, or a heterodimeric IL-2 receptor composed of only CD122 and CD132. Both forms of the IL-2R are potent mediators of T cell survival, proliferation, and overall activation status. IL-2 is generally produced by T cells and NK cells upon activation and mediates signaling in cis and trans in the local microenvironment. IL-2R signaling can induce differentiation of naïve T cells into effector and memory T cells, and can also stimulate suppressive regulatory T cells. Although the trimeric form of the IL-2R has a higher affinity for IL-2 than the dimeric form, both are reasonably high affinity and cause rapid receptor mediated internalization and degradation, resulting in an extremely short half-life. Recombinant human IL-2 (rhIL-2, Proleukin) is used clinically to treat renal cell carcinoma and malignant melanoma; however, it is associated with severe toxicity. Vascular leak syndrome is a major toxicity concern for cancer patients treated with Proleukin due to the effects of IL-2 signaling on endothelial cells that express the high affinity IL-2R.

T cells are activated through ligation of their TCR with a neighboring cell presenting MHC with complementary peptide bound, causing clustering of the TCR complex and signaling through NFAT. Co-stimulation of T cells through CD28 is driven by CD80 and CD86, which enhances T cell activation. After the initial activation, T cells upregulate a variety of proteins, including cytokine receptors as well as many co-stimulatory and checkpoint receptors that serve to modulate the T cell response.

Durable anti-tumor clinical responses have recently been reported for antagonist antibodies to checkpoint receptors, such as CTLA-4, PD-1, and PD-L1. However, even in the most responsive indications the response rate is limited to about 30% of patients. Accordingly, there is a need for improved T cell modulating therapeutics.

### SUMMARY

Provided herein are polypeptides comprising a modified IL-2 comprising at least one substitution at at least one amino acid position. In some embodiments, the modified IL-2 has reduced binding affinity for CD25, CD122, and/or an IL-2R relative to wild type IL-2. In some embodiments, the modified IL-2 has reduced activity on resting or activated T cells relative to wild type IL-2.

The following embodiments are provided herein:

Embodiment 1. A polypeptide comprising a modified IL-2, wherein the modified IL-2 comprises a D84Y substitution.

Embodiment 2. The polypeptide of embodiment 1, wherein the modified IL-2 has reduced affinity for CD122 compared to wild-type IL-2.

Embodiment 3. The polypeptide of any one of embodiments 1-2, wherein the modified IL-2 comprises at least one substitution at at least one amino acid position selected from H16, L19, M23, N88, and E95.

Embodiment 4. The polypeptide of embodiment 3, wherein the modified IL-2 comprises a substitution at amino acid position H16.

Embodiment 5. The polypeptide of embodiment 4, wherein the substitution is selected from H16A H16N, H16V, and H16T.

Embodiment 6. The polypeptide of any one of embodiments 1-5, wherein the modified IL-2 comprises a substitution at amino acid position L19.

Embodiment 7. The polypeptide of embodiment 6, wherein the substitution is selected from L19A, L19P, L19Q, L19Y, L19N, L19S, L19T, L19V.

Embodiment 8. The polypeptide of any one of embodiments 1-7, wherein the modified IL-2 comprises a substitution at amino acid position M23.

Embodiment 9. The polypeptide of embodiment 8, wherein the substitution is selected from M23A, M23G, M23S, M23T, M23V, M23D, M23E, M23I, M23K, M23L, M23N, M23Q, M23R, and M23Y.

Embodiment 10. The polypeptide of any one of embodiments 1-9, wherein the modified IL-2 comprises a substitution at amino acid position N88.

Embodiment 11. The polypeptide of embodiment 10, wherein the substitution is selected from N88T, N88A, and N88S.

Embodiment 12. The polypeptide of any one of embodiments 1-11, wherein the modified IL-2 comprises a substitution at amino acid position E95.

Embodiment 13. The polypeptide of embodiment 12, wherein the substitution is selected from E95Q, E95G, E95T, E95V, E95P, E95H, E95N, and E95Y.

Embodiment 14. The polypeptide of any one of embodiments 1-13, wherein the modified IL-2 comprises at least one substitution that reduces affinity for CD132 compared to wild-type IL-2.

Embodiment 15. The polypeptide of any one of embodiments 1-14, wherein the modified IL-2 comprises at least one substitution at at least one amino acid position selected from Q22, R120, T123, Q126, S127, I129, and S130.

Embodiment 16. The polypeptide of embodiment 15, wherein the modified IL-2 comprises a substitution at amino acid position Q22.

Embodiment 17. The polypeptide of embodiment 16, wherein the substitution is selected from Q22A, Q22D, Q22G, Q22H, Q22K, Q22N, Q22R, Q22S, Q22T, Q22V, and Q22Y.

Embodiment 18. The polypeptide of any one of embodiments 15-17, wherein the modified IL-2 comprises a substitution at amino acid position R120.

Embodiment 19. The polypeptide of embodiment 18, wherein the substitution is selected from R120A, R120D, R120E, R120F, R120G, R120H, R120K, R120N, R120Q, R120S, R120V, and R120Y.

Embodiment 20. The polypeptide of any one of embodiments 15-19, wherein the modified IL-2 comprises a substitution at amino acid position T123.

Embodiment 21. The polypeptide of embodiment 20, wherein the substitution is selected from T123D, T123E, T123H, T123K, T123N, T123Q, and T123R.

Embodiment 22. The polypeptide of any one of embodiments 15-21, wherein the modified IL-2 comprises a substitution at amino acid position Q126.

Embodiment 23. The polypeptide of embodiment 22, wherein the substitution is selected from Q126N, Q126A, and Q126Y.

Embodiment 24. The polypeptide of any one of embodiments 15-23, wherein the modified IL-2 comprises a substitution at amino acid position 5127.

Embodiment 25. The polypeptide of embodiment 24, wherein the substitution is selected from S127D, S127E, S127H, S127K, S127N, S127P, and S127R.

Embodiment 26. The polypeptide of any one of embodiments 15-25, wherein the modified IL-2 comprises a substitution at amino acid position I129.

Embodiment 27. The polypeptide of embodiment 26, wherein the substitution is selected from I129A, I129H, I129R, and I129S.

Embodiment 28. The polypeptide of any one of embodiments 15-27, wherein the modified IL-2 comprises a substitution at amino acid position S130.

Embodiment 29. The polypeptide of embodiment 28, wherein the substitution is selected from S130E, S130K, S130N, S130P, S130Q, and S130R.

Embodiment 30. A polypeptide comprising a modified IL-2, wherein the modified IL-2 comprises at least one substitution at at least one amino acid position selected from Q22, R120, T123, S127, and S130.

Embodiment 31. The polypeptide of embodiment 30, wherein the modified IL-2 has reduced affinity for CD132 compared to wild-type IL-2.

Embodiment 32. The polypeptide of any one of embodiments 30-31, wherein the modified IL-2 comprises a substitution at amino acid position Q22.

Embodiment 33. The polypeptide of embodiment 32, wherein the substitution is selected from Q22A, Q22D, Q22E, Q22G, Q22H, Q22K, Q22N, Q22P, Q22R, Q22S, Q22T, Q22V, and Q22Y.

Embodiment 34. The polypeptide of any one of embodiments 30-33, wherein the modified IL-2 comprises a substitution at amino acid position R120.

Embodiment 35. The polypeptide of embodiment 34, wherein the substitution is selected from R120A, R120D, R120E, R120F, R120G, R120H, R120K, R120N, R120P, R120Q, R120S, R120V, and R120Y.

Embodiment 36. The polypeptide of embodiment 30-35, wherein the modified IL-2 comprises a substitution at amino acid position T123.

Embodiment 37. The polypeptide of embodiment 36, wherein the substitution is selected from T123D, T123E, T123H, T123K, T123N, T123Q, and T123R.

Embodiment 38. The polypeptide of any one of embodiments 30-37, wherein the modified IL-2 comprises a substitution at amino acid position 5127.

Embodiment 39. The polypeptide of embodiment 38, wherein the substitution is selected from S127D, S127E, S127H, S127K, S127N, S127P, S127Q, and S127R.

Embodiment 40. The polypeptide of any one of embodiments 30-39, wherein the modified IL-2 comprises a substitution at amino acid position S130.

Embodiment 41. The polypeptide of embodiment 40, wherein the substitution is selected from S130D, S130E, S130H, S130K, S130N, S130P, S130Q, and S130R.

Embodiment 42. The polypeptide of any one of embodiments 30-41, wherein the modified IL-2 comprises at least one substitution that reduces affinity for CD122 compared to wild-type IL-2.

Embodiment 43. The polypeptide of embodiment 42, wherein the modified IL-2 comprises at least one substitution at at least one amino acid position selected from H16, L19, M23, D84, N88, and E95.

Embodiment 44. The polypeptide of embodiment 43, wherein the modified IL-2 comprises a substitution at amino acid position H16.

Embodiment 45. The polypeptide of embodiment 44, wherein the substitution is selected from H16A, H16T, H16V, and H16N.

Embodiment 46. The polypeptide of any one of embodiments 43-45, wherein the modified IL-2 comprises a substitution at amino acid position L19.

Embodiment 47. The polypeptide of embodiment 46, wherein the substitution is selected from L19A, L19P, L19Q, L19Y, L19N, L19S, L19T, L19V.

Embodiment 48. The polypeptide of any one of embodiments 43-47, wherein the modified IL-2 comprises a substitution at amino acid position M23.

Embodiment 49. The polypeptide of embodiment 48, wherein the substitution is selected from M23A, M23G, M23S, M23T, M23V, M23D, M23E, M23I, M23K, M23L, M23N, M23Q, M23R, and M23Y.

Embodiment 50. The polypeptide of any one of embodiments 43-49, wherein the modified IL-2 comprises a substitution at amino acid position D84.

Embodiment 51. The polypeptide of embodiment 50, wherein the substitution is selected from D84S, D84G, D84A, D84T, D84V, D84Y, and D84N

Embodiment 52. The polypeptide of any one of embodiments 43-51, wherein the modified IL-2 comprises a substitution at amino acid position N88.

Embodiment 53. The polypeptide of embodiment 52, wherein the substitution is selected from N88T, N88A, and N88S.

Embodiment 54. The polypeptide of any one of embodiments 43-53, wherein the modified IL-2 comprises a substitution at amino acid position E95.

Embodiment 55. The polypeptide of embodiment 54, wherein the substitution is selected from E95Q, E95G, E95T, E95V, E95P, E95H, E95N, and E95Y.

Embodiment 56. The polypeptide of any one of embodiments 1-55, wherein the modified IL-2 comprises at least one substitution that reduces affinity for CD25 compared to wild-type IL-2.

Embodiment 57. The polypeptide of any one of embodiments 1-56, wherein the modified IL-2 comprises at least one substitution at at least one amino acid position selected from K43, Y45, E61, I114, P65, F42, R38, and L72, and/or wherein the modified IL-2 comprises a deletion of amino acid F42.

Embodiment 58. The polypeptide of embodiment 57, wherein the modified IL-2 comprises a substitution at amino acid F42 or comprises a deletion of amino acid F42.

Embodiment 59. The polypeptide of embodiment 58, wherein the modified IL-2 comprise a substitution at amino acid position F42 selected from F42K, F42A, F42R, F42G, F42S, and F42T.

Embodiment 60. The polypeptide of embodiment 58, wherein the modified IL-2 comprise a deletion of amino acid F42.

Embodiment 61. The polypeptide of any one of embodiments 57-60, wherein the modified IL-2 comprises a substitution at amino acid position K43.

Embodiment 62. The polypeptide of embodiment 61, wherein the substitution is selected from K43E and K43D.

Embodiment 63. The polypeptide of any one of embodiments 57-62, wherein the modified IL-2 comprises a substitution at amino acid position Y45.

Embodiment 64. The polypeptide of embodiment 63, wherein the substitution is selected from Y45R and Y45K.

Embodiment 65. The polypeptide of any one of embodiments 57-64, wherein the modified IL-2 comprises a substitution at amino acid position E61.

Embodiment 66. The polypeptide of embodiment 65, wherein the substitution is selected from E61R, E61G, E61H, E61N, E61P, E61S, E61T, E61Y, E61A, E61Q, and E61K.

Embodiment 67. The polypeptide of any one of embodiments 57-66, wherein the modified IL-2 comprises a substitution at amino acid position I114.

Embodiment 68. The polypeptide of embodiment 67, wherein the substitution is I114F, I114Y, or I114W.

Embodiment 69. The polypeptide of any one of embodiments 57-68, wherein the modified IL-2 comprises a substitution at amino acid position P65.

Embodiment 70. The polypeptide of embodiment 69, wherein the substitution is selected from P65R, P65E, P65K, P65H, P65Y, P65Q, P65D, and P65N.

Embodiment 71. The polypeptide of any one of embodiments 57-70, wherein the modified IL-2 comprises a substitution at amino acid position R38.

Embodiment 72. The polypeptide of embodiment 71, wherein the substitution at R38 is selected from R38A and R38G.

Embodiment 73. The polypeptide of any one of embodiments 57-72, wherein the modified IL-2 comprises a substitution at amino acid position L72.

Embodiment 74. The polypeptide of embodiment 73, wherein the substitution at L72 is and L72G.

Embodiment 75. The polypeptide of any one of embodiments 1-74, wherein the modified IL-2 comprises substitution Q22A, or substitution R120A, or substitutions Q22A and R120A.

Embodiment 76. The polypeptide of any one of embodiments 1-75, wherein the modified IL-2 comprises substitutions P65R and R38A or substitution P65R and E61R.

Embodiment 77. The polypeptide of any one of embodiments 1-76, wherein the modified IL-2 comprises at least one substitution selected from H16A, L19A, L19N, M23A, D84S or D84Y, N88S, and E95Q.

Embodiment 78. The polypeptide of any one of embodiments 1-77, wherein the modified IL-2 comprises substitutions at amino acid positions P65, H16, and D84.

Embodiment 79. The polypeptide of embodiment 78, wherein the modified IL-2 comprises substitutions P65R, H16A, and D84S; or substitutions P65R, H16A, and D84Y.

Embodiment 80. The polypeptide of any one of embodiments 1-79, wherein the modified IL-2 comprises at least one substitution at at least one amino acid position selected from T3 and C125, and/or comprises a deletion of the first five amino acids of IL-2.

Embodiment 81. The polypeptide of embodiment 80, wherein the modified IL-2 comprises at least one substitution selected from T3A, C125A, C125V, and C125S.

Embodiment 82. The polypeptide of embodiment 81, wherein the modified IL-2 comprises T3A and C125S substitutions, or T3A and C125V substitutions.

Embodiment 83. The polypeptide of embodiment 81, wherein the modified IL-2 comprises a deletion of the first five amino acids of IL-2 and a C125S substitution or C125V substitution.

Embodiment 84. The polypeptide of any one of the preceding embodiments, wherein the modified IL-2 comprises a set of substitutions selected from [T3A, H16A, E61R, P65R, D84Y, C125S], [T3A, H16A, M23T, E61R, P65R, D84Y, E95Q, C125S], [T3A, H16A, L19N, E61R, P65R, D84Y, C125S], [T3A, H16A, L19N, M23T, E61R, P65R, D84Y, E95Q, C125S], [T3A, H16A, E61R, P65R, D84Y, C125S, S127D], [T3A, H16A, M23T, E61R, P65R, D84Y, E95Q, C125S, S127D], [T3A, H16A, L19N, E61R, P65R, D84Y, C125S, S127D], and [T3A, H16A, L19N, M23T, E61R, P65R, D84Y, E95Q, C125S, S127D].

Embodiment 85. A modified polypeptide comprising a modified IL-2, wherein the modified IL-2 comprises a set of substitutions selected from [T3A, H16A, E61R, P65R, D84Y, C125S], [T3A, H16A, M23T, E61R, P65R, D84Y, E95Q, C125S], [T3A, H16A, L19N, E61R, P65R, D84Y, C125S], [T3A, H16A, L19N, M23T, E61R, P65R, D84Y, E95Q, C125S], [T3A, H16A, E61R, P65R, D84Y, C125S, S127D], [T3A, H16A, M23T, E61R, P65R, D84Y, E95Q, C125S, S127D], [T3A, H16A, L19N, E61R, P65R, D84Y, C125S, S127D], and [T3A, H16A, L19N, M23T, E61R, P65R, D84Y, E95Q, C125S, S127D].

Embodiment 86. The polypeptide of any one of the preceding embodiments, wherein the modified IL-2 comprises the indicated substitutions, and does not comprise any additional substitutions.

Embodiment 87. The polypeptide of any one of the preceding embodiments, wherein the modified IL-2 is a modified human IL-2.

Embodiment 88. The polypeptide of any one of the preceding embodiments, wherein the amino acid positions correspond to the amino acid positions in SEQ ID NO: 1.

Embodiment 89. The polypeptide of any one of the preceding embodiments, wherein the modified IL-2 comprises an amino acid sequence at least 90%, 91%, 92%, 93%, 94%, 95%, 96%, 97%, 98%, or 99% identical to SEQ ID NO: 84 and comprising corresponding substitutions of an amino acid sequence selected from SEQ ID NOs: 105-290.

Embodiment 90. The polypeptide of any one of the preceding embodiments, wherein the modified IL-2 comprises an amino acid sequence at least 90%, 91%, 92%, 93%, 94%, 95%, 96%, 97%, 98%, 99%, or 100% identical to an amino acid sequence selected from SEQ ID NOs: 270-277 and comprises substitution D84Y.

Embodiment 91. The polypeptide of any one of the preceding embodiments, wherein the modified IL-2 comprises an amino acid sequence selected from SEQ ID NOs: 105-290.

Embodiment 92. The polypeptide of any one of the preceding embodiments, wherein the modified IL-2 comprises an amino acid sequence selected from SEQ ID NOs: 270-277.

Embodiment 93. The polypeptide of any one of the preceding embodiments, wherein the polypeptide comprises an Fc region.

Embodiment 94. The polypeptide of embodiment 93, wherein the modified IL-2 is fused to the N-terminus or the C-terminus of the Fc region.

Embodiment 95. The polypeptide of embodiment 93 or embodiment 94, wherein the Fc region comprises a substitution at Kabat amino acid position T366.

Embodiment 96. The polypeptide of embodiment 95, wherein the Fc region comprises a T366W substitution.

Embodiment 97. The polypeptide of embodiment 93 or embodiment 94, wherein the Fc region comprises at least one substitution at at least one Kabat amino acid position selected from T366, L368, and Y407.

Embodiment 98. The polypeptide of embodiment 108, wherein the Fc region comprises T366S, L368A, and Y407V mutations.

Embodiment 99. The polypeptide of any one of embodiments 93-98, wherein the Fc region comprises a substitution at a Kabat position selected from S354 and Y349.

Embodiment 100. The polypeptide of embodiment 99, wherein the Fc region comprises a S354C or a Y349C substitution.

Embodiment 101. The polypeptide of any one of embodiments 93-100, wherein the Fc region comprises a substitution at Kabat amino acid position H435.

Embodiment 102. The polypeptide of embodiment 101, wherein the Fc region comprises a substitution selected from H435R and H435K.

Embodiment 103. The polypeptide of any one of embodiments 93-102, wherein the Fc region comprises at least one substitution at at least one Kabat amino acid position selected from M252 and M428.

Embodiment 104. The polypeptide of embodiment 103, wherein the Fc region comprises M252Y and M428V substitutions.

Embodiment 105. The polypeptide of any one of embodiments 93-104, wherein the Fc region comprises a deletion of Kabat amino acids E233, L234, and L235.

Embodiment 106. The polypeptide of any one of embodiments 93-104, wherein the Fc region comprises at least one substitution at at least one amino acid position selected from L234, L235, and P329.

Embodiment 107. The polypeptide of embodiment 106, wherein the Fc region comprises L234A, L235A, and P329G substitutions.

Embodiment 108. The polypeptide of any one of embodiments 93-107, wherein the Fc region comprises an amino acid sequence at least 90%, 91%, 92%, 93%, 94%, 95%, 96%, 97%, 98%, 99%, or 100% identical to an amino acid sequence selected from SEQ ID NOs: 47-83, 292, and 293.

Embodiment 109. The polypeptide of any one of embodiments 93-108, wherein the Fc region is part of a heavy chain constant region.

Embodiment 110. The polypeptide of embodiment 109, wherein the heavy chain constant region is an IgG constant region.

Embodiment 111. The polypeptide of embodiment 110, wherein the heavy chain constant region is an IgG1, IgG2, IgG3, or IgG4 constant region.

Embodiment 112. The polypeptide of any one of embodiments 93-111, wherein the modified IL-2 is fused to the C-terminus of the Fc region or heavy chain constant region.

Embodiment 113. The polypeptide of embodiment 112, wherein the modified IL-2 is fused to the C-terminus of the Fc region or heavy chain constant region via a linker comprising 1-20 amino acids.

Embodiment 114. The polypeptide of embodiment 113, wherein the linker comprises glycine amino acids.

Embodiment 115. The polypeptide of embodiment 114, wherein the linker comprises glycine and serine amino acids.

Embodiment 116. The polypeptide of any one of embodiments 113-115, wherein a majority, or all, of the amino acids in the linker are glycine and serine.

Embodiment 117. The polypeptide of any one of embodiments 93-116, wherein the polypeptide comprises an amino acid sequence selected from SEQ ID NOs: 105-290 and an amino acid sequence selected from SEQ ID NOs: 48, 64, 292, and 293.

Embodiment 118. The polypeptide of any one of the preceding embodiments, wherein the polypeptide comprises at least one antigen binding domain.

Embodiment 119. The polypeptide of embodiment 118, wherein the polypeptide comprises two, three, or four antigen binding domains.

Embodiment 120. The polypeptide of embodiment 118 or embodiment 119, wherein at least one antigen binding domain specifically binds to a T-cell antigen or a natural killer cell antigen.

Embodiment 121. The polypeptide of any one of embodiments 118-120, wherein at least one antigen binding domain specifically binds to a CD4⁺ T-cell antigen or a CD8⁺ T-cell antigen.

Embodiment 122. The polypeptide of embodiment 121, wherein the at least one antigen binding domain specifically binds to an antigen on an activated CD4⁺ T-cell or an activated CD8⁺ T-cell.

Embodiment 123. The polypeptide of any one of embodiments 118-122, wherein at least one antigen binding domain is an agonist.

Embodiment 124. The polypeptide of any one of embodiments 118-122, wherein the antigen binding domain is an antagonist.

Embodiment 125. The polypeptide of any one of embodiments 118-124, wherein at least one antigen binding domain specifically binds to PD-1, CTLA-4, LAG3, TIM3, 4-1BB, OX40, GITR, CD8a, CD8b, CD4, NKp30, NKG2A, TIGIT, TGFβR1, TGFβR2, Fas, NKG2D, NKp46, PD-L1, CD107a, ICOS, TNFR2, CD16a, or γδTCR.

Embodiment 126. The polypeptide of any one of embodiments 118-124, wherein at least one antigen binding domain specifically binds to PD-1.

Embodiment 127. The polypeptide of any one of embodiments 118-126, wherein at least one antigen binding domain is a human or humanized antigen binding domain.

Embodiment 128. The polypeptide of embodiment 127, wherein each antigen binding domain is, independently, a human or humanized antigen binding domain.

Embodiment 129. The polypeptide of any one of embodiments 118-128, wherein at least one antigen binding domain comprises a VHH domain.

Embodiment 130. The polypeptide of embodiment 129, wherein each antigen binding domain comprises a VHH domain.

Embodiment 131. The polypeptide of any one of embodiments 118-128, wherein at least one antigen binding domain comprises a VH domain and a VL domain.

Embodiment 132. The polypeptide of embodiment 131, wherein at least one antigen binding domain comprises the VH domain and the VL domain of an antibody selected from pembrolizumab, nivolumab, AMP-514, TSR-042, STI-A1110, ipilimumab, tremelimumab, urelumab, utomilumab, atezolizumab, and durvalumab.

Embodiment 133. The polypeptide of embodiment 131 or 132, wherein the at least one antigen binding domain comprises a single chain Fv (scFv).

Embodiment 134. The polypeptide of embodiment 131 or 132, wherein the polypeptide comprises a heavy chain constant region, wherein the VH domain is fused to the heavy chain constant region, and wherein the VL domain is associated with the VH domain.

Embodiment 135. The polypeptide of embodiment 134, wherein the VL domain is fused to a light chain constant region.

Embodiment 136. The polypeptide of embodiment 135, wherein the light chain constant region is selected from kappa and lambda.

Embodiment 137. The polypeptide of any one of embodiments 118-136, wherein each of the antigen binding domains are the same.

Embodiment 138. The polypeptide of embodiment 118-137, wherein each of the antigen binding domains specifically bind to the same antigen.

Embodiment 139. The polypeptide of embodiment 118-136, wherein at least one of the antigen binding domains specifically binds to a different antigen than at least one of the other antigen binding domains.

Embodiment 140. The polypeptide of embodiment 139, wherein at least one antigen binding domain specifically binds to PD-1 and at least one other antigen binding domain specifically binds to a T-cell antigen or natural killer cell antigen other than PD-1.

Embodiment 141. The polypeptide of any one of embodiments 118-140, wherein at least one antigen binding domain binds to PD-1, CTLA-4, LAG3, TIM3, 4-1BB, OX40, GITR, CD8a, CD8b, CD4, NKp30, NKG2A, TIGIT, TGFβR1, TGFβR2, Fas, NKG2D, NKp46, PD-L1, CD107a, ICOS, TNFR2, CD16a, DNAM1, or γδTCR (Vγ9, Vγ2, VM).

Embodiment 142. The polypeptide of any one of embodiments 93-141, wherein the polypeptide forms a homodimer under physiological conditions.

Embodiment 143. The polypeptide of any one of the preceding embodiments, wherein the modified IL-2 binds a human IL-2R with an affinity at least 2-fold, 3-fold, 4-fold, 5-fold, 6-fold, 7-fold, 8-fold, 9-fold, at least 10-fold, at least 20-fold, at least 30-fold, at least 50-fold, or at least 100-fold lower than the affinity of human wild type IL-2 for the IL-2R.

Embodiment 144. A complex comprising a first polypeptide and a second polypeptide, wherein the first polypeptide is the polypeptide of any one of the preceding embodiments.

Embodiment 145. The complex of embodiment 144, wherein the first polypeptide comprises a first Fc region and the second polypeptide comprises a second Fc region.

Embodiment 146. The complex of embodiment 144 or embodiment 145, wherein each Fc region is an isotype selected from human IgG1, IgG2, IgG3, an IgG4.

Embodiment 147. The complex of embodiment 146, wherein each Fc region is a human IgG1.

Embodiment 148. The complex of any one of embodiments 144-147, wherein each Fc region comprises a deletion of amino acids E233, L234, and L235.

Embodiment 149. The complex of any one of embodiments 144-148, wherein each Fc region comprises a H435R or H435K mutation.

Embodiment 150. The complex of any one of embodiments 155-160, wherein the Fc region comprises a mutations M252Y and M428L or mutations M252Y and M428V.

Embodiment 151. The complex of any one of embodiments 144-150, wherein the first Fc region or the second Fc region comprises a T366W mutation, and the other Fc region comprises mutations T366S, L368A, and Y407V.

Embodiment 152. The complex of embodiment 151, wherein the first Fc region or the second Fc region comprises a S354C mutation.

Embodiment 153. The complex of any one of embodiments 144-152, wherein each Fc region independently comprises an amino acid sequence at least 90%, 91%, 92%, 93%, 94%, 95%, 96%, 97%, 98%, 99%, or 100% identical to an amino acid sequence selected from SEQ ID NOs: 47-83, 292, and 293.

Embodiment 154. The complex of any one of embodiments 144-153, wherein the second polypeptide does not comprise a modified IL-2.

Embodiment 155. The complex of any one of embodiments 144-154, wherein the first polypeptide comprises at least one antigen binding domain.

Embodiment 156. The complex of any one of embodiments 144-155, wherein the second polypeptide comprises at least one antigen binding domain.

Embodiment 157. The complex of any one of embodiments 144-156, wherein the first polypeptide comprises a first antigen binding domain, an Fc region, and a modified IL-2.

Embodiment 158. The complex of embodiment 157, wherein the first antigen binding domain is fused to the N-terminus of the Fc region and the modified IL-2 is fused to the C-terminus of the Fc region.

Embodiment 159. The complex of embodiment 157 or embodiment 158, wherein the second polypeptide comprises a second antigen binding domain and an Fc region.

Embodiment 160. The complex of embodiment 159, wherein the first antigen binding domain and the second antigen binding domain are the same or different.

Embodiment 161. The complex of embodiment 160, wherein:

a. the first antigen binding domain and the second antigen binding domain both bind PD-1;

b. the first antigen binding domain binds PD-1, and the second antigen binding domain binds LAG3;

c. the first antigen binding domain binds PD-1, and the second antigen binding domain binds CTLA-4;

d. the first antigen binding domain binds PD-1, and the second antigen binding domain binds 4-1BB;

e. the first antigen binding domain binds PD-1, and the second antigen binding domain binds OX40;

f. the first antigen binding domain binds PD-1, and the second antigen binding domain binds GITR;

g. the first antigen binding domain binds PD-1, and the second antigen binding domain binds CD8a;

h. the first antigen binding domain binds PD-1, and the second antigen binding domain binds CD8b;

i. the first antigen binding domain binds PD-1, and the second antigen binding domain binds CD4;

j. the first antigen binding domain binds PD-1, and the second antigen binding domain binds NKp30;

k. the first antigen binding domain binds PD-1, and the second antigen binding domain binds NKG2A;

l. the first antigen binding domain binds PD-1, and the second antigen binding domain binds TIGIT;

m. the first antigen binding domain binds PD-1, and the second antigen binding domain binds NKG2D;

n. the first antigen binding domain binds PD-1, and the second antigen binding domain binds TGFBR2;

o. the first antigen binding domain binds PD-1, and the second antigen binding domain binds Fas;

p. the first antigen binding domain binds PD-1, and the second antigen binding domain binds CD107a;

q. the first antigen binding domain binds PD-1, and the second antigen binding domain binds NKp46;

r. the first antigen binding domain binds CD8a, and the second antigen binding domain binds TGFRβR2;

s. the first antigen binding domain binds CD8a, and the second antigen binding domain binds Fas;

t. the first antigen binding domain binds NKG2D, and the second antigen binding domain binds TGFRβR2;

u. the first antigen binding domain binds NKG2D, and the second antigen binding domain binds Fas;

v. the first antigen binding domain binds NKG2A, and the second antigen binding domain binds TGFRβR2;

w. the first antigen binding domain binds NKG2A, and the second antigen binding domain binds Fas;

x. the first antigen binding domain binds NKp46, and the second antigen binding domain binds TGFRβR2;

y. the first antigen binding domain binds NKp46, and the second antigen binding domain binds Fas;

z. the first antigen binding domain binds CTLA-4, and the second antigen binding domain binds LAGS;

aa. the first antigen binding domain binds CTLA-4, and the second antigen binding domain binds Tim3;

bb. the first antigen binding domain binds CTLA-4, and the second antigen binding domain binds OX40;

cc. the first antigen binding domain binds CTLA-4, and the second antigen binding domain binds GITR;

dd. the first antigen binding domain binds CTLA-4, and the second antigen binding domain binds CD107a;

ee. the first antigen binding domain binds CTLA-4, and the second antigen binding domain binds NKp46;

ff. the first antigen binding domain binds ICOS, and the second antigen binding domain binds TNFR2;

gg. the first antigen binding domain binds γδTCR, and the second antigen binding domain binds NKG2D;

hh. the first antigen binding domain binds γδTCR, and the second antigen binding domain binds DNAM1;

ii. the first antigen binding domain binds γδTCR, and the second antigen binding domain binds TIGIT;

jj. the first antigen binding domain binds γδTCR, and the second antigen binding domain binds 4-1BB;

kk. the first antigen binding domain binds γδTCR, and the second antigen binding domain binds Fas;

ll. the first antigen binding domain binds γδTCR, and the second antigen binding domain binds NKG2A; or

mm. the first antigen binding domain binds γδTCR, and the second antigen binding domain binds CD16a.

Embodiment 162. The complex of any one of embodiments 144-161, wherein the modified IL-2 binds a human IL-2R with an affinity at least 2-fold, 3-fold, 4-fold, 5-fold, 6-fold, 7-fold, 8-fold, 9-fold, at least 10-fold, at least 20-fold, at least 30-fold, at least 50-fold, or at least 100-fold lower than the affinity of human wild type IL-2 for the IL-2R.

Embodiment 163. A pharmaceutical composition comprising a polypeptide of any one of embodiments 1-154 or the complex of any one of embodiments 144-162 and a pharmaceutically acceptable carrier.

Embodiment 164. An isolated nucleic acid the encodes a polypeptide of any one of embodiments 1-143 or the complex of any one of embodiments 144-162.

Embodiment 165. An expression vector comprising the nucleic acid of embodiment 164.

Embodiment 166. An isolated host cell comprising the nucleic acid of embodiment 164 or the expression vector of embodiment 165.

Embodiment 167. An isolated host cell that expresses the polypeptide of any one of embodiments 1-143 or the complex of any one of embodiments 144-162.

Embodiment 168. A method of producing the polypeptide of any one of embodiments 1-143 or the complex of any one of embodiments 144-162 comprising incubating the host cell of embodiment 166 or embodiment 167 under conditions suitable to express the polypeptide or complex.

Embodiment 169. The method of embodiment 168, further comprising isolating the polypeptide or complex.

Embodiment 170. A method of increasing CD4+ and/or CD8+ T cell proliferation comprising contacting T cells with the polypeptide of any one of embodiments 1-154 or the complex of any one of embodiments 144-162.

Embodiment 171. The method of embodiment 170, wherein the CD4+ and/or CD8+ T cells are in vitro.

Embodiment 172. The method of embodiment 170, wherein the CD4+ and/or CD8+ T cells are in vivo.

Embodiment 173. The method of any one of embodiments 170-172, wherein the increase is at least 1.5-fold, at least 2-fold, at least 3-fold, or by at least 5-fold.

Embodiment 174. A method of increasing NK cell proliferation comprising contacting NK cells with the polypeptide of any one of embodiments 1-143 or the complex of any one of embodiments 144-162.

Embodiment 175. The method of embodiment 174, wherein the increase is at least 1.5-fold, at least 2-fold, at least 3-fold, or by at least 5-fold.

Embodiment 176. A method of treating cancer comprising administering to a subject with cancer a pharmaceutically effective amount of the polypeptide of any one of embodiments 1-143 or the complex of any one of embodiments 144-162, or the pharmaceutical composition of embodiment 163.

Embodiment 177. The method of embodiment 176, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; and chronic myeloblastic leukemia.

Embodiment 178. The method of embodiment 176 or 177, further comprising administering an additional therapeutic agent.

Embodiment 179. The method of embodiment 178, wherein the additional therapeutic agent is an anti-cancer agent.

Embodiment 180. The method of embodiment 179, wherein the anti-cancer agent is selected from a chemotherapeutic agent, an anti-cancer biologic, radiation therapy, CAR-T therapy, and an oncolytic virus.

Embodiment 181. The method of embodiment 179 or embodiment 180, wherein the additional therapeutic agent is an anti-cancer biologic.

Embodiment 182. The method of embodiment 181, wherein the anti-cancer biologic is an agent that inhibits PD-1 and/or PD-L1.

Embodiment 183. The method of embodiment 181, wherein the anti-cancer biologic is an agent that inhibits VISTA, gpNMB, B7H3, B7H4, HHLA2, CTLA4, or TIGIT.

Embodiment 184. The method of any one of embodiments 179-183, wherein the anti-cancer agent is an antibody.

Embodiment 185. The method of embodiment 181, wherein the anti-cancer biologic is a cytokine.

Embodiment 186. The method of embodiment 179, wherein the anti-cancer agent is CAR-T therapy.

Embodiment 187. The method of embodiment 179, wherein the anti-cancer agent is an oncolytic virus.

Embodiment 188. The method of any one of embodiments 176-187, further comprising tumor resection and/or radiation therapy.

### BRIEF DESCRIPTION OF THE FIGURES

FIG. 1A-1H show schematics of various IL-2 fusion protein formats. FIG. 1A shows IL-2 linked to the N-terminus of a heterodimeric, knob-in-hole IgG1 Fc. FIG. 1B shows IL-2 linked to the C-terminus of a heterodimeric IgG1 Fc of a single domain antibody. FIG. 1C-1E show IL-2 linked to one VHH (FIG. 1E), two identical VHHs (FIG. 1C), or two different VHHs (FIG. 1D). FIG. 1F shows IL-2 linked to the C-terminus of a homodimeric heavy chain constant region of a conventional antibody. FIG. 1G shows IL-2 linked to the C-terminus of a heterodimeric heavy chain constant region of a conventional antibody. FIG. 1H shows IL-2 fused to the C-terminus of a heterodimeric scFv antibody.

FIG. 2A-2C show binding of IL-2 fusion proteins comprising wild type IL-2 (FIG. 2A) or a modified IL-2 (FIG. 2A-2C) fused to the N-terminus of a heterodimeric Fc, as shown in FIG. 1A, to 293F cells transiently transfected with various combinations of the IL-2 receptor (CD25, CD122, and CD132), as measured by flow cytometry. “UT 293F” indicates untransfected 293F cells.

FIG. 3A-3B show binding of fusion proteins comprising wild type IL-2 or a modified IL-2 fused to the N-terminus of a heterodimeric Fc, as shown in FIG. 1A, to 293F cells transiently transfected with CD25 and CD122, as measured by flow cytometry.

FIG. 4A-4B show binding of fusion proteins comprising wild type IL-2 or a modified IL-2 fused to the N-terminus of a heterodimeric Fc, as shown in FIG. 1A, to 293F cells transiently transfected with CD122 and CD132; or CD25, CD122, and CD132, as measured by flow cytometry.

FIG. 5A-5B show binding of fusion proteins comprising wild type IL-2 or a modified IL-2 fused to the C-terminus of a non-targeting VHH linked to a heterodimeric Fc, as shown in FIG. 1B, to resting and activated CD4+ T cells, as measured by flow cytometry. “Isotype control” indicates a control protein that does not comprise IL-2.

FIG. 6A-6B show binding of fusion proteins comprising wild type IL-2 or a modified IL-2 fused to the C-terminus of a non-targeting VHH linked to a heterodimeric Fc, as shown in FIG. 1B, to enriched regulatory T cells (Tregs, FIG. 6A), induced regulatory T cells (induced Tregs, FIG. 6B), and enriched responder CD4+ T cells (Tresps, FIG. 6C), as measured by flow cytometry.

FIG. 7A-7D show the activity of fusion proteins comprising wild type IL-2 or a modified IL-2 fused to the C-terminus of a non-targeting VHH linked to a heterodimeric Fc, as shown in FIG. 1B, on resting CD4+ and CD8+ T cells. Proliferation (FIGS. 7A and 7C) and CD71 levels (FIGS. 7B and 7D) were measured. FIG. 7E-7F show activity of wild type IL-2 or a modified IL-2 fused to the C-terminus of a non-targeting VHH linked to a heterodimeric Fc, as shown in FIG. 1B, on resting CD4+ and CD8+ T cells as measured by flow cytometric detection of intracellular phosphorylated STATS levels. “Isotype” indicates a control protein that does not comprise IL-2.

FIG. 8A-8B show the proliferation and CD25 levels as a marker of activation of enriched Tregs following treatment for 7 days with a fusion protein comprising wild type IL-2 or a modified IL-2 fused to the C-terminus of a non-targeting VHH linked to a heterodimeric Fc, as shown in FIG. 1B.

FIG. 9A-9D show activity and binding of pembrolizumab, an analog of pembrolizumab with IL-2-RAS fused to the heavy chain C-terminus, as shown in FIG. 1F, and IL-2-RAS alone (FIGS. 9C and 9D) on CD8+ and CD4+ T cells. Activity on CD8+ (FIG. 9A) and CD4+ (FIG. 9B) T-cells was measured by flow cytometric detection of CellTrace™ Violet. Extent of binding to CD8+ T cells (FIG. 9C) and CD4+ T cells (FIG. 9D) was measured by flow cytometry.

FIG. 10A-10D show dependency of induction of CD8+ and CD4+ T cell proliferation on IL-2. Effects of pembrolizumab, non-targeted IL-2-RAS, and an analog of pembrolizumab with IL-2-RAS fused to the heavy chain C-terminus, as shown in FIG. 1F, on CD8+ (FIGS. 10A and 10C) or CD4+ (FIGS. 10B and 10D) T cell proliferation without pre-blocking (FIGS. 10A and 10B) or pre-blocked with a saturating concentration of pembrolizumab (FIGS. 10C and 10D) are shown.

FIG. 11 shows the recovery of CD4+T responder (Tresp) cell proliferation by an analog of pembrolizumab with IL-2-RAS fused to the heavy chain C-terminus, as shown in FIG. 1F, as well as IL-2-RAS fused to the C-terminus of a non-targeted VHH, as shown in FIG. 1B and wild type IL-2 fused to the C-terminus of a non-targeted VHH, as shown in FIG. 1B. Tresp proliferation was induced by CD3 engagement (Tresp+beads), then suppressed using autologous regulatory T cells (Treg). “Tresp+beads” line shows baseline Tresp cell proliferation with CD3 engagement in the absence of Treg cells. “No Ab” line shows baseline Tresp cell proliferation with CD3 engagement in the presence of Treg cells.

FIG. 12A-12B show the trans-activation of T cells by plate-bound non-targeted wild type IL-2 (“IL-2 WT”) or IL-2-RAS fused to the C-terminus of a non-targeted VHH, as shown in FIG. 1B. T cell activation was measured by flow cytometric detection of intracellular phosphorylated STATS levels. CD8+ T cell (FIG. 12A) and CD4+ T cell (FIG. 12B) responses are shown.

FIG. 13A-131 show activity and binding of IL-2-RAS fused to the C-terminus of a heterodimeric scFv antibody targeting NKp46, as shown in FIG. 1H, the heterodimeric scFv antibody targeting NKp46 alone, and fusion proteins comprising wild type IL-2 or IL-2-RAS fused to the C-terminus of a non-targeting VHH linked to a heterodimeric Fc, as shown in FIG. 1B, on NK cells, CD8+ T cells, and CD4+ T cells. Proliferation of NK cells (FIG. 13A), CD8+ T cells (FIG. 13B), and CD4+ T cells (FIG. 13C) and pSTAT levels of NK cells (FIG. 13D), CD8+ T cells (FIG. 13E), and CD4+ T cells (FIG. 13F) were measured by flow cytometry. Binding of the indicated polypeptides to NK cells (FIG. 13G), CD8+ T cells (FIG. 13H) and CD4+ T cells (FIG. 13I) was also measured by flow cytometry.

FIG. 14A-14H show activity and binding on CD8+ or CD4+ T cells of IL-2-RAS fused to the C-terminus of an anti-LAG3 heterodimeric conventional antibody (MAb), as shown in FIG. 1G, IL-2-RAS fused to the C-terminus of an anti-LAG3 VHH with a heterodimeric Fc, as shown in FIG. 1B, IL-2-RAS fused to the C-terminus of a non-targeted VHH, as shown in FIG. 1B, wild type IL-2 fused to the C-terminus of a non-targeted heterodimeric Fc, as shown in FIG. 1B, or LAGS-targeted MAb or LAGS-targeted VHH-Fc molecules without IL-2. Proliferation of CD8+ T cells (FIG. 14A) and CD4+ T cells (FIG. 14B) and expression of activation markers CD25 (FIGS. 14C and 14D) and CD71 (FIGS. 14E and 14F) on CD8+ T cells (FIGS. 14C and 14E) and CD4+ T cells (FIGS. 14D and 14F) were measured by flow cytometry. FIGS. 14G and 14H show binding to pre-activated CD8+ T cells (FIG. 14G) and CD4+ T cells (FIG. 14H).

FIG. 15 shows activity of fusion proteins comprising the indicated modified IL-2 fused to the C-terminus of a VHH with a heterodimeric Fc, as shown in FIG. 1B, on HEK-Blue IL-2 reporter cells that do not express the VHH's target antigen and therefore rely solely on binding of the modified IL-2 to the overexpressed IL-2 receptor for induction of the reporter gene. The activity of secreted embryonic alkaline phosphatase expressed in response to IL-2 receptor-mediated induction of pSTAT5 signaling in the reporter cell was measured.

FIG. 16A-16B show activities of polypeptides comprising the indicated modified IL-2 on HEK-Blue IL-2 reporter cells that do not express PD-1 (FIG. 16A) and on IL-2 reporter cells that express PD-1 (FIG. 16B). “RAS” as used in FIG. 16A-16B means IL-2 mutations T3A, P65R, H16A, D84S, and C125S.

FIG. 17A-17C show activities of polypeptides comprising the indicated modified IL-2 on HEK-Blue IL-2 reporter cells that do not express PD-1 in the presence and absence of a CD25 blocking antibody. “RAS” as used in FIG. 17A-17C means IL-2 mutations T3A, P65R, H16A, D84S, and C125S.

FIG. 18A-18B show activities of polypeptides comprising the indicated modified IL-2 on HEK-Blue IL-2 reporter cells that do not express PD-1 (FIG. 18A) and on IL-2 reporter cells that express PD-1 (FIG. 18B). “RAS” as used in FIG. 18A-18B means IL-2 mutations T3A, P65R, H16A, D84S, and C125S. “INBRX-108” as used in FIG. 18B means modified IL-2 comprising the amino acid sequence of SEQ ID NO: 28.

FIG. 19A-19E show activities of polypeptides comprising the indicated modified IL-2 on IL-2 reporter cells that express PD-1 or that do not express PD-1, as indicated. “NT_mutant” as used in FIG. 19A-19E means the polypeptide comprises the IL-2 mutations listed in the panel title and was tested on cells that do not express PD-1. “PD1 mutant” as used in FIG. 19A-19E means the polypeptide comprises the IL-2 mutations listed in the panel title and was tested on cells that do express PD-1. “RAS” as used in FIG. 19A-19E means IL-2 mutations T3A, P65R, H16A, D84S, and C125S. “NT_INBRX-108” as used in FIG. 19A-19E means the polypeptide comprises a modified IL-2 comprising the amino acid sequence of SEQ ID NO: 28 and was tested on cells that do not express PD-1. “PD1_INBRX-108” as used in FIG. 19A-19E means the polypeptide comprises a modified IL-2 comprising the amino acid sequence of SEQ ID NO: 28 and was tested on cells that do express PD-1. The polypeptides, including the IL-2 mutations, are also described in Example 19 and Table 7.

FIG. 20A-20E show activities of polypeptides comprising the indicated modified IL-2 on IL-2 reporter cells that express PD-1 or that do not express PD-1, as indicated. “NT_mutant” as used in FIG. 20A-20E means the polypeptide comprises the IL-2 mutations listed in the panel title and was tested on cells that do not express PD-1. “PD1_mutant” as used in FIG. 20A-20E means the polypeptide comprises the IL-2 mutations listed in the panel title and was tested on cells that do express PD-1. “RAS” as used in FIG. 20A-20E means IL-2 mutations T3A, P65R, H16A, D84S, and C125S. “NT_INBRX-108” as used in FIG. 20A-20E means the polypeptide comprises a modified IL-2 comprising the amino acid sequence of SEQ ID NO: 28 and was tested on cells that do not express PD-1. “PD1_INBRX-108” as used in FIG. 20A-20E means the polypeptide comprises a modified IL-2 comprising the amino acid sequence of SEQ ID NO: 28 and was tested on cells that do express PD-1. The polypeptides, including the IL-2 mutations, are also described in Example 20 and Table 8.

FIG. 21A-21D show activities of polypeptides comprising the indicated modified IL-2 on IL-2 reporter cells that express PD-1 or that do not express PD-1, as indicated. “NT_mutant” as used in FIG. 21A-21D means the polypeptide comprises the IL-2 mutations listed in the panel title and was tested on cells that do not express PD-1. “PD1_mutant” as used in FIG. 21A-21D means the polypeptide comprises the IL-2 mutations listed in the panel title and was tested on cells that do express PD-1. “RAS” as used in FIG. 21A-21D means IL-2 mutations T3A, P65R, H16A, D84S, and C125S. “NT_INBRX-108” as used in FIG. 21A-21D means the polypeptide comprises a modified IL-2 comprising the amino acid sequence of SEQ ID NO: 28 and was tested on cells that do not express PD-1. “PD1_INBRX-108” as used in FIG. 21A-21D means the polypeptide comprises a modified IL-2 comprising the amino acid sequence of SEQ ID NO: 28 and was tested on cells that do express PD-1. The polypeptides, including the IL-2 mutations, are also described in Example 21 and Table 9.

FIG. 22A-22B show activities of polypeptides comprising the indicated modified IL-2 on HEK-Blue IL-2 reporter cells that do not express PD-1 (FIG. 22A) and on IL-2 reporter cells that express PD-1 (FIG. 22B). “RAS” as used in FIG. 22A-22B means IL-2 mutations T3A, P65R, H16A, D84S, and C125S. Additionally, all of the indicated modified IL-2 mutations include T3A and C125S as well as the listed mutations.

FIG. 23A-23B show activities of polypeptides comprising the indicated modified IL-2 on HEK-Blue IL-2 reporter cells that do not express PD-1 (FIG. 23A) and on IL-2 reporter cells that express PD-1 (FIG. 23B). “RAS” as used in FIG. 23A-23B means IL-2 mutations T3A, P65R, H16A, D84S, and C125S.

FIG. 24A-24B show activities of the indicated polypeptides comprising a modified IL-2 comprising the indicated mutations on HEK-Blue IL-2 reporter cells that do not express CD8a (FIG. 24A) and on IL-2 reporter cells that do express CD8a (FIG. 24B). “RAS” as used in FIG. 24A-24B means IL-2 mutations T3A, P65R, H16A, D84S, and C125S. “RAY” as used in FIG. 24A-24B means IL-2 mutations T3A, P65R, H16A, D84Y, and C125S.

FIG. 25A-25C show activities of the indicated polypeptides comprising the indicated modified or wild-type IL-2 on HEK-Blue IL-2 reporter cells (FIG. 25A), on IL-2 reporter cells that express NKp46 (FIG. 25B), and on IL-2 reporter cells that express CD8a (FIG. 25C). “OprIxIL-2-WT tgcs” as used in FIG. 25A-C means a polypeptide comprising an OprI binding domain, which does not bind a target on the IL-2 reporter cells, and an IL-2 comprising T3G and C125S mutations.

FIG. 26A-26E show analysis of activities of various polypeptides comprising an IL-2 mutant. FIG. 26A shows the non-targeted activities of various IL-2 mutants. FIG. 26B-24C are plots of targeted EC₅₀s for PD-1 (FIG. 26B) or NKp46 (FIG. 26C) versus the non-targeted EC₅₀s. FIG. 26D-26E show the PD-1 (FIG. 26D) and NKp46 (FIG. 26E) targeted windows, which show the concentration ranges at which targeted activity is achieved while avoiding non-targeted IL-2 activity.

FIG. 27A-27H show pSTAT5 levels following treatment of PBMCs with a fusion protein comprising a modified IL-2 fused to the C-terminus of a γδTCR-binding VHH with a heterodimeric Fc, where the γδTCR binding domain is monovalent or bivalent, as indicated. FIGS. 27A, 27C, 27E, and 27G show the median fluorescence intensity of intracellular pSTAT5 staining on γδT cells, NK cells, and αβT cells following treatment with the indicated polypeptides. FIGS. 27B, 27D, 27F, and 27H show the percent of γδT cells, NK cells, and αβT cells with pSTAT5 staining after treatment with the indicated polypeptides.

FIG. 28A-28D show γδT cell proliferation (FIG. 28A), γδT cell accumulation (FIG. 28B), αβT cell proliferation (FIG. 28C), and αβT cell accumulation (FIG. 28D) following treatment of PBMCs with a fusion protein comprising a modified IL-2 fused to the C-terminus of a γδTCR-binding VHH with a heterodimeric Fc.

FIG. 29A-29B show pSTAT5 levels following treatment of PBMCs with wild type IL-2 and an untargeted polypeptide comprising a modified IL-2. FIG. 29A shows the percent of cells with pSTAT5 staining on CD56bringht CD16− NK cells

### DETAILED DESCRIPTION

Embodiments provided herein relate to polypeptides comprising a modified IL-2 that modulates the activity of T cells and their use in various methods of treating cancer.

### Definitions and Various Embodiments

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991). The “EU index as in Kabat” refers to the residue numbering of the human IgG1 EU antibody.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The phrase “reference sample”, “reference cell”, or “reference tissue”, denote a sample with at least one known characteristic that can be used as a comparison to a sample with at least one unknown characteristic. In some embodiments, a reference sample can be used as a positive or negative indicator. A reference sample can be used to establish a level of protein and/or mRNA that is present in, for example, healthy tissue, in contrast to a level of protein and/or mRNA present in the sample with unknown characteristics. In some embodiments, the reference sample comes from the same subject, but is from a different part of the subject than that being tested. In some embodiments, the reference sample is from a tissue area surrounding or adjacent to the cancer. In some embodiments, the reference sample is not from the subject being tested, but is a sample from a subject known to have, or not to have, a disorder in question (for example, a particular cancer or T cell related disorder). In some embodiments, the reference sample is from the same subject, but from a point in time before the subject developed cancer. In some embodiments, the reference sample is from a benign cancer sample, from the same or a different subject. When a negative reference sample is used for comparison, the level of expression or amount of the molecule in question in the negative reference sample will indicate a level at which one of skill in the art will appreciate, given the present disclosure, that there is no and/or a low level of the molecule. When a positive reference sample is used for comparison, the level of expression or amount of the molecule in question in the positive reference sample will indicate a level at which one of skill in the art will appreciate, given the present disclosure, that there is a level of the molecule.

The terms “benefit”, “clinical benefit”, “responsiveness”, and “therapeutic responsiveness” as used herein in the context of benefiting from or responding to administration of a therapeutic agent, can be measured by assessing various endpoints, e.g., inhibition, to some extent, of disease progression, including slowing down and complete arrest; reduction in the number of disease episodes and/or symptoms; reduction in lesion size; inhibition (that is, reduction, slowing down or complete stopping) of disease cell infiltration into adjacent peripheral organs and/or tissues; inhibition (that is, reduction, slowing down or complete stopping) of disease spread; relief, to some extent, of one or more symptoms associated with the disorder; increase in the length of disease-free presentation following treatment, for example, progression-free survival; increased overall survival; higher response rate; and/or decreased mortality at a given point of time following treatment. A subject or cancer that is “non-responsive” or “fails to respond” is one that has failed to meet the above noted qualifications to be “responsive”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification. “Amino acid sequence” refers to the linear sequence of amino acids comprised in a polypeptide or protein.

“IL-2” or “Interleukin-2” as used herein refers to any native, mature IL-2 that results from processing of an IL-2 precursor in a cell. The term includes IL-2 from any vertebrate source, including mammals such as primates (e.g., humans and cynomolgus or rhesus monkeys) and rodents (e.g., mice and rats), unless otherwise indicated. The term also includes naturally-occurring variants of IL-2, such as splice variants or allelic variants. A nonlimiting exemplary human IL-2 amino acid sequence is shown, e.g., in GenBank Accession No. NP 000577.2. See SEQ ID NO. 1 (mature form).

“Modified IL-2” as used herein refers to a polypeptide that differs from a wild type IL-2 amino acid sequence by a substitution at at least one amino acid position.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antigen binding domain “specifically binds” or “preferentially binds” to an antigen if it binds with greater affinity, avidity, more readily, and/or with greater duration than it binds to other substances. For example, a sdAb or VHH-containing polypeptide that specifically or preferentially binds to an epitope is a sdAb or VHH-containing polypeptide that binds this epitope with greater affinity, avidity, more readily, and/or with greater duration than it binds to other epitopes on the same target antigen or epitopes on other target antigens. It is also understood by reading this definition that; for example, an antigen binding domain that specifically or preferentially binds to a first antigen may or may not specifically or preferentially bind to a second antigen. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, the term “modulate” with regard to the activity of IL-2 refers to a change in the activity of IL-2. In some embodiments, “modulate” refers to an increase in IL-2 activity.

As used herein, the term “epitope” refers to a site on a target molecule (for example, an antigen, such as a protein, nucleic acid, carbohydrate or lipid) to which an antigen binding molecule (for example, an antigen binding domain-containing polypeptide) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen binding molecule. In some embodiments, an epitope can be identified by the above distance, and further limited to those residues involved in a bond (for example, a hydrogen bond) between a residue of the antigen binding molecule and an antigen residue. An epitope can be identified by various scans as well, for example an alanine or arginine scan can indicate one or more residues that the antigen binding molecule can interact with. Unless explicitly denoted, a set of residues as an epitope does not exclude other residues from being part of the epitope for a particular antigen binding domain or molecule. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

A “nonlinear epitope” or “conformational epitope” comprises noncontiguous polypeptides, amino acids and/or sugars within the antigenic protein to which an antigen binding molecule (for example, an antigen binding domain-containing polypeptide) specific to the epitope binds. In some embodiments, at least one of the residues will be noncontiguous with the other noted residues of the epitope; however, one or more of the residues can also be contiguous with the other residues.

A “linear epitope” comprises contiguous polypeptides, amino acids and/or sugars within the antigenic protein to which an antigen-binding molecule (for example, an antigen binding domain-containing polypeptide) specific to the epitope binds. It is noted that, in some embodiments, not every one of the residues within the linear epitope need be directly bound (or involved in a bond) by the antigen binding molecule. In some embodiments, linear epitopes can be from immunizations with a peptide that effectively consisted of the sequence of the linear epitope, or from structural sections of a protein that are relatively isolated from the remainder of the protein (such that the antigen binding molecule can interact, at least primarily), just with that sequence section.

The terms “antibody” and “antigen binding molecule” are used interchangeably in the broadest sense and encompass various polypeptides that comprise antigen binding domains, including but not limited to conventional antibodies (typically comprising at least one heavy chain and at least one light chain), single-domain antibodies (sdAbs, comprising just one chain, which is typically similar to a heavy chain), VHH-containing polypeptides (polypeptides comprising at least one heavy chain only antibody variable domain, or VHH), and fragments of any of the foregoing so long as they exhibit the desired antigen binding activity. In some embodiments, an antibody comprises a dimerization domain. Such dimerization domains include, but are not limited to, heavy chain constant domains (comprising CH1, hinge, CH2, and CH3, where CH1 typically pairs with a light chain constant domain, CL, while the hinge mediates dimerization) and Fc regions (comprising hinge, CH2, and CH3, where the hinge mediates dimerization). The term antibody also includes, but is not limited to, chimeric antibodies, humanized antibodies, and antibodies of various species such as camelid (including llama), shark, mouse, human, cynomolgus monkey, etc.

The terms “single domain antibody” and “sdAb” are used interchangeably herein to refer to an antibody having a single, monomeric domain, typically a heavy chain (or VHH), without a light chain.

The term “VHH” or “VHH domain” or “VHH antigen binding domain” as used herein refers to the antigen binding portion of a single-domain antibody, such as a camelid antibody or shark antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains antigen binding and specificity.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc region. In some such embodiments, the VHH polypeptide may form a dimer. Nonlimiting structures of VHH-containing polypeptides include VHH₁-Fc, VHH₁-VHH₂-Fc, and VHH₁-VHH₂-VHH₃-Fc, wherein VHH₁, VHH₂, and VHH₃ may be the same or different. In some embodiments of such structures, one VHH may be connected to another VHH by a linker, or one VHH may be connected to the Fc by a linker. In some such embodiments, the linker comprises 1-20 amino acids, preferably 1-20 amino acids predominantly composed of glycine and, optionally, serine. In some embodiments, when a VHH-containing polypeptide comprises an Fc, it forms a dimer. Thus, the structure VHH₁-VHH₂-Fc, if it forms a dimer, is considered to be tetravalent (i.e., the dimer has four VHH domains). Similarly, the structure VHH₁-VHH₂-VHH₃-Fc, if it forms a dimer, is considered to be hexavalent (i.e., the dimer has six VHH domains).

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3.

The term “heavy chain constant region” as used herein refers to a region comprising at least three heavy chain constant domains, C_{H}1, hinge, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an a constant region is an IgA antibody. Further, an antibody comprising a μ constant region is an IgM antibody, and an antibody comprising an c constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

A “Fc region” as used herein refers to a portion of a heavy chain constant region comprising CH2 and CH3. In some embodiments, an Fc region comprises a hinge, CH2, and CH3. In various embodiments, when an Fc region comprises a hinge, the hinge mediates dimerization between two Fc-containing polypeptides. An Fc region may be of any antibody heavy chain constant region isotype discussed herein. In some embodiments, an Fc region is an IgG1, IgG2, IgG3, or IgG4.

An “acceptor human framework” as used herein is a framework comprising the amino acid sequence of a heavy chain variable domain (VH) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all of the human frameworks in a single antigen binding domain, such as a VHH.

“Affinity” refers to the strength of the sum total of noncovalent interactions between a single binding site of a molecule (for example, an antibody or VHH-containing polypeptide) and its binding partner (for example, an antigen). The affinity or the apparent affinity of a molecule X for its partner Y can generally be represented by the dissociation constant (K_{D}) or the K_{D}-apparent, respectively. Affinity can be measured by common methods known in the art (such as, for example, ELISA K_{D}, KinExA, flow cytometry, and/or surface plasmon resonance devices), including those described herein. Such methods include, but are not limited to, methods involving BIAcore®, Octet®, or flow cytometry.

The term “K_{D}”, as used herein, refers to the equilibrium dissociation constant of an antigen binding molecule/antigen interaction. When the term “K_{D}” is used herein, it includes K_{D} and K_{D-apparent}.

In some embodiments, the K_{D} of the antigen binding molecule is measured by flow cytometry using an antigen-expressing cell line and fitting the mean fluorescence measured at each antibody concentration to a non-linear one-site binding equation (Prism Software graphpad). In some such embodiments, the K_{D} is K_{D-apparent}.

The term “biological activity” refers to any one or more biological properties of a molecule (whether present naturally as found in vivo, or provided or enabled by recombinant means). Biological properties include, but are not limited to, binding a ligand, inducing or increasing cell proliferation (such as T cell proliferation), and inducing or increasing expression of cytokines.

The term “IL-2 activity” or “biological activity” of IL-2, as used herein, includes any biological effect or at least one of the biologically relevant functions of IL-2. In some embodiments, IL-2 activity includes the ability of IL-2 to induce T cell proliferation and/or activate natural killer (NK) cells. Nonlimiting exemplary IL-2 activities include increasing pSTAT5 expression, increasing proliferation of CD4⁺ and/or CD8⁺ T cells, increasing CD71 expression on T cells, and reducing the suppressive activity of Treg cells on CD4⁺ and CD8⁺ T cell activation and proliferation.

An “agonist” or “activating” antibody (such as a sdAb or VHH-containing polypeptide) is one that increases and/or activates a biological activity of the target antigen. In some embodiments, the agonist antibody binds to an antigen and increases its biologically activity by at least about 20%, 40%, 60%, 80%, 85% or more.

An “antagonist”, a “blocking” or “neutralizing” antibody is one that decreases and/or inactivates a biological activity of the target antigen. In some embodiments, the neutralizing antibody binds to an antigen and reduces its biologically activity by at least about 20%, 40%, 60%, 80%, 85% 90%, 95%, 99% or more.

An “affinity matured” VHH-containing polypeptide refers to a VHH-containing polypeptide with one or more alterations in one or more CDRs compared to a parent VHH-containing polypeptide that does not possess such alterations, such alterations resulting in an improvement in the affinity of the VHH-containing polypeptide for antigen.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to further refine and optimize VHH or VHH-containing polypeptide performance. In some embodiments, a humanized VHH-containing polypeptide comprises a human Fc region. As will be appreciated, a humanized sequence can be identified by its primary sequence and does not necessarily denote the process by which the antibody was created.

A “functional Fc region” possesses an “effector function” of a native sequence Fc region. Exemplary “effector functions” include Fc receptor binding; Clq binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. For example, the term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen or receptor binding, reduced antigen or receptor binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293F and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

A “disease” or “disorder” as used herein refers to a condition where treatment is needed and/or desired.

The term “tumor cell”, “cancer cell”, “cancer”, “tumor”, and/or “neoplasm”, unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases.

The terms “cancer” and “tumor” encompass solid and hematological/lymphatic cancers and also encompass malignant, pre-malignant, and benign growth, such as dysplasia. Also, included in this definition are cells having abnormal proliferation that is not impeded (e.g. immune evasion and immune escape mechanisms) by the immune system (e.g. virus infected cells). Exemplary cancers include, but are not limited to: basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

The term “non-tumor cell” as used herein refers to a normal cells or tissue. Exemplary non-tumor cells include, but are not limited to: T cells, B-cells, natural killer (NK) cells, natural killer T (NKT) cells, dendritic cells, monocytes, macrophages, epithelial cells, fibroblasts, hepatocytes, interstitial kidney cells, fibroblast-like synoviocytes, osteoblasts, and cells located in the breast, skeletal muscle, pancreas, stomach, ovary, small intestines, placenta, uterus, testis, kidney, lung, heart, brain, liver, prostate, colon, lymphoid organs, bone, and bone-derived mesenchymal stem cells. The term “a cell or tissue located in the periphery” as used herein refers to non-tumor cells not located near tumor cells and/or within the tumor microenvironment.

The term “cells or tissue within the tumor microenvironment” as used herein refers to the cells, molecules, extracellular matrix and/or blood vessels that surround and/or feed a tumor cell. Exemplary cells or tissue within the tumor microenvironment include, but are not limited to: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T cells; regulatory T cells (Treg cells); macrophages; neutrophils; myeloid-derived suppressor cells (MDSCs) and other immune cells located proximal to a tumor. Methods for identifying tumor cells, and/or cells/tissues located within the tumor microenvironment are well known in the art, as described herein, below.

In some embodiments, an “increase” or “decrease” refers to a statistically significant increase or decrease, respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of a test agent. This can be determined in any suitable manner and/or using any suitable assay known per se or described herein, depending on the target involved.

As used herein, “an immune response” is meant to encompass cellular and/or humoral immune responses that are sufficient to inhibit or prevent onset or ameliorate the symptoms of disease (for example, cancer or cancer metastasis). “An immune response” can encompass aspects of both the innate and adaptive immune systems.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering a therapeutic agent. “Ameliorating” also includes shortening or reduction in duration of a symptom.

The term “anti-cancer agent” is used herein in its broadest sense to refer to agents that are used in the treatment of one or more cancers. Exemplary classes of such agents in include, but are not limited to, chemotherapeutic agents, anti-cancer biologics (such as cytokines, receptor extracellular domain-Fc fusions, and antibodies), radiation therapy, CAR-T therapy, therapeutic oligonucleotides (such as antisense oligonucleotides and siRNAs) and oncolytic viruses.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” or “reference” refers to a composition known to not contain an analyte (“negative control”) or to contain an analyte (“positive control”). A positive control can comprise a known concentration of analyte.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time, but just over the time period being measured.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effects of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached, for example, to an antibody or antigen to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Exemplary Modified IL-2-Containing Polypeptides

Polypeptides comprising a modified IL-2 are provided herein. In some embodiments, the modified IL-2 comprises at least one amino acid substitution that reduces the affinity of the modified IL-2 for an IL-2 receptor compared to a wild type IL-2. In various embodiments, the polypeptide comprising a modified IL-2 provided herein is an agonist of an IL-2R. In some embodiments, the modified IL-2 is a modified human IL-2, and the IL-2R is a human IL-2R. In some embodiments, the modified IL-2 binds a human IL-2R with an affinity at least 2-fold, 3-fold, 4-fold, 5-fold, 6-fold, 7-fold, 8-fold, 9-fold, at least 10-fold, at least 20-fold, at least 30-fold, at least 50-fold, or at least 100-fold lower than the affinity of human wild type IL-2 for the IL-2R.

In various embodiments, the polypeptides comprising a modified IL-2 comprise at least one antigen binding domain that binds a T cell or natural killer (NK) cell antigen. In some embodiments, a polypeptide comprising a modified IL-2 provided herein comprises one, two, three, four, five, six, seven, or eight antigen binding domains, wherein at least one, or all, bind a T cell or natural killer cell antigen. In some embodiments, a polypeptide comprising a modified IL-2 provided herein comprises one, two, three, or four antigen binding domains, wherein at least one, or all, bind a T cell or natural killer cell antigen. In some embodiments, the modified IL-2 containing polypeptide does not bind or activate IL-2R in the absence of an antigen binding domain. In some embodiments, the modified IL-2 containing polypeptide binds and/or activates IL-2R on a cell only when the polypeptide comprises an antigen binding domain that is bound to an antigen on the same cell as the IL-2R.

In various embodiments, a modified IL-2 comprises at least one substitution at at least one amino acid position selected from L19, Q22, R38, E61, N88, R120, T123, Q126, 5127, 1129, and S130. In some embodiments, a modified IL-2 comprises at least one substitution at at least one amino acid position selected from T3, P65, H16, D84, M23, E95, and C125. In some embodiments, a modified IL-2 comprises substitutions at at least one amino acid position selected from L19, Q22, R38, E61, N88, R120, T123, Q126, S127, I129, and S130, and at amino acid positions T3, P65, H16, D84, and C125. In some such embodiments, the modified IL-2 further comprises substitutions at amino acid positions M23 and/or E95.

In some embodiments, the substitution at amino acid position L19 is selected from L19A, L19N, L19P, L19Q, L19Y, L19S, L19T, and L19V. In some embodiments, the substitution at amino acid position Q22 is selected from Q22A, Q22D, Q22G, Q22H, Q22K, Q22N, Q22R, Q22S, Q22T, Q22V, and Q22Y. In some embodiments, the substitution at amino acid position R38 is R38A or R38G. In some embodiments, the substitution at amino acid position E61 is selected from E61A, E61P, E61G, E61H, E61Q, E61N, E61R, E61S, E61T, E61K, and E61Y. In some embodiments, the substitution at amino acid position N88 is selected from N88A, N88S, and N88T. In some embodiments, the substitution at amino acid position R120 is selected from R120A, R120D, R120G, R120H, R120E, R120F, R120K, R120N, R120P, R120Q, R120S, R120V, and R120Y. In some embodiments, the substitution at amino acid position T123 is selected from T123D, T123E, T123H, T123K, T123N, T123R, and T123Q. In some embodiments, the substitution at amino acid position Q126 is selected from Q126A, Q126N, and Q126Y. In some embodiments, the substitution at amino acid position 5127 is selected from S127E, S127D, S127N, S127H, S127P, S127Q, and, S127R. In some embodiments, the substitution at amino acid position I129 is selected from I129A, I129H, I129R, and I129S. In some embodiments, the substitution at amino acid position S130 is selected from S130D, S130P, S130E, S130K, S130N, S130R, S130H, and S130Q.

In some embodiments, the substitution at amino acid position P65 is selected from P65R, P65E, P65K, P65H, P65Y, P65Q, P65D, and P65N. In some embodiments, the substitution at amino acid position H16 is selected from H16A, H16N, H16T, and H16V. In some embodiments, the substitution at amino acid position D84 is selected from D84S, D84N, D84G, D84A, D84T, D84V, and D84Y. In some embodiments, the substitution at amino acid position M23 is selected from M23A, M23R, M23Q, M23N, M23L, M23K, M23G, M23E, M23D, M23S, M23T, and M23V. In some embodiments, the substitution at amino acid position E95 is selected from E95Q, E95Y, E95G, E95T, E95V, E95P, E95H, and E95N.

In some embodiments, the substitution at amino acid position T3 is selected from T3A and T3G. In some embodiments, the substitution at amino acid position C125 is selected from C125A and C125S.

In some embodiments, the modified IL-2 further comprises a substitution at amino acid position F42. In some such embodiments, the substitution at F42 is selected from F42K, F42A, F42R, F42G, F42S, and F42T.

In some embodiments, the modified IL-2 further comprises at least one substitution at at least one amino acid position selected from Y45 and L72. In some such embodiments, the modified IL-2 comprises at least one substitution selected from Y45R, Y45K, and L72G.

In some embodiments, the modified IL-2 comprises substitutions T3A, P65R, H16A, D84Y, and C125S. In some embodiments, the modified IL-2 comprises substitutions T3A, P65R, H16A, D84Y, and C125S and comprises one or more substitutions at at least one position selected from L19, Q22, R38, E61, N88, R120, T123, Q126, S127, I129, and S130. In some embodiments, the modified IL-2 comprises substitutions T3A, P65R, H16A, D84S, and C125S and comprises one or more substitutions at at least one position selected from L19, Q22, R38, E61, N88, R120, T123, Q126, S127, I129, and S130. In some embodiments, the modified IL-2 comprises substitutions T3A, P65R, H16A, D84Y, C125S, and E61R. In some embodiments, the modified IL-2 comprises substitutions T3A, P65R, H16A, D84Y, C125S, and E61R and comprises at least one substitution selected from L19N, M23T, E95Q, and S127D. In some embodiments, the modified IL-2 comprises substitutions

a) T3A, H16A, E61R, P65R, D84Y, and C125S,

b) T3A, H16A, M23T, E61R, P65R, D84Y, E95Q, and C125S,

c) T3A, H16A, L19N, E61R, P65R, D84Y, and C125S,

d) T3A, H16A, L19N, M23T, E61R, P65R, D84Y, E95Q, and C125S,

e) T3A, H16A, E61R, P65R, D84Y, C125S, and S127D,

f) T3A, H16A, M23T, E61R, P65R, D84Y, E95Q, C125S, and S127D,

g) T3A, H16A, L19N, E61R, P65R, D84Y, C125S, and S127D, or

h) T3A, H16A, L19N, M23T, E61R, P65R, D84Y, E95Q, C125S, and S127D.

In any of the embodiments described herein, the modified IL-2 may be a modified human IL-2. In various embodiments, the amino acid positions of the substitutions correspond to the amino acid positions in SEQ ID NO: 1.

In some embodiments, the modified IL-2 comprises an amino acid sequence at least 90%, 91%, 92%, 93%, 94%, 95%, 96%, 97%, 98%, or 99% identical to SEQ ID NO: 84, and including amino acid substitutions corresponding to the substitutions in an amino acid sequence selected from SEQ ID NOs: 105-277 listed in the Description column of the Table of Certain Sequences. A corresponding substitution means the same amino acid when the two sequences are aligned. For example, SEQ ID NO: 106 comprises substitutions T3A, E61R, P65R, and C125S. If SEQ ID NO: 84 and SEQ ID NO: 106 are aligned, position E61 in SEQ ID NO: 106 corresponds to sequential position E51 in SEQ ID NO: 84. T3 of SEQ ID NO: 106 has no corresponding position in SEQ ID NO: 84, because the corresponding amino acid is not present. Therefore, SEQ ID NO: 84 comprising the substitutions of SEQ ID NO: 106 at positions corresponding to those of SEQ ID NO: 106 comprises mutations corresponding to E61R, P65R, and C125S, which in SEQ ID NO: 84 would be E51R, P55R, and C115S, respectively. In some embodiments, the modified IL-2 comprises an amino acid sequence at least 90%, 91%, 92%, 93%, 94%, 95%, 96%, 97%, 98%, or 99% identical to SEQ ID NO: 84, and including a substitution at at least one position corresponding to a position selected from L19, Q22, R38, E61, N88, R120, T123, Q126, S127, I129, and S130 of SEQ ID NO: 1.

In some embodiments, the modified IL-2 comprises an amino acid sequence at least 90%, 91%, 92%, 93%, 94%, 95%, 96%, 97%, 98%, 99%, or 100% identical to an amino acid sequence selected from SEQ ID NOs: 105-290, and including the substitutions indicated in the Description in the Table of Certain Sequences for the respective amino acid sequence. In some embodiments, the modified IL-2 comprises an amino acid sequence at least 90%, 91%, 92%, 93%, 94%, 95%, 96%, 97%, 98%, 99%, or 100% identical to an amino acid sequence selected from SEQ ID NOs: 105-290, and including a substitution at at least one position selected from L19, Q22, R38, E61, N88, R120, T123, Q126, S127, I129, and S130. In some embodiments, the modified IL-2 comprises an amino acid sequence selected from SEQ ID NOs: 105-290. In some embodiments, the modified IL-2 comprises an amino acid sequence selected from SEQ ID NOs: 270-277.

In some embodiments, a modified IL-2 containing polypeptide comprises at least one antigen binding domain that binds a T cell or natural killer cell antigen and an Fc region. In some embodiments, a modified IL-2 containing polypeptide provided herein comprises one, two, three, or four antigen binding domains and an Fc region. In some embodiments, an Fc region mediates dimerization of the modified IL-2 containing polypeptide at physiological conditions such that a dimer is formed that doubles the number of antigen binding sites. For example, a modified IL-2 containing polypeptide comprising three antigen binding domains and an Fc region is trivalent as a monomer, but at physiological conditions, the Fc region may mediate dimerization, such that the modified IL-2 containing polypeptide exists as a hexavalent dimer under such conditions.

In various embodiments, a polypeptide comprising a modified IL-2 comprises a sequence selected from SEQ ID NOs: 105-290. In various embodiments, a polypeptide comprising a modified IL-2 comprises a sequences selected from SEQ ID NO: 270-277. In some embodiments, the polypeptide further comprises an antigen binding domain. In some embodiments, the antigen binding domain is humanized.

In some embodiments, the at least one antigen binding domain is a natural or native cognate binding partner, an Anticalin (engineered lipocalin), a Darpin, a Fynomer, a Centyrin (engineered fibroneticin III domain), a cystine-knot domain, an Affilin, an Affibody, or an engineered CH3 domain. In some embodiments, the natural cognate binding partner comprises a ligand or an extracellular domain, or binding fragment thereof, of the native cognate binding partner of the tumor associated antigen (TAA), or a variant thereof that exhibits binding activity to the TAA.

In some embodiments, the polypeptide comprising the modified IL-2 and at least one antigen binding domain enhances anti-tumor T cell responses or natural killer cell responses while avoiding Tregs, peripheral T cells, and endothelial cells. In some such embodiments, the at least one antigen binding domain targets the modified IL-2 to activated T cells. In some embodiments, the modified IL-2 binds and modulates an IL-2R only when the IL-2R is on the same cell as the antigen bound by the at least one antigen binding domain. In some embodiments, the modified IL-2 does not bind or activate an IL-2R when the IL-2R is on a different cell than the cell expressing the antigen bound by the at least one antigen binding domain.

In various embodiments, the antigen-binding domain binds to a protein selected from PD-1, CTLA-4, LAG3, TIM3, 4-1BB, OX40, GITR, CD8a, CD8b, CD4, NKp30, NKG2A, TIGIT, TGFβR1, TGFβR2, Fas, NKG2D, NKp46, PD-L1, CD107a, ICOS, TNFR2, CD16a, and γδTCR. In some embodiments, the polypeptide comprising a modified IL-2 comprises an antigen-binding domain of nivolumab (BMS; PD-1); pembrolizumab (Merck; PD-1); AMP-514 (Amplimmune; PD-1); TSR-042 (Tesaro/AnaptysBio, ANB-011; PD-1); STI-A1110 (Sorrento Therapeutics; PD-1), ipilimumab (BMS; CTLA-4); tremelimumab (AstraZeneca, CTLA-4); urelumab (BMS, 4-1BB); utomilumab (Pfizer, 4-1BB); atezolizumab (Roche, PD-L1), durvalumab (AstraZeneca, PD-L1); monalizumab (NKG2A, Innate Pharma and AstraZeneca); BMS-986016 (Bristo-Meyers Squibb, LAG-3).

In some embodiments, the polypeptide comprises at least one antigen binding domain that specifically binds to PD-1. In some embodiments, the polypeptide comprises at least one antigen binding domain that specifically binds to LAG3. In some embodiments, the polypeptide comprises at least one antigen binding domain that specifically binds to NKp46. In some embodiments, the polypeptide comprises at least one antigen binding domain that specifically binds to NKG2D. In some embodiments, the polypeptide comprises at least one antigen binding domain that specifically binds to CD8a.

In some embodiments, an antigen binding domain may be humanized. Polypeptides comprising humanized antigen binding domains (such as VHH-containing polypeptides) are useful as therapeutic molecules because humanized antigen binding domains and humanized antibodies reduce or eliminate the human immune response to non-human antibodies, which can result in an immune response to an antibody therapeutic, and decreased effectiveness of the therapeutic. Generally, a humanized antigen binding domain or humanized antibody comprises one or more variable domains in which CDRs, (or portions thereof) are derived from a non-human antibody, and FRs (or portions thereof) are derived from human antibody sequences. A humanized antigen binding domain or humanized antibody optionally will also comprise at least a portion of a human constant region. In some embodiments, some FR residues in a humanized antigen binding domain or humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008)Front. Biosci. 13: 1619-1633, and are further described, for example, in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008)Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271:22611-22618). Typically, the FR regions of a VHH are replaced with human FR regions to make a humanized VHH. In some embodiments, certain FR residues of the human FR are replaced in order to improve one or more properties of the humanized VHH. VHH domains with such replaced residues are still referred to herein as “humanized.”

In various embodiments, an Fc region included in a modified IL-2 containing polypeptide is a human Fc region, or is derived from a human Fc region.

In some embodiments, an Fc region included in a modified IL-2 containing polypeptide is derived from a human Fc region, and comprises a three amino acid deletion in the lower hinge corresponding to IgG1 E233, L234, and L235, herein referred to as “Fc xELL” Fc xELL polypeptides do not engage FcγRs and thus are referred to as “effector silent” or “effector null”, however in some embodiments, xELL Fc regions bind FcRn and therefore have extended half-life and transcytosis associated with FcRn mediated recycling.

In some embodiments, the Fc region included in a modified IL-2 containing polypeptide is derived from a human Fc region and comprises mutations M252Y and M428V, herein referred to as “Fc-YV”. In some embodiments, the Fc region included in a modified IL-2 containing polypeptide is derived from a human Fc region and comprises mutations M252Y and M428L, herein referred to as “Fc-YL”. In some embodiments, such mutations enhance binding to FcRn at the acidic pH of the endosome (near 6.5), while losing detectable binding at neutral pH (about 7.2), allowing for enhanced FcRn mediated recycling and extended half-life.

In some embodiments, the Fc region included in a modified IL-2 containing polypeptide herein is derived from a human Fc region and comprises mutations designed for heterodimerization, herein referred to as “knob” and “hole”. In some embodiments, the “knob” Fc region comprises the mutation T366W. In some embodiments, the “hole” Fc region comprises mutations T366S, L368A, and Y407V. In some embodiments, Fc regions used for heterodimerization comprise additional mutations, such as the mutation S354C on a first member of a heterodimeric Fc pair that forms an asymmetric disulfide with a corresponding mutation Y349C on the second member of a heterodimeric Fc pair. In some embodiments, one member of a heterodimeric Fc pair comprises the modification H435R or H435K to prevent protein A binding while maintaining FcRn binding. In some embodiments, one member of a heterodimeric Fc pair comprises the modification H435R or H435K, while the second member of the heterodimeric Fc pair is not modified at H435. In various embodiments, the hole Fc region comprises the modification H435R or H435K (referred to as “hole-R” in some instances when the modification is H435R), while the knob Fc region does not. In some instances, the hole-R mutation improves purification of the heterodimer over homodimeric hole Fc regions that may be present.

Nonlimiting exemplary Fc regions that may be used in a modified IL-2 containing polypeptide include Fc regions comprising the amino acid sequences of SEQ ID NOs: 47-83, 292, and 293.

In some embodiments, a modified IL-2 containing polypeptide that comprises at least one antigen binding domain and an Fc region comprises an amino acid sequence selected from SEQ ID NOs: 105-290 and an Fc region fused to the C-terminus of that amino acid sequence. In some embodiments, a modified IL-2 containing polypeptide that comprises at least one antigen binding domain and an Fc region comprises an amino acid sequence selected from SEQ ID NOs: 270-277 and an Fc region fused to the C-terminus of that amino acid sequence. In some embodiments, a modified IL-2 containing polypeptide that comprises at least one antigen binding domain and an Fc region comprises an amino acid sequence selected from SEQ ID NOs: 105-290 and an Fc region fused to the N-terminus of that amino acid sequence. In some embodiments, a modified IL-2 containing polypeptide that comprises at least one antigen binding domain and an Fc region comprises an amino acid sequence selected from SEQ ID NOs: 270-277 and an Fc region fused to the N-terminus of that amino acid sequence. In some embodiments, a modified IL-2 containing polypeptide that comprises at least one antigen binding domain and an Fc region comprises an amino acid sequence selected from SEQ ID NOs: 105-290 and an amino acid sequence selected from SEQ ID NOs: 47-83, 292, and 293. In some embodiments, a modified IL-2 containing polypeptide that comprises at least one antigen binding domain and an Fc region comprises an amino acid sequence selected from SEQ ID NOs: 105-290 and an amino acid sequence selected from SEQ ID NOs: 48, 64, 292, and 293. In some embodiments, a modified IL-2 containing polypeptide that comprises at least one antigen binding domain and an Fc region comprises an amino acid sequence selected from SEQ ID NOs: 270-277 and an amino acid sequence selected from SEQ ID NOs: 48, 64, 292, and 293. In some embodiments the polypeptide comprises an amino acid sequences selected from SEQ ID NOs: 105-290 and an antigen binding domain that binds an antigen expressed on a T cell or natural killer cell. In some embodiments, the polypeptide comprises an amino acid sequence selected from SEQ ID NOs: 270-277 and an antigen binding domain that binds an antigen expressed on a T cell or natural killer cell.

### Exemplary Activities of Modified IL-2 Containing Polypeptides

In various embodiments, the modified IL-2 containing polypeptides provided herein are agonists of IL-2R activity. Agonist activity may be determined, in some embodiments, using the methods provided in the Examples herein, such as using 293F cells or similar cells. In some embodiments, the modified IL-2 containing polypeptides provided herein are agonists of IL-2R activity when targeted to T cells, but show little or no agonist activity in the absence of targeting. In some embodiments, the modified IL-2 containing polypeptides provided herein are agonists of IL-2R activity when targeted to NK cells and/or T cells, but show little or no agonist activity in the absence of targeting. In some embodiments, the modified IL-2 containing polypeptides that target T cells or NK cells comprise at least one antigen binding domain that specifically binds to an antigen expressed on T cells or NK cells.

In some embodiments, the modified IL-2 containing polypeptides provided herein increase proliferation of CD4⁺ and/or CD8⁺ T cells in vitro and/or in vivo. In some embodiments, the polypeptide increases CD4⁺ and/or CD8⁺ T cell proliferation in the presence of Treg cells. In some such embodiments, the CD4⁺ and/or CD8⁺ T cells are activated CD4⁺ and/or CD8⁺ T cells. In some embodiments, a modified IL-2 containing polypeptide provided herein increases activated CD4⁺ and/or CD8⁺ T cells proliferation in vitro. In some embodiments, the modified IL-2 containing polypeptide increases activated CD4⁺ and/or CD8⁺ T cells proliferation by at least 1.5-fold, at least 2-fold, at least 3-fold, or by at least 5-fold relative to CD4⁺ and/or CD8⁺ T cell proliferation in the absence of the polypeptide. In some embodiments, the polypeptide increases proliferation of activated CD4⁺ and/or CD8⁺ T cells by at least 1.5-fold, at least 2-fold, at least 3-fold, or by at least 5-fold and does not substantially increase the proliferation of resting CD4⁺ and/or CD8⁺ T cells, relative to the proliferation observed in the absence of the polypeptide.

In some embodiments, the modified IL-2 containing polypeptides provided herein increase proliferation of NK cells in vitro and/or in vivo. In some such embodiments, the NK cells are activated NK cells. In some embodiments, a modified IL-2 containing polypeptide provided herein increases activated NK cells proliferation in vitro. In some embodiments, the modified IL-2 containing polypeptide increases activated NK cells proliferation by at least 1.5-fold, at least 2-fold, at least 3-fold, or by at least 5-fold relative to NK cell proliferation in the absence of the polypeptide. In some embodiments, the polypeptide increases proliferation of activated NK cells by at least 1.5-fold, at least 2-fold, at least 3-fold, or by at least 5-fold and does not substantially increase the proliferation of resting NK cells, relative to the proliferation observed in the absence of the polypeptide.

The increase in proliferation of activated CD4⁺ and/or CD8⁺ T cells may be determined by any method in the art, such as for example, the methods provided in the Examples herein. A nonlimiting exemplary assay is as follows. CD4⁺ and/or CD8⁺ T cells may be isolated from one or more healthy human donors. The T cells are stained with CellTrace Violet (CTV) and activated with anti-CD3 antibody, contacted with a polypeptide comprising a modified IL-2, and then analyzed by FACS. Loss of CTV staining indicates proliferation. In some embodiments, an increase in CD4⁺ and/or CD8⁺ T cell proliferation is determined as an average from a set of experiments or from pooled T cells, such as by measuring proliferation of CD4⁺ and/or CD8⁺ T cells isolated from different healthy human donors. In some embodiments, an increase in CD4⁺ and/or CD8⁺ T cell proliferation is determined as an average from experiments carried out using T cells from at least five or at least ten different healthy donors, or from a pool of T cells from at least five or at least ten different healthy donors. In some embodiments, the modified IL-2 containing polypeptides provided herein increase proliferation of CD4⁺ and/or CD8⁺ T cells even in the presence of Treg cells.

In some embodiments, the modified IL-2 containing polypeptides provided herein increase CD71 expression on CD4+ and/or CD8+ T cells in vitro and/or in vivo. CD71 expression indicates T cell activation. In some embodiments, a modified IL-2 containing polypeptide provided herein increases CD71 expression on CD4⁺ and/or CD8⁺ T cells in vitro. In some embodiments, the modified IL-2 containing polypeptide increases CD71 expression on CD4⁺ and/or CD8⁺ T cells by at least 1.5-fold, at least 2-fold, at least 3-fold, or by at least 5-fold relative to CD71 expression in the absence of the polypeptide. In some embodiments, the polypeptide increases CD71 expression on activated CD4⁺ and/or CD8⁺ T cells by at least 1.5-fold, at least 2-fold, at least 3-fold, or by at least 5-fold and does not substantially increase CD71 expression on resting CD4⁺ and/or CD8⁺ T cells, relative to the CD71 expression observed in the absence of the polypeptide. In some embodiments, the polypeptide increases CD71 expression on CD4⁺ and/or CD8⁺ T cells in the presence of Treg cells.

The increase in CD71 expression on CD4⁺ and/or CD8⁺ T cells may be determined by any method in the art, such as for example, the methods provided in the Examples herein. A nonlimiting exemplary assay is as follows. CD4⁺ and/or CD8⁺ T cells may be isolated from one or more healthy human donors and stimulated with an anti-CD3 antibody, contacted with a modified IL-2 containing polypeptide, and then analyzed by FACS for CD71 expression. In some embodiments, an increase in CD71 expression on CD4⁺ and/or CD8⁺ T cells is determined as an average from a set of experiments or from pooled T cells, such as by measuring CD71 expression on CD4⁺ and/or CD8⁺ T cells isolated from different healthy human donors. In some embodiments, an increase in CD71 expression on CD4⁺ and/or CD8⁺ T cells is determined as an average from experiments carried out using T cells from at least five or at least ten different healthy donors, or from a pool of T cells from at least five or at least ten different healthy donors. In some embodiments, the modified IL-2 containing polypeptides provided herein increase CD71 expression on CD4⁺ and/or CD8⁺ T cells even in the presence of Treg cells.

In some embodiments, the modified IL-2 containing polypeptides provided herein increase pSTAT5 expression in CD4⁺ and/or CD8⁺ T cells in vitro and/or in vivo.

pSTAT5 expression indicates T cell activation. In some embodiments, a modified IL-2 containing polypeptide provided herein increases pSTAT5 expression in CD4⁺ and/or CD8⁺ T cells in vitro. In some embodiments, the modified IL-2 containing polypeptide increases pSTAT5 expression on CD4⁺ and/or CD8⁺ T cells by at least 1.5-fold, at least 2-fold, at least 3-fold, or by at least 5-fold relative to pSTAT5 expression in the absence of the polypeptide. In some embodiments, the polypeptide increases pSTAT5 expression on CD4⁺ and/or CD8⁺ T cells in the presence of Treg cells. The increase in pSTAT5 expression in CD4⁺ and/or CD8⁺ T cells may be determined by any method in the art, such as for example, the methods provided in the Examples herein. In some embodiments, the modified IL-2 containing polypeptides provided herein increase pSTAT5expression in CD4⁺ and/or CD8⁺ T cells even in the presence of Treg cells.

In some embodiments, the modified IL-2 containing polypeptides provided herein increase pSTAT5 expression in NK cells in vitro and/or in vivo. pSTAT5 expression indicates NK cell activation. In some embodiments, a modified IL-2 containing polypeptide provided herein increases pSTAT5 expression in NK cells in vitro. In some embodiments, the modified IL-2 containing polypeptide increases pSTAT5 expression on NK cells by at least 1.5-fold, at least 2-fold, at least 3-fold, or by at least 5-fold relative to pSTAT5 expression in the absence of the polypeptide. In some embodiments, the polypeptide increases pSTAT5 expression in NK cells in the presence of Treg cells. The increase in pSTAT5 expression in NK cells may be determined by any method in the art, such as for example, the methods provided in the Examples herein.

In some embodiments, the modified IL-2 containing polypeptides provided herein reduce or attenuate suppressive activity of regulatory T cells (Tregs). In some embodiments, the modified IL-2 containing polypeptides reduce Treg suppressive activity on CD4⁺ and/or CD8⁺ T cells by at least 10%, at least 20%, at least 30%, or by at least 50%. The decrease in Treg suppressive activity on conventional CD4⁺ and/or CD8⁺ T cells may be determined by any method in the art, such as for example, the methods provided in the Examples herein. A nonlimiting exemplary assay is as follows. Tregs and CD4⁺ T cells are differentially labeled with fluorescent proliferative cellular dyes following isolation from healthy human donor PBMCs. CD4⁺ T cells are stimulated with an anti-CD3 antibody, while Treg cells are incubated in the presence of a modified IL-2 containing polypeptide provided herein. The two T cell populations are co-cultured for 3 days and proliferation and activation of CD4⁺ T cells is monitored by flow cytometry. In some embodiments, the modified IL-2 containing polypeptides provided herein increase CD4⁺ and/or CD8⁺ T cell activation and proliferation in the presence of Treg cells, for example, compared to CD4⁺ and/or CD8⁺ T cell activation and proliferation in the presence of Treg cells but the absence of a modified IL-2 containing polypeptide provided herein.

### Polypeptide Expression and Production

Nucleic acid molecules comprising polynucleotides that encode a modified IL-2 containing binding polypeptide are provided. Thus, in various embodiments, nucleic acid molecules are provided that encode a polypeptide comprising a modified IL-2. In some embodiments, the nucleic acid molecule encodes a modified IL-2 and at least one antigen binding domain. In various embodiments, the nucleic acid molecule encodes a modified IL-2 and an Fc region and, optionally, at least one antigen binding domain. In some embodiments, the Fc region comprises mutations designed for heterodimerization, such as “knob” or “hole” mutations. In some embodiments, a nucleic acid molecule is provided that encodes a modified IL-2 containing polypeptide that comprises a modified IL-2, at least one antigen binding domain, and an Fc region, wherein the Fc region is fused to the C-terminus of the at least one antigen binding domain, and the modified IL-2 is fused to the C-terminus of the Fc region. In any of the foregoing embodiments, the nucleic acid molecule may also encode a leader sequence that directs secretion of the modified IL-2 containing polypeptide, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode the modified IL-2 containing polypeptides described herein are provided. Such vectors include, but are not limited to, DNA vectors, phage vectors, viral vectors, retroviral vectors, etc. In some embodiments, a vector is selected that is optimized for expression of polypeptides in a desired cell type, such as 293F, CHO, or CHO-derived cells, or in NSO cells. Exemplary such vectors are described, for example, in Running Deer et al., Biotechnol. Prog. 20:880-889 (2004).

In some embodiments, a modified IL-2 containing polypeptide may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293F cells; CHO cells, including CHO-S, DG44. Lec13 CHO cells, and FUT8 CHO cells; PER.C6® cells (Crucell); and NSO cells. In some embodiments, the modified IL-2 containing polypeptides may be expressed in yeast. See, e.g., U.S. Publication No. US 2006/0270045 A1. In some embodiments, a particular eukaryotic host cell is selected based on its ability to make desired post-translational modifications to the polypeptide. For example, in some embodiments, CHO cells produce polypeptides that have a higher level of sialylation than the same polypeptide produced in 293F cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses a modified IL-2 containing polypeptide described herein is provided. The modified IL-2 containing polypeptides expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify a modified IL-2 containing polypeptide that comprises an Fc region. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

In some embodiments, the modified IL-2 containing polypeptide is produced in a cell-free system. Nonlimiting exemplary cell-free systems are described, for example, in Sitaraman et al., Methods Mot Biol. 498: 229-44 (2009); Spirin, Trends Biotechnol. 22: 538-45 (2004); Endo et al., Biotechnol. Adv. 21: 695-713 (2003).

In some embodiments, modified IL-2 containing polypeptides prepared by the methods described above are provided. In some embodiments, the modified IL-2 containing polypeptide is prepared in a host cell. In some embodiments, the modified IL-2 containing polypeptide is prepared in a cell-free system. In some embodiments, the modified IL-2 containing polypeptide is purified. In some embodiments, a cell culture media comprising a modified IL-2 containing polypeptide is provided.

In some embodiments, compositions comprising antibodies prepared by the methods described above are provided. In some embodiments, the composition comprises a modified IL-2 containing polypeptide prepared in a host cell. In some embodiments, the composition comprises a modified IL-2 containing polypeptide prepared in a cell-free system. In some embodiments, the composition comprises a purified modified IL-2 containing polypeptide.

### Exemplary Methods of Treating Diseases Using Modified IL-2 Containing Polypeptides

In some embodiments, methods of treating disease in an individual comprising administering a modified IL-2 containing polypeptide are provided. Such diseases include any disease that would benefit from increase proliferation and activation of CD4⁺ and/or CD8⁺ T cells. In some embodiments, methods for treating cancer in an individual are provided. The method comprises administering to the individual an effective amount of a modified IL-2 containing polypeptide provided herein. Such methods of treatment may be in humans or animals. In some embodiments, methods of treating humans are provided. Nonlimiting exemplary cancers that may be treated with modified IL-2 containing polypeptides provided herein include basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; and chronic myeloblastic leukemia.

The modified IL-2 containing polypeptides can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of a modified IL-2 containing polypeptides is administered to a subject one or more times. In some embodiments, an effective dose of a modified IL-2 containing polypeptide is administered to the subject daily, semiweekly, weekly, every two weeks, once a month, etc. An effective dose of a modified IL-2 containing polypeptide is administered to the subject at least once. In some embodiments, the effective dose of a modified IL-2 containing polypeptide may be administered multiple times, including multiple times over the course of at least a month, at least six months, or at least a year.

In some embodiments, pharmaceutical compositions comprising a modified IL-2 containing polypeptide are administered in an amount effective for treating (including prophylaxis of) cancer and/or increasing T cell proliferation. The therapeutically effective amount is typically dependent on the weight of the subject being treated, his or her physical or health condition, the extensiveness of the condition to be treated, or the age of the subject being treated. In general, polypeptides may be administered in an amount in the range of about 0.05 mg/kg body weight to about 100 mg/kg body weight per dose. In some embodiments, polypeptides may be administered in an amount in the range of about 10 μg/kg body weight to about 100 mg/kg body weight per dose. In some embodiments, polypeptides may be administered in an amount in the range of about 50 μg/kg body weight to about 5 mg/kg body weight per dose. In some embodiments, polypeptides may be administered in an amount in the range of about 100 μg/kg body weight to about 10 mg/kg body weight per dose. In some embodiments, polypeptides may be administered in an amount in the range of about 100 μg/kg body weight to about 20 mg/kg body weight per dose. In some embodiments, polypeptides may be administered in an amount in the range of about 0.5 mg/kg body weight to about 20 mg/kg body weight per dose. In some embodiments, polypeptides may be administered in an amount in the range of about 0.5 mg/kg body weight to about 10 mg/kg body weight per dose. In some embodiments, polypeptides may be administered in an amount in the range of about 0.05 mg/kg body weight to about 20 mg/kg body weight per dose. In some embodiments, polypeptides may be administered in an amount in the range of about 0.05 mg/kg body weight to about 10 mg/kg body weight per dose. In some embodiments, polypeptides may be administered in an amount in the range of about 5 mg/kg body weight or lower, for example less than 4, less than 3, less than 2, or less than 1 mg/kg of the antibody.

In some embodiments, modified IL-2 containing polypeptides can be administered in vivo by various routes, including, but not limited to, intravenous, intra-arterial, parenteral, intraperitoneal or subcutaneous. The appropriate formulation and route of administration may be selected according to the intended application.

In some embodiments, a therapeutic treatment using a modified IL-2 containing polypeptide is achieved by increasing T cell proliferation and/or activation. In some embodiments, increasing T cell proliferation and/or activation inhibits growth of cancer.

### Pharmaceutical Compositions

In some embodiments, compositions comprising modified IL-2 containing polypeptides are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, a pharmaceutical composition comprises a modified IL-2 containing polypeptide at a concentration of at least 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 125 mg/mL, 150 mg/mL, 175 mg/mL, 200 mg/mL, 225 mg/mL, or 250 mg/mL.

### Combination Therapy

Modified IL-2 containing polypeptides can be administered alone or in combination with other modes of treatment, such as other anti-cancer agents. They can be provided before, substantially contemporaneous with, or after other modes of treatment (i.e., concurrently or sequentially). In some embodiments, the method of treatment described herein can further include administering: radiation therapy, chemotherapy, vaccination, targeted tumor therapy, CAR-T therapy, oncolytic virus therapy, cancer immunotherapy, cytokine therapy, surgical resection, chromatin modification, ablation, cryotherapy, an antisense agent against a tumor target, a siRNA agent against a tumor target, a microRNA agent against a tumor target or an anti-cancer/tumor agent, or a biologic, such as an antibody, cytokine, or receptor extracellular domain-Fc fusion.

In some embodiments, a modified IL-2 containing polypeptide provided herein is given concurrently with a second therapeutic agent, for example, a PD-1 antibody. Examples of PD-1 antibodies include nivolumab (BMS); pembrolizumab (Merck); AMP-514 (Amplimmune); TSR-042 (Tesaro/AnaptysBio, ANB-011); STI-A1110 (Sorrento Therapeutics); and other agents that are directed against programmed death-1 (PD-1).

In some embodiments, a modified IL-2 containing polypeptide provided herein is given concurrently with a second therapeutic agent, for example, a PD-L1 therapy. Examples of PD-L1 therapies include pidilizumab (CureTech, CT-011); durvalumab (Medimmune/AstraZeneca); atezolizumab (Genentech/Roche); avelumab (Pfizer); AMP-224 (Amplimmune); BMS-936559 (Bristol-Myers Squibb); STI-A1010 (Sorrento Therapeutics); and other agents directed against programmed dealth-1 ligand (PD-L1).

In some embodiments, a modified IL-2 containing polypeptide provided herein is given concurrently with CAR-T (chimeric antigen receptor T cell) therapy, oncolytic virus therapy, cytokine therapy, and/or agents that target other checkpoint molecules, such as VISTA, gpNMB, B7H3, B7H4, HHLA2, CD73, CTLA4, TIGIT, etc.

### Nonlimiting Exemplary Methods of Diagnosis and Treatment

In some embodiments, the methods described herein are useful for evaluating a subject and/or a specimen from a subject (e.g. a cancer patient). In some embodiments, evaluation is one or more of diagnosis, prognosis, and/or response to treatment.

In some embodiments, the methods described herein comprise evaluating a presence, absence, or level of a protein. In some embodiments, the methods described herein comprise evaluating a presence, absence, or level of expression of a nucleic acid. The compositions described herein may be used for these measurements. For example, in some embodiments, the methods described herein comprise contacting a specimen of the tumor or cells cultured from the tumor with a therapeutic agent as described herein.

In some embodiments, the evaluation may direct treatment (including treatment with the polypeptides described herein). In some embodiments, the evaluation may direct the use or withholding of adjuvant therapy after resection. Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease. In some embodiments, the polypeptides are used as an adjuvant therapy in the treatment of a cancer. In some embodiments, the antibodies are used as the sole adjuvant therapy in the treatment of a cancer. In some embodiments, the antibodies described herein are withheld as an adjuvant therapy in the treatment of a cancer. For example, if a patient is unlikely to respond to an antibody described herein or will have a minimal response, treatment may not be administered in the interest of quality of life and to avoid unnecessary toxicity from ineffective chemotherapies. In such cases, palliative care may be used.

In some embodiments the polypeptides are administered as a neoadjuvant therapy prior to resection. In some embodiments, neoadjuvant therapy refers to therapy to shrink and/or downgrade the tumor prior to any surgery. In some embodiments, neoadjuvant therapy means chemotherapy administered to cancer patients prior to surgery. In some embodiments, neoadjuvant therapy means a polypeptide is administered to cancer patients prior to surgery. Types of cancers for which neoadjuvant chemotherapy is commonly considered include, for example, breast, colorectal, ovarian, cervical, bladder, and lung. In some embodiments, the antibodies are used as a neoadjuvant therapy in the treatment of a cancer. In some embodiments, the use is prior to resection.

In some embodiments, the tumor microenvironment contemplated in the methods described herein is one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T cells; regulatory T cells; macrophages; neutrophils; and other immune cells located proximal to a tumor.

### Kits

Also provided are articles of manufacture and kits that include any of the modified IL-2 containing polypeptides as described herein, and suitable packaging. In some embodiments, the invention includes a kit with (i) a modified IL-2 containing polypeptide, and (ii) instructions for using the kit to administer the modified IL-2 containing polypeptide to an individual.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the antibodies generally include information as to dosage, dosing schedule, and route of administration for the intended treatment or industrial use. The kit may further comprise a description of selecting an individual suitable or treatment.

The containers may be unit doses, bulk packages (e.g., multi-dose packages) or sub-unit doses. For example, kits may also be provided that contain sufficient dosages of molecules disclosed herein to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of molecules and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous suspension of polypeptide.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: P65R Mutation of IL-2 Essentially Eliminates CD25 Binding

IL-2 mutants were designed to disrupt the CD25 interface through steric occlusion (P65R and P65E), and were tested for binding to 293F cells transiently transfected with one or more components of the IL-2 receptor (CD25, CD122, and/or CD132). The mutants were compared to IL-2-F42K, a mutant reported to have reduced affinity to CD25. Increasing concentrations of a fusion protein comprising wild type human IL-2 (SEQ ID NO: 32), IL-2-F42K (SEQ ID NO: 33), IL-2-P65R (SEQ ID NO: 35), or IL-2-P65E (SEQ ID NO: 34) fused to the N-terminus of a “knob” Fc and complexed with a “hole” Fc (SEQ ID NO: 44) were added to the transfected 293F cells and incubated at 4° C. for 45 minutes.

Binding was analyzed by flow cytometry, substantially as follows. Cells were washed once in 200 μL of FACS buffer (PBS, 2% FBS, 0.05% sodium azide) and cell pellets were resuspended in 100 μl of a surface marker staining solution (containing A647-conjugated anti-human Fcg secondary antibody at 1:300 dilution in FACS buffer). Cells were incubated for 45 minutes at 4° C. before the final wash, and analyzed on a flow cytometer. Cellular debris was excluded by FSC/SSC size exclusion, and dead cells were excluded based on their positive propidium iodide signal. Single cells were selected using FSC-A/FSC-H doublet and aggregate exclusion. Transiently transfected cells also expressed cytoplasmic EGFP, and cells that were FL1 positive were analyzed. Increasing MFI levels of anti-human secondary antibody indicated IL-2 binding. FlowJo software was used for analysis of the cell populations. Raw mean fluorescence intensities (“MFI”) for each marker were then exported and analyzed using Excel and GraphPad PRISM. Values were graphed, and titration curves were fitted to assess a dose-response relationship using the non-linear regression One-site—Total curve fit.

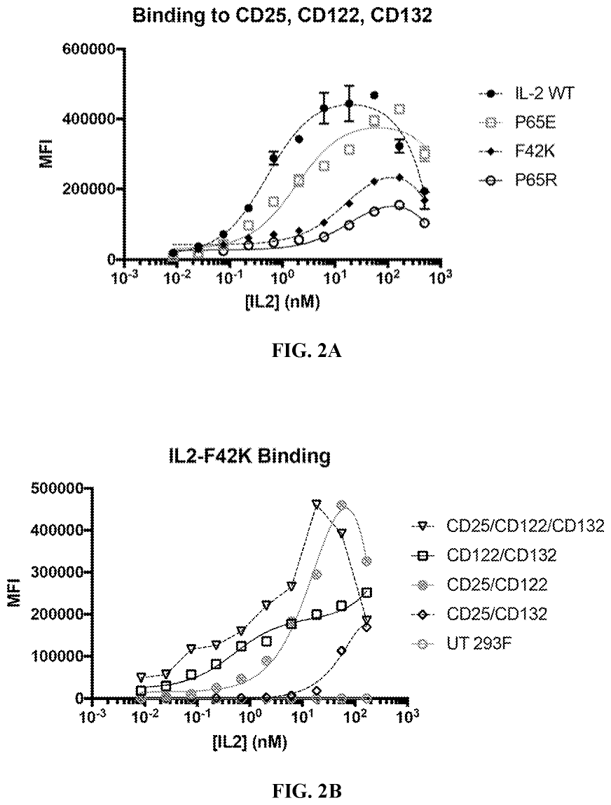

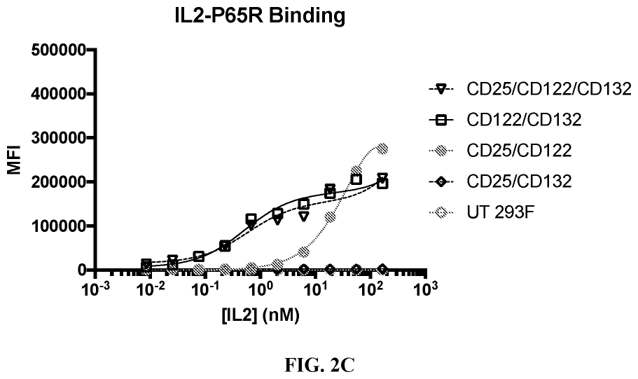

As shown in FIG. 2A-C, the fusion protein comprising the IL-2-P65E variant exhibited slightly reduced affinity for IL-2R relative to the fusion protein comprising wild type IL-2. The fusion protein comprising IL-2 F42K exhibited lower affinity than the fusion protein comprising IL-2-P65E, while the fusion protein comprising IL-2-P65R exhibited the lowest affinity for heterotrimeric IL-2R (FIG. 2A). Moreover, the fusion protein comprising IL-2-P65R exhibited no detectable binding to CD25/CD132 and only weakly bound CD25/CD122 (FIG. 2C), while the fusion protein comprising IL-2-F42K retained some affinity for CD25/CD132 (FIG. 2B) and bound CD25/CD122 with greater affinity than the fusion protein comprising IL-2-P65R (FIGS. 2B and 2C). Thus, IL-2 mutated at P65R significantly reduced binding to CD25 containing IL-2 receptors.

### Example 2: IL-2 Modifications that Reduce Affinity for CD122

As noted in Example 1, the P65R IL-2 mutation was designed to disrupt the CD25 interface through steric occlusion. In addition, IL-2 mutations were designed to reduce affinity for the CD122 interface through elimination of certain contact residue interactions (e.g., D84S, E95Q, M23A, H16A, and E15S). Single or double mutants were fused to the N-terminus of the “knob” half of a heterodimeric Fc (disulfide stabilized knob into hole comprising “hole” Fc SEQ ID NO: 44) for monovalent IL-2 binding to IL-2R. Relative binding affinities were assessed by transiently transfecting 293F cells with CD25 and CD122 (co-transfection with CD132 showed similar results, although the additional binding avidity reduced the differences in affinity observed). Bound IL-2-Fc fusion proteins were detected with fluorescent anti-human secondary antibody and analyzed by flow cytometry, substantially as described in Example 1.

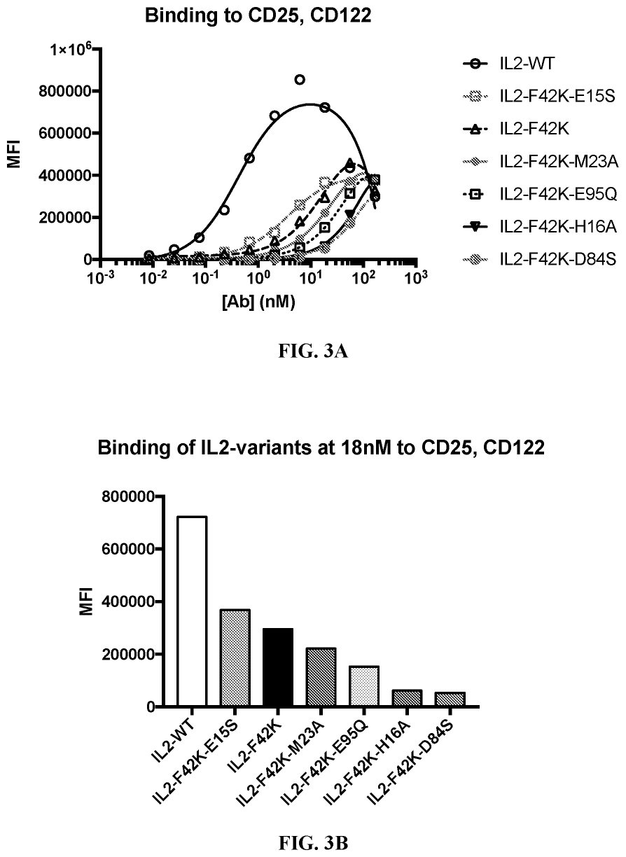

As shown in FIG. 3A-3B, all of the fusion proteins comprising double IL-2 mutants incorporating F42K with mutations in the CD122 interface (SEQ ID NOs: 36-39 and) showed reduced binding affinity for CD25/CD122 relative to those comprising the single mutant IL-2-F42K (SEQ ID NO: 33), with the exception of IL-2-F42K-E15S (SEQ ID NO: 85).

### Example 3: IL-2-RAS (P65R, H16A, and D84S) has Reduced Affinity for CD122 in the Context of Trimeric and Dimeric Forms of IL-2R

Mutations to reduce CD122 affinity described in Example 2 were combined with the P65R mutation to construct IL-2 double and triple mutants. The IL-2 mutants were fused to the N-terminus of the “knob” half of a heterodimeric Fc and paired with a “hole” Fc comprising SEQ ID NO: 44 for monovalent IL-2 binding to IL-2 receptor (IL-2R). Relative binding affinities of the resulting fusion proteins were assessed on 293F cells transiently transfected with IL-2R subunits, substantially as described in Example 1.

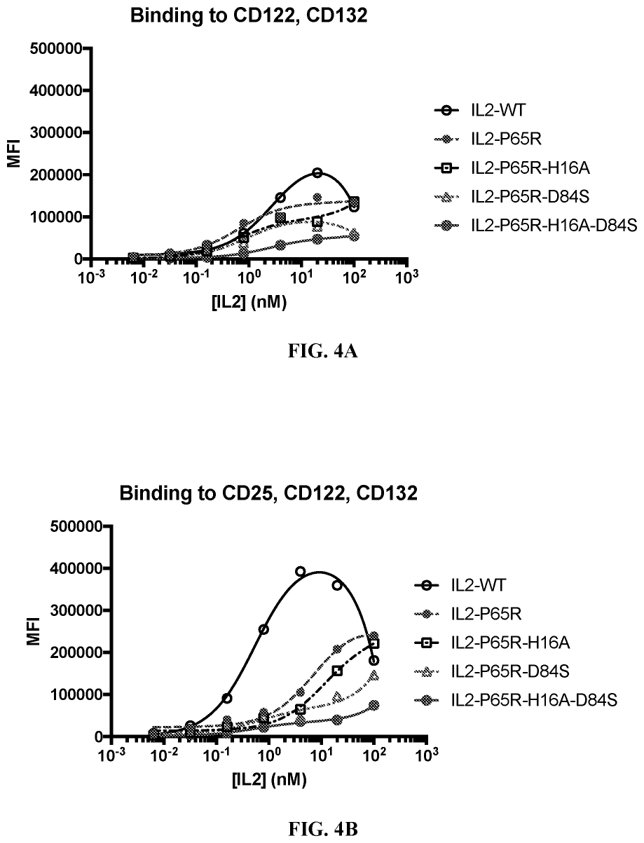

Relative to the fusion protein comprising wild type IL-2 (SEQ ID NO: 32), the fusion protein comprising IL-2-P65R-H16A (SEQ ID NO: 41) and the fusion protein comprising IL-2-P65R-D84S (SEQ ID NO: 42) had reduced affinity to both CD122/CD132 (heterodimeric IL-2R) (FIG. 4A) and the heterotrimeric IL-2R (FIG. 4B), while the affinity of the fusion protein comprising triple mutant, IL-2 P65R-H16A-D84S (“IL-2-RAS”, SEQ ID NO: 43), was even more attenuated (FIG. 4A-4B). The shifts in binding observed for these IL-2 mutants in both maximal binding and EC₅₀ suggest that these mutations reduced the on-rate (right shift in EC₅₀) and the off-rate (reduced maximal binding).

### Example 4. IL-2-RAS has Reduced Affinity for Resting T Cells and Pre-Activated T Cells

To isolate T cells, non-T cell populations were labeled with biotinylated anti-lineage marker antibodies against CD14, CD16, CD19, CD20, CD36, CD56, CD123, TCRγ/δ (BioLegend) for 20 minutes at room temperature. Non-T cell populations were then depleted by incubating for 20 minutes at room temperature with magnetic streptavidin particles (500 μl bead slurry plus 500 μl cell suspension per 100×10⁶, 2×8 minutes incubation on the magnet). The unbound cell supernatant contained isolated T cells.

Some of the isolated T cells (5.5×10⁶ in 3 mL) were activated by incubating in a 6-well plate pre-coated with 1 μg/ml anti-CD3 OKT3 antibody (BD Biosciences) for 2 days, then washed with PBS/2% FBS, and rested at 2×10⁶/mL in RPMI+10% FBS for 1 day. Resting or pre-activated T cells were used directly in the binding assay. Binding of a non-targeting VHH-Fc isotype control and fusion proteins comprising IL-2-RAS or wild type IL-2 fused to the C-terminus of a non-targeted VHH linked to a heterodimeric Fc to resting or pre-activated T cells was measured by flow cytometry, substantially as described in Example 1 except that the following secondary antibodies were used: AF647 anti-human Fc (1:1000), PI (1:2000), BV785-CD4 (1:300), APC/Fire-CD8 (1:500) and PE/Cy7-CD25 (1:100).

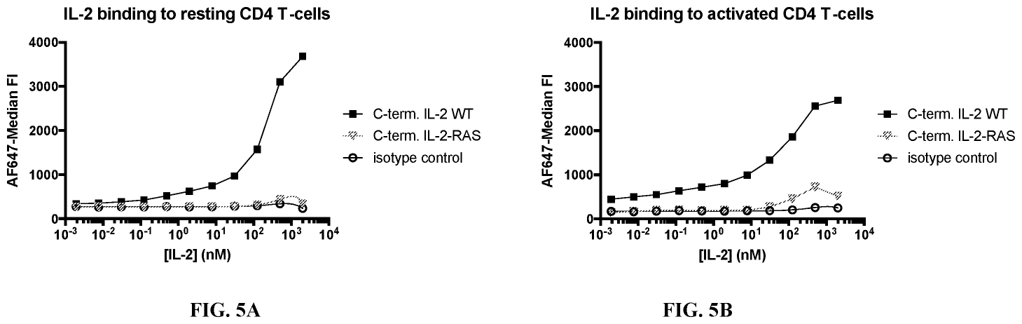

The non-targeted IL-2-RAS fusion protein (comprising SEQ ID NO: 46) bound with reduced affinity to resting (FIG. 5A) and pre-activated (FIG. 5B) T cells compared to the fusion protein comprising a non-targeting VHH domain and wild-type IL-2 (comprising SEQ ID NO: 45). An isotype control comprising no IL-2 did not bind resting or pre-activated T cells, as shown in FIGS. 5A and 5B.

### Example 5: IL-2-RAS has Reduced Affinity for Tregs

Regulatory T cells (“Tregs”) have high endogenous expression of CD25, as well as of CD122 and CD132, and are highly responsive to wild type IL-2. Binding to Tregs of a fusion protein comprising wild type IL-2 (comprising SEQ ID NO: 45) or the IL-2-RAS triple mutant (comprising SEQ ID NO: 46) fused to the C-terminus of the “knob” half of a heterodimeric Fc (disulfide stabilized knob into hole) of a non-targeted VHH was measured.

Tregs and CD4+T responder cells (Tresp) were enriched and isolated from fresh, healthy donor PBMCs by using an EasySep Human CD4⁺CD127^{low}CD25⁺ regulatory T cell isolation kit (Stemcell) following the manufacturer's instructions. Tregs were generated from naive CD4+ T cells via 7 day culture in ImmunoCult-XF T Cell Expansion Medium supplemented with rhTGF-B1, all-trans retinoic acid, CD3/CD28 T Cell Activator and IL-2.

In order to distinguish the two populations of cells, enriched Tregs and CD4⁺ responder T cells were labeled with the proliferative dyes CellTrace Violet (CTV) and CFSE, respectively, for 10 minutes at 37° C. After washing, Tregs and CD4⁺ T cells were resuspended to 1.5×10⁶ cells/ml in RPMI supplemented with 10% FBS and 1× antibiotic/antimycotic. Tregs were seeded in 50 μl volume yielding 75,000 Tregs/well in a 96-well round-bottom plate. Tregs were incubated overnight at 37° C. in the presence of 10 nM of IL-2-RAS by flow cytometry as described in Example 1.

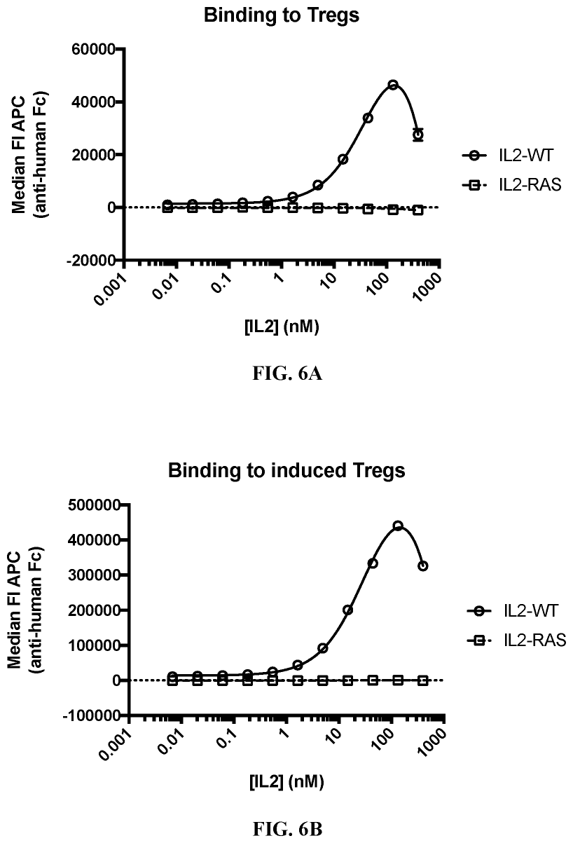

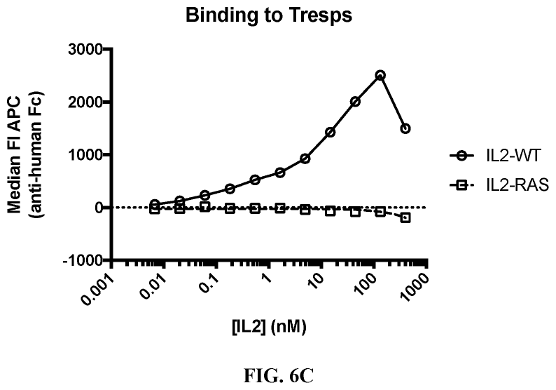

As shown in FIG. 6, in contrast to the fusion protein comprising wild type IL-2, the fusion protein comprising IL-2-RAS showed no observable binding to Tregs enriched from PBMCs (FIG. 6A), induced Tregs (FIG. 6B), or CD4+ Tresponders (FIG. 6C).

### Example 6: IL-2-RAS has Reduced Activity on Resting T Cells

T cells were isolated by magnetic bead separation, substantially as described in Example 4, labeled with CellTrace Violet (CTV), and treated with a fusion protein comprising wild type IL-2 (comprising SEQ ID NO: 45) or IL-2-RAS (comprising SEQ ID NO: 46) fused to the C-terminus of a non-targeted VHH linked to a heterodimeric Fc. Levels of CD4, CD8, CD71, and CTV were measured by flow cytometry. Proliferating T cells have reduced CTV levels.

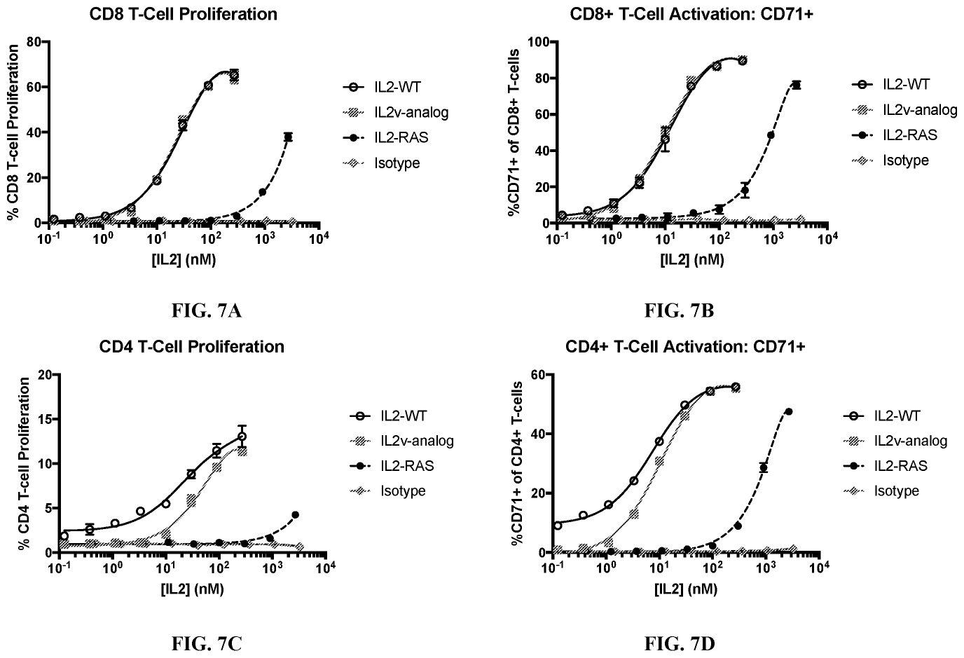

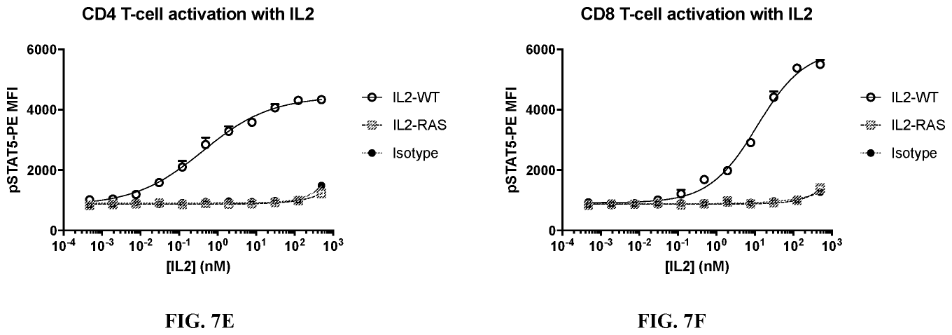

As shown in FIG. 7A and FIG. 7C, the concentration of the fusion protein comprising IL-2-RAS required to induce resting CD4+ and CD8+ T cell proliferation was over 100 times greater than the concentration of a fusion protein comprising wild type IL-2 or the concentration of a fusion protein comprising IL-2v-analog required to achieve the same induction of proliferation.

As shown in FIG. 7B and FIG. 7D, the concentration of the fusion protein comprising IL-2-RAS required to induce CD71 expression, a marker of T cell activation, on CD8+ and CD4+ T cells, was at least 100 times greater than the concentration of the fusion protein comprising wild type IL-2 or IL-2v-analog required to achieve the same induction of activation.

T cell activation can also be measured by phosphorylated STATS levels, which are increased in activated T cells. T cells were isolated by magnetic bead separation and treated with the fusion protein comprising wild-type IL-2 (comprising SEQ ID NO: 45) fused to the C-terminus of a non-targeted VHH comprising a heterodimeric Fc or the fusion protein comprising IL-2-RAS (comprising SEQ ID NO: 46) fused to the C-terminus of a non-targeted VHH comprising a heterodimeric Fc for 15 minutes. Cells were fixed with BD Cytofix/Cytoperm™ (BD Biosciences), permeabilized in 90% ice-cold methanol, and levels of phosphorylated STATS (“pSTAT5”) on CD4+ and CD8+ T cells were measured using flow cytometry using an anti-pSTAT5-PE antibody (1:70). Cells were co-stained with the following antibodies: anti-CD3-FITC (1:200), CD56-BV421 (1:100), CD4-BV785 (1:200), CD8-APC-Fire (1:300).

As shown in FIG. 7E and FIG. 7F, the non-targeted IL-2-RAS fusion protein achieved minimal phosphorylation of STATS in resting CD4+ and CD8+ T cells even at the highest concentration tested, while the non-targeted IL-2-wild type fusion protein induced STATS phosphorylation at a concentration more than 1000 times less than the highest concentration tested.

### Example 7: IL-2 Mutants have Reduced Activity on Tregs

Tregs were isolated from PBMCs using the EasySep™ Human CD4+CD127lowCD25+ Regulatory T cell Isolation Kit (Stemcell). Tregs were labeled with CellTrace Violet and plated at 0.15×10⁶ cells per well (96-well, U-bottom) in 100 μl of RPMI/10% FBS. Cells were combined with 100 μl of a fusion protein titration starting at 100 nM, titrated 1:4. Cells were incubated for 7 days. On day 7, proliferation and activation marker CD25 were measured by flow cytometry (Novocyte) substantially as described in Example 1, except that the following antibodies were used: BV785-CD4 (1:300), APC/Fire-CD8 (1:500) PE/Cy7-CD25 (1:100), PI (1:2000).

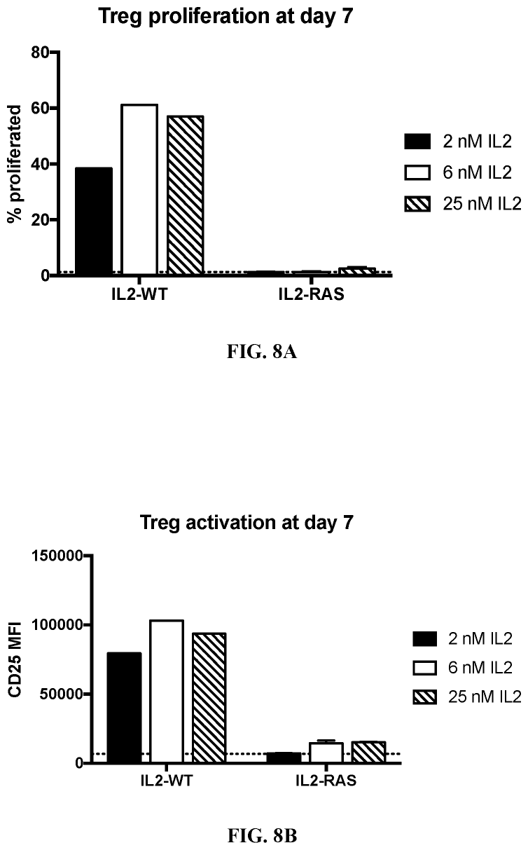

As shown in FIGS. 8A and 8B, fusion protein comprising wild type IL-2 (comprising SEQ ID NO: 45) fused to the C-terminus of a non-targeted VHH linked to heterodimeric Fc, but not fusion protein comprising IL-2-RAS in place of wild type IL-2 (comprising SEQ ID NO: 46), induced Treg proliferation and expression of the activation marker CD25.

### Example 8: Activated T Cells Expressing PD-1 are Stimulated by PD-1-Targeted IL RAS

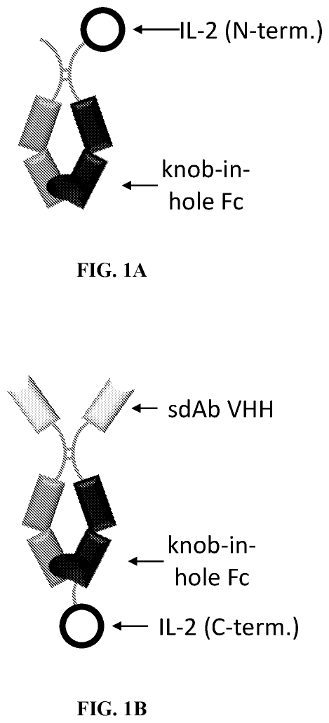

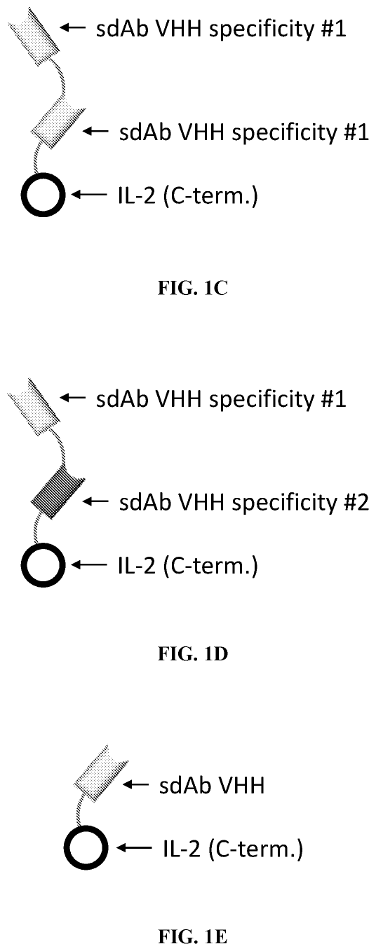

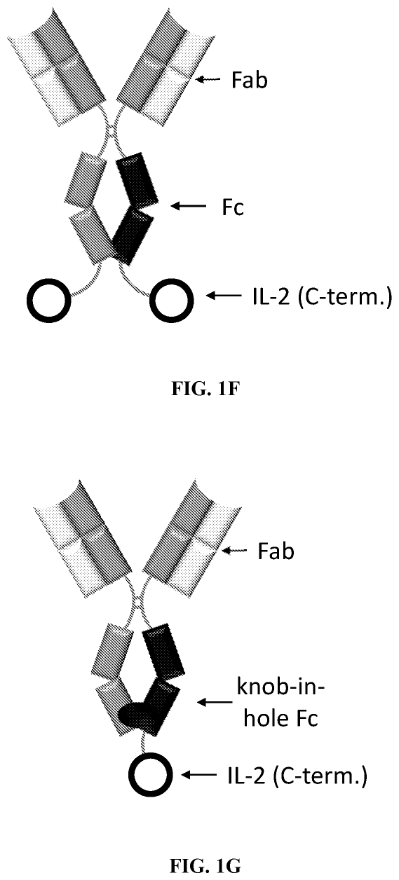

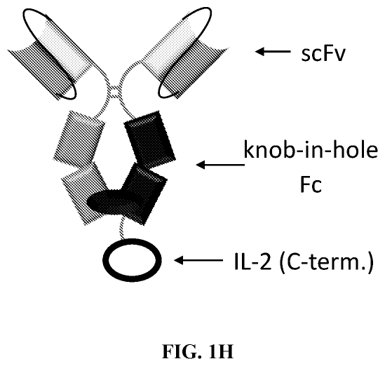

The ability to bind to and stimulate PD-1 expressing T cells was tested using pembrolizumab (an anti-PD-1 conventional antibody) and a fusion protein comprising a pembrolizumab analog and IL-2-RAS linked to the C-terminus of the heavy chain (see FIG. 1F).

Enriched T cells from a healthy donor were activated, substantially as described in Example 4. 6-well plates were coated overnight with 1 μg/ml OKT3 antibody at 4° C. The next day, plates were washed two times to remove unbound OKT3 antibody. Enriched T cells were thawed using CTL media and resuspended to 5.5×10⁶ cells/mL in complete RPMI and seeded in 3 mL per well in the coated plates. Two days later, the activated T cells were collected and washed once before plating in media without OKT3 antibody for 24 hours to rest. Cells were labeled with the proliferative dye CellTrace™ Violet (CTV). The T cells were counted, then resuspended to 2×10⁶ cells/mL. 100 μL of resuspended cells were seeded per well in a 96-well round-bottom plate. Pembrolizumab or a pembrolizumab analog-IL-2-RAS fusion was added starting at a final concentration of 100 nM and titrated 1:5. On day three, T cells were stained for 20 min at room temperature with the viability marker PI and the following fluorescently labeled antibodies: CD4-BV785, CD8-APC/Fire, CD25-PE/Cy7, CD71-FITC, and CD69-APC. The plate was read on the Novocyte flow cytometer substantially as described in Example 7 for measurement of proliferation and as in Example 1 for binding and data was exported into Excel for further analysis.

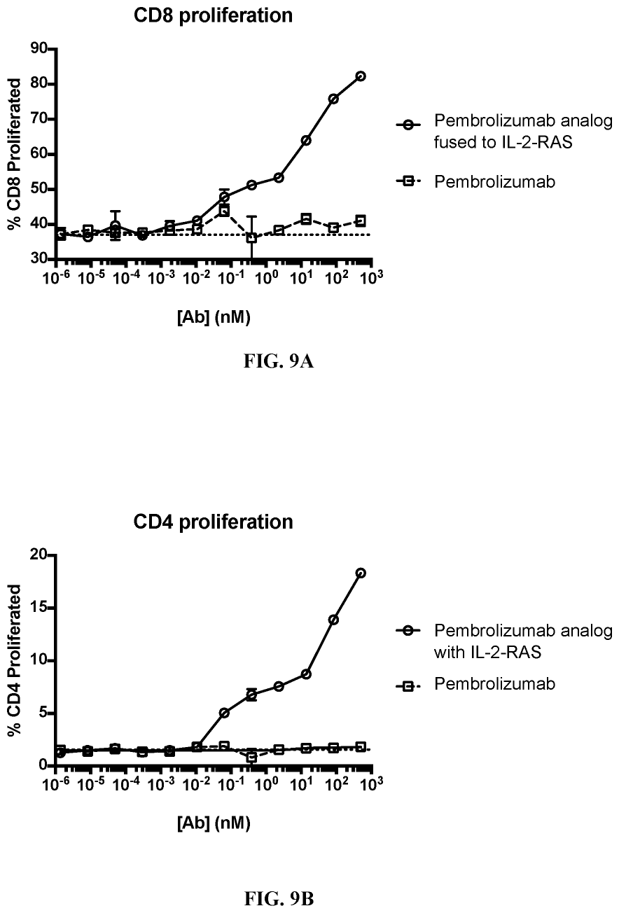

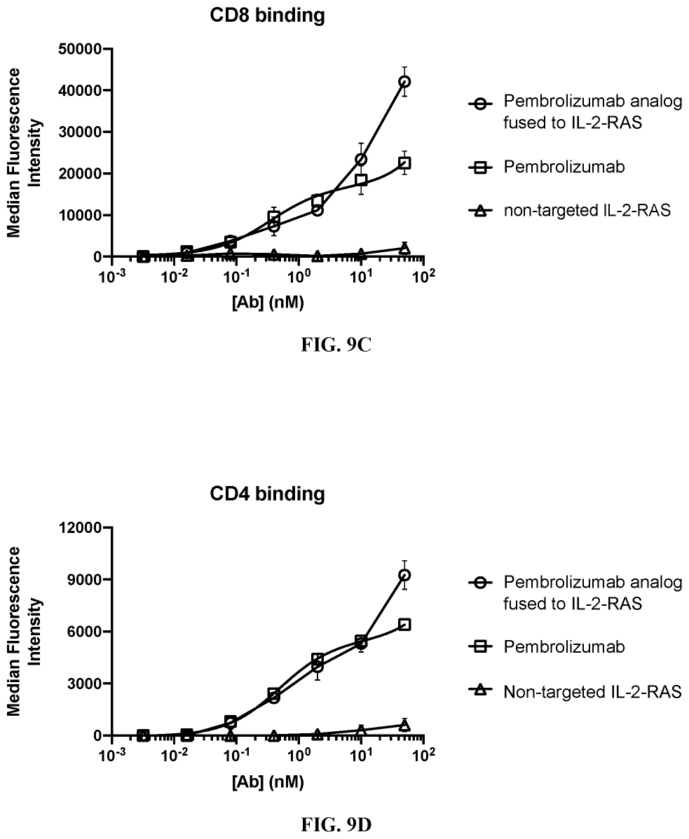

As shown in FIG. 9, the pembrolizumab analog-IL-2-RAS fusion protein stimulated CD8+ T cell proliferation (FIG. 9A) and CD4+ T cell proliferation (FIG. 9B), while pembrolizumab alone did not. Without intending to be bound by any particular theory, the biphasic nature of the observed proliferation may suggest that the activity at low concentration is due to PD-1-targeted activity and the increased activity at higher concentration is due to non-targeted activity. As shown in FIGS. 9C and 9D, both pembrolizumab and pembrolizumab analog-IL-2-RAS bound activated CD8+ and CD4+ T cells with similar affinities, except that additional binding was observed for the fusion protein comprising IL-2-RAS at the upper end of the dilution range above 10 nM, which may have been mediated by IL-2-RAS binding to IL-2R.

### Example 9: Pre-Blocking PD-1 on Activated T Cells Prevents Signaling by PD-1 Targeted IL-2-RAS

T cells were isolated and enriched from a healthy donor by magnetic bead separation, and incubated on plates coated with OKT3 antibody to activate them, substantially as described in Example 4. The cells were labeled with CTV. The pre-activated T cells were incubated with pembrolizumab, an anti-PD-1 antibody, to block PD-1 binding sites, or a non-targeted antibody as a control. The cells were then incubated with a fusion protein comprising IL-2-RAS fused to a pembrolizumab analog, or a fusion protein comprising IL-2-RAS fused to a non-targeting antibody as a control, for 3 days. The extent of IL-2 signaling was evaluated by measuring CD4+ and CD8+ T cell proliferation by flow cytometry, substantially as described in Example 7.

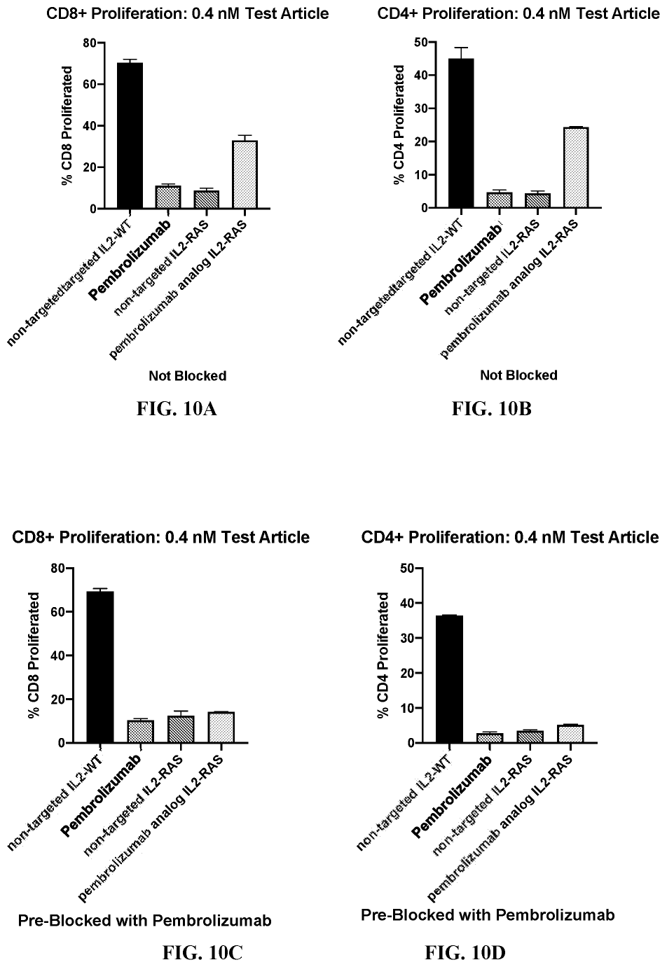

As shown in FIG. 10A-10D, wild type IL-2 induced robust proliferation of both CD8+ and CD4+ T cells, while CD4+ T cells and CD8+ T cells treated with pembrolizumab or the fusion protein comprising IL-2-RAS and the non-targeting antibody exhibited low levels of proliferation that was not affected by pre-blocking of PD-1. In contrast, both CD4+ T cells (FIGS. 10B and 10D) and CD8+ T cells (FIGS. 10A and 10C) treated with the fusion protein comprising IL-2-RAS and a pembrolizumab analog exhibited significant PD-1 dependent proliferation (FIGS. 10A and 10B), which was blocked by pre-incubation with an anti-PD-1 antibody (FIGS. 10C and 10D). Thus, a fusion protein comprising IL-2-RAS and an anti-PD-1 antibody activated T cells only when PD-1 was both expressed and accessible on the T cells.

### Example 10: PD-1-Targeted IL-2-RAS Overcomes Treg Suppression

CD4+T responder cells and Tregs were isolated as described in Example 5. The CD4+ responder cells were labeled with CTV, mixed with isolated Tregs at a ratio of 2:1 and activated with anti-CD3 beads (1 bead per 2 T cells). The resulting mixture was treated with a dilution series of a wild type IL-2 fused to the C-terminus of a non-targeted VHH, as shown in FIG. 1B, a fusion protein comprising IL-2-RAS fused to the C-terminus of a non-targeted VHH, as shown in FIG. 1B, or with a fusion protein comprising IL-2-RAS fused to an anti-PD-1 antibody (pembrolizumab analog-IL-2-RAS) for 7 days. Proliferation was measured by flow cytometry, substantially as described in Example 7.

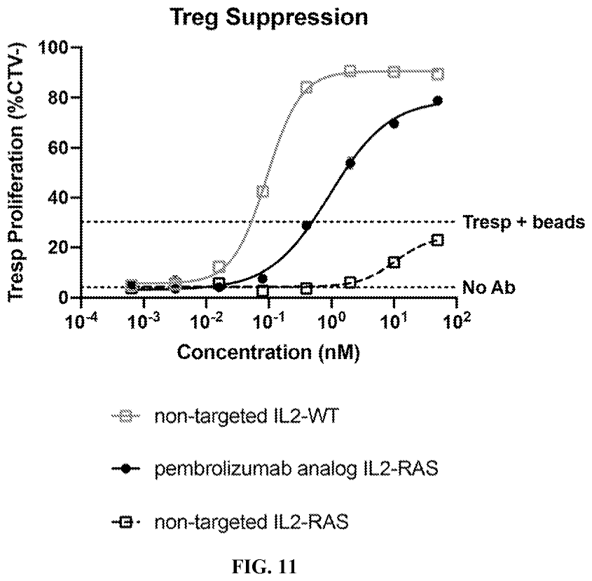

As shown in FIG. 11, Tresponder cells were suppressed by Tregs, but non-targeted wild type IL-2 and the fusion protein comprising IL-2-RAS and an anti-PD-1 antibody (pembrolizumab analog-IL-2-RAS) induced CD4+T responder cell proliferation despite the presence of Tregs. Treating cells with a fusion protein comprising IL-2-RAS and a non-targeted antibody did not rescue proliferation to a similar extent. The non-targeted IL-2-RAS was only able to counter the suppressive effects of Tregs on Tresponders at much higher concentrations than the PD-1 targeted IL-2-RAS fusion protein. Thus, PD-1-targeted IL-2-RAS overcame the suppressive effects of Tregs, and this activity was dependent on binding PD-1 expressed on the T cells.

### Example 11: PD-1 Targeted IL-2-RAS does not Signal in Trans

Beads are coated with 200 μg PD-1 antigen per 4×10⁸ beads according to the manufacturer's recommended coating procedure. In brief, beads are washed once in buffer 1 (0.1 M sodium phosphate buffer, pH 7.4-8.0) and then incubated in a tube rotator for 18 hours at room temperature in buffer 1 containing PD-1 antigen. Beads are then washed 4 times with buffer 2 (PBS, 0.1% BSA, 2 mM EDTA pH 7.4). Free tosyl groups are deactivated by incubation of beads for 4 hours at 37° C. in buffer 3 (0.2 M Tris, 0.1% BSA, pH 8.5). Beads are then washed once in buffer 2 and resuspended to a concentration of 400×10⁶ beads/mL.

Coated beads are incubated with a fusion protein comprising wild typeIL-2 or IL-2-RAS fused to an anti-PD-1 antibody and washed. The beads are then incubated with isolated resting T cells. IL-2 signaling is evaluated by measuring pSTAT5 levels via flow cytometry.

The fusion protein comprising wild typeIL-2 bound to the beads robustly activates CD8+ T cells and CD4+ T cells, while the fusion protein comprising IL-2-RAS bound to the beads has no activity up to the highest concentration tested on either CD4+ or CD8+ T cells. Thus, T cell targeting of IL-2-RAS is required for IL-2 signaling, and signaling of targeted IL-2-RAS does not occur in trans.

### Example 12: IL-2-RAS does not Signal in Trans

Dilution series of non-targeted wild type IL-2 and of non-targeted IL-2-RAS, starting at 1000 nM and diluted 1:4, were coated on assay plates, incubated overnight, and washed. T cells were added and incubated at 37° C. for 30 minutes. Activation of CD8+ and CD4+ T cells was measured by detecting phosphorylated STATS levels, substantially as described in Example 6.

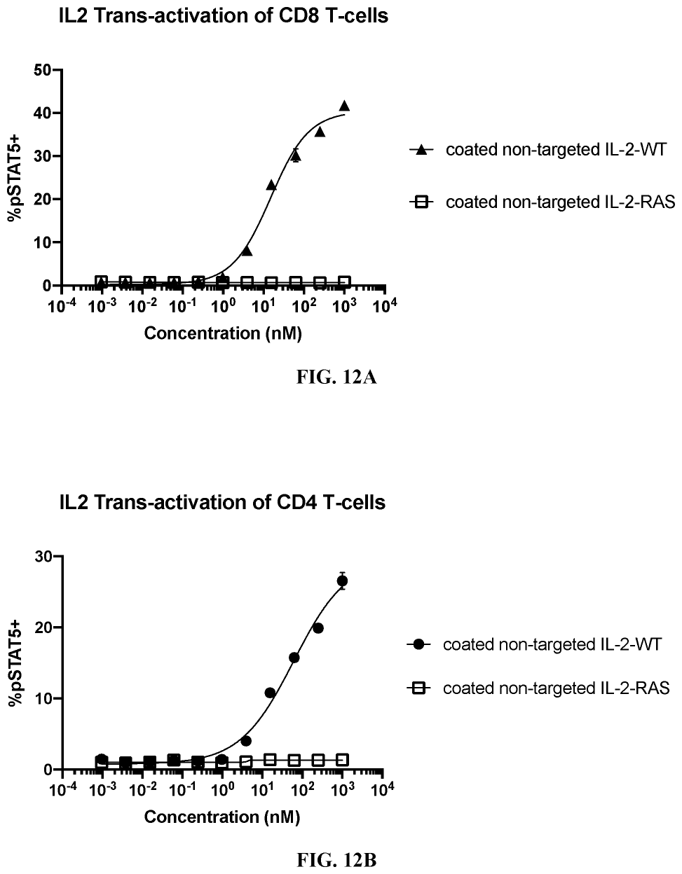

As shown in FIGS. 12A and 12B, CD8+ and CD4+ T cells were activated by wild type IL-2 in trans, as measured by pSTAT5 induction; however, non-targeted IL-2-RAS was unable to activate in trans. Without intending to be bound by any particular theory, the reduced affinities of IL-2-RAS for the IL-2 receptor may have prevented efficient binding and clustering of the IL-2R to induce downstream signaling. Thus, only targeted IL-2-RAS fusion proteins drive pSTAT5 signaling.

### Example 13: NKp46 Targeted IL-2-RAS Specifically Drives NK Cell Proliferation

The effects of a fusion protein comprising IL-2-RAS fused to the C-terminus of a heterodimeric scFv antibody targeting NKp46, as shown in FIG. 1H, fusion proteins comprising wild type IL-2 or IL-2-RAS fused to the C-terminus of a non-targeted VHH linked to a heterodimeric Fc, as shown in FIG. 1B, and the heterodimeric scFv antibody targeting NKp46 alone on NK cells, CD4+ T cells, and CD8+ T cells were determined.

Fresh PBMCs from a healthy donor were labeled with CellTrace™ Violet and plated in a 96-well round bottom plate at 200,000 cells/well. Dilutions of the fusion proteins and NKp46 scFv-Fc control were added to the plated cells and incubated at 37° C. for 7 days. On day 7, cell proliferation was measured, substantially as described in Example 7, except that the following antibodies were used: anti-CD3-BV785 (1:200), anti-CD56-APC (1:100), anti-CD4-PE (1:200), anti-CD8-APC-Fire (1:300) and PI (1:2000).

In addition, fresh PBMCs from a healthy donor were treated with the same fusions proteins or NKp46 scFv-Fc control, and incubated at 37° C. for 15 minutes. pSTAT5 levels in CD8+ T cells, CD4+ T cells, and NK cells (CD3−, CD56+) were measured by detecting phosphorylated STATS levels, substantially as described in Example 6.

Binding of the fusion proteins and the NKp46 scFV-Fc control to fresh PBMCs from a healthy donor was measured, substantially as described in Example 1, except that the following antibodies were used: anti-CD3-FITC (1:100), anti-CD56-BV421 (1:100), anti-CD4-BV785 (1:200), anti-CD8-APC-Fire (1:300), anti-human IgG-Alexa Fluor 647 (1:500), and PI (1:2000).

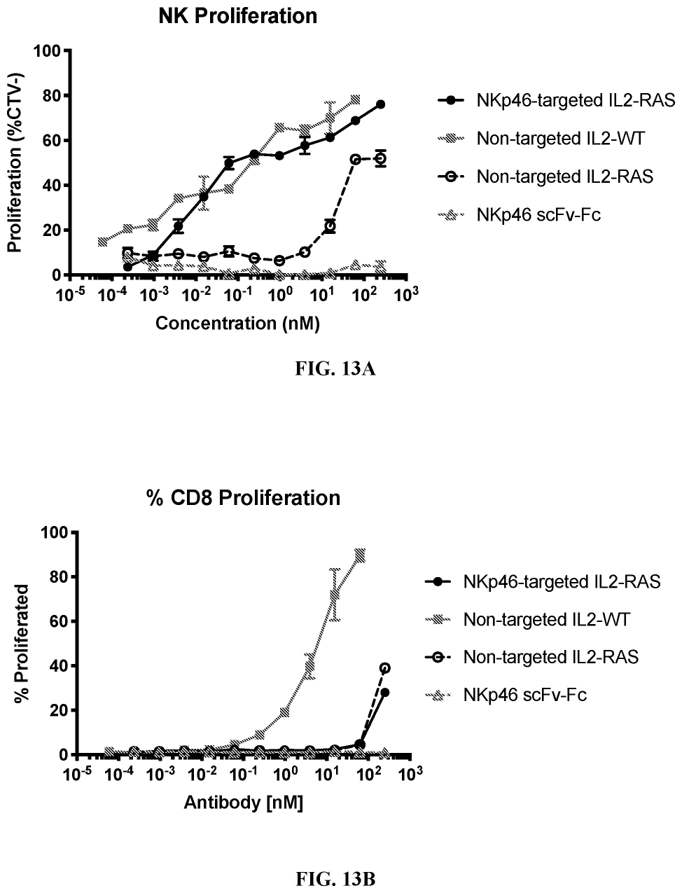

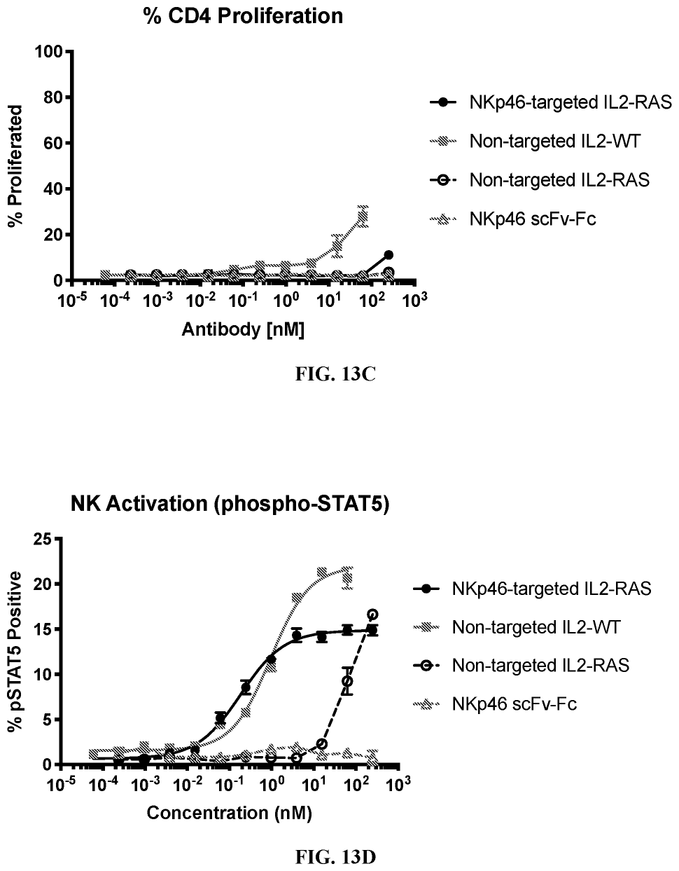

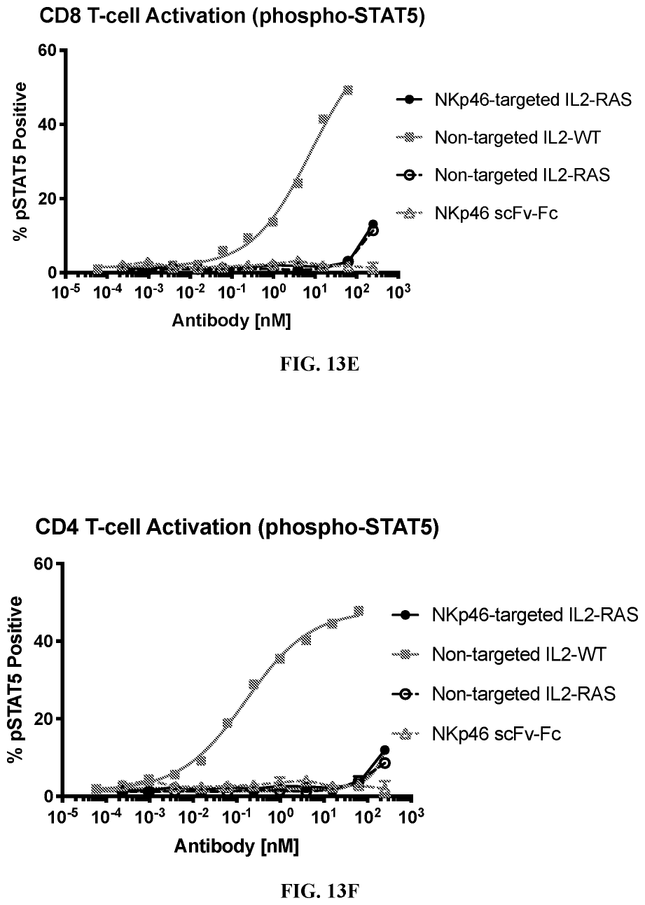

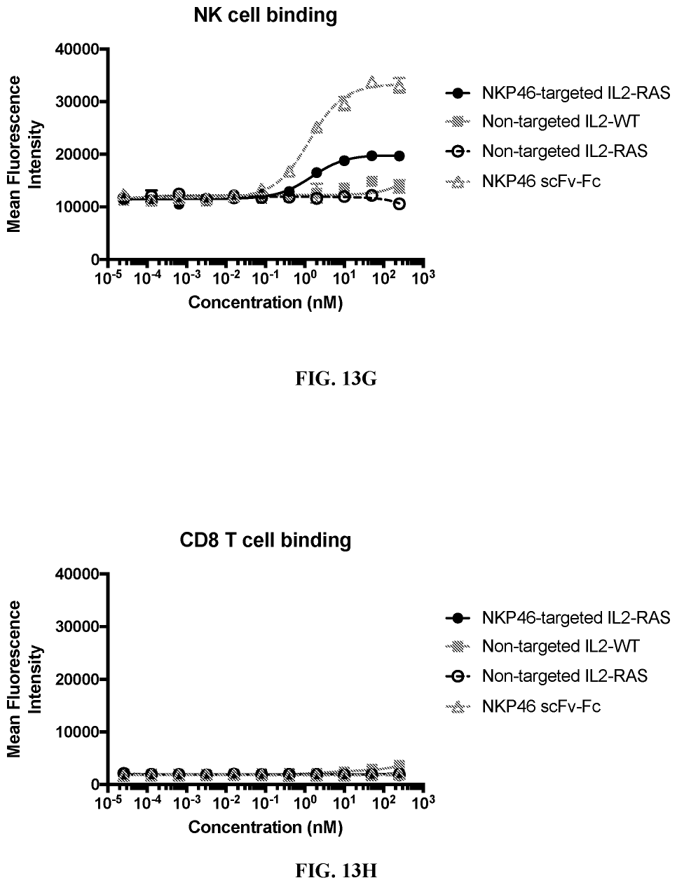

As shown in FIG. 13A-13I, NKp46-targeted IL-2-RAS potently activated NK cell proliferation and activation, while not affecting CD4+ or CD8+ T cells. In contrast, non-targeted wild type IL-2 drove proliferation and activation of all lymphocytes tested (NK, CD4+, and CD8+ T cells). Binding of the NKp46 scFV-Fc (without IL-2-RAS) did not drive NK proliferation or pSTAT5 induction. Thus, NKp46-targeted IL-2-RAS drove cis signaling of IL-2 on NK cells, but did not activate CD4+ or CD8+ T cells in trans.

### Example 14: LAG3 Targeted IL-2-RAS Stimulates Pre-Activated LAG3+ T-Cells

The effects on CD4+ T cells and CD8+ T cells of fusion proteins comprising IL-2-RAS fused to the C-terminus of an anti-LAG3 heterodimeric conventional antibody (MAb), as shown in FIG. 1G, fused to an anti-LAG3 VHH with an heterodimeric Fc as shown in FIG. 1B, fused to a non-targeted VHH, as shown in FIG. 1B, or a fusion protein comprising wild type IL-2 fused to the C-terminus of a non-targeted heterodimeric Fc, as shown in FIG. 1B, or a LAG3-targeted Mab (control), or a LAG3-targeted VHH-Fc (control) were assayed.

Enriched T cells from a healthy donor were stimulated for 48 hours with 1 μg/mL coated anti-CD3 (OKT3) and 10 μg/mL soluble anti-CD28, then allowed to rest for 24 hours. The pre-activated cells were labeled with CellTrace™ Violet and seeded at 200,000 cells/well. Dilutions of the fusion proteins and control proteins were added and incubated for 3 days. Proliferation and expression of activation markers CD25 and CD71 were measured, substantially as in Example 7, but with these additional antibodies: anti-CD25-FITC (1:100) and anti-CD71-PE/Cy7.

Stimulated CD8+ T cells upregulated LAG3 to 45% of CD8+ T cells, while CD4+ T cells upregulated LAG3 to 22% of CD4+ T cells. In contrast, non-stimulated T cells are close to 0% positive for LAG3 expression on either CD8+ or CD4+ T cells.

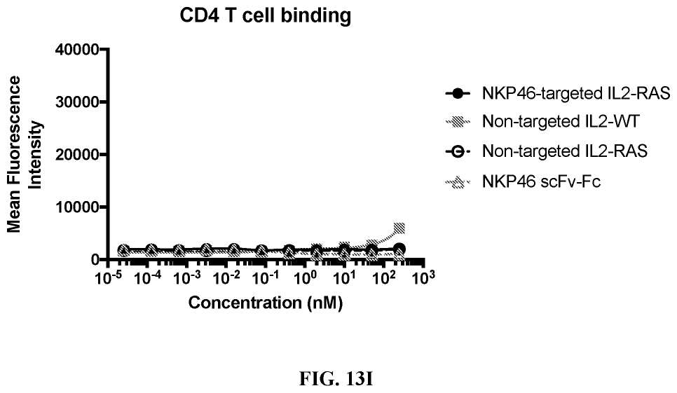

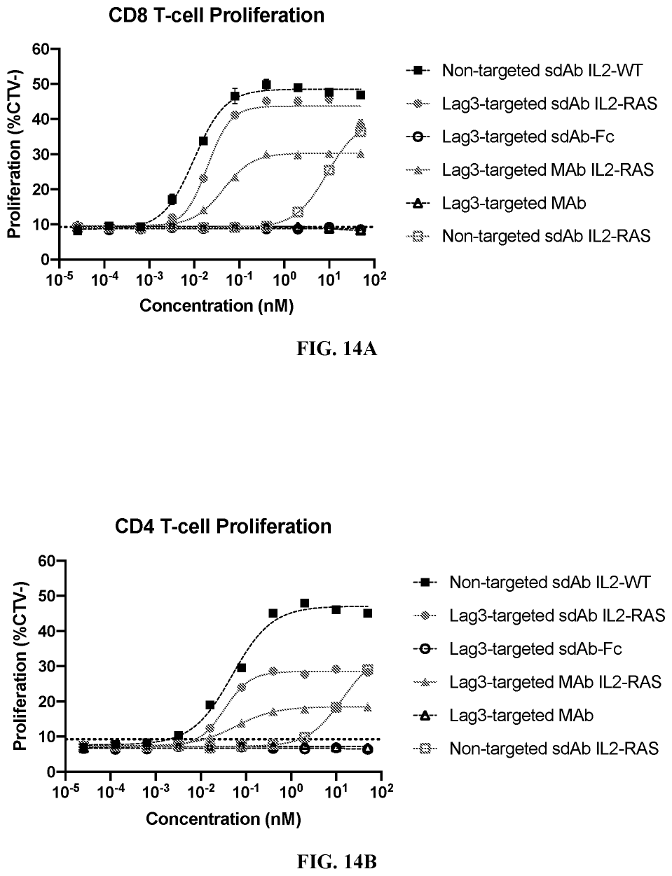

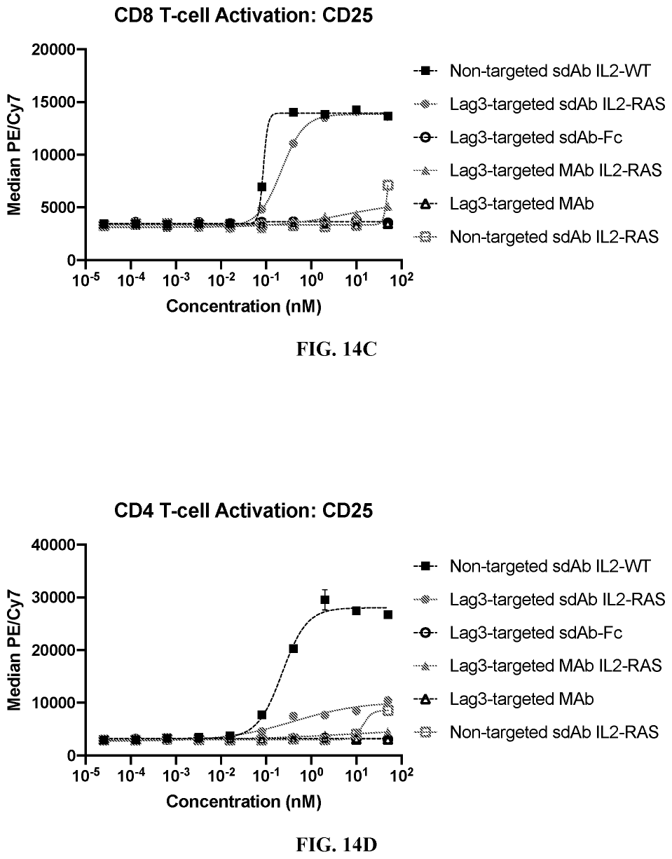

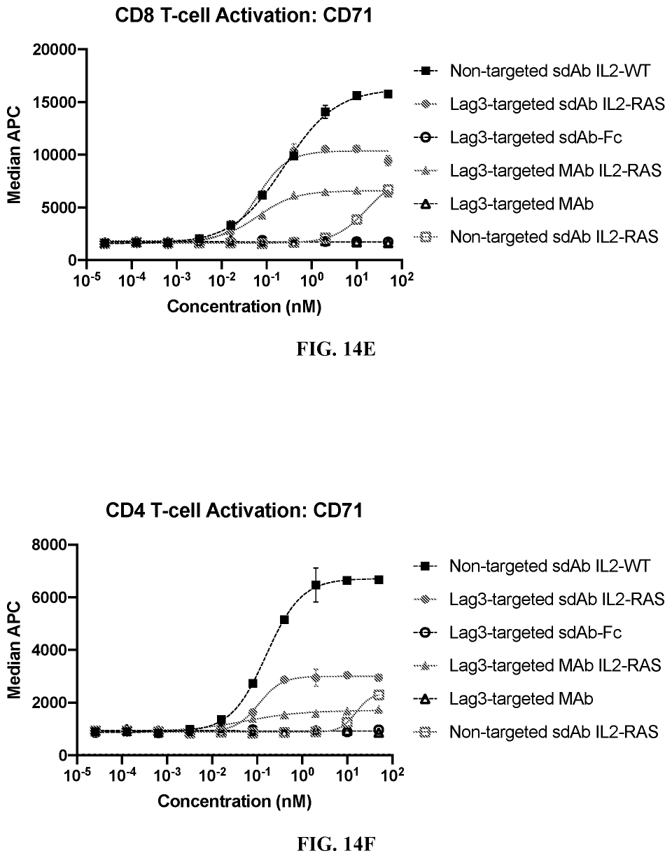

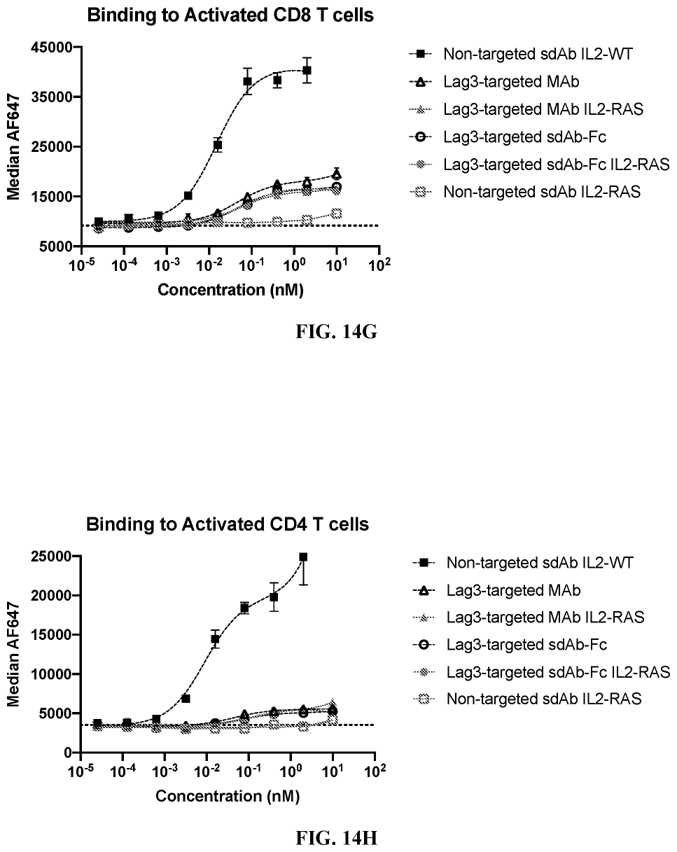

As shown in FIG. 14A-14D, both anti-LAG3 Mab-IL-2-RAS and anti-LAG3 VHH-IL-2-RAS increased CD8+ and CD4+ proliferation (FIGS. 14A and 14B) and activation as indicated by CD25 (FIGS. 14C and 14D) and CD71 (FIGS. 14E and 14F) expression levels. Non-targeted wild type IL-2 was a strong inducer of CD8+ and CD4+ T cell proliferation and activation, and bound stimulated T cells with higher affinity and saturation.

### Example 15: Combination Mutants of IL-2 Further Reduce Non-Targeted Activity

HEK-Blue IL-2 reporter cells (InvivoGen) were used to measure the relative activities of non-targeted IL-2 mutants. Reporter cells were treated with dilutions of IL-2-mutants fused to the C-terminus of a non-targeted VHH and incubated for 20 hours before quantification of the IL-2 induced secretion of alkaline phosphatase by the reporter cells using a colorimetric enzyme substrate, QUANTI-Blue™ (Invivogen).

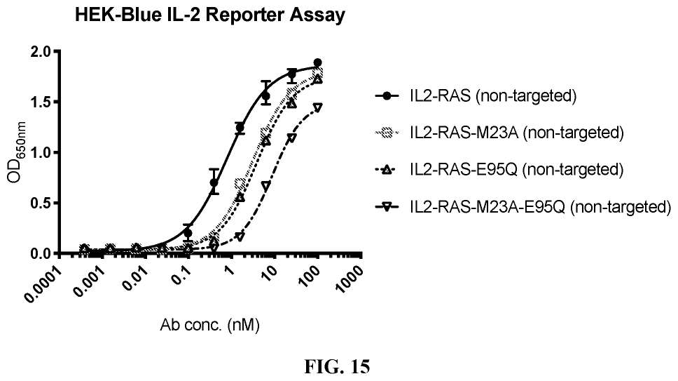

As shown in FIG. 15, the IL-2 mutants showed a range of activities. Experiments described above showed that IL-2-RAS (P65R, H16A, and D84S) had dramatically reduced binding to IL-2Rs compared to wild type IL-2 (see FIG. 4-6), and reduced activity compared to wild type IL-2 (see FIG. 7A-7E). IL-2-RAS with an additional M23A mutation and IL-2-RAS with an additional E95Q mutation both showed reduced activity compared to IL-2-RAS, and the combination of IL-2-RAS with both M23A and E95Q had even further attenuated activity. In experiments with PD-1-expressing reporter cells, these reduced affinity IL-2 mutants all showed comparable PD-1-targeted activity (data not shown), suggesting that high affinity binding to PD-1 in cis can compensate for reduced affinity of IL-2 mutants to IL-2R. While the HEK-Blue IL-2 reporter system was useful for relative activity measurement, the observed EC₅₀ for IL-2 mutants in the reporter system was shifted significantly to the left compared to primary lymphocytes, likely due to the overexpression of IL-2R components in the reporter cell compared to lower IL-2R levels on primary cells.

### Example 16: Activities of Polypeptides Comprising a Modified IL-2

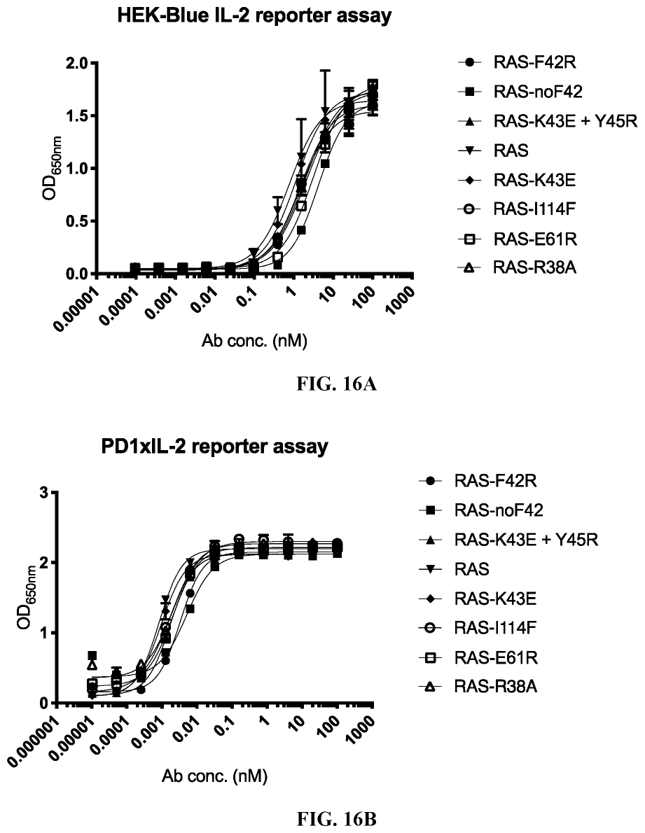

HEK-Blue IL-2 reporter cells (InvivoGen) or modified IL-2 reporter cell clones expressing PD-1 were used to measure were used to measure the relative activities of polypeptides comprising a modified IL-2 fused to the C-terminus of a PD-1-binding VHH. The IL-2 modifications and SEQ ID NO of each modified IL-2 are shown in Tables 2 and 3. Control polypeptides comprising IL-2 comprising only T3G and C125S mutations or wild type IL-2 were also tested. Untargeted IL-2 activities were measured using the reporter cells that did not express PD-1. The reporter cells were treated with dilutions of the polypeptides and incubated 20 hours before Quanti-Blue analysis, as described in Example 15. The tested polypeptides' EC₅₀s on the cells lacking PD-1 were calculated using a four parameter nonlinear curve fit in Prism 8 ([Agonist] vs response—Variable slope) and are shown in Table 2 below, and the dose response curves are shown in FIG. 16A.

TABLE 2
EC₅₀s of polypeptides comprising a modified IL-2 on cells lacking
PD-1

| | EC₅₀ | SEQ ID |
|---|---|---|
| IL-2 modifications | (nM) | NO |
| T3A, H16A, F42R, P65R, D84S, C125S | 2.1 | 282 |
| T3A, H16A, Δ42, P65R, D84S, C125S | 4.2 | 278 |
| T3A, H16A, K43E, Y45R, P65R, D84S, C125S | 1.5 | 279 |
| T3A, H16A, P65R, D84S, C125S (INBRX-108) | 0.9 | 28 |
| T3A, H16A, K43E, P65R, D84S, C125S | 1.1 | 280 |
| T3A, H16A, P65R, D84S, I114F, C125S | 1.4 | 281 |
| T3A, H16A, E61R, P65R, D84S, C125S | 3.2 | 108 |
| T3A, H16A, R38A, P65R, D84S, C125S | 2.0 | 109 |

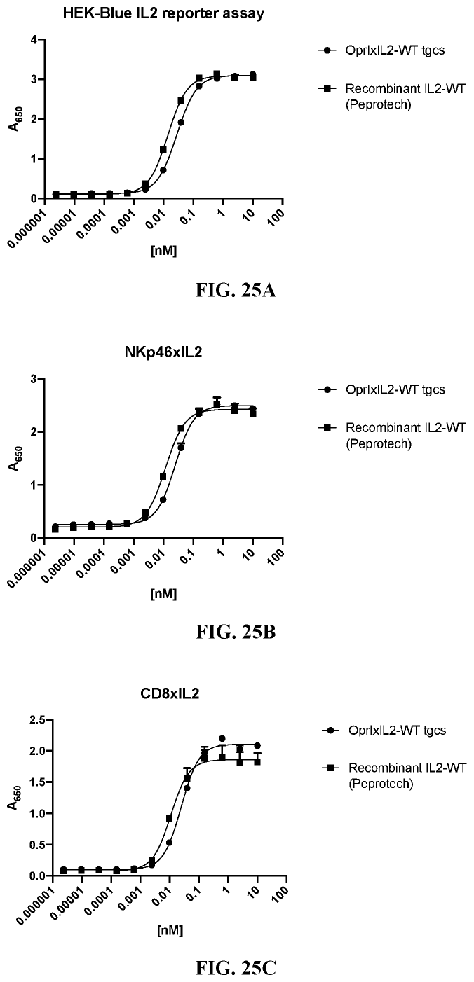

As shown in Table 2 and FIG. 16A, the tested polypeptides comprising a modified IL-2 showed a range of activities. IL-2-RAS-T3A-C125S with an additional F42R, 442, K43E and/or Y45R, I114F, E61R, or R38A modification showed reduced activity compared to IL-2-RAS-T3A-C125S. As shown in FIG. 25A-C, the reporter cells are responsive to wild type IL-2 and IL-2 comprising T3G and C125S mutations.

The PD-1 targeted IL-2 activities of the same polypeptides were tested in the PD-1-expressing IL-2 reporter cells. The EC₅₀s on the cells expressing PD-1 were calculated using a four parameter nonlinear curve fit in Prism 8 ([Agonist] vs response—Variable slope) and are shown in Table 3 below, and the dose response curves are shown in FIG. 16B.

TABLE 3
EC₅₀s of polypeptides comprising a modified IL-2 on cells expressing
PD-1

| | EC₅₀ | SEQ ID |
|---|---|---|
| IL-2 modifications | (nM) | NO. |
| T3A, H16A, F42R, P65R, D84S, C125S | 0.003 | 282 |
| T3A, H16A, Δ42, P65R, D84S, C125S | 0.005 | 278 |
| T3A, H16A, K43E, Y45R, P65R, D84S, C125S | 0.002 | 279 |
| T3A, H16A, P65R, D84S, C125S (INBRX-108) | 0.001 | 28 |
| T3A, H16A, K43E, P65R, D84S, C125S | 0.001 | 280 |
| T3A, H16A, P65R, D84S, I114F, C125S | 0.002 | 281 |
| T3A, H16A, E61R, P65R, D84S, C125S | 0.002 | 108 |
| T3A, H16A, R38A, P65R, D84S, C125S | 0.002 | 109 |

As shown in Table 3 and FIG. 16B, the PD-1 targeted IL-2 activities of polypeptides comprising IL-2-RAS-T3A-C125S with an additional F42R, 442, K43E and/or Y45R, I114F, E61R, or R38A modification showed activity within 2- to 5-fold of the activity of IL-2-RAS-T3A-C125S.

### Example 17: Effect of Blocking CD25 on Activities of Polypeptides Comprising a Modified IL-2

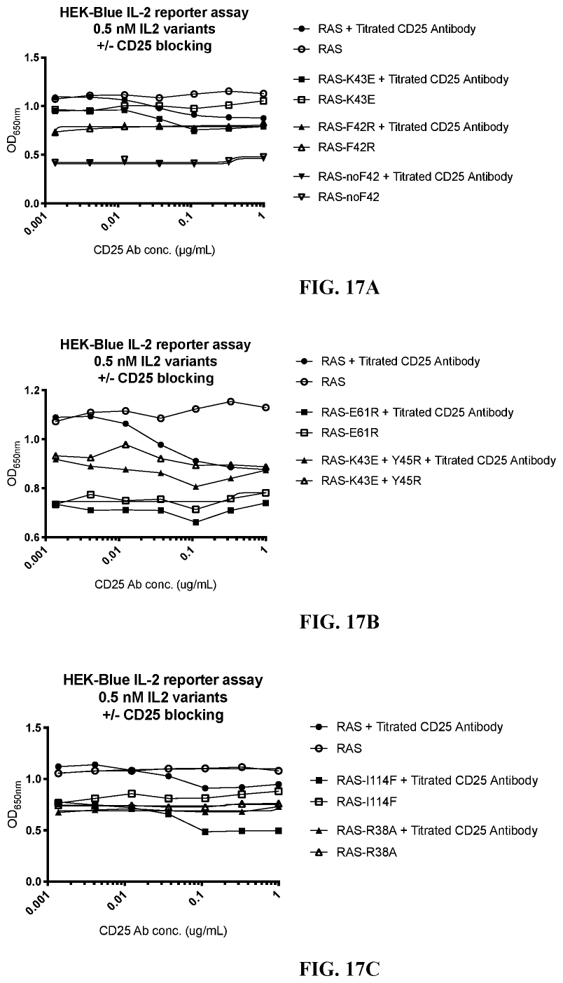

HEK-Blue IL-2 reporter cells (InvivoGen) that did not express PD-1 were used to measure the relative activities of polypeptides comprising a modified IL-2 fused to the C-terminus of a PD-1-binding VHH in the presence or absence of a CD25 antibody that blocks binding of IL-2 to CD25. The IL-2 modifications and SEQ ID NOs of the modified IL-2s are shown in Table 4. The reporter cells were treated with dilutions of the polypeptides and incubated 20 hours before Quanti-Blue analysis, as described in Example 15. The dose response curves are shown in FIG. 17A-C, and effects of CD25 blocking on IL-2 activities are listed in Table 4 below.

TABLE 4
Effects of blocking CD25 on cells lacking PD-1

| | Activity | |
|---|---|---|
| | blocked | SEQ |
| | by CD25 | ID |
| IL-2 modifications | antibody? | NO. |
| T3A, H16A, F42R, P65R, D84S, C125S | No | 282 |
| T3A, H16A, Δ42, P65R, D84S, C125S | No | 278 |
| T3A, H16A, K43E, Y45R, P65R, D84S, C125S | No | 279 |
| T3A, H16A, P65R, D84S, C125S (INBRX-108) | Yes | 28 |
| T3A, H16A, K43E, P65R, D84S, C125S | Yes | 280 |
| T3A, H16A, P65R, D84S, I114F, C125S | Yes | 281 |
| T3A, H16A, E61R, P65R, D84S, C125S | No | 108 |
| T3A, H16A, R38A, P65R, D84S, C125S | No | 109 |

As shown in Table 4 and FIG. 17A-C, polypeptides comprising IL-2-RAS-T3A-C125S or comprising IL-2-RAS-T3A-C125S with an additional K43E or I114F modification did not retain full IL-2 activity in the presence of a CD25 blocking antibody, indicating that CD25 was necessary for their activities. Polypeptides comprising IL-2-RAS-T3A-C125S with an additional F42R, 442, E61R, or R38A modification or comprising additional K43E and Y45R modifications retained full activity in the presence of a CD25 blocking antibody, indicating that CD25 was not necessary for their activities.

### Example 18: Activities of Polypeptides Comprising a Modified IL-2

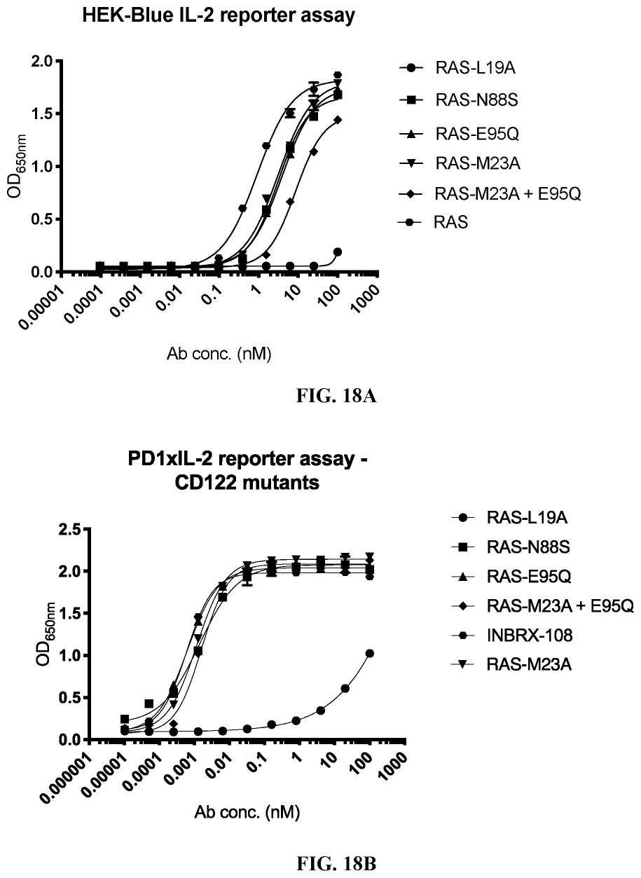

HEK-Blue IL-2 reporter cells described in Example 16 were used to measure the relative activities of polypeptides comprising a modified IL-2 fused to the C-terminus of a PD-1-binding VHH. The IL-2 modifications and SEQ ID NO of each modified IL-2 are shown in Table 5. Untargeted IL-2 activities were measured using the reporter cells that did not express PD-1. The reporter cells were treated with dilutions of the polypeptides and incubated 20 hours before Quanti-Blue analysis, as described in Example 15. The tested polypeptides' EC₅₀s on the cells lacking PD-1 were calculated using a four parameter nonlinear curve fit in Prism 8 ([Agonist] vs response—Variable slope) and are shown in Table 5 below, and the dose response curves are shown in FIG. 18A.

TABLE 5
EC₅₀s of polypeptides comprising a modified IL-2 on cells lacking
PD-1

| | EC₅₀ | SEQ ID |
|---|---|---|
| IL-2 modifications | (nM) | NO. |
| T3A, H16A, L19A, P65R, D84S, C125S | >100 | 110 |
| T3A, H16A, P65R, D84S, N88S, C125S | 3.1 | 111 |
| T3A, H16A, P65R, D84S, E95Q, C125S | 3.9 | 112 |
| T3A, H16A, M23A, P65R, D84S, C125S | 3.1 | 113 |
| T3A, H16A, M23A, P65R, D84S, E95Q, C125S | 8.7 | 114 |
| T3A, H16A, P65R, D84S, C125S (INBRX-108) | 0.9 | 28 |

As shown in Table 5 and FIG. 18A, the tested polypeptides comprising a modified IL-2 showed a range of activities. IL-2-RAS-T3A-C125S with an additional N88S, E95Q, and/or M23A modification showed reduced activity compared to IL-2-RAS-T3A-C125S. Addition of a L19A modification resulted in greatly reduced IL-2 activity.

The PD-1 targeted IL-2 activities of the same polypeptides were tested in the PD-1-expressing IL-2 reporter cells. The EC₅₀s on cells expressing PD-1 were calculated using a four parameter nonlinear curve fit in Prism 8 ([Agonist] vs response—Variable slope) and are shown in Table 6 below, and the dose response curves are shown in FIG. 18B.

TABLE 6
EC₅₀s of polypeptides comprising a modified IL-2 on cells expressing
PD-1

| | EC₅₀ | SEQ ID |
|---|---|---|
| IL-2 modifications | (nM) | NO. |
| T3A, H16A, L19A, P65R, D84S, C125S | >100 | 110 |
| T3A, H16A, P65R, D84S, N88S, C125S | 0.001 | 111 |
| T3A, H16A, P65R, D84S, E95Q, C125S | 0.0006 | 112 |
| T3A, H16A, M23A, P65R, D84S, C125S | 0.001 | 113 |
| T3A, H16A, M23A, P65R, D84S, E95Q, C125S | 0.002 | 114 |
| T3A, H16A, P65R, D84S, C125S | 0.001 | 28 |
| (INBRX-108) | | |

As shown in Table 6 and FIG. 18B, the PD-1 targeted IL-2 activities of polypeptides comprising IL-2-RAS-T3A-C125S with an additional N88S, M23A, or M23A and E95Q modification(s) showed activity within 2-fold of the activity of IL-2-RAS-T3A-C125S. Addition of a L19A modification resulted in greatly reduced IL-2 activity despite the presence of PD-1.

### Example 19: Activities of Polypeptides Comprising a Modified IL-2

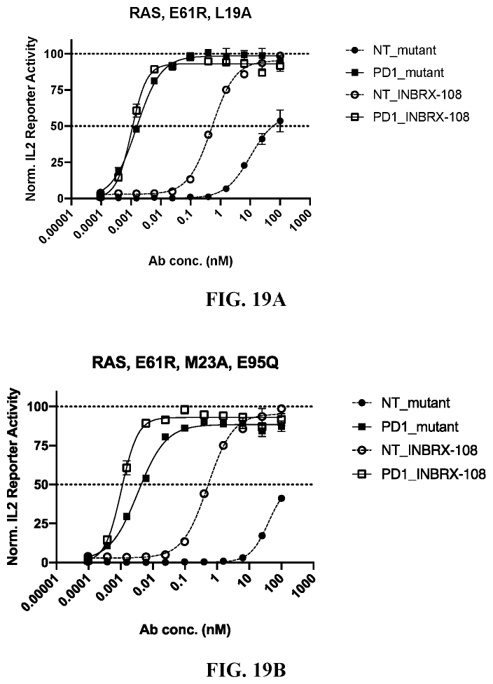

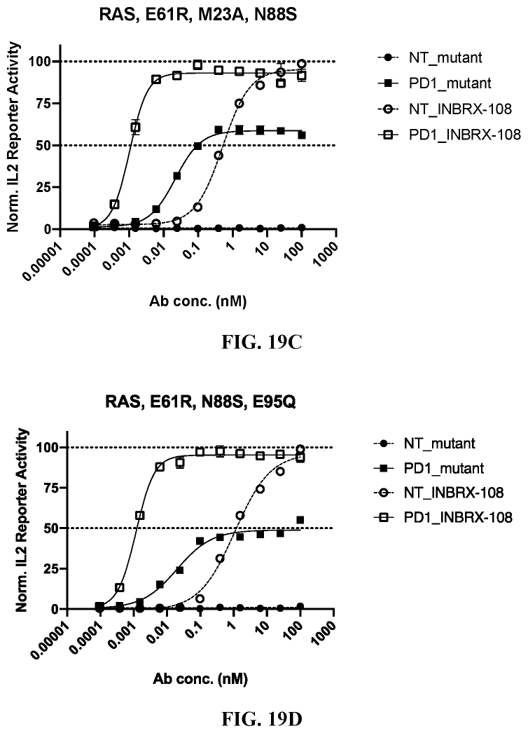

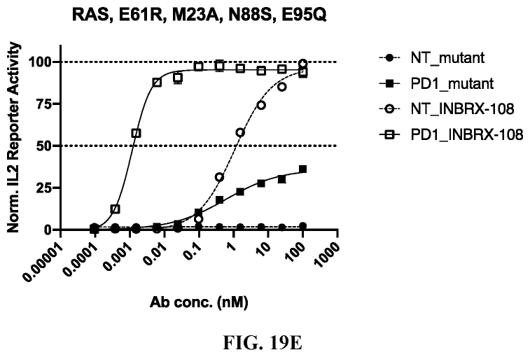

HEK-Blue IL-2 reporter cells described in Example 16 were used to measure the relative activities of polypeptides comprising a modified IL-2 fused to the C-terminus of a PD-1-binding VHH. The reporter cells were treated with dilutions of the polypeptides and incubated 20 hours before Quanti-Blue analysis, as described in Example 15. The identities and SEQ ID NOs of the modified IL-2s and EC₅₀s of the tested polypeptides are shown in Table 7 below, and the dose response curves are shown in FIG. 19A-E.

TABLE 7
EC₅₀s of Polypeptides comprising a modified IL-2

| | | | SEQ |
|---|---|---|---|
| | | EC₅₀ | ID |
| Cell Line | IL-2 modifications | (nM) | NO. |
| HEK-Blue IL-2 + PD-1 | T3A, H16A, L19A, E61R, | 0.002 | 115 |
| HEK-Blue IL-2 | P65R, D84S, C125S | 10.13 | |
| HEK-Blue IL-2 + PD-1 | T3A, H16A, M23A, E61R, | 0.003 | 116 |
| HEK-Blue IL-2 | P65R, D84S, E95Q, C125S | 39.42 | |
| HEK-Blue IL-2 + PD-1 | T3A, H16A, M23A, E61R, | 0.02 | 117 |
| HEK-Blue IL-2 | P65R, D84S, N88S, C125S | >100 | |
| HEK-Blue IL-2 + PD-1 | T3A, H16A, E61R, P65R, | 0.02 | 118 |
| HEK-Blue IL-2 | D84S, N88S, E95Q, C125S | >100 | |
| HEK-Blue IL-2 + PD-1 | T3A, H16A, M23A, E61R, | 0.6 | 119 |
| HEK-Blue IL-2 | P65R, D84S, N88S, E95Q, | >100 | |
| | C125S | | |
| HEK-Blue IL-2 + PD-1 | T3A, H16A, P65R, D84S, | 0.001 | 28 |
| HEK-Blue IL-2 | C125S (INBRX-108) | 0.512 | |

As shown in Table 7 and FIG. 19A-E, the tested polypeptides comprising a modified IL-2 showed a range of activities. While all of the polypeptides comprising IL-2-RAS-T3A-C125S with additional modifications exhibited significantly reduced non-targeted activity compared to polypeptides comprising IL-2-RAS-T3A-C125S, polypeptides comprising IL RAS-T3A-C125S with additional L19A and E61R modifications and those with additional M23A, E61R, and E95Q modifications exhibited comparable PD-1 targeted activity to polypeptides comprising IL-2-RAS-T3A-C125S. Example 18 showed that the L19A mutation, in the absence of the E61R mutation, reduced the non-targeted and targeted activities of polypeptides comprising IL-2-RAS-T3A-C125S; however, these results show that the addition of the E61R mutation restored PD-1 targeted activity. Thus, the effects of combining individual mutations are difficult to predict.

### Example 20: Activities of Polypeptides Comprising a Modified IL-2

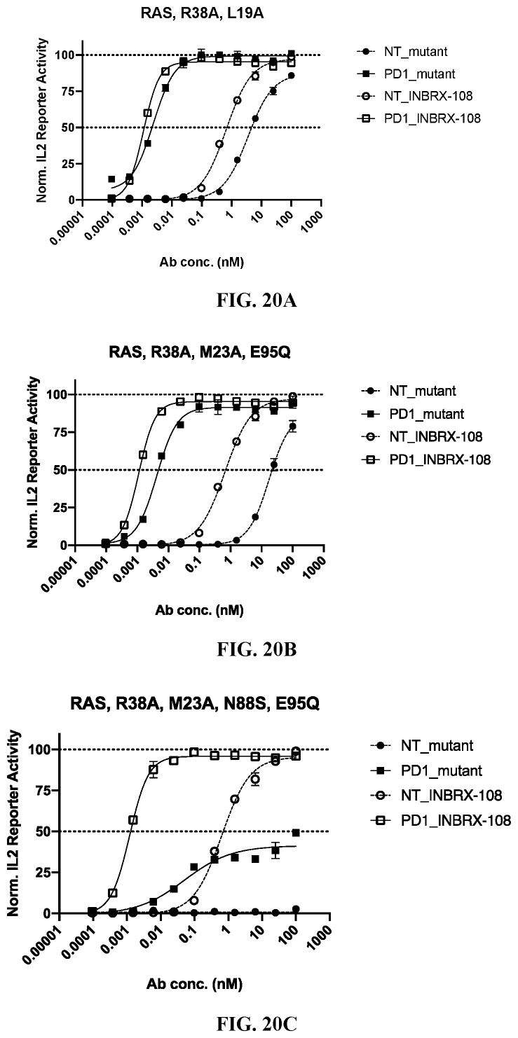

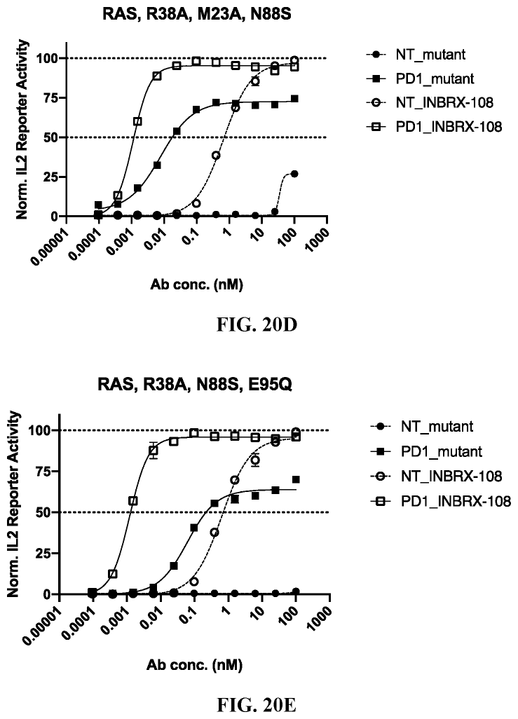

HEK-Blue IL-2 reporter cells described in Example 16 were used to measure the relative activities of polypeptides comprising a modified IL-2 fused to the C-terminus of a PD-1-binding VHH. The reporter cells were treated with dilutions of the polypeptides and incubated 20 hours before Quanti-Blue analysis, as described in Example 15. The identities and SEQ ID NOs of the modified IL-2s and EC₅₀s of the tested polypeptides are shown in Table 8 below, and the dose response curves are shown in FIG. 20A-E.

TABLE 8
EC₅₀s of Polypeptides comprising a modified IL-2

| | | | SEQ |
|---|---|---|---|
| | | EC₅₀ | ID |
| Cell line | IL-2 modifications | (nM) | NO. |
| HEK-Blue IL-2 + PD-1 | T3A, H16A, L19A, R38A, | 0.002 | 120 |
| HEK-Blue IL-2 | P65R, D84S, C125S | 3.6 | |
| HEK-Blue IL-2 + PD-1 | T3A, H16A, M23A, R38A, | 0.004 | 121 |
| HEK-Blue IL-2 | P65R, D84S, E95Q, C125S | 17.5 | |
| HEK-Blue IL-2 + PD-1 | T3A, H16A, M23A, R38A, | 0.05 | 124 |
| HEK-Blue IL-2 | P65R, D84S, N88S, E95Q, | >100 | |
| | C125S | | |
| HEK-Blue IL-2 + PD-1 | T3A, H16A, M23A, R38A, | 0.008 | 122 |
| HEK-Blue IL-2 | P65R, D84S, N88S, C125S | >100 | |
| HEK-Blue IL-2 + PD-1 | T3A, H16A, R38A, P65R, | 0.06 | 123 |
| HEK-Blue IL-2 | D84S, N88S, E95Q, C125S | >100 | |
| HEK-Blue IL-2 + PD-1 | T3A, H16A, P65R, D84S, | 0.001 | 28 |
| HEK-Blue IL-2 | C125S (INBRX-108) | 0.512 | |

As shown in Table 8 and FIG. 20A-E, the tested polypeptides comprising a modified IL-2 showed a range of activities. All of the polypeptides comprising IL-2-RAS-T3A-C125S with additional modifications exhibited reduced non-targeted activity compared to polypeptides comprising IL-2-RAS-T3A-C125S, and some exhibited no non-targeted activity. Polypeptides comprising IL-2-RAS-T3A-C125S with additional L19A and R38A modifications and those with additional M23A, R38A, and E95Q modifications exhibited comparable PD-1 targeted activity to polypeptides comprising IL-2-RAS-T3A-C125S.

### Example 21: Activities of Polypeptides Comprising a Modified IL-2

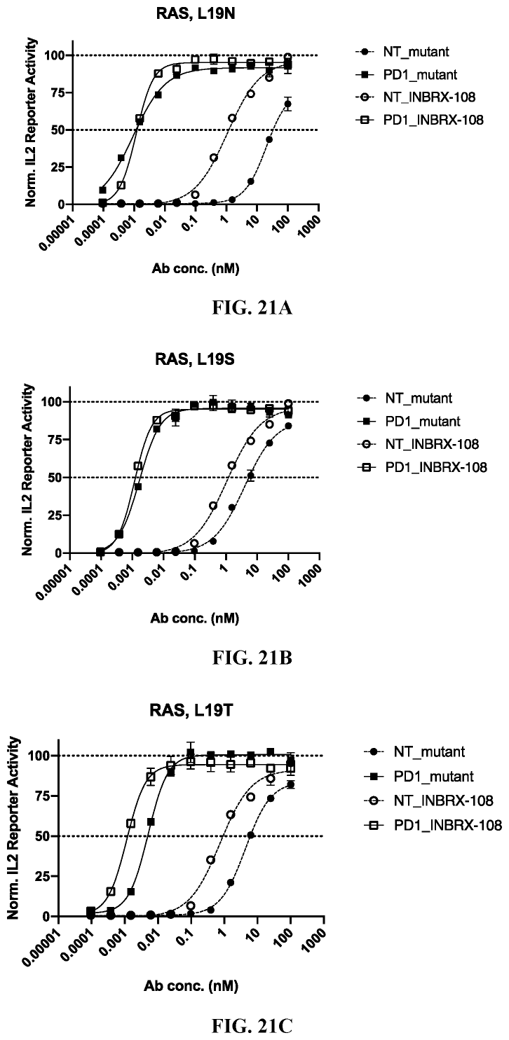

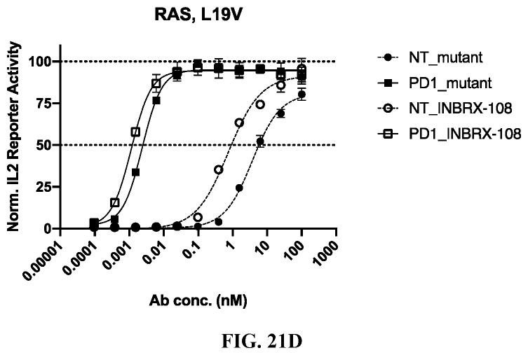

HEK-Blue IL-2 reporter cells described in Example 16 were used to measure the relative activities of polypeptides comprising a modified IL-2 fused to the C-terminus of a PD-1-binding VHH. The reporter cells were treated with dilutions of the polypeptides and incubated 20 hours before Quanti-Blue analysis, as described in Example 15. The identities and SEQ ID NOs of the modified IL-2s and EC₅₀s of the tested polypeptides are shown in Table 9 below, and the dose response curves are shown in FIG. 21A-D.

TABLE 9
EC₅₀s of Polypeptides comprising a modified IL-2

| | | | SEQ |
|---|---|---|---|
| | | EC₅₀ | ID |
| Cell line | IL-2 modifications | (nM) | NO. |
| HEK-Blue IL-2 + PD-1 | T3A, H16A, L19N, P65R, | 0.001 | 125 |
| HEK-Blue IL-2 | D84S, C125S | 20.1 | |
| HEK-Blue IL-2 + PD-1 | T3A, H16A, L19S, P65R, | 0.002 | 126 |
| HEK-Blue IL-2 | D84S, C125S | 4.02 | |
| HEK-Blue IL-2 + PD-1 | T3A, H16A, L19T, P65R, | 0.005 | 127 |
| HEK-Blue IL-2 | D84S, C125S | 4.37 | |
| HEK-Blue IL-2 + PD-1 | T3A, H16A, L19V, P65R, | 0.002 | 128 |
| HEK-Blue IL-2 | D84S, C125S | 3.72 | |
| HEK-Blue IL-2 + PD-1 | T3A, H16A, P65R, D84S, | 0.001 | 28 |
| HEK-Blue IL-2 | C125S (INBRX-108) | 0.512 | |

As shown in Table 9 and FIG. 21A-D, the polypeptides comprising IL-2-RAS-T3A-C125S with additional L19 modification exhibited comparable PD-1-targeted activity to polypeptides comprising IL-2-RAS-T3A-C125S, while the non-targeted activities of the tested polypeptides ranged from slightly to significantly reduced compared to polypeptides comprising IL-2-RAS-T3A-C125S.

### Example 22: Activities of Polypeptides Comprising a Modified IL-2

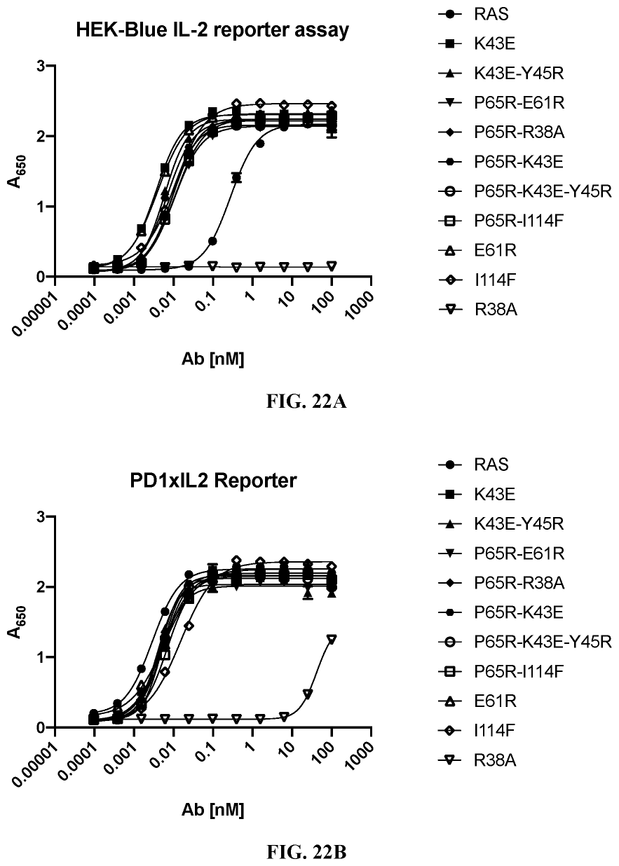

HEK-Blue IL-2 reporter cells described in Example 16, were used to measure the relative activities of polypeptides comprising a modified IL-2 fused to the C-terminus of a PD-1-binding VHH. The polypeptides were modified with IL-2 mutations designed to disrupt the IL-2-CD25 interface, and they were compared to control polypeptides comprising IL-2-RAS-T3A-C125S. The reporter cells were treated with dilutions of the polypeptides and incubated 20 hours before Quanti-Blue analysis, as described in Example 15. The identities and SEQ ID NOs of the modified IL-2s and EC₅₀s of the tested polypeptides are shown in Table 10 below, and the dose response curves are shown in FIG. 22A (cells lacking PD-1) and in FIG. 22B (cells expressing PD-1).

TABLE 10
EC₅₀s of Polypeptides comprising a modified IL-2

| | | | SEQ |
|---|---|---|---|
| | | EC₅₀ | ID |
| Cell line | IL-2 modifications | (nM) | NO. |
| HEK-Blue IL-2 + PD-1 | T3A, H16A, P65R, D84S, | 0.003 | 28 |
| HEK-Blue IL-2 | C125S (INBRX-108) | 0.3 | |
| HEK-Blue IL-2 + PD-1 | T3A, K43E, C125S | 0.005 | 286 |
| HEK-Blue IL-2 | | 0.004 | |
| HEK-Blue IL-2 + PD-1 | T3A, K43E, Y45R, C125S | 0.004 | 283 |
| HEK-Blue IL-2 | | 0.006 | |
| HEK-Blue IL-2 + PD-1 | T3A, E61R, P65R, C125S | 0.005 | 106 |
| HEK-Blue IL-2 | | 0.01 | |
| HEK-Blue IL-2 + PD-1 | T3A, R38A, P65R, C125S | 0.006 | 107 |
| HEK-Blue IL-2 | | 0.01 | |
| HEK-Blue IL-2 + PD-1 | T3A, K43E, P65R, C125S | 0.005 | 288 |
| HEK-Blue IL-2 | | 0.007 | |
| HEK-Blue IL-2 + PD-1 | T3A, K43E, Y45R, P65R, | 0.005 | 289 |
| HEK-Blue IL-2 | C125S | 0.009 | |
| HEK-Blue IL-2 + PD-1 | T3A, P65R, I114F, C125S | 0.007 | 287 |
| HEK-Blue IL-2 | | 0.01 | |
| HEK-Blue IL-2 + PD-1 | T3A, E61R, C125S | 0.006 | 284 |
| HEK-Blue IL-2 | | 0.004 | |
| HEK-Blue IL-2 + PD-1 | T3A, I114F, C125S | 0.02 | 285 |
| HEK-Blue IL-2 | | >100 | |
| HEK-Blue IL-2 + PD-1 | T3A, R38A, C125S | 0.02 | 290 |
| HEK-Blue IL-2 | | 0.01 | |

As shown in Table 10 and FIG. 22A, the polypeptide comprising IL-2-T3A-R38A-C125S exhibited no non-targeted activity, and the remaining polypeptides tested exhibited significantly increased non-targeted activity compared to the polypeptide comprising IL-2-RAS-T3A-C125S. As shown in FIG. 22B, the polypeptide comprising IL-2-T3A-R38A-C125S exhibited some PD-1-targeted activity that was significantly reduced compared to the polypeptide comprising IL-2-RAS-T3A-C125S. The remaining polypeptides tested exhibited comparable PD-1-targeted activity to the polypeptide comprising IL-2-RAS-T3A-C125S.

### Example 23: Activities of Polypeptides Comprising a Modified IL-2

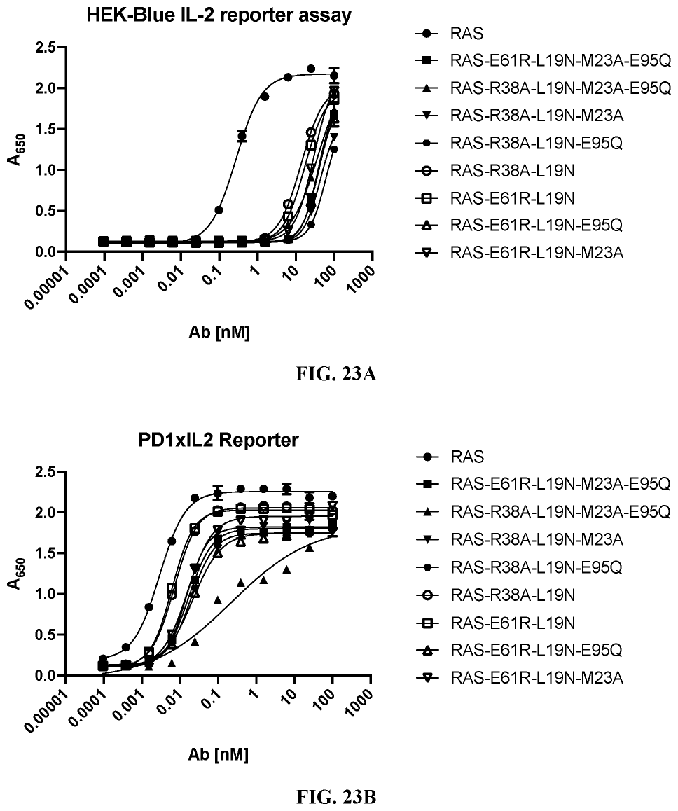

IL-2 reporter cells described in Example 16 were used to measure the relative activities of polypeptides comprising a modified IL-2 fused to the C-terminus of a PD-1-binding VHH. The reporter cells were treated with dilutions of the polypeptides and incubated 20 hours before Quanti-Blue analysis, as described in Example 15. The identities and SEQ ID NOs of the modified IL-2s and EC₅₀s of the tested polypeptides are shown in Table 11 below, and the dose response curves are shown in FIG. 23A (cells lacking PD-1) and in FIG. 23B (cells expressing PD-1).

TABLE 11
EC₅₀s of Polypeptides comprising a modified IL-2

| | | | SEQ |
|---|---|---|---|
| | | EC₅₀ | ID |
| Cell line | IL-2 modifications | (nM) | NO. |
| HEK-Blue IL-2 + PD-1 | T3A, H16A, P65R, D84S, | 0.003 | 28 |
| HEK-Blue IL-2 | C125S (INBRX-108) | 0.3 | |
| HEK-Blue IL-2 + PD-1 | T3A, H16A, L19N, M23A, | 0.017 | 129 |
| HEK-Blue IL-2 | E61R, P65R, D84S, E95Q, | 38.46 | |
| | C125S | | |
| HEK-Blue IL-2 + PD-1 | T3A, H16A, L19N, M23A, | 0.044 | 130 |
| HEK-Blue IL-2 | R38A, P65R, D84S, E95Q, | 37.98 | |
| | C125S | | |
| HEK-Blue IL-2 + PD-1 | T3A, H16A, L19N, M23A, | 0.015 | 131 |
| HEK-Blue IL-2 | R38A, P65R, D84S, C125S | 43.33 | |
| HEK-Blue IL-2 + PD-1 | T3A, H16A, L19N, R38A, | 0.019 | 132 |
| HEK-Blue IL-2 | P65R, D84S, E95Q C125S | 60.14 | |
| HEK-Blue IL-2 + PD-1 | T3A, H16A, L19N, R38A, | 0.007 | 133 |
| HEK-Blue IL-2 | P65R, D84S, C125S | 13.81 | |
| HEK-Blue IL-2 + PD-1 | T3A, H16A, L19N, E61R, | 0.006 | 134 |
| HEK-Blue IL-2 | P65R, D84S, C125S | 17.23 | |
| HEK-Blue IL-2 + PD-1 | T3A, H16A, L19N, E61R, | 0.022 | 135 |
| HEK-Blue IL-2 | P65R, D84S, E95Q, C125S | 40.08 | |
| HEK-Blue IL-2 + PD-1 | T3A, H16A, L19N, M23A, | 0.016 | 136 |
| HEK-Blue IL-2 | E61R, P65R, D84S, C125S | 29.27 | |

As shown in Table 11 and FIG. 23A, the tested polypeptides comprising IL-2-RAS-T3A-C125S with additional modifications exhibited significantly reduced non-targeted activity compared to polypeptides comprising IL-2-RAS-T3A-C125S. As shown in FIG. 23B, the tested polypeptides comprising IL-2-RAS-T3A-C125S with additional modifications exhibited a range of PD-1-targeted activities, from slightly reduced to significantly reduced compared to polypeptides comprising the polypeptides comprising IL-2-RAS-T3A-C125S. Polypeptides comprising IL-2-RAS-T3A-C125S with additional L19N and R38A or E61R mutations exhibited the highest PD-1-targeted activity of the tested polypeptides comprising IL-2-RAS-T3A-C125S with additional modifications.

### Example 24: CD25 Binding by Polypeptides Comprising a Modified IL-2

Polypeptides comprising a modified IL-2 fused to the C-terminus of a PD-1 binding VHH and control polypeptides were tested for binding to CD25 using Biolayer Interferometry (ForteBio). Histidine-tagged CD25 was immobilized, and 300 nM of a polypeptide listed in the table below was added. The resulting binding results are shown in Table 12 below.

TABLE 12
Polypeptides tested for binding to CD25

| | Binding | SEQ |
|---|---|---|
| | to | ID |
| Polypeptide | CD25 | NO. |
| IL-2-T3A-RAS-C125S | Yes | 28 |
| WT IL-2 | Yes | 1 |
| WT IL-15 | No | 284 |
| IL-2-T3A-H16A-Δ42-P65R-D84S-C125S | No | 278 |
| IL-2-T3A-H16A-E61R-P65R-D84S-C125S | No | 108 |
| IL-2-T3A-H16A-F42R-P65R-D84S-C125S | Yes | 282 |
| IL-2-T3A-H16A-R38A-P65R-D84S-C125S | No | 109 |
| IL-2-T3A-H16A-K43E-Y45R-P65R-D84S-C125S | No | 279 |
| PD-1-IL-2v fusion | No | 2 |
| IL-2-T3A-H16A-K43E-P65R-D84S-C125S | No | 280 |
| IL-2-T3A-H16A-P65R-D84S-I114F-C125S | No | 281 |

The polypeptide comprising IL-2-RAS-T3A-C125S bound CD25 despite comprising mutations designed to disrupt the CD25-IL-2 interface. The polypeptides comprising IL-2 comprising E61R and P65R mutations exhibited significantly decreased or no CD25 binding compared to the polypeptide comprising IL-2 comprising only RAS-T3A-C125S mutations.

### Example 25: Activities of Polypeptides Comprising a Modified IL-2

The relative activities of polypeptides comprising a modified IL-2 fused to the C-terminus of a PD-1-binding VHH were measured using HEK-Blue IL-2 reporter cells (InvivoGen) or modified cell clones expressing PD-1, as described in Example 16. The IL-2 modifications and SEQ ID NOs of the modified IL-2s are shown in Table 13.

The relative activities of polypeptides comprising a modified IL-2 fused to the C-terminus of a NKp46-binding VHH were measured using HEK-Blue IL-2 reporter cells (InvivoGen) or modified cell clones expressing cells expressing NKp46. The IL-2 modifications and SEQ ID NOs of the modified IL-2s are shown in Table 13.

The relative activities of polypeptides comprising a modified IL-2 fused to the C-terminus of a CD8a-binding VHH were measured using HEK-Blue IL-2 reporter cells (InvivoGen) or modified cell clones expressing cells expressing CD8a. The IL-2 modifications and SEQ ID NOs of the modified IL-2s are shown in Table 14.

Non-targeted activities were measured using cells lacking PD-1, lacking NKp46, and lacking CD8a expression.

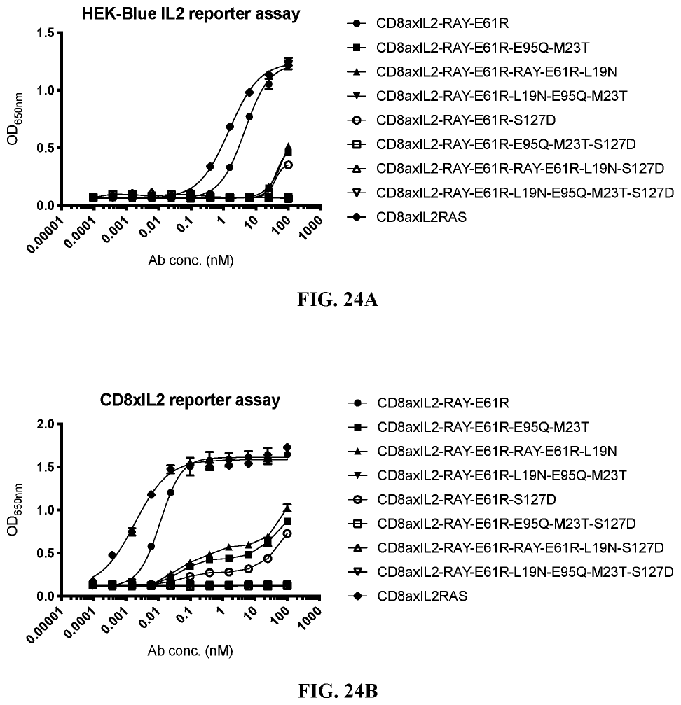

The results are shown in Tables 13 and 14 below, and the dose response curves corresponding to Table 14 are shown in FIG. 24A (cells lacking CD8a) and in FIG. 24B (cells expressing CD8a). A maximum value of 50.000 nM was used for the non-targeted EC₅₀ entries, including for non-targeted EC₅₀s that could not be calculated due to poor activity and poor fit to the nonlinear regression calculation. The PD-1 targeted EC₅₀s were calculated using the polypeptides comprising a PD-1-binding VHH and the reporter cells expressing PD-1. The NKp46 targeted EC₅₀s were calculated using the polypeptides comprising a NKp46-binding VHH and the reporter cells expressing NKp46. The CD8a targeted EC₅₀s were calculated using the polypeptides comprising a CD8a-binding VHH and the reporter cells expressing CD8a. The “window” is the non-targeted EC₅₀ divided by the targeted EC₅₀ and represents the concentration differential of the targeted IL-2 activity. A blank entry indicates that the polypeptide was not tested in the corresponding experiment.

TABLE 13
EC₅₀s of polypeptides comprising a modified IL-2

| | Non- | PD-1 | NKp46 | | | |
|---|---|---|---|---|---|---|
| | targeted | Targeted | Targeted | | | |
| | EC₅₀ | EC₅₀ | EC₅₀ | PD-1 | NKp46 | SEQ |
| IL-2 Modifications | (nM) | (nM) | (nM) | Window | Window | ID NO. |
| T3G, C125S | 0.020 | 0.020 | | 1.056 | | 105 |
| None (IL-2 WT) | 0.018 | 0.020 | | 0.943 | | 1 |
| T3A, E61R, P65R, C125S | 0.010 | 0.004 | | 2.308 | | 106 |
| T3A, R38A, P65R, C125S | 0.010 | 0.005 | | 1.786 | | 107 |
| T3A, H16A, E61R, P65R, | 3.178 | 0.002 | | 1570.934 | | 108 |
| D84S, C125S | | | | | | |
| T3A, H16A, R38A, P65R, | 1.971 | 0.002 | | 977.679 | | 109 |
| D84S, C125S | | | | | | |
| T3A, H16A, L19A, P65R, | 50.000 | 50.000 | | 1.000 | | 110 |
| D84S, C125S | | | | | | |
| T3A, H16A, P65R, D84S, | 3.089 | 0.001 | | 2214.337 | | 111 |
| N88S, C125S | | | | | | |
| T3A, H16A, P65R, D84S, | 3.859 | 0.001 | | 6093.479 | | 112 |
| E95Q, C125S | | | | | | |
| T3A, H16A, M23A, | 3.125 | 0.001 | | 2698.618 | | 113 |
| P65R, D84S, C125S | | | | | | |
| T3A, H16A, M23A, | 8.695 | 0.002 | | 5420.823 | | 114 |
| P65R, D84S, E95Q, | | | | | | |
| C125S | | | | | | |
| T3A, H16A, L19A, E61R, | 10.130 | 0.002 | 0.017 | 6481.126 | 581.850 | 115 |
| P65R, D84S, C125S | | | | | | |
| T3A, H16A, M23A, | 39.420 | 0.003 | 0.020 | 11522.947 | 1976.931 | 116 |
| E61R, P65R, D84S, | | | | | | |
| E95Q, C125S | | | | | | |
| T3A, H16A, M23A, | 50.000 | 0.022 | | 2289.377 | | 117 |
| E61R, P65R, D84S, N88S, | | | | | | |
| C125S | | | | | | |
| T3A, H16A, E61R, P65R, | 50.000 | 0.020 | | 2515.091 | | 118 |
| D84S, N88S, E95Q, | | | | | | |
| C125S | | | | | | |
| T3A, H16A, M23A, | 50.000 | 0.592 | | 84.517 | | 119 |
| E61R, P65R, D84S, N88S, | | | | | | |
| E95Q, C125S | | | | | | |
| T3A, H16A, L19A, R38A, | 3.552 | 0.002 | | 1481.852 | | 120 |
| P65R, D84S, C125S | | | | | | |
| T3A, H16A, M23A, | 17.460 | 0.004 | 0.017 | 4100.517 | 1006.920 | 121 |
| R38A, P65R, D84S, | | | | | | |
| E95Q, C125S | | | | | | |
| T3A, H16A, M23A, | 50.000 | 0.008 | 0.098 | 6344.373 | 509.892 | 122 |
| R38A, P65R, D84S, | | | | | | |
| N88S, C125S | | | | | | |
| T3A, H16A, R38A, P65R, | 50.000 | 0.062 | | 806.712 | | 123 |
| D84S, N88S, E95Q, | | | | | | |
| C125S | | | | | | |
| T3A, H16A, M23A, | 50.000 | 0.050 | | 992.654 | | 124 |
| R38A, P65R, D84S, | | | | | | |
| N88S, E95Q, C125S | | | | | | |
| T3A, H16A, L19N, P65R, | 20.090 | 0.001 | | 21808.511 | | 125 |
| D84S, C125S | | | | | | |
| T3A, H16A, L19S, P65R, | 4.017 | 0.002 | | 2376.923 | | 126 |
| D84S, C125S | | | | | | |
| T3A, H16A, L19T, P65R, | 4.372 | 0.005 | | 858.939 | | 127 |
| D84S, C125S | | | | | | |
| T3A, H16A, L19V, P65R, | 3.716 | 0.002 | | 1554.812 | | 128 |
| D84S, C125S | | | | | | |
| T3A, H16A, L19N, | 38.460 | 0.017 | 0.051 | 2293.381 | 748.686 | 129 |
| M23A, E61R, P65R, | | | | | | |
| D84S, E95Q, C125S | | | | | | |
| T3A, H16A, L19N, | 37.980 | 0.044 | | 854.059 | | 130 |
| M23A, R38A, P65R, | | | | | | |
| D84S, E95Q, C125S | | | | | | |
| T3A, H16A, L19N, | 43.330 | 0.015 | | 2975.962 | | 131 |
| M23A, R38A, P65R, | | | | | | |
| D84S, C125S | | | | | | |
| T3A, H16A, L19N, R38A, | 50.000 | 0.019 | 0.102 | 2597.403 | 491.159 | 132 |
| P65R, D84S, E95Q, | | | | | | |
| C125S | | | | | | |
| T3A, H16A, L19N, R38A, | 13.810 | 0.007 | | 1943.155 | | 133 |
| P65R, D84S, C125S | | | | | | |
| T3A, H16A, L19N, E61R, | 17.230 | 0.006 | 0.053 | 2784.421 | 324.482 | 134 |
| P65R, D84S, C125S | | | | | | |
| T3A, H16A, L19N, E61R, | 40.080 | 0.022 | 0.100 | 1852.982 | 400.000 | 135 |
| P65R, D84S, E95Q, | | | | | | |
| C125S | | | | | | |
| T3A, H16A, L19N, | 29.270 | 0.016 | | 1854.880 | | 136 |
| M23A, E61R, P65R, | | | | | | |
| D84S, C125S | | | | | | |
| T3A, H16A, L19N, Q22A, | 50.000 | | 0.021 | | 2373.042 | 137 |
| E61R, P65R, D84S, | | | | | | |
| C125S | | | | | | |
| T3A, H16A, L19N, E61R, | 50.000 | | 0.021 | | 2394.636 | 138 |
| P65R, D84S, R120A, | | | | | | |
| C125S | | | | | | |
| T3A, H16A, L19N, E61R, | 50.000 | | 0.043 | | 1170.412 | 139 |
| P65R, D84S, C125S, | | | | | | |
| Q126A | | | | | | |
| T3A, H16S, L19N, E61R, | 0.211 | | | | | 140 |
| P65R, D84S, C125S | | | | | | |
| T3A, H16T, L19N, E61R, | 7.801 | | 0.486 | | 16.048 | 141 |
| P65R, D84S, C125S | | | | | | |
| T3A, H16V, L19N, E61R, | 7.606 | | 0.803 | | 9.478 | 142 |
| P65R, D84S, C125S | | | | | | |
| T3A, H16N, L19N, E61R, | 16.260 | | 0.071 | | 228.467 | 143 |
| P65R, D84S, C125S | | | | | | |
| T3A, H16A, L19N, E61R, | 3.944 | | 0.019 | | 210.684 | 144 |
| P65R, D84T, C125S | | | | | | |
| T3A, H16A, L19N, E61R, | 5.757 | | 0.032 | | 178.567 | 145 |
| P65R, D84V, C125S | | | | | | |
| T3A, H16A, D20S, E61R, | 7.658 | | 4.445 | | 1.723 | 146 |
| P65R, D84S, C125S | | | | | | |
| T3A, H16A, D20T, E61R, | 6.151 | | 3.657 | | 1.682 | 147 |
| P65R, D84S, C125S | | | | | | |
| T3A, H16A, D20N, E61R, | 5.554 | | 5.952 | | 0.933 | 148 |
| P65R, D84S, C125S | | | | | | |
| T3A, H16A, D20A, E61R, | 2.994 | | 2.638 | | 1.135 | 149 |
| P65R, D84S, C125S | | | | | | |
| T3A, H16A, L19N, E61R, | 17.260 | | 0.019 | | 930.458 | 150 |
| P65R, D84N, C125S | | | | | | |
| T3A, H16A, E61R, P65R, | 50.000 | | 0.510 | | 98.116 | 151 |
| D84S, N88T C125S | | | | | | |
| T3A, H16A, E61R, P65R, | 50.000 | | 0.146 | | 341.997 | 152 |
| D84S, N88AC125S | | | | | | |
| T3A, H16P, L19A, E61R, | 50.000 | | 3.707 | | 13.488 | 153 |
| P65R, D84S, C125S | | | | | | |
| T3A, H16A, L19H, E61R, | 17.320 | | 0.048 | | 360.833 | 154 |
| P65R, D84S, C125S | | | | | | |
| T3A, H16A, L19P, E61R, | 50.000 | | 50.000 | | 1.000 | 155 |
| P65R, D84S, C125S | | | | | | |
| T3A, H16A, L19Q, E61R, | 17.870 | | 0.042 | | 423.560 | 156 |
| P65R, D84S, C125S | | | | | | |
| T3A, H16A, L19Y, E61R, | 3.936 | | 0.017 | | 228.704 | 157 |
| P65R, D84S, C125S | | | | | | |
| T3A, H16A, L19A, E61R, | 0.122 | | 50.000 | | 0.002 | 158 |
| P65R, D84P, C125S | | | | | | |
| T3A, H16A, L19A, E61R, | 2.822 | | 0.003 | | 857.230 | 159 |
| P65R, D84V, C125S | | | | | | |
| T3A, H16A, L19A, E61R, | 3.185 | | 0.001 | | 4696.255 | 160 |
| P65R, D84Y, C125S | | | | | | |
| T3A, H16A, M23A, | 7.203 | | 0.012 | | 603.772 | 161 |
| E61R, P65R, D84S, | | | | | | |
| E95H, C125S | | | | | | |
| T3A, H16A, M23A, | 34.230 | | 0.022 | | 1583.989 | 162 |
| E61R, P65R, D84S, | | | | | | |
| E95N, C125S | | | | | | |
| T3A, H16A, M23A, | 4.867 | | 0.002 | | 2919.616 | 163 |
| E61R, P65R, D84S, E95P, | | | | | | |
| C125S | | | | | | |
| T3A, H16A, M23A, | 50.000 | | 50.000 | | 1.000 | 164 |
| E61R, P65R, D84S, E95S, | | | | | | |
| C125S | | | | | | |
| T3A, H16A, M23A, | 13.220 | | 0.012 | | 1111.859 | 165 |
| E61R, P65R, D84S, E95T, | | | | | | |
| C125S | | | | | | |
| T3A, H16A, M23A, | 13.850 | | 0.064 | | 216.711 | 166 |
| E61R, P65R, D84S, | | | | | | |
| E95V, C125S | | | | | | |
| T3A, H16A, M23A, | 20.410 | | 0.088 | | 230.726 | 167 |
| E61R, P65R, D84S, | | | | | | |
| E95Y, C125S | | | | | | |
| T3A, H16A, L19A, E61A, | 2.067 | | 0.001 | | 2145.304 | 168 |
| P65R, D84S, C125S | | | | | | |
| T3A, H16A, L19A, E61G, | 3.263 | | 0.000 | | 12202.693 | 169 |
| P65R, D84S, C125S | | | | | | |
| T3A, H16A, L19A, E61H, | 4.534 | | 0.001 | | 8458.955 | 170 |
| P65R, D84S, C125S | | | | | | |
| T3A, H16A, L19A, E61N, | 1.997 | | 0.000 | | 12044.632 | 171 |
| P65R, D84S, C125S | | | | | | |
| T3A, H16A, L19A, E61P, | 8.436 | | 0.002 | | 5137.637 | 172 |
| P65R, D84S, C125S | | | | | | |
| T3A, H16A, L19A, E61Q, | 2.384 | | 0.002 | | 1129.323 | 173 |
| P65R, D84S, C125S | | | | | | |
| T3A, H16A, L19A, E61S, | 3.269 | | 0.001 | | 2534.109 | 174 |
| P65R, D84S, C125S | | | | | | |
| T3A, H16A, L19A, E61T, | 3.233 | | 0.001 | | 3725.942 | 175 |
| P65R, D84S, C125S | | | | | | |
| T3A, H16A, L19A, E61V, | 0.643 | | 0.003 | | 195.797 | 176 |
| P65R, D84S, C125S | | | | | | |
| T3A, H16A, L19A, E61Y, | 4.446 | | 0.004 | | 992.632 | 177 |
| P65R, D84S, C125S | | | | | | |
| T3A, H16A, M23D, | 50.000 | | 0.217 | | 230.627 | 178 |
| E61R, P65R, D84S, | | | | | | |
| E95Q, C125S | | | | | | |
| T3A, H16A, M23E, E61R, | 24.940 | | 0.020 | | 1275.703 | 179 |
| P65R, D84S, E95Q, | | | | | | |
| C125S | | | | | | |
| T3A, H16A, M23G, | 19.120 | | 0.063 | | 301.958 | 180 |
| E61R, P65R, D84S, | | | | | | |
| E95Q, C125S | | | | | | |
| T3A, H16A, M23I, E61R, | 17.790 | | 0.011 | | 1648.749 | 181 |
| P65R, D84S, E95Q, | | | | | | |
| C125S | | | | | | |
| T3A, H16A, M23K, | 50.000 | | 0.015 | | 3405.995 | 182 |
| E61R, P65R, D84S, | | | | | | |
| E95Q, C125S | | | | | | |
| T3A, H16A, M23L, E61R, | 5.657 | | 0.004 | | 1589.045 | 183 |
| P65R, D84S, E95Q, | | | | | | |
| C125S | | | | | | |
| T3A, H16A, M23N, | 50.000 | | 0.013 | | 3808.073 | 184 |
| E61R, P65R, D84S, | | | | | | |
| E95Q, C125S | | | | | | |
| T3A, H16A, M23P, E61R, | 50.000 | | 50.000 | | 1.000 | 185 |
| P65R, D84S, E95Q, | | | | | | |
| C125S | | | | | | |
| T3A, H16A, M23Q, | 18.060 | | 0.024 | | 740.771 | 186 |
| E61R, P65R, D84S, | | | | | | |
| E95Q, C125S | | | | | | |
| T3A, H16A, M23R, | 50.000 | | 0.031 | | 1627.604 | 187 |
| E61R, P65R, D84S, | | | | | | |
| E95Q, C125S | | | | | | |
| T3A, H16A, M23T, E61R, | 22.720 | | 0.030 | | 746.632 | 188 |
| P65R, D84S, E95Q, | | | | | | |
| C125S | | | | | | |
| T3A, H16A, M23V, | 8.393 | | 0.019 | | 449.545 | 189 |
| E61R, P65R, D84S, | | | | | | |
| E95Q, C125S | | | | | | |
| T3A, H16A, Q22A, E61R, | 6.656 | | 0.052 | | 128.222 | 190 |
| P65R, D84S, C125S | | | | | | |
| T3A, H16A, Q22D, E61R, | 8.855 | | 0.065 | | 136.042 | 191 |
| P65R, D84S, C125S | | | | | | |
| T3A, H16A, Q22E, E61R, | 50.000 | | 14.320 | | 3.492 | 192 |
| P65R, D84S, C125S | | | | | | |
| T3A, H16A, Q22G, E61R, | 8.201 | | 0.083 | | 98.310 | 193 |
| P65R, D84S, C125S | | | | | | |
| T3A, H16A, Q22H, E61R, | 6.329 | | 0.064 | | 98.721 | 194 |
| P65R, D84S, C125S | | | | | | |
| T3A, H16A, Q22K, E61R, | 2.178 | | 0.020 | | 110.111 | 195 |
| P65R, D84S, C125S | | | | | | |
| T3A, H16A, Q22N, E61R, | 3.134 | | 0.022 | | 140.854 | 196 |
| P65R, D84S, C125S | | | | | | |
| T3A, H16A, Q22P, E61R, | 50.000 | | 45.710 | | 1.094 | 197 |
| P65R, D84S, C125S | | | | | | |
| T3A, H16A, Q22R, E61R, | 2.745 | | 0.017 | | 159.315 | 198 |
| P65R, D84S, C125S | | | | | | |
| T3A, H16A, Q22S, E61R, | 3.131 | | 0.024 | | 129.273 | 199 |
| P65R, D84S, C125S | | | | | | |
| T3A, H16A, Q22T, E61R, | 3.338 | | 0.031 | | 108.061 | 200 |
| P65R, D84S, C125S | | | | | | |
| T3A, H16A, Q22V, E61R, | 4.837 | | 0.050 | | 96.759 | 201 |
| P65R, D84S, C125S | | | | | | |
| T3A, H16A, Q22Y, E61R, | 5.823 | | 0.090 | | 65.061 | 202 |
| P65R, D84S, C125S | | | | | | |
| T3A, H16A, E61R, P65R, | 1.628 | | 0.011 | | 153.440 | 203 |
| D84S, R120A, C125S | | | | | | |
| T3A, H16A, E61R, P65R, | 3.164 | | 0.049 | | 65.210 | 204 |
| D84S, R120D, C125S | | | | | | |
| T3A, H16A, E61R, P65R, | 1.115 | | 0.014 | | 82.531 | 205 |
| D84S, R120E, C125S | | | | | | |
| T3A, H16A, E61R, P65R, | 2.017 | | 0.018 | | 114.278 | 206 |
| D84S, R120F, C125S | | | | | | |
| T3A, H16A, E61R, P65R, | 1.829 | | 0.013 | | 141.345 | 207 |
| D84S, R120G, C125S | | | | | | |
| T3A, H16A, E61R, P65R, | 1.512 | | 0.012 | | 130.570 | 208 |
| D84S, R120H, C125S | | | | | | |
| T3A, H16A, E61R, P65R, | 2.594 | | 0.023 | | 114.323 | 209 |
| D84S, R120K, C125S | | | | | | |
| T3A, H16A, E61R, P65R, | 3.724 | | 0.040 | | 92.522 | 210 |
| D84S, R120N, C125S | | | | | | |
| T3A, H16A, E61R, P65R, | 50.000 | | 50.000 | | 1.000 | 211 |
| D84S, R120P, C125S | | | | | | |
| T3A, H16A, E61R, P65R, | 2.308 | | 0.026 | | 90.227 | 212 |
| D84S, R120Q, C125S | | | | | | |
| T3A, H16A, E61R, P65R, | 3.528 | | 0.032 | | 110.250 | 213 |
| D84S, R120S, C125S | | | | | | |
| T3A, H16A, E61R, P65R, | 50.000 | | 50.000 | | 1.000 | 214 |
| D84S, R120T, C125S | | | | | | |
| T3A, H16A, E61R, P65R, | 4.939 | | 0.062 | | 80.231 | 215 |
| D84S, R120V, C125S | | | | | | |
| T3A, H16A, E61R, P65R, | 2.385 | | 0.021 | | 116.341 | 216 |
| D84S, R120Y, C125S | | | | | | |
| T3A, H16A, E61R, P65R, | 13.010 | | 0.041 | | 320.364 | 217 |
| D84S, T123D, C125S | | | | | | |
| T3A, H16A, E61R, P65R, | 2.191 | | 0.019 | | 115.865 | 218 |
| D84S, T123E, C125S | | | | | | |
| T3A, H16A, E61R, P65R, | 18.790 | | 0.053 | | 352.798 | 219 |
| D84S, T123H, C125S | | | | | | |
| T3A, H16A, E61R, P65R, | 11.010 | | 0.050 | | 219.279 | 220 |
| D84S, T123K, C125S | | | | | | |
| T3A, H16A, E61R, P65R, | 18.150 | | 0.094 | | 192.389 | 221 |
| D84S, T123N, C125S | | | | | | |
| T3A, H16A, E61R, P65R, | 2.337 | | 0.036 | | 64.665 | 222 |
| D84S, T123Q, C125S | | | | | | |
| T3A, H16A, E61R, P65R, | 1.934 | | 0.043 | | 44.634 | 223 |
| D84S, T123R, C125S | | | | | | |
| T3A, H16A, E61R, P65R, | 50.000 | | 50.000 | | 1.000 | 224 |
| D84S, C125S, S130D | | | | | | |
| T3A, H16A, E61R, P65R, | 50.000 | | 0.064 | | 784.437 | 225 |
| D84S, C125S, S130E | | | | | | |
| T3A, H16A, E61R, P65R, | 11.950 | | 0.033 | | 361.683 | 226 |
| D84S, C125S, S130K | | | | | | |
| T3A, H16A, E61R, P65R, | 10.390 | | 0.028 | | 374.549 | 227 |
| D84S, C125S, S130N | | | | | | |
| T3A, H16A, E61R, P65R, | 7.463 | | 0.022 | | 340.776 | 228 |
| D84S, C125S, S130Q | | | | | | |
| T3A, H16A, E61R, P65R, | 8.598 | | 0.029 | | 291.853 | 229 |
| D84S, C125S, S130R | | | | | | |
| T3A, H16A, E61R, P65R, | 50.000 | | 0.085 | | 591.296 | 230 |
| D84S, C125S, S127D | | | | | | |
| T3A, H16A, E61R, P65R, | 5.134 | | 0.015 | | 351.403 | 231 |
| D84S, C125S, S127E | | | | | | |
| T3A, H16A, E61R, P65R, | 7.078 | | 0.020 | | 349.531 | 232 |
| D84S, C125S, S127H | | | | | | |
| T3A, H16A, E61R, P65R, | 50.000 | | 0.017 | | 2915.452 | 233 |
| D84S, C125S, S127P | | | | | | |
| T3A, H16A, E61R, P65R, | 50.000 | | 50.000 | | 1.000 | 234 |
| D84S, C125S, S127Q | | | | | | |
| T3A, H16A, E61R, P65R, | 4.921 | | 50.000 | | 0.098 | 235 |
| D84S, C125S, S127R | | | | | | |
| T3A, H16A, L19A, Q22A, | 5.448 | | 0.017 | | 315.278 | 236 |
| R38A, P65R, D84S, | | | | | | |
| C125S | | | | | | |
| T3A, H16A, L19A, R38A, | 5.476 | | 0.035 | | 154.340 | 237 |
| P65R, D84S, R120A, | | | | | | |
| C125S | | | | | | |
| T3A, H16A, L19A, Q22A, | 7.176 | | 0.045 | | 158.691 | 238 |
| R38A, P65R, D84S, | | | | | | |
| R120A, C125S | | | | | | |
| T3A, H16A, Q22A, | 50.000 | | 0.184 | | 272.331 | 239 |
| M23A, R38A, P65R, | | | | | | |
| D84S, E95Q, C125S | | | | | | |
| T3A, H16A, M23A, | 21.330 | | 0.057 | | 372.707 | 240 |
| R38A, P65R, D84S, | | | | | | |
| E95Q, R120A, C125S | | | | | | |
| T3A, H16A, Q22A, | 50.000 | | 0.274 | | 182.216 | 241 |
| M23A, R38A, P65R, | | | | | | |
| D84S, E95Q, R120A, | | | | | | |
| C125S | | | | | | |
| T3A, H16A, L19N, Q22A, | 50.000 | | 0.524 | | 95.511 | 242 |
| R38A, P65R, D84S, | | | | | | |
| C125S | | | | | | |
| T3A, H16A, L19N, R38A, | 41.050 | | 0.064 | | 643.720 | 243 |
| P65R, D84S, R120A, | | | | | | |
| C125S | | | | | | |
| T3A, H16A, L19N, Q22A, | 50.000 | | 0.197 | | 253.936 | 244 |
| R38A, P65R, D84S, | | | | | | |
| R120A, C125S | | | | | | |
| A1G, P2G, T3S, S4G, | 4.674 | | 0.018 | | 256.531 | 245 |
| S5G, H16A, L19A, R38A, | | | | | | |
| P65R, D84S, C125S | | | | | | |
| Δ1-5, H16A, L19A, | 5.030 | | 0.015 | | 328.758 | 246 |
| R38A, P65R, D84S, | | | | | | |
| C125S | | | | | | |
| Δ1-5, H16A, L19A, | 5.170 | | 0.015 | | 349.561 | 247 |
| R38A, P65R, D84S, | | | | | | |
| C125V | | | | | | |
| T3A, L19N, R38A, P65R, | 10.300 | | 0.021 | | 499.515 | 248 |
| D84S, C125S | | | | | | |
| T3A, H16A, L19N, R38A, | 2.102 | | 0.008 | | 276.216 | 249 |
| P65R, C125S | | | | | | |
| T3A, L19N, Q22A, R38A, | 15.060 | | 0.048 | | 314.208 | 250 |
| P65R, D84S, C125S | | | | | | |
| T3A, H16A, L19N, Q22A, | 3.184 | | 0.017 | | 189.186 | 251 |
| R38A, P65R, C125S | | | | | | |
| T3A, L19N, Q22A, R38A, | 13.730 | | 0.073 | | 188.910 | 252 |
| P65R, D84S, R120A, | | | | | | |
| C125S | | | | | | |
| T3A, H16A, L19N, Q22A, | 2.064 | | 0.021 | | 98.993 | 253 |
| R38A, P65R, R120A, | | | | | | |
| C125S | | | | | | |
| T3A, Q22A, M23A, | 10.370 | | 0.140 | | 73.966 | 254 |
| R38A, P65R, D84S, | | | | | | |
| E95Q, C125S | | | | | | |
| T3A, Q22A, M23A, | 2.063 | | 0.028 | | 73.364 | 255 |
| R38A, P65R, E95Q, | | | | | | |
| C125S | | | | | | |
| T3A, Q22A, M23A, | 10.600 | | 0.148 | | 71.767 | 256 |
| R38A, P65R, D84S, | | | | | | |
| E95Q, R120A, C125S | | | | | | |
| T3A, H16A, Q22A, | 4.556 | | 0.126 | | 36.187 | 257 |
| M23A, R38A, P65R, | | | | | | |
| E95Q, R120A, C125S | | | | | | |
| T3A, L19A, Q22A, R38A, | 2.091 | | 0.054 | | 38.946 | 258 |
| P65R, D84S, C125S | | | | | | |
| T3A, H16A, L19A, Q22A, | 0.503 | | 0.024 | | 21.148 | 259 |
| R38A, P65R, C125S | | | | | | |
| T3A, L19A, Q22A, R38A, | 1.796 | | 0.054 | | 33.265 | 260 |
| P65R, D84S, R120A, | | | | | | |
| C125S | | | | | | |
| T3A, H16A, L19A, Q22A, | 0.327 | | 0.013 | | 24.837 | 261 |
| R38A, P65R, R120A, | | | | | | |
| C125S | | | | | | |
| T3A, H16A, L19A, E61R, | 6.083 | | 0.023 | | 264.134 | 115 |
| P65R, D84S, C125S | | | | | | |
| T3A, H16A, E61R, P65R, | 2.062 | | 0.0422 | | 49 | 262 |
| D84S, C125S, I129A | | | | | | |
| T3A, H16A, E61R, P65R, | 1.435 | | 0.0697 | | 21 | 263 |
| D84S, C125S, I129H | | | | | | |
| T3A, H16A, E61R, P65R, | 26.220 | | 0.0648 | | 405 | 264 |
| D84S, C125S, H29R | | | | | | |
| T3A, H16A, E61R, P65R, | 16.170 | | 0.0561 | | 288 | 265 |
| D84S, C125S, I129S | | | | | | |
| T3A, H16A, E61R, P65R, | 2.062 | | 0.042 | | 49 | 266 |
| D84S, C125C, Q126N | | | | | | |
| T3A, H16A, E61R, P65R, | 1.435 | | 0.070 | | 21 | 267 |
| D84S, C125C, Q126Y | | | | | | |
| T3A, H16A, E61R, P65R, | 26.220 | | 0.065 | | 405 | 268 |
| D84S, C125C, S127N | | | | | | |
| T3A, H16A, E61R, P65R, | 16.170 | | 0.056 | | 288 | 269 |
| D84S, C125C, S130P | | | | | | |

TABLE 14
EC₅₀s of polypeptides comprising a modified IL-2

| | Non- | CD8a | | |
|---|---|---|---|---|
| | tar- | Tar- | | |
| | geted | geted | CD8a | SEQ |
| | EC₅₀ | EC₅₀ | Win- | ID |
| IL-2 Modifications | (nM) | (nM) | dow | NO. |
| T3A, H16A, E61R, P65R, D84Y, C125S | 4.515 | 0.011 | 400 | 270 |
| T3A, H16A, M23T, E61R, P65R, D84Y, | 50.000 | 0.057 | 871 | 271 |
| E95Q, C125S | | | | |
| T3A, H16A, L19N, E61R, P65R, D84Y, | 50.000 | 0.074 | 673 | 272 |
| C125S | | | | |
| T3A, H16A, L19N, M23T, E61R, P65R, | 50.000 | 50.000 | 1 | 273 |
| D84Y, E95Q, C125S | | | | |
| T3A, H16A, E61R, P65R, D84Y, | 50.000 | 0.088 | 568 | 274 |
| C125S, S127D | | | | |
| T3A, H16A, M23T, E61R, P65R, D84Y, | 50.000 | 50.000 | 1 | 275 |
| E95Q, C125S, S127D | | | | |
| T3A, H16A, L19N, E61R, P65R, D84Y, | 50.000 | 50.000 | 1 | 276 |
| C125S, S127D | | | | |
| T3A, H16A, L19N, M23T, E61R, P65R, | 50.000 | 50.000 | 1 | 277 |
| D84Y, E95Q, C125S, S127D | | | | |

### Example 26: Non-Targeted Vs Targeted Activities of Polypeptides Comprising a Modified IL-2

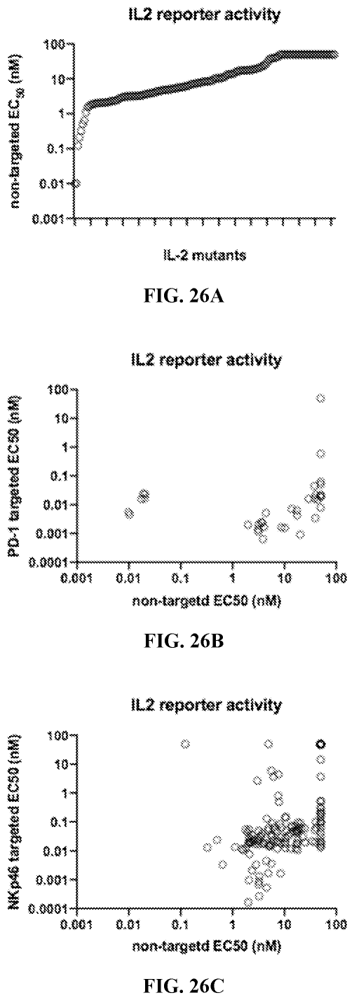

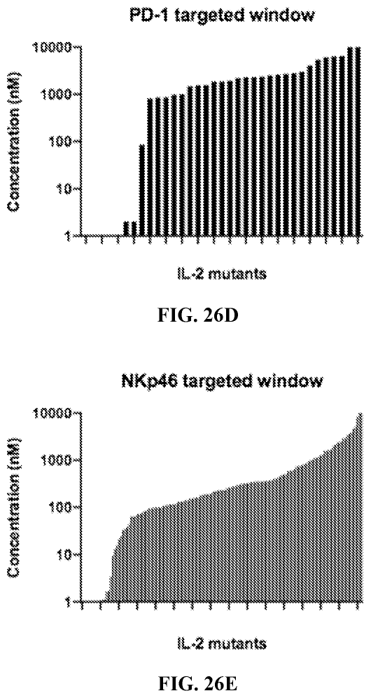

The non-targeted and targeted IL-2 reporter activities of polypeptides described herein were analyzed and compared by plotting non-targeted and targeted EC₅₀s determined in the IL-2 reporter assays described above. FIG. 26A shows the non-targeted activities of IL-2 mutants included in the analysis. This graph shows of the reduction of affinity of the IL-2 mutants for the trimeric IL-2 receptor. FIGS. 26B and 26C show plots of targeted EC₅₀s for PD-1 and NKp46, respectively, versus the non-targeted EC₅₀s. These comparisons show the enhanced abilities of the reduced affinity IL-2 mutants to signal when targeted to PD-1 or NKp46 by a high affinity VHH. FIGS. 26D and 26E show the PD-1 and NKp46 targeted windows, respectively, which are calculated as described in Example 25. These graphs show the concentration ranges at which targeted activity is achieved while avoiding non-targeted IL-2 activity.

### Example 27: Activities of Polypeptides Comprising a Modified IL-2

The relative activities of polypeptides comprising a modified IL-2 fused to the C-terminus of a γδTCR-binding VHH were determined by measuring pSTAT5 levels of PBMCs via flow cytometry. The polypeptides comprise Fc regions selected to form monovalent or bivalent γδTCR-binding polypeptides. The structures of the polypeptides are indicated in the table below.

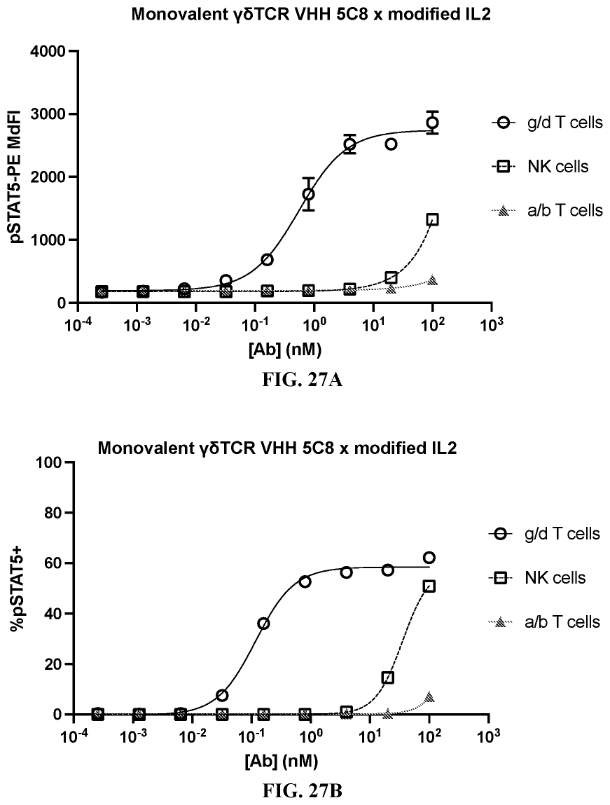

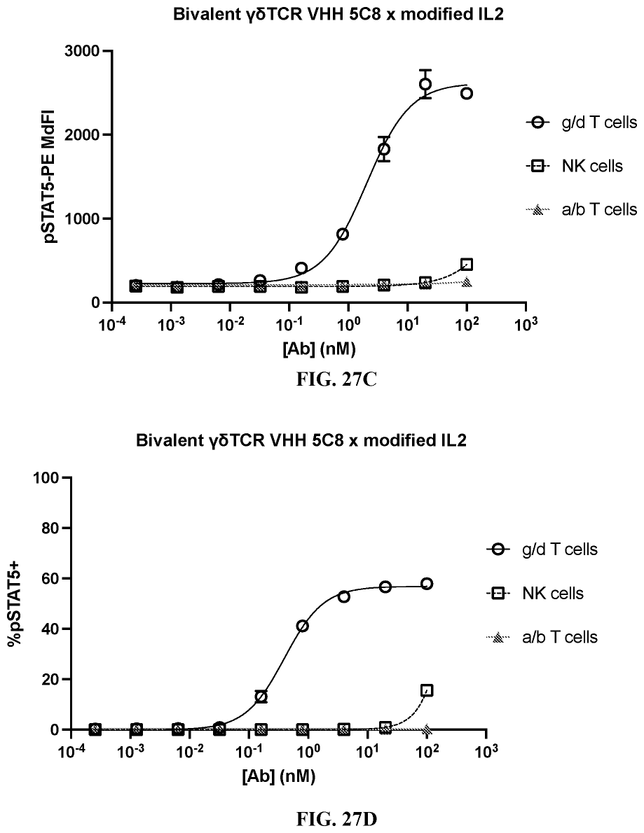

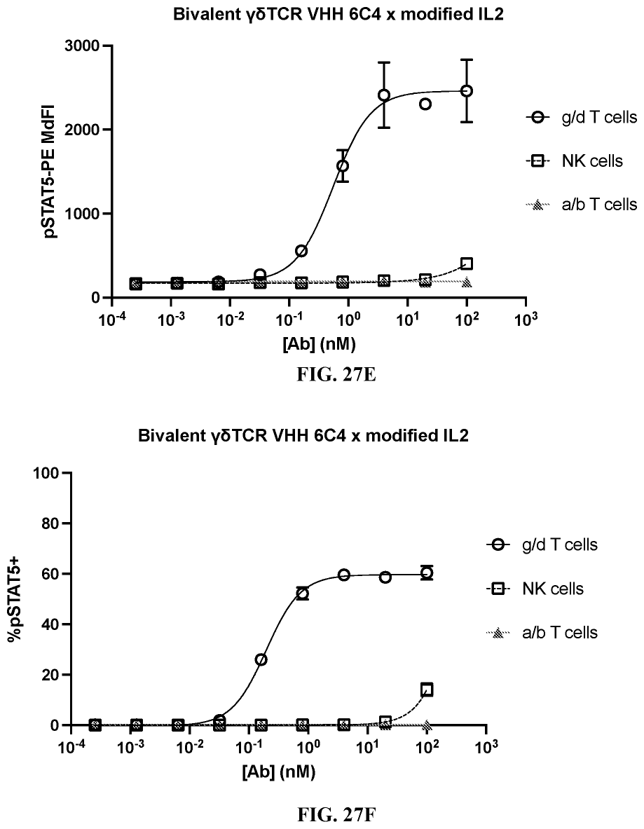

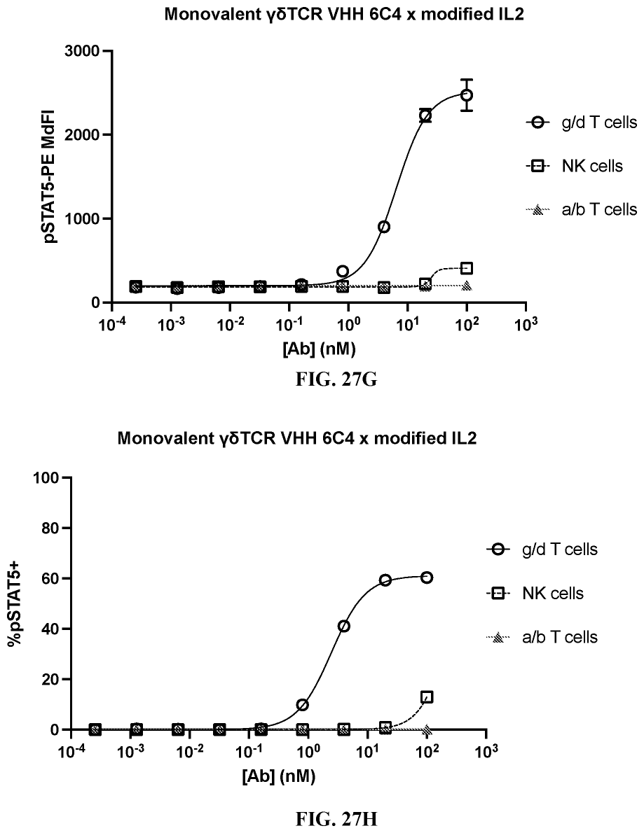

TABLE 15
γδTCR-targeted polypeptides comprising modified IL-2

| | | SEQ |
|---|---|---|
| | Va- | ID |
| Polypeptide | lency | NO(s). |
| γδTCR 5C8 VHH-xELL hole T366S, L368A, Y407V, | mono- | 294, |
| H435RFc; xELL knob Fc-IL-2-T3G-P65R-H16A- | valent | 295 |
| D84S-C125S; FIGS. 27A, B | | |
| γδTCR 5C8 VHH-xELL hole T366S, L368A, Y407V, | bi- | 295, |
| H435R Fc; γδTCR 5C8 VHH-xELL knob Fc-IL-2- | valent | 296 |
| T3A-H16A-E61R-P65R-D84Y-C125S; FIGS. 27C, D | | |
| γδTCR 6C4 VHH-xELL hole T366S, L368A, Y407V | bi- | 298, |
| H435R Fc; γδTCR 6C4 VHH-xELL-knob Fc-IL-2- | valent | 299 |
| T3A-H16A-E61R-P65R-D84Y-C125S; FIGS. 27E, F | | |
| 410A9vl (untargeted) VHH-xELL hole T366S, L368A, | mono- | 298, |
| Y407V H435R Fc; γδTCR 6C4 VHH-xELL-knob Fc- | valent | 300 |
| IL-2-T3A-H16A- E61R-P65R-D84Y-C125S; FIGS. | | |
| 27G, H | | |

PBMCs were isolated from healthy donor leukopaks by lymphoprep density gradient centrifugation. The cells were labeled for 20 minutes at room temperature with the following fluorescently conjugated antibodies: non-competing anti-γδTCR-FITC, anti-CD3-BV785, and anti-CD56-BV421. After washing, 200,000 PBMCs per well were seeded in a 96-well plate. The cells were treated with a titration of a fusion protein listed in the table above, starting at an initial concentration of 100 nM and titrating across the plate 1:5, in duplicate. The plates were incubated for 20 minutes at 37° C./5% CO₂. The cells were fixed with BD Cytofix/Cytoperm™ (BD Biosciences), permeabilized in 90% ice-cold methanol, and levels of phosphorylated STAT5 (“pSTAT5”) on γδT cells were measured using flow cytometry using a phospho-specific anti-pSTAT5-PE antibody (1:70).

As shown in FIGS. 27A-27H, treatment with various bivalent or monovalent γδTCR targeted modified IL-2 polypeptides led to a dose-dependent increase in the percentage of pSTAT5+γδT cells and the pSTAT5 median fluorescent intensity specifically on γδT cells. In contrast, there was little pSTAT5 activation on NK or αβT cells below 100 nM.

### Example 28: Activities of Polypeptides Comprising a Modified IL-2

The relative activities of polypeptides comprising a modified IL-2 fused to the C-terminus of a γδTCR-binding VHH or a non-targeted VHH were determined by measuring γδT cell and αβT cell proliferation and accumulation. The polypeptides comprise Fc regions selected to form monovalent or bivalent polypeptides. The structures of the polypeptides are indicated in the table below.

TABLE 16
γδTCR-targeted and non-targeted polypeptides comprising modified
IL-2

| | | SEQ |
|---|---|---|
| | | ID |
| Polypeptide | Valency | NO(s). |
| γδTCR 5C8 VHH-xELL knob Fc-IL-2-T3A-H16A- | mono- | 64, |
| E61R-P65R-D84Y-C125S; xELL hole Fc H435R, | valent | 296 |
| T366S, L368A, Y407V | | |
| Untargeted VHH-xELL hole T366S, L368A, Y407V | N/A | 300, |
| H435R Fc; Untargeted VHH-xELL knob Fc-IL-2- | | 301 |
| T3A-H16A- E61R-P65R-D84Y-C125S | | |

IL-2 promotes the activation and proliferation of T cell populations. In order to assess the effect of γδTCR-targeted modified IL-2 on T cell proliferation, PBMCs were isolated from healthy donor leukopaks using lymphoprep density gradient medium. The cells were labeled with CellTrace Violet proliferative dye for 10 minutes at 37° C. After washing, the cells were resuspended in RPMI+10% FBS and 300,000 cells per well were added to a 96-well plate. The cells were treated with a titration of a fusion protein listed in the table above, starting at an initial concentration of 100 nM and titrating across the plate 1:5, in duplicate. The plates were incubated at 37° C./5% CO₂ for 7 days. The cells were labeled with the following fluorescently conjugated antibodies: CD3-BV785, γδTCR-FITC, and the viability dye propidium iodide for 30 minutes at 4° C. The cells were washed and analyzed by flow cytometry.

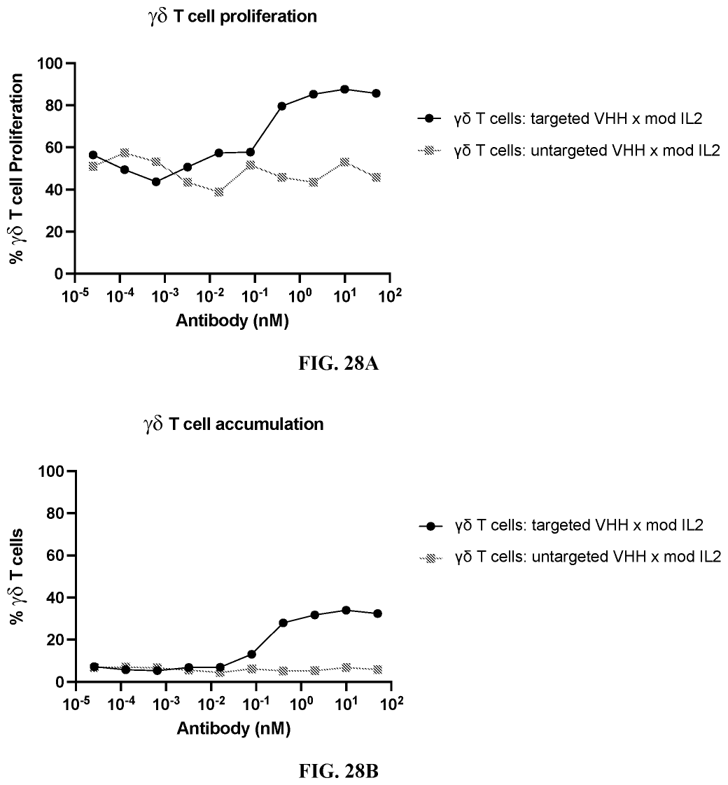

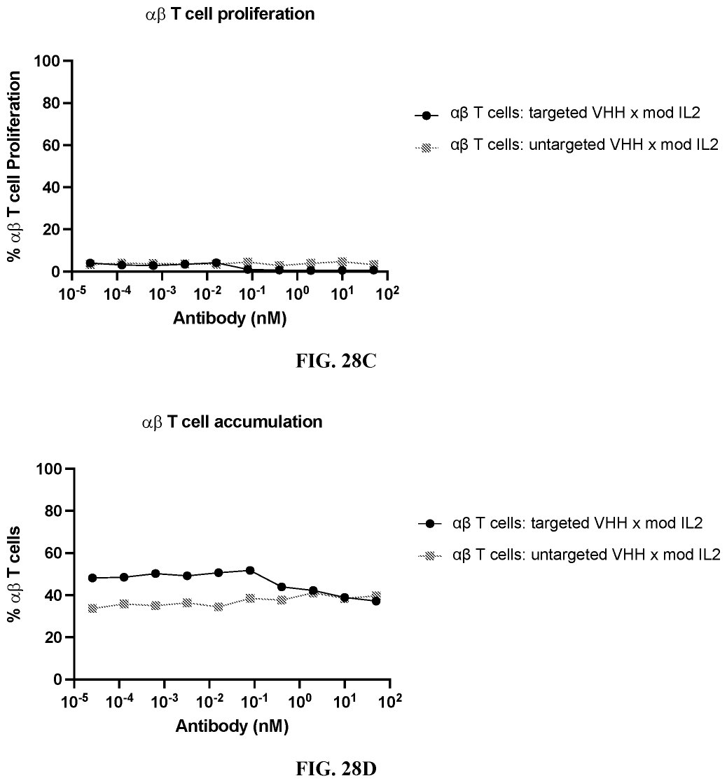

As shown in FIGS. 28A-28D, treatment with a γδTCR targeted modified IL-2 led to a dose-dependent increase in the proliferation and accumulation of γδT cells (FIG. 28A and FIG. 28B). The percentage of γδT cells among the total CD3⁺ population also increased with γδTCR targeted modified IL-2 treatment, along with a concomitant decrease in T cells. The effect was specific for γδT cells as the αβT cell population did not proliferate or accumulate in response to treatment with the γδTCR targeted modified IL-2 (FIG. 28C and FIG. 28D). The non-targeted VHH fused to the modified IL-2 did not promote the proliferation of either γδT cells or αβT cells at the doses tested, demonstrating target specificity of the targeted modified IL-2 polypeptides.

### Example 29: Activities of Polypeptides Comprising a Modified IL-2

The relative activities of polypeptides comprising a modified IL-2 were determined by measuring pSTAT5 levels of PBMCs via flow cytometry. The structures of the polypeptides are indicated in the table below.

TABLE 17
Polypeptides comprising modified IL-2

| | SEQ |
|---|---|
| | ID |
| Polypeptide | NO(s). |
| Wild type IL-2 | 1 |
| Untargeted VHH-xELL hole T366S, L368A, Y407V H435R | 300, |
| Fc; 410A9v1 Untargeted VHH-xELL knob Fc-IL-2-T3A- | 301 |
| H16A-E61R-P65R-D84Y-C125S | |

Levels of pSTAT5 were measured by intracellular flow cytometry as a proximal readout of IL-2 receptor engagement and signaling. Human PBMCs were plated in a 96-well plate at 1,000,000 cells per well in complete growth media (RPMI, 10% FBS, 1% antibiotic-antimycotic). Test polypeptides were then diluted, and a 5-fold serial dilution was made. Serial dilutions were added to the cells and incubated for 15 minutes at 37° C. Cells were then fixed in 100 μL of Cytofix fixation buffer (BD) for 30 minutes at 4° C. Cells were then washed once in 200 μL FACS buffer and permeabilized in Perm buffer III (BD Phosflow) for 30 minutes at 4° C. Permeabilized cells were washed a total of three times in Permeabilization Buffer (eBioscience) and then incubated in Permeabilization Buffer containing fluorescently labeled antibodies against CD4 (OKT4, 1:100), CD3 (SP34-2, 1:50), CD16 (3G8, 1:1000), pSTAT5 (SRBCZX, 1:70), CD56 (NCAM16.2, 1:500), and CD8 (RPA-T8, 1:4000) overnight at 4° C. The next day, cells were washed with 150 μL FACS buffer and analyzed using an ACEA Biosciences Novocyte-Quanteon Flow Cytometer. IL-2 signaling was quantified via increases in the frequency and median fluorescence intensity levels of the fluorescently labeled antibody detecting pSTAT5 on NK cells (CD3−CD56brightCD16−). The data were plotted and analyzed using GraphPad Prism analysis software.

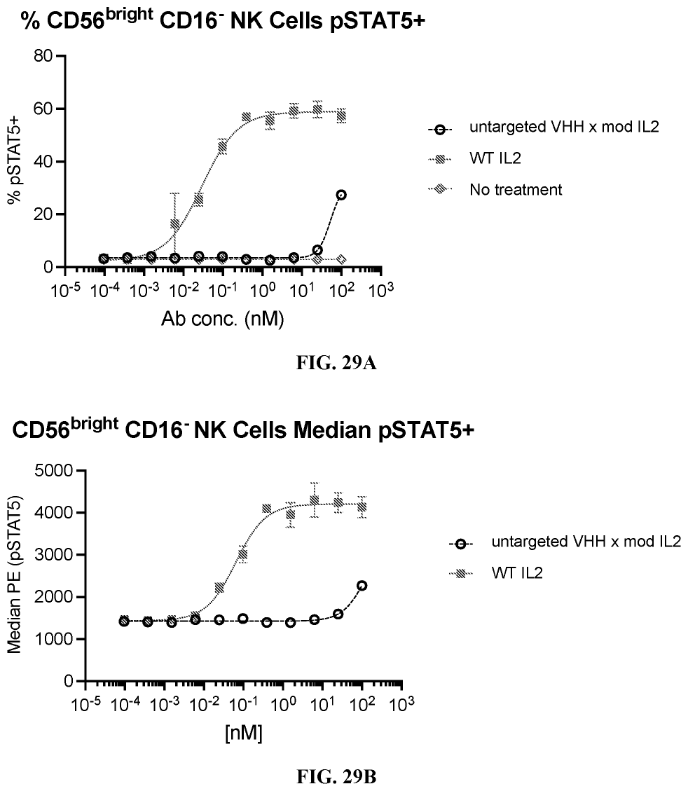

As shown in FIGS. 29A-29B, the wild type IL-2 activated the cells in a dose-dependent manner, with an EC₅₀ of wild-type recombinant IL-2 of approximately 0.06 nM. The polypeptide comprising an IL-2 variant comprising T3A, H16A, E61R, P65R, D84Y, and C125S mutations showed significantly attenuated activity compared to the wild type IL-2.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

TABLE of
Certain Sequences

| SEQ | | |
|---|---|---|
| ID | | |
| NO | Description | Sequence |
| 1 | Wild type Human | APTSSSTKKTQLQLEHLLLDLQMILNGINNYKNPKLTRMLTFKFYMPKKATEL |
| | IL-2 | KHLQCLEEELKPLEEVLNLAQSKNFHLRPRDLISNINVIVLELKGSETTFMCE |
| | | YADETATIVEFLNRWITFCQSIISTLT |
| 2 | IL-2v | 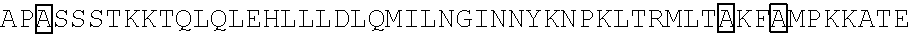 |
| | | 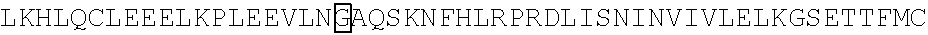 |
| | | 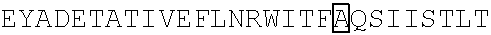 |
| 3 | IL-2-P65R | APTSSSTKKTQLQLEHLLLDLQMILNGINNYKNPKLTRMLTFKFYMPKKATEL |
| | | 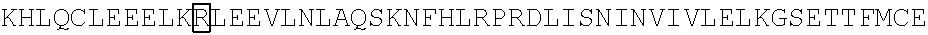 |
| | | YADETATIVEFLNRWITFCQSIISTLT |
| 4 | IL-2-H16A | 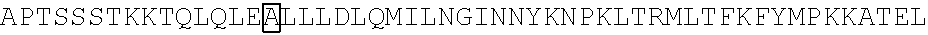 |
| | | KHLQCLEEELKPLEEVLNLAQSKNFHLRPRDLISNINVIVLELKGSETTFMCE |
| | | YADETATIVEFLNRWITFCQSIISTLT |
| 5 | IL-2-D84S | APTSSSTKKTQLQLEHLLLDLQMILNGINNYKNPKLTRMLTFKFYMPKKATEL |
| | | 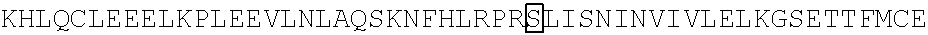 |
| | | YADETATIVEFLNRWITFCQSIISTLT |
| 6 | IL-2-E15S | 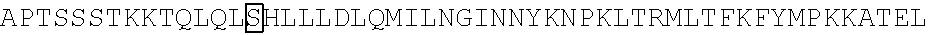 |
| | | KHLQCLEEELKPLEEVLNLAQSKNFHLRPRDLISNINVIVLELKGSETTFMCE |
| | | YADETATIVEFLNRWITFCQSIISTLT |
| 7 | IL-2-M23A | 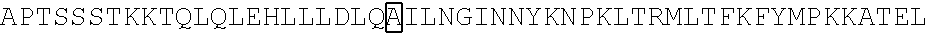 |
| | | KHLQCLEEELKPLEEVLNLAQSKNFHLRPRDLISNINVIVLELKGSETTFMCE |
| | | YADETATIVEFLNRWITFCQSIISTLT |
| 8 | IL-2-E95Q | APTSSSTKKTQLQLEHLLLDLQMILNGINNYKNPKLTRMLTFKFYMPKKATEL |
| | | 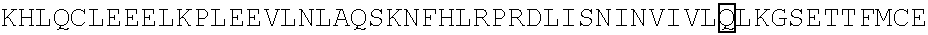 |
| | | YADETATIVEFLNRWITFCQSIISTLT |
| 9 | IL-2-P65E | APTSSSTKKTQLQLEHLLLDLQMILNGINNYKNPKLTRMLTFKFYMPKKATEL |
| | | 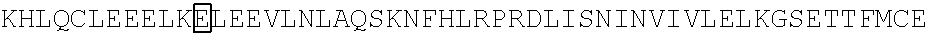 |
| | | YADETATIVEFLNRWITFCQSIISTLT |
| 10 | IL-2-F42K | 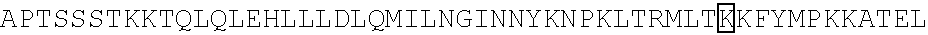 |
| | | KHLQCLEEELKPLEEVLNLAQSKNFHLRPRDLISNINVIVLELKGSETTFMCE |
| | | YADETATIVEFLNRWITFCQSIISTLT |
| 11 | IL-2-H16A-F42K | 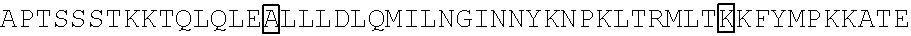 |
| | | LKHLQCLEEELKPLEEVLNLAQSKNFHLRPRDLISNINVIVLELKGSETTFMC |
| | | EYADETATIVEFLNRWITFCQSIISTLT |
| 12 | IL-2-D84S-F42K | 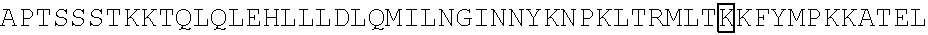 |
| | | 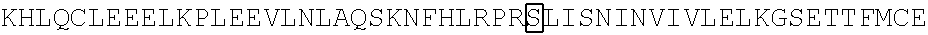 |
| | | YADETATIVEFLNRWITFCQSIISTLT |
| 13 | IL-2-E15S-F42K | 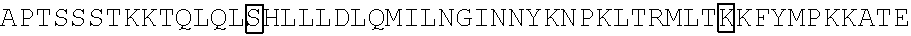 |
| | | LKHLQCLEEELKPLEEVLNLAQSKNFHLRPRDLISNINVIVLELKGSETTFMC |
| | | EYADETATIVEFLNRWITFCQSIISTLT |
| 14 | IL-2-M23A-F42K | 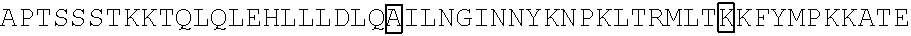 |
| | | LKHLQCLEEELKPLEEVLNLAQSKNFHLRPRDLISNINVIVLELKGSETTFMC |
| | | EYADETATIVEFLNRWITFCQSIISTLT |
| 15 | IL-2-E95Q-F42K | 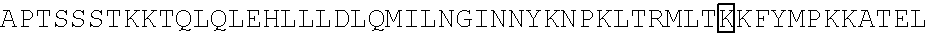 |
| | | 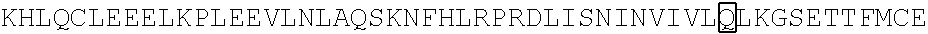 |
| | | YADETATIVEFLNRWITFCQSIISTLT |
| 16 | IL-2-P65R-H16A | 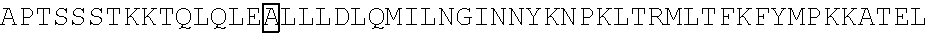 |
| | | 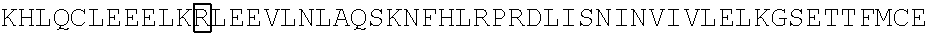 |
| | | YADETATIVEFLNRWITFCQSIISTLT |
| 17 | IL-2-P65R-D84S | APTSSSTKKTQLQLEHLLLDLQMILNGINNYKNPKLTRMLTFKFYMPKKATEL |
| | | 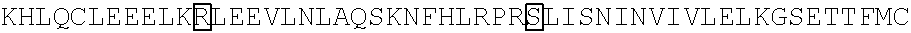 |
| | | EYADETATIVEFLNRWITFCQSIISTLT |
| 18 | IL-2-P65R-E15S | 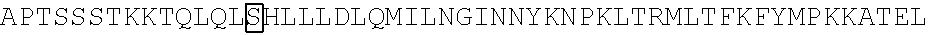 |
| | | 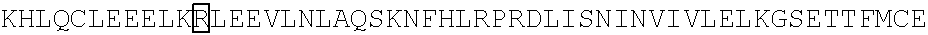 |
| | | YADETATIVEFLNRWITFCQSIISTLT |
| 19 | IL-2-P65R-M23A | 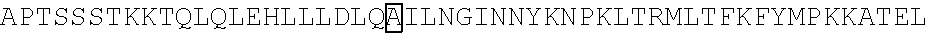 |
| | | 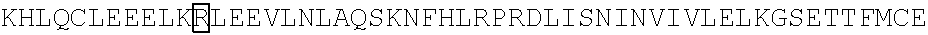 |
| | | YADETATIVEFLNRWITFCQSIISTLT |
| 20 | IL-2-P65R-E95Q | APTSSSTKKTQLQLEHLLLDLQMILNGINNYKNPKLTRMLTFKFYMPKKATEL |
| | | 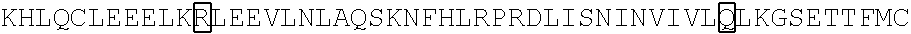 |
| | | EYADETATIVEFLNRWITFCQSIISTLT |
| 21 | IL-2-P65R-H16A- | 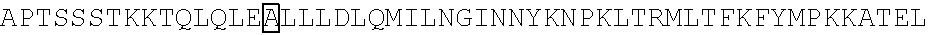 |
| | D84S (IL-2-RAS) | 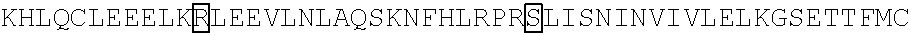 |
| | | EYADETATIVEFLNRWITFCQSIISTLT |
| 22 | IL-2-T3A-C125S | 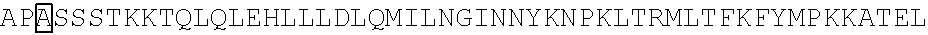 |
| | | KHLQCLEEELKPLEEVLNLAQSKNFHLRPRDLISNINVIVLELKGSETTFMCE |
| | | 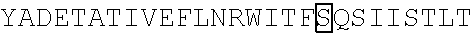 |
| 23 | IL-2-T3A-P65R- | 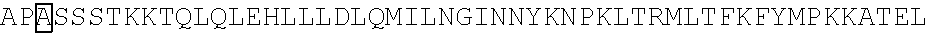 |
| | C125S | 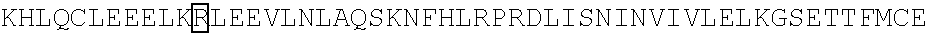 |
| | | 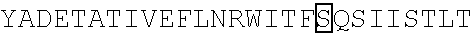 |
| 24 | IL-2-T3A-H16A- | 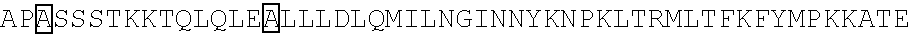 |
| | C125S | LKHLQCLEEELKPLEEVLNLAQSKNFHLRPRDLISNINVIVLELKGSETTFMC |
| | | 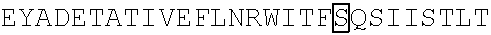 |
| 25 | IL-2-T3A-D84S- | 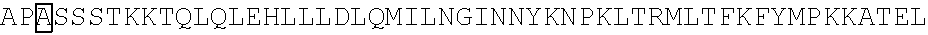 |
| | C125S | 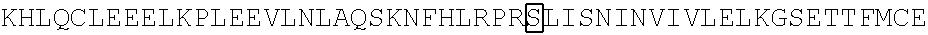 |
| | | 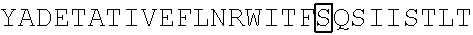 |
| 26 | IL-2-T3A-H16A- | 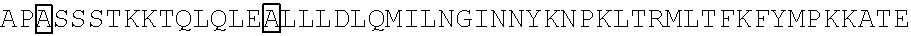 |
| | P65R-C125S | 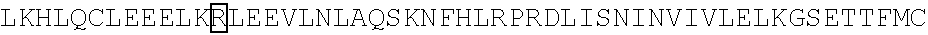 |
| | | 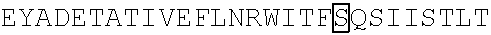 |
| 27 | IL-2-T3A-P65R- | 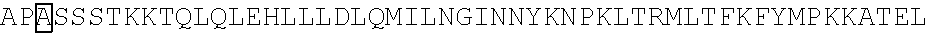 |
| | D84S-C125S | 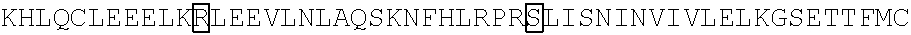 |
| | | 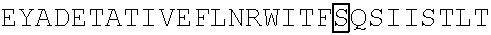 |
| 28 | IL-2-T3A-H16A- | 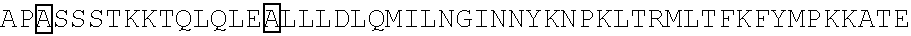 |
| | P65R-D84S-C125S | 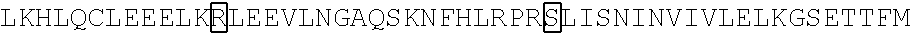 |
| | | 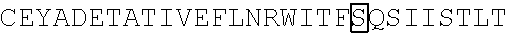 |
| 29 | IL-2-no9-H16A- | 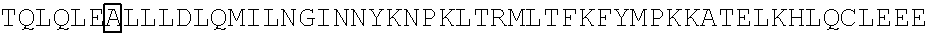 |
| | P65R-C125S | 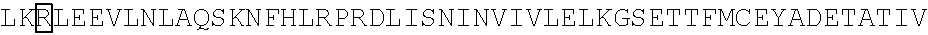 |
| | | 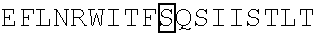 |
| 30 | IL-2-no9-P65R- | TQLQLEHLLLDLQMILNGINNYKNPKLTRMLTFKFYMPKKATELKHLQCLEEE |
| | D84S-C125S | 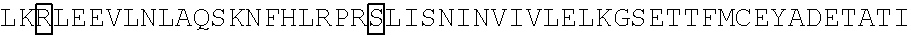 |
| | | 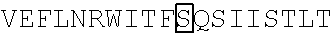 |
| 31 | IL-2-no9-H16A- | 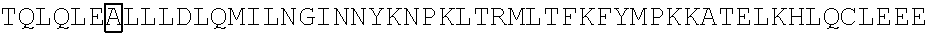 |
| | P65R-D84S-C125S | 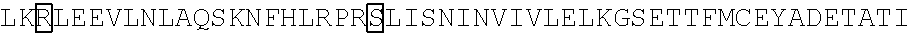 |
| | | 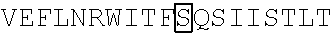 |
| 32 | Wild type IL-2- | APTSSSTKKTQLQLEHLLLDLQMILNGINNYKNPKLTRMLTFKFYMPKKATEL |
| | xELL “knob” Fc | KHLQCLEEELKPLEEVLNLAQSKNFHLRPRDLISNINVIVLELKGSETTFMCE |
| | | YADETATIVEFLNRWITFCQSIISTLTPGGGGDKTHTCPPCPAPGGPSVFLFP |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | LPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 33 | IL-2-F42K-xELL | APTSSSTKKTQLQLEHLLLDLQMILNGINNYKNPKLTRMLTKKFYMPKKATEL |
| | “knob” Fc | KHLQCLEEELKPLEEVLNLAQSKNFHLRPRDLISNINVIVLELKGSETTFMCE |
| | | YADETATIVEFLNRWITFCQSIISTLTPGGGGDKTHTCPPCPAPGGPSVFLFP |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | LPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 34 | IL-2-P65E-xELL | APTSSSTKKTQLQLEHLLLDLQMILNGINNYKNPKLTRMLTFKFYMPKKATEL |
| | “knob” Fc | KHLQCLEEELKELEEVLNLAQSKNFHLRPRDLISNINVIVLELKGSETTFMCE |
| | | YADETATIVEFLNRWITFCQSIISTLTPGGGGDKTHTCPPCPAPGGPSVFLFP |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | LPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 35 | IL-2-P65R-xELL | APTSSSTKKTQLQLEHLLLDLQMILNGINNYKNPKLTRMLTFKFYMPKKATEL |
| | “knob” Fc | KHLQCLEEELKRLEEVLNLAQSKNFHLRPRDLISNINVIVLELKGSETTFMCE |
| | | YADETATIVEFLNRWITFCQSIISTLTPGGGGDKTHTCPPCPAPGGPSVFLFP |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | LPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 36 | IL-2-F42K-D84S- | APTSSSTKKTQLQLEHLLLDLQMILNGINNYKNPKLTRMLTKKFYMPKKATEL |
| | xELL “knob” Fc | KHLQCLEEELKPLEEVLNLAQSKNFHLRPRSLISNINVIVLELKGSETTFMCE |
| | | YADETATIVEFLNRWITFCQSIISTLTPGGGGDKTHTCPPCPAPGGPSVFLFP |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | LPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 37 | IL-2-F42K-E95Q- | APTSSSTKKTQLQLEHLLLDLQAILNGINNYKNPKLTRMLTKKFYMPKKATEL |
| | xELL “knob” Fc | KHLQCLEEELKPLEEVLNLAQSKNFHLRPRDLISNINVIVLELKGSETTFMCE |
| | | YADETATIVEFLNRWITFCQSIISTLTPGGGGDKTHTCPPCPAPGGPSVFLFP |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | LPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 38 | IL-2-F42K-M23A- | APTSSSTKKTQLQLEHLLLDLQAILNGINNYKNPKLTRMLTKKFYMPKKATEL |
| | xELL “knob” Fc | KHLQCLEEELKPLEEVLNLAQSKNFHLRPRDLISNINVIVLELKGSETTFMCE |
| | | YADETATIVEFLNRWITFCQSIISTLTPGGGGDKTHTCPPCPAPGGPSVFLFP |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | LPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 85 | IL-2-F42K-E15S- | APTSSSTKKTQLQLSHLLLDLQMILNGINNYKNPKLTRMLTKKFYMPKKATEL |
| | xELL “knob” Fc | KHLQCLEEELKPLEEVLNLAQSKNFHLRPRDLISNINVIVLELKGSETTFMCE |
| | | YADETATIVEFLNRWITFCQSIISTLTPGGGGDKTHTCPPCPAPGGPSVFLFP |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | LPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 39 | IL-2-H16A-F42K- | 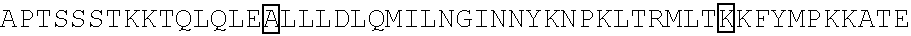 |
| | xELL “knob” Fc | LKHLQCLEEELKPLEEVLNLAQSKNFHLRPRDLISNINVIVLELKGSETTFMC |
| | | EYADETATIVEFLNRWITFCQSIISTLTPGGGGDKTHTCPPCPAPGGPSVFLF |
| | | PPKPKDTLMISRTPEVTCVVVDVSHEDPEVKFNWYVDGVEVHNAKTKPREEQY |
| | | NSTYRWSVLTVLHQDWLNGKEYKCKVSNKALPAPIEKTISKAKGQPREPQVY |
| | | TLPPCRDELTKNQVSLWCLVKGFYPSDIAVEWESNGQPENNYKTTPPVLDSDG |
| | | SFFLYSKLTVDKSRWQQGNVFSCSVMHEALHNHYTQKSLSLSPGK |
| 40 | IL-2-H16A-xELL | 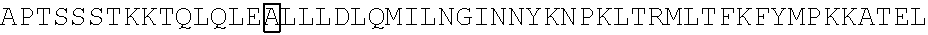 |
| | “knob” Fc | LKHLQCLEEELKPLEEVLNLAQSKNFHLRPRDLISNINVIVLELKGSETTFMCE |
| | | 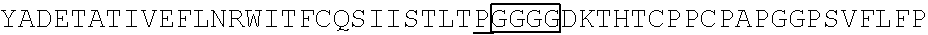 |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | 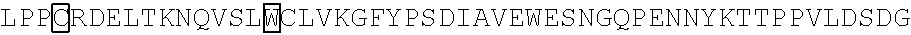 |
| | | SFFLYSKLTVDKSRWQQGNVFSCSVMHEALHNHYTQKSLSLSPGK |
| 41 | IL-2-P65R-H16A- | APTSSSTKKTQLQLEALLLDLQMILNGINNYKNPKLTRMLTFKFYMPKKATEL |
| | xELL “knob” Fc | KHLQCLEEELKRLEEVLNLAQSKNFHLRPRDLISNINVIVLELKGSETTFMCE |
| | | YADETATIVEFLNRWITFCQSIISTLTPGGGGDKTHTCPPCPAPGGPSVFLFP |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | LPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 42 | IL-2-P65R-D84S- | APTSSSTKKTQLQLEHLLLDLQMILNGINNYKNPKLTRMLTFKFYMPKKATEL |
| | xELL “knob” Fc | KHLQCLEEELKRLEEVLNLAQSKNFHLRPRSLISNINVIVLELKGSETTFMCE |
| | | YADETATIVEFLNRWITFCQSIISTLTPGGGGDKTHTCPPCPAPGGPSVFLFP |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | LPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 43 | IL-2-RAS-xELL | APTSSSTKKTQLQLEALLLDLQMILNGINNYKNPKLTRMLTFKFYMPKKATEL |
| | “knob” Fc | KHLQCLEEELKRLEEVLNLAQSKNFHLRPRSLISNINVIVLELKGSETTFMCE |
| | | YADETATIVEFLNRWITFCQSIISTLTPGGGGDKTHTCPPCPAPGGPSVFLFP |
| | | PKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYT |
| | | LPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 44 | Linker-EVN xELL | KPGGGGDKTHTCPPCPAPGGPSVFLFPPKPKDTLMRSRTPEVTCVVVDVSHED |
| | “hole” Fc | PEVKFNWYVDGVEVHNAKTKPREEQYNSTYRVVSVLTVLHQDWLNGKEYKCKV |
| | | SNKALPAPIEKTISKAKGQPREPQVCTLPPSRDELTKNQVSLSCAVKGFYPSD |
| | | IAVEWESNGQPENNYKTTPPVLDSDGSFFLVSKLTVDKSRWQQGNVFSCSVMH |
| | | EALHNHYTQKSLSLSPGK |
| 45 | xELL “knob” Fc- | DKTHTCPPCPAPGGPSVFLFPPKPKDTLMISRTPEVTCVVVDVSHEDPEVKFN |
| | IL-2-T3G-C125S | WYVDGVEVHNAKTKPREEQYNSTYRVVSVLTVLHQDWLNGKEYKCKVSNKALP |
| | | APIEKTISKAKGQPREPQVYTLPPCRDELTKNQVSLWCLVKGFYPSDIAVEWE |
| | | SNGQPENNYKTTPPVLDSDGSFFLYSKLTVDKSRWQQGNVFSCSVMHEALHNH |
| | | YTQKSLSLSPGGSGGSAPGSSSTKKTQLQLEHLLLDLQMILNGINNYKNPKLT |
| | | RMLTFKFYMPKKATELKHLQCLEEELKPLEEVLNLAQSKNFHLRPRDLISNIN |
| | | VIVLELKGSETTFMCEYADETATIVEFLNRWITFSQSIISTLT |
| 46 | xELL “knob” Fc- | DKTHTCPPCPAPGGPSVFLFPPKPKDTLMISRTPEVTCVVVDVSHEDPEVKFN |
| | IL-2-RAS-T3G- | WYVDGVEVHNAKTKPREEQYNSTYRVVSVLTVLHQDWLNGKEYKCKVSNKALP |
| | C125S | APIEKTISKAKGQPREPQVYTLPPCRDELTKNQVSLWCLVKGFYPSDIAVEWE |
| | | SNGQPENNYKTTPPVLDSDGSFFLYSKLTVDKSRWQQGNVFSCSVMHEALHNH |
| | | YTQKSLSLSPGGSGGSAPGSSSTKKTQLQLEALLLDLQMILNGINNYKNPKLT |
| | | RMLTFKFYMPKKATELKHLQCLEEELKRLEEVLNLAQSKNFHLRPRSLISNIN |
| | | VIVLELKGSETTFMCEYADETATIVEFLNRWITFSQSIISTLT |
| 47 | Fc region 1 (human | DKTHTCPPCPAPELLGGPSVFLFPPKPKDTLMISRTPEVTCVVVDVSHEDPEV |
| | wild type IgG1) | KFNWYVDGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVMHEAL |
| | | HNHYTQKSLSLSPG |
| 48 | Fc region 2 (human | DKTHTCPPCPAPGGPSVFLFPPKPKDTLMISRTPEVTCVVVDVSHEDPEVKFN |
| | IgG1 xELL | WYVDGVEVHNAKTKPREEQYNSTYRVVSVLTVLHQDWLNGKEYKCKVSNKALP |
| | “knob”) | APIEKTISKAKGQPREPQVYTLPPCRDELTKNQVSLWCLVKGFYPSDIAVEWE |
| | | SNGQPENNYKTTPPVLDSDGSFFLYSKLTVDKSRWQQGNVFSCSVMHEALHNH |
| | | YTQKSLSLSPG |
| 49 | Fc region 3 (human | DKTHTCPPCPAPGGPSVFLFPPKPKDTLMRSRTPEVTCVVVDVSHEDPEVKFN |
| | IgG1 EVN xELL | WYVDGVEVHNAKTKPREEQYNSTYRVVSVLTVLHQDWLNGKEYKCKVSNKALP |
| | “hole” I253R) | APIEKTISKAKGQPREPQVCTLPPSRDELTKNQVSLSCAVKGFYPSDIAVEWE |
| | | SNGQPENNYKTTPPVLDSDGSFFLVSKLTVDKSRWQQGNVFSCSVMHEALHNH |
| | | YTQKSLSLSPG |
| 50 | Fc region | DKTHTCPPCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVMHEAL |
| | | HNHYTQKSLSLSPG |
| 51 | Fc region xELL | DKTHTCPPCPAPGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVSNKALP |
| | | APIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVMHEALHNH |
| | | YTQKSLSLSPG |
| 52 | Fc region xELL | DKTHTCPPCPAPGGPSVFLFPPKPKDTLMISRTPETCVVVDVSHEDPEVKFNW |
| | H435R | YVDGVEVHNAKTKPREEQYNSTYRVVSVLTVLHQDWLNGKEYKCKVSNKALPA |
| | | PIEKTISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEALHNRY |
| | | TQKSLSLSPG |
| 53 | Fc region xELL | DKTHTCPPCPAPGGPSVFLFPPKPKDTLYISRTPEVTCVVVDVSHEDPEVKFN |
| | M252Y and | WYVDGVEVHNAKTKPREEQYNSTYRVVSVLTVLHQDWLNGKEYKCKVSNKALP |
| | M428V (YV) | APIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVVHEALHNH |
| | | YTQKSLSLSPG |
| 54 | Fc region xELL | DKTHTCPPCPAPGGPSVFLFPPKPKDTLYISRTPEVTCVVVDVSHEDPEVKFN |
| | M252Y and | WYVDGVEVHNAKTKPREEQYNSTYRVVSVLTVLHQDWLNGKEYKCKVSNKALP |
| | M428L (YL) | APIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVLHEALHNH |
| | | YTQKSLSLSPG |
| 55 | Fc region xELL | DKTHTCPPCPAPGGPSVFLFPPKPKDTLYISRTPEVTCVVVDVSHEDPEVKFN |
| | M252Y, M428L, | WYVDGVEVHNAKTKPREEQYNSTYRVVSVLTVLHQDWLNGKEYKCKVSNKALP |
| | H435R (YLR) | APIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVLHEALHNR |
| | | YTQKSLSLSPG |
| 56 | Fc region xELL | DKTHTCPPCPAPGGPSVFLFPPKPKDTLYISRTPEVTCVVVDVSHEDPEVKFN |
| | M252Y, M428V, | WYVDGVEVHNAKTKPREEQYNSTYRVVSVLTVLHQDWLNGKEYKCKVSNKALP |
| | H435R (YVR) | APIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVVHEALHNR |
| | | YTQKSLSLSPG |
| 57 | Fc region xELL | DKTHTCPPCPAPGGPSVFLFPPKPKDTLMISRTPEVTCVVVDVSHEDPEVKFN |
| | S354C T366W | WYVDGVEVHNAKTKPREEQYNSTYRVVSVLTVLHQDWLNGKEYKCKVSNKALP |
| | knob | APIEKTISKAKGQPREPQVYTLPPCRDELTKNQVSLWCLVKGFYPSDIAVEWE |
| | | SNGQPENNYKTTPPVLDSDGSFFLYSKLTVDKSRWQQGNVFSCSVMHEALHNH |
| | | YTQKSLSLSPG |
| 58 | Fc region xELL | DKTHTCPPCPAPGGPSVFLFPPKPKDTLMISRTPEVTCVVVDVSHEDPEVKFN |
| | H435RS354C | WYVDGVEVHNAKTKPREEQYNSTYRVVSVLTVLHQDWLNGKEYKCKVSNKALP |
| | T366W knob | APIEKTISKAKGQPREPQVYTLPPCRDELTKNQVSLWCLVKGFYPSDIAVEWE |
| | | SNGQPENNYKTTPPVLDSDGSFFLYSKLTVDKSRWQQGNVFSCSVMHEALHNR |
| | | YTQKSLSLSPG |
| 59 | Fc region xELL | DKTHTCPPCPAPGGPSVFLFPPKPKDTLYISRTPEVTCVVVDVSHEDPEVKFN |
| | M252Y and | WYVDGVEVHNAKTKPREEQYNSTYRVVSVLTVLHQDWLNGKEYKCKVSNKALP |
| | M428V (YV) | APIEKTISKAKGQPREPQVYTLPPCRDELTKNQVSLWCLVKGFYPSDIAVEWE |
| | S354C T366W | SNGQPENNYKTTPPVLDSDGSFFLYSKLTVDKSRWQQGNVFSCSVVHEALHNH |
| | knob | YTQKSLSLSPG |
| 60 | Fc region xELL | DKTHTCPPCPAPGGPSVFLFPPKPKDTLYISRTPEVTCVVVDVSHEDPEVKFN |
| | M252Y and | WYVDGVEVHNAKTKPREEQYNSTYRVVSVLTVLHQDWLNGKEYKCKVSNKALP |
| | M428L (YL) | APIEKTISKAKGQPREPQVYTLPPCRDELTKNQVSLWCLVKGFYPSDIAVEWE |
| | S354C T366W | SNGQPENNYKTTPPVLDSDGSFFLYSKLTVDKSRWQQGNVFSCSVLHEALHNH |
| | knob | YTQKSLSLSPG |
| 61 | Fc region xELL | DKTHTCPPCPAPGGPSVFLFPPKPKDTLYISRTPEVTCVVVDVSHEDPEVKFN |
| | M252Y, M428L, | WYVDGVEVHNAKTKPREEQYNSTYRVVSVLTVLHQDWLNGKEYKCKVSNKALP |
| | H435R (YLR) | APIEKTISKAKGQPREPQVYTLPPCRDELTKNQVSLWCLVKGFYPSDIAVEWE |
| | S354C T366W | SNGQPENNYKTTPPVLDSDGSFFLYSKLTVDKSRWQQGNVFSCSVLHEALHNR |
| | knob | YTQKSLSLSPG |
| 62 | Fc region xELL | DKTHTCPPCPAPGGPSVFLFPPKPKDTLYISRTPEVTCVVVDVSHEDPEVKFN |
| | M252Y, M428V, | WYVDGVEVHNAKTKPREEQYNSTYRVVSVLTVLHQDWLNGKEYKCKVSNKALP |
| | H435R (YVR) | APIEKTISKAKGQPREPQVYTLPPCRDELTKNQVSLWCLVKGFYPSDIAVEWE |
| | S354C T366W | SNGQPENNYKTTPPVLDSDGSFFLYSKLTVDKSRWQQGNVFSCSVVHEALHNR |
| | knob | YTQKSLSLSPG |
| 63 | Fc region xELL | DKTHTCPPCPAPGGPSVFLFPPKPKDTLMISRTPEVTCVVVDVSHEDPEVKFN |
| | T366S, L368A, | WYVDGVEVHNAKTKPREEQYNSTYRVVSVLTVLHQDWLNGKEYKCKVSNKALP |
| | Y407V hole | APIEKTISKAKGQPREPQVCTLPPSRDELTKNQVSLSCAVKGFYPSDIAVEWE |
| | | SNGQPENNYKTTPPVLDSDGSFFLVSKLTVDKSRWQQGNVFSCSVMHEALHNH |
| | | YTQKSLSLSPG |
| 64 | Fc region xELL | DKTHTCPPCPAPGGPSVFLFPPKPKDTLMISRTPEVTCVVVDVSHEDPEVKFN |
| | H435R, T366S, | WYVDGVEVHNAKTKPREEQYNSTYRVVSVLTVLHQDWLNGKEYKCKVSNKALP |
| | L368A, Y407V | APIEKTISKAKGQPREPQVCTLPPSRDELTKNQVSLSCAVKGFYPSDIAVEWE |
| | hole | SNGQPENNYKTTPPVLDSDGSFFLVSKLTVDKSRWQQGNVFSCSVMHEALHNR |
| | | YTQKSLSLSPG |
| 65 | Fc region xELL | DKTHTCPPCPAPGGPSVFLFPPKPKDTLYISRTPEVTCVVVDVSHEDPEVKFN |
| | M252Y and | WYVDGVEVHNAKTKPREEQYNSTYRVVSVLTVLHQDWLNGKEYKCKVSNKALP |
| | M428V (YV) | APIEKTISKAKGQPREPQVCTLPPSRDELTKNQVSLSCAVKGFYPSDIAVEWE |
| | T366S, L368A, | SNGQPENNYKTTPPVLDSDGSFFLVSKLTVDKSRWQQGNVFSCSVVHEALHNH |
| | Y407V hole | YTQKSLSLSPG |
| 66 | Fc region xELL | DKTHTCPPCPAPGGPSVFLFPPKPKDTLYISRTPEVTCVVVDVSHEDPEVKFN |
| | M252Y and | WYVDGVEVHNAKTKPREEQYNSTYRVVSVLTVLHQDWLNGKEYKCKVSNKALP |
| | M428L (YL) | APIEKTISKAKGQPREPQVCTLPPSRDELTKNQVSLSCAVKGFYPSDIAVEWE |
| | T366S, L368A, | SNGQPENNYKTTPPVLDSDGSFFLVSKLTVDKSRWQQGNVFSCSVLHEALHNH |
| | Y407V hole | YTQKSLSLSPG |
| 67 | Fc region xELL | DKTHTCPPCPAPGGPSVFLFPPKPKDTLYISRTPEVTCVVVDVSHEDPEVKFN |
| | M252Y, M428L, | WYVDGVEVHNAKTKPREEQYNSTYRVVSVLTVLHQDWLNGKEYKCKVSNKALP |
| | H435R (YLR) | APIEKTISKAKGQPREPQVCTLPPSRDELTKNQVSLSCAVKGFYPSDIAVEWE |
| | T366S, L368A, | SNGQPENNYKTTPPVLDSDGSFFLVSKLTVDKSRWQQGNVFSCSVLHEALHNR |
| | Y407V hole | YTQKSLSLSPG |
| 68 | Fc region xELL | DKTHTCPPCPAPGGPSVFLFPPKPKDTLYISRTPEVTCVVVDVSHEDPEVKFN |
| | M252Y, M428V, | WYVDGVEVHNAKTKPREEQYNSTYRVVSVLTVLHQDWLNGKEYKCKVSNKALP |
| | H435R (YVR) | APIEKTISKAKGQPREPQVCTLPPSRDELTKNQVSLSCAVKGFYPSDIAVEWE |
| | T366S, L368A, | SNGQPENNYKTTPPVLDSDGSFFLVSKLTVDKSRWQQGNVFSCSVVHEALHNR |
| | Y407V hole | YTQKSLSLSPG |
| 69 | Fc region H435R | DKTHTCPPCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVMHEAL |
| | | HNRYTQKSLSLSPG |
| 70 | Fc region M252Y | DKTHTCPPCPAPELLGGPSVFLFPPKPKDTLYISRTPEVTCVVVDVSHEDPEV |
| | and M428V (YV) | KFNWYVDGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVVHEAL |
| | | HNHYTQKSLSLSPG |
| 71 | Fc region M252Y | DKTHTCPPCPAPELLGGPSVFLFPPKPKDTLYISRTPEVTCVVVDVSHEDPEV |
| | and M428L (YL) | KFNWYVDGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVLHEAL |
| | | HNHYTQKSLSLSPG |
| 72 | Fc region | DKTHTCPPCPAPELLGGPSVFLFPPKPKDTLYISRTPEVTCVVVDVSHEDPEV |
| | M252Y, M428L, | KFNWYVDGVEVHNAKTKPREEQYNSTYRVVSVLTVLHQDWLNGKEYKCKVSNK |
| | H435R (YLR) | ALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVLHEAL |
| | | HNRYTQKSLSLSPG |
| 73 | Fc region | DKTHTCPPCPAPELLGGPSVFLFPPKPKDTLYISRTPEVTCVVVDVSHEDPEV |
| | M252Y, M428V, | KFNWYVDGVEVHNAKTKPREEQYNSTYRVVSVLTVLHQDWLNGKEYKCKVSNK |
| | H435R (YVR) | ALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVVHEAL |
| | | HNRYTQKSLSLSPG |
| 74 | Fc region S354C | DKTHTCPPCPAPELLGGPSVFLFPPKPKDTLMISRTPEVTCVVVDVSHEDPEV |
| | T366W knob | KFNWYVDGVEVHNAKTKPREEQYNSTYRVVSVLTVLHQDWLNGKEYKCKVSNK |
| | | ALPAPIEKTISKAKGQPREPQVYTLPPCRDELTKNQVSLWCLVKGFYPSDIAV |
| | | EWESNGQPENNYKTTPPVLDSDGSFFLYSKLTVDKSRWQQGNVFSCSVMHEAL |
| | | HNHYTQKSLSLSPG |
| 75 | Fc region H435R | DKTHTCPPCPAPELLGGPSVFLFPPKPKDTLMISRTPEVTCVVVDVSHEDPEV |
| | S354C T366W | KFNWYVDGVEVHNAKTKPREEQYNSTYRVVSVLTVLHQDWLNGKEYKCKVSNK |
| | knob | ALPAPIEKTISKAKGQPREPQVYTLPPCRDELTKNQVSLWCLVKGFYPSDIAV |
| | | EWESNGQPENNYKTTPPVLDSDGSFFLYSKLTVDKSRWQQGNVFSCSVMHEAL |
| | | HNRYTQKSLSLSPG |
| 76 | Fc region M252Y | DKTHTCPPCPAPELLGGPSVFLFPPKPKDTLYISRTPEVTCVVVDVSHEDPEV |
| | and M428L (YL) | KFNWYVDGVEVHNAKTKPREEQYNSTYRVVSVLTVLHQDWLNGKEYKCKVSNK |
| | S354C T366W | ALPAPIEKTISKAKGQPREPQVYTLPPCRDELTKNQVSLWCLVKGFYPSDIAV |
| | knob | EWESNGQPENNYKTTPPVLDSDGSFFLYSKLTVDKSRWQQGNVFSCSVLHEAL |
| | | HNHYTQKSLSLSPG |
| 77 | Fc region | DKTHTCPPCPAPELLGGPSVFLFPPKPKDTLYISRTPEVTCVVVDVSHEDPEV |
| | M252Y, M428L, | KFNWYVDGVEVHNAKTKPREEQYNSTYRVVSVLTVLHQDWLNGKEYKCKVSNK |
| | H435R (YLR) | ALPAPIEKTISKAKGQPREPQVYTLPPCRDELTKNQVSLWCLVKGFYPSDIAV |
| | S354C T366W | EWESNGQPENNYKTTPPVLDSDGSFFLYSKLTVDKSRWQQGNVFSCSVLHEAL |
| | knob | HNRYTQKSLSLSPG |
| 78 | Fc region | DKTHTCPPCPAPELLGGPSVFLFPPKPKDTLYISRTPEVTCVVVDVSHEDPEV |
| | M252Y, M428V, | KFNWYVDGVEVHNAKTKPREEQYNSTYRVVSVLTVLHQDWLNGKEYKCKVSNK |
| | H435R (YVR) | ALPAPIEKTISKAKGQPREPQVYTLPPCRDELTKNQVSLWCLVKGFYPSDIAV |
| | S354C T366W | EWESNGQPENNYKTTPPVLDSDGSFFLYSKLTVDKSRWQQGNVFSCSVVHEAL |
| | knob | HNRYTQKSLSLSPG |
| 79 | Fc region T366S, | DKTHTCPPCPAPELLGGPSVFLFPPKPKDTLMISRTPEVTCVVVDVSHEDPEV |
| | L368A, Y407V | KFNWYVDGVEVHNAKTKPREEQYNSTYRVVSVLTVLHQDWLNGKEYKCKVSNK |
| | hole | ALPAPIEKTISKAKGQPREPQVCTLPPSRDELTKNQVSLSCAVKGFYPSDIAV |
| | | EWESNGQPENNYKTTPPVLDSDGSFFLVSKLTVDKSRWQQGNVFSCSVMHEAL |
| | | HNHYTQKSLSLSPG |
| 80 | Fc region H435R, | DKTHTCPPCPAPELLGGPSVFLFPPKPKDTLMISRTPEVTCVVVDVSHEDPEV |
| | T366S, L368A, | KFNWYVDGVEVHNAKTKPREEQYNSTYRVVSVLTVLHQDWLNGKEYKCKVSNK |
| | Y407V hole | ALPAPIEKTISKAKGQPREPQVCTLPPSRDELTKNQVSLSCAVKGFYPSDIAV |
| | | EWESNGQPENNYKTTPPVLDSDGSFFLVSKLTVDKSRWQQGNVFSCSVMHEAL |
| | | HNRYTQKSLSLSPG |
| 81 | Fc region M252Y | DKTHTCPPCPAPELLGGPSVFLFPPKPKDTLYISRTPEVTCVVVDVSHEDPEV |
| | and M428V (YV) | KFNWYVDGVEVHNAKTKPREEQYNSTYRVVSVLTVLHQDWLNGKEYKCKVSNK |
| | T366S, L368A, | ALPAPIEKTISKAKGQPREPQVCTLPPSRDELTKNQVSLSCAVKGFYPSDIAV |
| | Y407V hole | EWESNGQPENNYKTTPPVLDSDGSFFLVSKLTVDKSRWQQGNVFSCSVVHEAL |
| | | HNHYTQKSLSLSPG |
| 82 | Fc region M252Y | DKTHTCPPCPAPELLGGPSVFLFPPKPKDTLYISRTPEVTCVVVDVSHEDPEV |
| | and M428L (YL) | KFNWYVDGVEVHNAKTKPREEQYNSTYRVVSVLTVLHQDWLNGKEYKCKVSNK |
| | T366S, L368A, | ALPAPIEKTISKAKGQPREPQVCTLPPSRDELTKNQVSLSCAVKGFYPSDIAV |
| | Y407V hole | EWESNGQPENNYKTTPPVLDSDGSFFLVSKLTVDKSRWQQGNVFSCSVLHEAL |
| | | HNHYTQKSLSLSPG |
| 83 | Fc region | DKTHTCPPCPAPELLGGPSVFLFPPKPKDTLYISRTPEVTCVVVDVSHEDPEV |
| | M252Y, M428L, | KFNWYVDGVEVHNAKTKPREEQYNSTYRVVSVLTVLHQDWLNGKEYKCKVSNK |
| | H435R (YLR) | ALPAPIEKTISKAKGQPREPQVCTLPPSRDELTKNQVSLSCAVKGFYPSDIAV |
| | T366S, L368A, | EWESNGQPENNYKTTPPVLDSDGSFFLVSKLTVDKSRWQQGNVFSCSVLHEAL |
| | Y407V hole | HNRYTQKSLSLSPG |
| 84 | Truncated wild- | QLQLEHLLLDLQMILNGINNYKNPKLTRMLTFKFYMPKKATELKHLQCLEEEL |
| | type human IL-2 | KPLEEVLNLAQSKNFHLRPRDLISNINVIVLELKGSETTFMCEYADETATIVE |
| | | FLNRWITFCQSII |
| 86 | xELL-Knob Fc-IL- | DKTHTCPPCPAPGGPSVFLFPPKPKDTLMISRTPEVTCVVVDVSHEDPEVKFN |
| | 2-T3A, C125S | WYVDGVEVHNAKTKPREEQYNSTYRVVSVLTVLHQDWLNGKEYKCKVSNKALP |
| | | 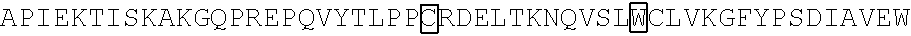 |
| | | ESNGQPENNYKTTPPVLDSDGSFFLYSKLTVDKSRWQQGNVFSCSVMHEALHN |
| | | 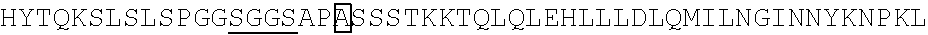 |
| | | TRMLTFKFYMPKKATELKHLQCLEEELKPLEEVLNLAQSKNFHLRPRDLISNI |
| | | 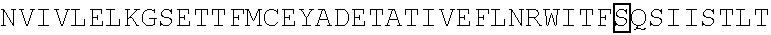 |
| 87 | xELL-Knob Fc-IL- | DKTHTCPPCPAPGGPSVFLFPPKPKDTLMISRTPEVTCVVVDVSHEDPEVKFN |
| | 2-RAS-T3A, | WYVDGVEVHNAKTKPREEQYNSTYRVVSVLTVLHQDWLNGKEYKCKVSNKALP |
| | C125S | 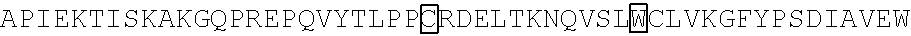 |
| | | ESNGQPENNYKTTPPVLDSDGSFFLYSKLTVDKSRWQQGNVFSCSVMHEALHN |
| | | 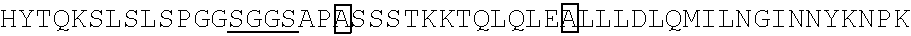 |
| | | 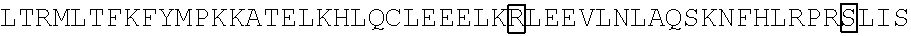 |
| | | 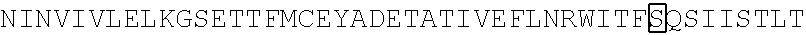 |
| 88 | Pembrolizumab | QVQLVQSGVEVKKPGASVKVSCKASGYTFTNYYMYWVRQAPGQGLEWMGGINP |
| | analog Knob Fc-IL- | SNGGTNFNEKFKNRVTLTTDSSTTTAYMELKSLQFDDTAVYYCARRDYRFDMG |
| | 2-RAS-T3A, | FDYWGQGTTVTVSSASTKGPSVFPLAPSSKSTSGGTAALGCLVKDYFPEPVTV |
| | C125S | SWNSGALTSGVHTFPAVLQSSGLYSLSSVVTVPSSSLGTQTYICNVNHKPSNT |
| | | KVDKKVEPKSCDKTHTCPPCPAPGGPSVFLFPPKPKDTLMISRTPEVTCVVVD |
| | | VSHEDPEVKFNWYVDGVEVHNAKTKPREEQYNSTYRVVSVLTVLHQDWLNGKE |
| | | 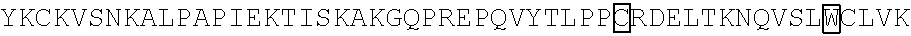 |
| | | 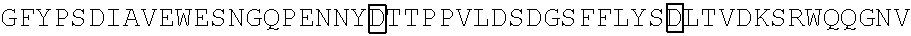 |
| | | 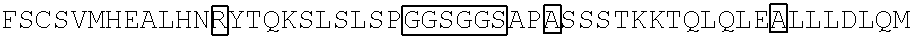 |
| | | 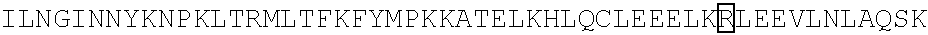 |
| | | 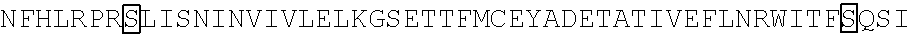 |
| | | ISTLT |
| 89 | Pembrolizumab | QVQLVQSGVEVKKPGASVKVSCKASGYTFTNYYMYWVRQAPGQGLEWMGGINP |
| | analog Hole Fc | SNGGTNFNEKFKNRVTLTTDSSTTTAYMELKSLQFDDTAVYYCARRDYRFDMG |
| | | FDYWGQGTTVTVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNHKPSNT |
| | | KVDKKVEPKSCDKTHTCPPCPAPGGPSVFLFPPKPKDTLMISRTPEVTCVVVD |
| | | VSHEDPEVKFNWYVDGVEVHNAKTKPREEQYNSTYRVVSVLTVLHQDWLNGKE |
| | | 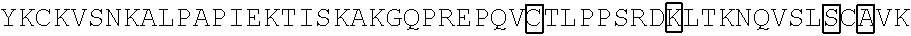 |
| | | 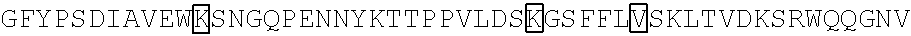 |
| | | FSCSVMHEALHNHYTQKSLSLSPGK |
| 90 | Pembrolizumab | EIVLTQSPATLSLSPGERATLSCRASKGVSTSGYSYLHWYQQKPGQAPRLLIY |
| | Light Chain analog | LASYLESGVPARFSGSGSGTDFTLTISSLEPEDFAVYYCQHSRDLPLTFGGGT |
| | | KVEIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVTKS |
| | | FNRGEC |
| 91 | Pembrolizumab | QVQLVQSGVEVKKPGASVKVSCKASGYTFTNYYMYWVRQAPGQGLEWMGGINP |
| | analog IL-2-RAS- | SNGGTNFNEKFKNRVTLTTDSSTTTAYMELKSLQFDDTAVYYCARRDYRFDMG |
| | T3G, C125S | FDYWGQGTTVTVSSASTKGPSVFPLAPCSRSTSESTAALGCLVKDYFPEPVTV |
| | | SWNSGALTSGVHTFPAVLQSSGLYSLSSVVTVPSSSLGTKTYTCNVDHKPSNT |
| | | KVDKRVESKYGPPCPPCPAPEFLGGPSVFLFPPKPKDTLMISRTPEVTCVVVD |
| | | VSQEDPEVQFNWYVDGVEVHNAKTKPREEQFNSTYRVVSVLTVLHQDWLNGKE |
| | | YKCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGNVFS |
| | | 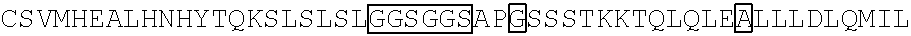 |
| | | 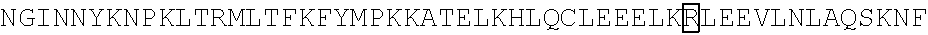 |
| | | 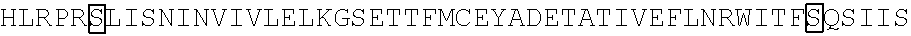 |
| | | TLT |
| 92 | NKp46-scFv | QVQLQQSGPELVKPGASVKMSCKASGYTFTDYVINWGKQRSGQGLEWIGEIYP |
| | xELL-Knob Fc-IL- | GSGTNYYNEKFKAKATLTADKSSNIAYMQLSSLTSEDSAVYFCARRGRYGLYA |
| | 2-RAS-T3A, C125S | 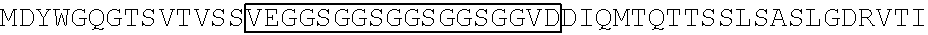 |
| | | SCRASQDISNYLNWYQQKPDGTVKLLIYYTSRLHSGVPSRFSGSGSGTDYSLT |
| | | 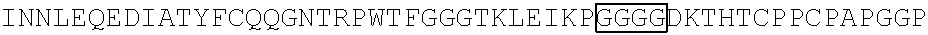 |
| | | SVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPR |
| | | 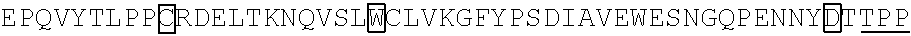 |
| | | 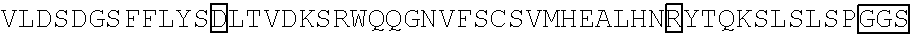 |
| | | 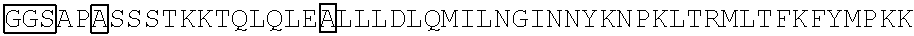 |
| | | 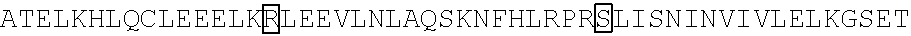 |
| | | 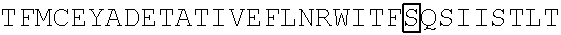 |
| 93 | NKp46-scFv | QVQLQQSGPELVKPGASVKMSCKASGYTFTDYVINWGKQRSGQGLEWIGEIYP |
| | xELL-Hole Fc | GSGTNYYNEKFKAKATLTADKSSNIAYMQLSSLTSEDSAVYFCARRGRYGLYA |
| | | 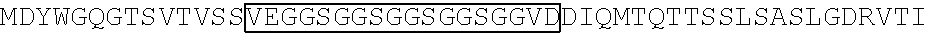 |
| | | SCRASQDISNYLNWYQQKPDGTVKLLIYYTSRLHSGVPSRFSGSGSGTDYSLT |
| | | 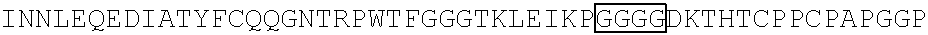 |
| | | SVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPR |
| | | 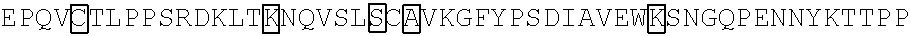 |
| | | 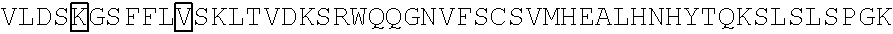 |
| 94 | NKp46-scFv | QVQLQQSGPELVKPGASVKMSCKASGYTFTDYVINWGKQRSGQGLEWIGEIYP |
| | xELL-Fc | GSGTNYYNEKFKAKATLTADKSSNIAYMQLSSLTSEDSAVYFCARRGRYGLYA |
| | | MDYWGQGTSVTVSSVEGGSGGSGGSGGSGGVDDIQMTQTTSSLSASLGDRVTI |
| | | SCRASQDISNYLNWYQQKPDGTVKLLIYYTSRLHSGVPSRFSGSGSGTDYSLT |
| | | INNLEQEDIATYFCQQGNTRPWTFGGGTKLEIKPGGGGDKTHTCPPCPAPELL |
| | | GGPSVFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSLSPGK |
| 95 | LAG3-MAb xELL- | QVQLVQSGAEVKKPGASVKVSCKASGYTFTGYYMHWVRQAPGQGLEWMGWINA |
| | Knob Fc-IL-2- | NSGGTNYAQKFQGRVTMTRDTSISTAYMELSRLRSDDTAVYYCARDIYDSSDQ |
| | RAS-TGCS | LNVWGQGTMVTVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLTVLHQDWLN |
| | | 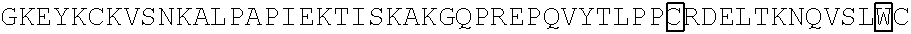 |
| | | LVKGFYPSDIAVEWESNGQPENNYKTTPPVLDSDGSFFLYSKLTVDKSRWQQG |
| | | 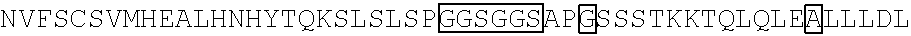 |
| | | 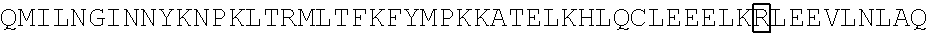 |
| | | 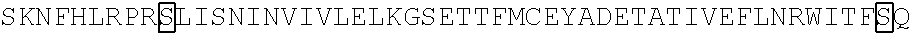 |
| | | SIISTLT |
| 96 | LAG3-MAb xELL- | QVQLVQSGAEVKKPGASVKVSCKASGYTFTGYYMHWVRQAPGQGLEWMGWINA |
| | Hole Fc | NSGGTNYAQKFQGRVTMTRDTSISTAYMELSRLRSDDTAVYYCARDIYDSSDQ |
| | | LNVWGQGTMVTVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNHKPSNT |
| | | 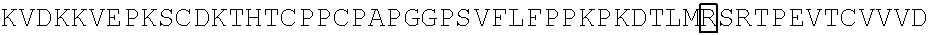 |
| | | VSHEDPEVKFNWYVDGVEVHNAKTKPREEQYNSTYRVVSVLTVLHQDWLNGKE |
| | | 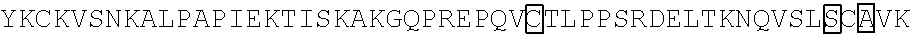 |
| | | 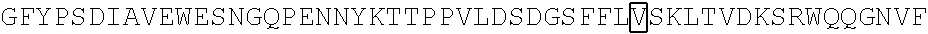 |
| | | SCSVMHEALHNHYTQKSLSLSPGK |
| 97 | LAG3-MAb Light | EIVLTQSPATLSLSPGERATLSCRASQSVSSYLAWYQQKPGQAPRLLIYDASN |
| | Chain | RATGIPARFSGSGSGTDFTLTISSLEPEDFAVYYCQQASIWPLTFGGGTKVEI |
| | | KRTVTtAPSVFIFPPSDEQLKSGTASWCLLNNFYPREAKVQWKVDNALQSGNS |
| | | QESVTEQDSKDSTYSLSSTLTLSKADYEKHKVYACEVTHQGLSSPVTKSFNRG |
| | | EC |
| 98 | LAG3-MAb IgG1 | QVQLVQSGAEVKKPGASVKVSCKASGYTFTGYYMHWVRQAPGQGLEWMGWINA |
| | | NSGGTNYAQKFQGRVTMTRDTSISTAYMELSRLRSDDTAVYYCARDIYDSSDQ |
| | | LNVWGQGTMVTVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLTVLHQDWLN |
| | | GKEYKCKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKSRWQQGN |
| | | VFSCSVMHEALHNHYTQKSLSLSPGK |
| 99 | LAG3-VHH xELL- | EVQLVESGGGWQPGGSLRLSCAASGRTFSDYVMGWFRQAPGKEREFVAAISES |
| | Knob Fc-IL-2- | GGRTHYADSVKGRFTISRDNSKNTLYLQMNSLRPEDTALYYCATTLLWWTSEY |
| | RAS-TGCS | 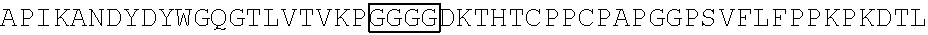 |
| | | MISRTPEVTCVVVDVSHEDPEVKFNWYVDGVEVHNAKTKPREEQYNSTYRVVS |
| | | 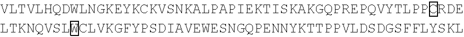 |
| | | 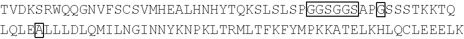 |
| | | 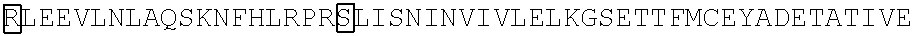 |
| | | 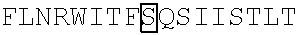 |
| 100 | LAG3-VHH xELL- | EVQLVESGGGWQPGGSLRLSCAASGRTFSDYVMGWFRQAPGKEREFVAAISES |
| | Hole T366S, L368A, | GGRTHYADSVKGRFTISRDNSKNTLYLQMNSLRPEDTALYYCATTLLWWTSEY |
| | Y407V, H435RFc | 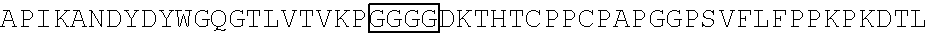 |
| | | MISRTPEVTCVVVDVSHEDPEVKFNWYVDGVEVHNAKTKPREEQYNSTYRVVS |
| | | 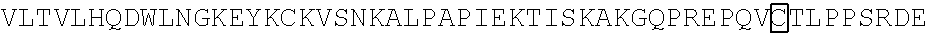 |
| | | 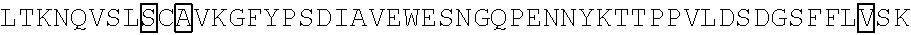 |
| | | 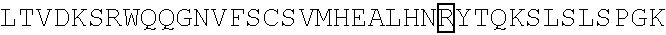 |
| 101 | LAG3-VHH xELL- | EVQLVESGGGWQPGGSLRLSCAASGRTFSDYVMGWFRQAPGKEREFVAAISES |
| | Knob Fc | GGRTHYADSVKGRFTISRDNSKNTLYLQMNSLRPEDTALYYCATTLLWWTSEY |
| | | 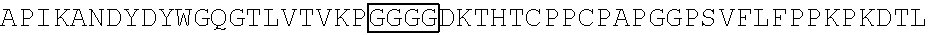 |
| | | MISRTPEVTCVVVDVSHEDPEVKFNWYVDGVEVHNAKTKPREEQYNSTYRVVS |
| | | 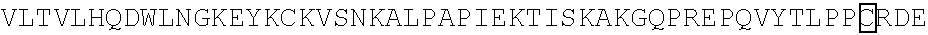 |
| | | 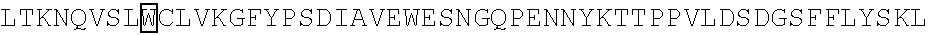 |
| | | TVDKSRWQQGNVFSCSVMHEALHNHYTQKSLSLSPGK |
| 102 | xELL-Knob Fc-IL- | DKTHTCPPCPAPGGPSVFLFPPKPKDTLYISRTPEVTCVVVDVSHEDPEVKFN |
| | 2-RAS-M23A- | WYVDGVEVHNAKTKPREEQYNSTYRVVSVLTVLHQDWLNGKEYKCKVSNKALP |
| | T3A, C125S | APIEKTISKAKGQPREPQVYTLPPCRDELTKNQVSLWCLVKGFYPSDIAVEWE |
| | | SNGQPENNYKTTPPVLDSDGSFFLYSKLTVDKSRWQQGNVFSCSVVHEALHNH |
| | | 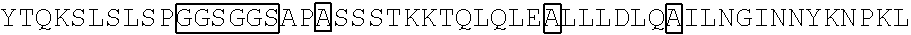 |
| | | 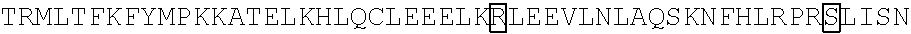 |
| | | 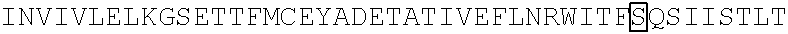 |
| 103 | xELL-Knob Fc-IL- | DKTHTCPPCPAPGGPSVFLFPPKPKDTLYISRTPEVTCVVVDVSHEDPEVKFN |
| | 2-RAS-E95Q-T3A, | WYVDGVEVHNAKTKPREEQYNSTYRVVSVLTVLHQDWLNGKEYKCKVSNKALP |
| | C125S | APIEKTISKAKGQPREPQVYTLPPCRDELTKNQVSLWCLVKGFYPSDIAVEWE |
| | | SNGQPENNYKTTPPVLDSDGSFFLYSKLTVDKSRWQQGNVFSCSVVHEALHNH |
| | | 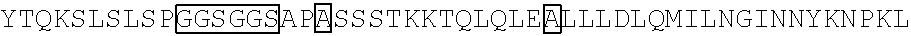 |
| | | 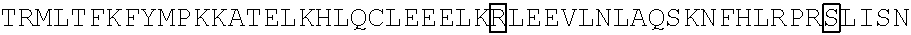 |
| | | 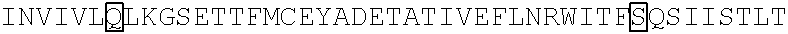 |
| 104 | xELL-Knob Fc-IL- | DKTHTCPPCPAPGGPSVFLFPPKPKDTLYISRTPEVTCVVVDVSHEDPEVKFN |
| | 2-RAS-M23A- | WYVDGVEVHNAKTKPREEQYNSTYRVVSVLTVLHQDWLNGKEYKCKVSNKALP |
| | E95Q-T3A, C125S | APIEKTISKAKGQPREPQVYTLPPCRDELTKNQVSLWCLVKGFYPSDIAVEWE |
| | | SNGQPENNYKTTPPVLDSDGSFFLYSKLTVDKSRWQQGNVFSCSVVHEALHNH |
| | | 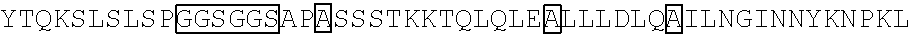 |
| | | 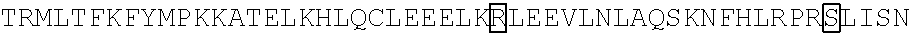 |
| | | 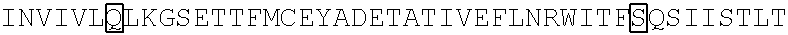 |
| 105 | IL-2-T3G-C125S | APGSSSTKKTQLQLEHLLLDLQMILNGINNYKNPKLTRMLTFKFYMPKKATEL |
| | | KHLQCLEEELKPLEEVLNLAQSKNFHLRPRDLISNINVIVLELKGSETTFMCE |
| | | YADETATIVEFLNRWITFSQSIISTLT |
| 106 | IL-2-T3A-E61R- | APASSSTKKTQLQLEHLLLDLQMILNGINNYKNPKLTRMLTFKFYMPKKATEL |
| | P65R-C125S | KHLQCLERELKRLEEVLNLAQSKNFHLRPRDLISNINVIVLELKGSETTFMCE |
| | | YADETATIVEFLNRWITFSQSIISTLT |
| 107 | IL-2-T3A-R38A- | APASSSTKKTQLQLEHLLLDLQMILNGINNYKNPKLTAMLTFKFYMPKKATEL |
| | P65R-C125S | KHLQCLEEELKRLEEVLNLAQSKNFHLRPRDLISNINVIVLELKGSETTFMCE |
| | | YADETATIVEFLNRWITFSQSIISTLT |
| 108 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S | YADETATIVEFLNRWITFSQSIISTLT |
| 109 | IL-2-T3A-H16A- | APASSSTKKTQLQLEALLLDLQMILNGINNYKNPKLTAMLTFKFYMPKKATEL |
| | R38A-P65R-D84S- | KHLQCLEEELKRLEEVLNLAQSKNFHLRPRSLISNINVIVLELKGSETTFMCE |
| | C125S | YADETATIVEFLNRWITFSQSIISTLT |
| 110 | IL-2-T3A-H16A- | APASSSTKKTQLQLEALLLALQMILNGINNYKNPKLTRMLTFKFYMPKKATEL |
| | L19A-P65R-D84S- | KHLQCLEEELKRLEEVLNLAQSKNFHLRPRSLISNINVIVLELKGSETTFMCE |
| | C125S | YADETATIVEFLNRWITFSQSIISTLT |
| 111 | IL-2-T3A-H16A- | APASSSTKKTQLQLEALLLDLQMILNGINNYKNPKLTRMLTFKFYMPKKATEL |
| | P65R-D84S-N88S- | KHLQCLEEELKRLEEVLNLAQSKNFHLRPRSLISSINVIVLELKGSETTFMCE |
| | C125S | YADETATIVEFLNRWITFSQSIISTLT |
| 112 | IL-2-T3A-H16A- | APASSSTKKTQLQLEALLLDLQMILNGINNYKNPKLTRMLTFKFYMPKKATEL |
| | P65R-D84S-E95Q- | KHLQCLEEELKRLEEVLNLAQSKNFHLRPRSLISNINVIVLQLKGSETTFMCE |
| | C125S | YADETATIVEFLNRWITFSQSIISTLT |
| 113 | IL-2-T3A-H16A- | APASSSTKKTQLQLEALLLDLQAILNGINNYKNPKLTRMLTFKFYMPKKATEL |
| | M23A-P65R- | KHLQCLEEELKRLEEVLNLAQSKNFHLRPRSLISNINVIVLELKGSETTFMCE |
| | D84S-C125S | YADETATIVEFLNRWITFSQSIISTLT |
| 114 | IL-2-T3A-H16A- | APASSSTKKTQLQLEALLLDLQAILNGINNYKNPKLTRMLTFKFYMPKKATEL |
| | M23A-P65R- | KHLQCLEEELKRLEEVLNLAQSKNFHLRPRSLISNINVIVLQLKGSETTFMCE |
| | D84S-E95Q-C125S | YADETATIVEFLNRWITFSQSIISTLT |
| 115 | IL-2-T3A-H16A- | APASSSTKKTQLQLEALLADLQMILNGINNYKNPKLTRMLTFKFYMPKKATEL |
| | L19A-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 116 | IL-2-T3A-H16A- | APASSSTKKTQLQLEALLLDLQAILNGINNYKNPKLTRMLTFKFYMPKKATEL |
| | M23A-E61R- | KHLQCLERELKRLEEVLNLAQSKNFHLRPRSLISNINVIVLQLKGSETTFMCE |
| | P65R-D84S-E95Q- | YADETATIVEFLNRWITFSQSIISTLT |
| | C125S | |
| 117 | IL-2-T3A-H16A- | APASSSTKKTQLQLEALLLDLQAILNGINNYKNPKLTRMLTFKFYMPKKATEL |
| | M23A-E61R- | KHLQCLERELKRLEEVLNLAQSKNFHLRPRSLISSINVIVLELKGSETTFMCE |
| | P65R-D84S-N88S- | YADETATIVEFLNRWITFSQSIISTLT |
| | C125S | |
| 118 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISSINVIVLQLKGSETTFMCE |
| | N88S-E95Q-C125S | YADETATIVEFLNRWITFSQSIISTLT |
| 119 | IL-2-T3A-H16A- | APASSSTKKTQLQLEALLLDLQAILNGINNYKNPKLTRMLTFKFYMPKKATEL |
| | M23A-E61R- | KHLQCLERELKRLEEVLNLAQSKNFHLRPRSLISSINVIVLQLKGSETTFMCE |
| | P65R-D84S-N88S- | YADETATIVEFLNRWITFSQSIISTLT |
| | E95Q-C125S | |
| 120 | IL-2-T3A-H16A- | APASSSTKKTQLQLEALLADLQMILNGINNYKNPKLTAMLTFKFYMPKKATEL |
| | L19A-R38A-P65R- | KHLQCLEEELKRLEEVLNLAQSKNFHLRPRSLISNINVIVLELKGSETTFMCE |
| | D84S-C125S | YADETATIVEFLNRWITFSQSIISTLT |
| 121 | IL-2-T3A-H16A- | APASSSTKKTQLQLEALLLDLQAILNGINNYKNPKLTAMLTFKFYMPKKATEL |
| | M23A-R38A- | KHLQCLEEELKRLEEVLNLAQSKNFHLRPRSLISNINVIVLQLKGSETTFMCE |
| | P65R-D84S-E95Q- | YADETATIVEFLNRWITFSQSIISTLT |
| | C125S | |
| 122 | IL-2-T3A-H16A- | APASSSTKKTQLQLEALLLDLQAILNGINNYKNPKLTAMLTFKFYMPKKATEL |
| | M23A-R38A- | KHLQCLEEELKRLEEVLNLAQSKNFHLRPRSLISSINVIVLELKGSETTFMCE |
| | P65R-D84S-N88S- | YADETATIVEFLNRWITFSQSIISTLT |
| | C125S | |
| 123 | IL-2-T3A-H16A- | APASSSTKKTQLQLEALLLDLQMILNGINNYKNPKLTAMLTFKFYMPKKATEL |
| | R38A-P65R-D84S- | KHLQCLEEELKRLEEVLNLAQSKNFHLRPRSLISSINVIVLQLKGSETTFMCE |
| | N88S-E95Q-C125S | YADETATIVEFLNRWITFSQSIISTLT |
| 124 | IL-2-T3A-H16A- | APASSSTKKTQLQLEALLLDLQAILNGINNYKNPKLTAMLTFKFYMPKKATEL |
| | M23A-R38A- | KHLQCLEEELKRLEEVLNLAQSKNFHLRPRSLISSINVIVLQLKGSETTFMCE |
| | P65R-IL-2-D84S- | YADETATIVEFLNRWITFSQSIISTLT |
| | N88S-E95Q-C125S | |
| 125 | IL-2-T3A-H16A- | APASSSTKKTQLQLEALLNDLQMILNGINNYKNPKLTRMLTFKFYMPKKATEL |
| | L19N-P65R-D84S- | KHLQCLEEELKRLEEVLNLAQSKNFHLRPRSLISNINVIVLELKGSETTFMCE |
| | C125S | YADETATIVEFLNRWITFSQSIISTLT |
| 126 | IL-2-T3A-H16A- | APASSSTKKTQLQLEALLSDLQMILNGINNYKNPKLTRMLTFKFYMPKKATEL |
| | L19S-P65R-D84S- | KHLQCLEEELKRLEEVLNLAQSKNFHLRPRSLISNINVIVLELKGSETTFMCE |
| | C125S | YADETATIVEFLNRWITFSQSIISTLT |
| 127 | IL-2-T3A-H16A- | APASSSTKKTQLQLEALLTDLQMILNGINNYKNPKLTRMLTFKFYMPKKATEL |
| | L19T-P65R-D84S- | KHLQCLEEELKRLEEVLNLAQSKNFHLRPRSLISNINVIVLELKGSETTFMCE |
| | C125S | YADETATIVEFLNRWITFSQSIISTLT |
| 128 | IL-2-T3A-H16A- | APASSSTKKTQLQLEALLVDLQMILNGINNYKNPKLTRMLTFKFYMPKKATEL |
| | L19V-P65R-D84S- | KHLQCLEEELKRLEEVLNLAQSKNFHLRPRSLISNINVIVLELKGSETTFMCE |
| | C125S | YADETATIVEFLNRWITFSQSIISTLT |
| 129 | IL-2-T3A-H16A- | APASSSTKKTQLQLEALLNDLQAILNGINNYKNPKLTRMLTFKFYMPKKATEL |
| | L19N-M23A- | KHLQCLERELKRLEEVLNLAQSKNFHLRPRSLISNINVIVLELQGSETTFMCE |
| | E61R-P65R-D84S- | YADETATIVEFLNRWITFSQSIISTLT |
| | E95Q-C125S | |
| 130 | IL-2-T3A-H16A- | APASSSTKKTQLQLEALLNDLQAILNGINNYKNPKLTAMLTFKFYMPKKATEL |
| | L19N-M23A- | KHLQCLEEELKRLEEVLNLAQSKNFHLRPRSLISNINVIVLQLKGSETTFMCE |
| | R38A-P65R-D84S- | YADETATIVEFLNRWITFSQSIISTLT |
| | E95Q-C125S | |
| 131 | IL-2-T3A-H16A- | APASSSTKKTQLQLEALLNDLQAILNGINNYKNPKLTAMLTFKFYMPKKATEL |
| | L19N-M23A- | KHLQCLEEELKRLEEVLNLAQSKNFHLRPRSLISNINVIVLELKGSETTFMCE |
| | R38A-P65R-D84S- | YADETATIVEFLNRWITFSQSIISTLT |
| | C125S | |
| 132 | IL-2-T3A-H16A- | APASSSTKKTQLQLEALLNDLQMILNGINNYKNPKLTAMLTFKFYMPKKATEL |
| | L19N-R38A-P65R- | KHLQCLEEELKRLEEVLNLAQSKNFHLRPRSLISNINVIVLQLKGSETTFMCE |
| | D84S-E95Q-C125S | YADETATIVEFLNRWITFSQSIISTLT |
| 133 | IL-2-T3A-H16A- | APASSSTKKTQLQLEALLNDLQMILNGINNYKNPKLTAMLTFKFYMPKKATEL |
| | L19N-R38A-P65R- | KHLQCLEEELKRLEEVLNLAQSKNFHLRPRSLISNINVIVLELKGSETTFMCE |
| | D84S-C125S | YADETATIVEFLNRWITFSQSIISTLT |
| 134 | IL-2-T3A-H16A- | APASSSTKKTQLQLEALLNDLQMILNGINNYKNPKLTRMLTFKFYMPKKATEL |
| | L19N-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 135 | IL-2-T3A-H16A- | APASSSTKKTQLQLEALLNDLQMILNGINNYKNPKLTRMLTFKFYMPKKATEL |
| | L19N-E61R-P65R- | KHLQCLERELKRLEEVLNLAQSKNFHLRPRSLISNINVIVLQLKGSETTFMCE |
| | D84S-E95Q-C125S | YADETATIVEFLNRWITFSQSIISTLT |
| 136 | IL-2-T3A-H16A- | APASSSTKKTQLQLEALLNDLQAILNGINNYKNPKLTRMLTFKFYMPKKATEL |
| | L19N-M23A- | KHLQCLERELKRLEEVLNLAQSKNFHLRPRSLISNINVIVLELKGSETTFMCE |
| | E61R-P65R-D84S- | YADETATIVEFLNRWITFSQSIISTLT |
| | C125S | |
| 137 | IL-2-T3A-H16A- | APASSSTKKTQLQLEALLNDLAMILNGINNYKNPKLTRMLTFKFYMPKKATEL |
| | L19N-Q22A- | KHLQCLERELKRLEEVLNLAQSKNFHLRPRSLISNINVIVLELKGSETTFMCE |
| | E61R-P65R-D84S- | YADETATIVEFLNRWITFSQSIISTLT |
| | C125S | |
| 138 | IL-2-T3A-H16A- | APASSSTKKTQLQLEALLNDLQMILNGINNYKNPKLTRMLTFKFYMPKKATEL |
| | L19N-E61R-P65R- | KHLQCLERELKRLEEVLNLAQSKNFHLRPRSLISNINVIVLELKGSETTFMCE |
| | D84S-R120A- | YADETATIVEFLNAWITFSQSIISTLT |
| | C125S | |
| 139 | IL-2-T3A-H16A- | APASSSTKKTQLQLEALLNDLQMILNGINNYKNPKLTRMLTFKFYMPKKATEL |
| | L19N-E61R-P65R- | KHLQCLERELKRLEEVLNLAQSKNFHLRPRSLISNINVIVLELKGSETTFMCE |
| | D84S-C125S- | YADETATIVEFLNRWITFSASIISTLT |
| | Q126A | |
| 140 | IL-2-T3A-H16S- | APASSSTKKTQLQLESLLNDLQMILNGINNYKNPKLTRMLTFKFYMPKKATEL |
| | L19N-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 141 | IL-2-T3A-H16T- | APASSSTKKTQLQLETLLNDLQMILNGINNYKNPKLTRMLTFKFYMPKKATEL |
| | L19N-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 142 | IL-2-T3A-H16V- | APASSSTKKTQLQLEVLLNDLQMILNGINNYKNPKLTRMLTFKFYMPKKATEL |
| | L19N-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 143 | IL-2-T3A-H16N- | APASSSTKKTQLQLENLLNDLQMILNGINNYKNPKLTRMLTFKFYMPKKATEL |
| | L19N-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 144 | IL-2-T3A-H16A- | APASSSTKKTQLQLEALLNDLQMILNGINNYKNPKLTRMLTFKFYMPKKATEL |
| | L19N-E61R-P65R- | KHLQCLERELKRLEEVLNLAQSKNFHLRPRTLISNINVIVLELKGSETTFMCE |
| | D84T-C125S | YADETATIVEFLNRWITFSQSIISTLT |
| 145 | IL-2-T3A-H16A- | APASSSTKKTQLQLEALLNDLQMILNGINNYKNPKLTRMLTFKFYMPKKATEL |
| | L19N-E61R-P65R- | KHLQCLERELKRLEEVLNLAQSKNFHLRPRVLISNINVIVLELKGSETTFMCE |
| | D84V-C125S | YADETATIVEFLNRWITFSQSIISTLT |
| 146 | IL-2-T3A-H16A- | APASSSTKKTQLQLEALLLSLQMILNGINNYKNPKLTRMLTFKFYMPKKATEL |
| | D20S-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 147 | IL-2-T3A-H16A- | APASSSTKKTQLQLEALLLTLQMILNGINNYKNPKLTRMLTFKFYMPKKATEL |
| | D20T-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 148 | IL-2-T3A-H16A- | APASSSTKKTQLQLEALLLNLQMILNGINNYKNPKLTRMLTFKFYMPKKATEL |
| | D20N-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 149 | IL-2-T3A-H16A- | APASSSTKKTQLQLEALLLALQMILNGINNYKNPKLTRMLTFKFYMPKKATEL |
| | D20A-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 150 | IL-2-T3A-H16A- | APASSSTKKTQLQLEALLNDLQMILNGINNYKNPKLTRMLTFKFYMPKKATEL |
| | L19N-E61R-P65R- | KHLQCLERELKRLEEVLNLAQSKNFHLRPRNLISNINVIVLELKGSETTFMCE |
| | D84N-C125S | YADETATIVEFLNRWITFSQSIISTLT |
| 151 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISTINVIVLELKGSETTFMCE |
| | N88T-C125S | YADETATIVEFLNRWITFSQSIISTLT |
| 152 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISAINVIVLELKGSETTFMCE |
| | N88A-C125S | YADETATIVEFLNRWITFSQSIISTLT |
| 153 | IL-2-T3A-H16P- | APASSSTKKTQLQLEPLLADLQMILNGINNYKNPKLTRMLTFKFYMPKKATEL |
| | L19A-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 154 | IL-2-T3A-H16A- | APASSSTKKTQLQLEALLHDLQMILNGINNYKNPKLTRMLTFKFYMPKKATEL |
| | L19H-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 155 | IL-2-T3A-H16A- | APASSSTKKTQLQLEALLPDLQMILNGINNYKNPKLTRMLTFKFYMPKKATEL |
| | L19P-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 156 | IL-2-T3A-H16A- | APASSSTKKTQLQLEALLQDLQMILNGINNYKNPKLTRMLTFKFYMPKKATEL |
| | L19Q-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 157 | IL-2-T3A-H16A- | APASSSTKKTQLQLEALLYDLQMILNGINNYKNPKLTRMLTFKFYMPKKATEL |
| | L19Y-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 158 | IL-2-T3A-H16A- | APASSSTKKTQLQLEALLADLQMILNGINNYKNPKLTRMLTFKFYMPKKATEL |
| | L19A-E61R-P65R- | KHLQCLERELKRLEEVLNLAQSKNFHLRPRPLISNINVIVLELKGSETTFMCE |
| | D84P-C125S | YADETATIVEFLNRWITFSQSIISTLT |
| 159 | IL-2-T3A-H16A- | APASSSTKKTQLQLEALLADLQMILNGINNYKNPKLTRMLTFKFYMPKKATEL |
| | L19A-E61R-P65R- | KHLQCLERELKRLEEVLNLAQSKNFHLRPRVLISNINVIVLELKGSETTFMCE |
| | D84V-C125S | YADETATIVEFLNRWITFSQSIISTLT |
| 160 | IL-2-T3A-H16A- | APASSSTKKTQLQLEALLADLQMILNGINNYKNPKLTRMLTFKFYMPKKATEL |
| | L19A-E61R-P65R- | KHLQCLERELKRLEEVLNLAQSKNFHLRPRYLISNINVIVLELKGSETTFMCE |
| | D84Y-C125S | YADETATIVEFLNRWITFSQSIISTLT |
| 161 | IL-2-T3A-H16A- | APASSSTKKTQLQLEALLLDLQAILNGINNYKNPKLTRMLTFKFYMPKKATEL |
| | M23A-E61R- | KHLQCLERELKRLEEVLNLAQSKNFHLRPRSLISNINVIVLHLKGSETTFMCE |
| | P65R-D84S-E95H- | YADETATIVEFLNRWITFSQSIISTLT |
| | C125S | |
| 162 | IL-2-T3A-H16A- | APASSSTKKTQLQLEALLLDLQAILNGINNYKNPKLTRMLTFKFYMPKKATEL |
| | M23A-E61R- | KHLQCLERELKRLEEVLNLAQSKNFHLRPRSLISNINVIVLNLKGSETTFMCE |
| | P65R-D84S-E95N- | YADETATIVEFLNRWITFSQSIISTLT |
| | C125S | |
| 163 | IL-2-T3A-H16A- | APASSSTKKTQLQLEALLLDLQAILNGINNYKNPKLTRMLTFKFYMPKKATEL |
| | M23A-E61R- | KHLQCLERELKRLEEVLNLAQSKNFHLRPRSLISNINVIVLPLKGSETTFMCE |
| | P65R-D84S-E95P- | YADETATIVEFLNRWITFSQSIISTLT |
| | C125S | |
| 164 | IL-2-T3A-H16A- | APASSSTKKTQLQLEALLLDLQAILNGINNYKNPKLTRMLTFKFYMPKKATEL |
| | M23A-E61R- | KHLQCLERELKRLEEVLNLAQSKNFHLRPRSLISNINVIVLSLKGSETTFMCE |
| | P65R-D84S-E95S- | YADETATIVEFLNRWITFSQSIISTLT |
| | C125S | |
| 165 | IL-2-T3A-H16A- | APASSSTKKTQLQLEALLLDLQAILNGINNYKNPKLTRMLTFKFYMPKKATEL |
| | M23A-E61R- | KHLQCLERELKRLEEVLNLAQSKNFHLRPRSLISNINVIVLTLKGSETTFMCE |
| | P65R-D84S-E95T- | YADETATIVEFLNRWITFSQSIISTLT |
| | C125S | |
| 166 | IL-2-T3A-H16A- | APASSSTKKTQLQLEALLLDLQAILNGINNYKNPKLTRMLTFKFYMPKKATEL |
| | M23A-E61R- | KHLQCLERELKRLEEVLNLAQSKNFHLRPRSLISNINVIVLVLKGSETTFMCE |
| | P65R-D84S-E95V- | YADETATIVEFLNRWITFSQSIISTLT |
| | C125S | |
| 167 | IL-2-T3A-H16A- | APASSSTKKTQLQLEALLLDLQAILNGINNYKNPKLTRMLTFKFYMPKKATEL |
| | M23A-E61R- | KHLQCLERELKRLEEVLNLAQSKNFHLRPRSLISNINVIVLYLKGSETTFMCE |
| | P65R-D84S-E95Y- | YADETATIVEFLNRWITFSQSIISTLT |
| | C125S | |
| 168 | IL-2-T3A-H16A- | APASSSTKKTQLQLEALLADLQMILNGINNYKNPKLTRMLTFKFYMPKKATEL |
| | L19A-E61A-P65R- | KHLQCLEAELKRLEEVLNLAQSKNFHLRPRSLISNINVIVLELKGSETTFMCE |
| | D84S-C125S | YADETATIVEFLNRWITFSQSIISTLT |
| 169 | IL-2-T3A-H16A- | APASSSTKKTQLQLEALLADLQMILNGINNYKNPKLTRMLTFKFYMPKKATEL |
| | L19A-E61G-P65R- | KHLQCLEGELKRLEEVLNLAQSKNFHLRPRSLISNINVIVLELKGSETTFMCE |
| | D84S-C125S | YADETATIVEFLNRWITFSQSIISTLT |
| 170 | IL-2-T3A-H16A- | APASSSTKKTQLQLEALLADLQMILNGINNYKNPKLTRMLTFKFYMPKKATEL |
| | L19A-E61H-P65R- | KHLQCLEHELKRLEEVLNLAQSKNFHLRPRSLISNINVIVLELKGSETTFMCE |
| | D84S-C125S | YADETATIVEFLNRWITFSQSIISTLT |
| 171 | IL-2-T3A-H16A- | APASSSTKKTQLQLEALLADLQMILNGINNYKNPKLTRMLTFKFYMPKKATEL |
| | L19A-E61N-P65R- | KHLQCLENELKRLEEVLNLAQSKNFHLRPRSLISNINVIVLELKGSETTFMCE |
| | D84S-C125S | YADETATIVEFLNRWITFSQSIISTLT |
| 172 | IL-2-T3A-H16A- | APASSSTKKTQLQLEALLADLQMILNGINNYKNPKLTRMLTFKFYMPKKATEL |
| | L19A-E61P-P65R- | KHLQCLEPELKRLEEVLNLAQSKNFHLRPRSLISNINVIVLELKGSETTFMCE |
| | D84S-C125S | YADETATIVEFLNRWITFSQSIISTLT |
| 173 | IL-2-T3A-H16A- | APASSSTKKTQLQLEALLADLQMILNGINNYKNPKLTRMLTFKFYMPKKATEL |
| | L19A-E61Q-P65R- | KHLQCLEQELKRLEEVLNLAQSKNFHLRPRSLISNINVIVLELKGSETTFMCE |
| | D84S-C125S | YADETATIVEFLNRWITFSQSIISTLT |
| 174 | IL-2-T3A-H16A- | APASSSTKKTQLQLEALLADLQMILNGINNYKNPKLTRMLTFKFYMPKKATEL |
| | L19A-E61S-P65R- | KHLQCLESELKRLEEVLNLAQSKNFHLRPRSLISNINVIVLELKGSETTFMCE |
| | D84S-C125S | YADETATIVEFLNRWITFSQSIISTLT |
| 175 | IL-2-T3A-H16A- | APASSSTKKTQLQLEALLADLQMILNGINNYKNPKLTRMLTFKFYMPKKATEL |
| | L19A-E61T-P65R- | KHLQCLETELKRLEEVLNLAQSKNFHLRPRSLISNINVIVLELKGSETTFMCE |
| | D84S-C125S | YADETATIVEFLNRWITFSQSIISTLT |
| 176 | IL-2-T3A-H16A- | APASSSTKKTQLQLEALLADLQMILNGINNYKNPKLTRMLTFKFYMPKKATEL |
| | L19A-E61V-P65R- | KHLQCLEVELKRLEEVLNLAQSKNFHLRPRSLISNINVIVLELKGSETTFMCE |
| | D84S-C125S | YADETATIVEFLNRWITFSQSIISTLT |
| 177 | IL-2-T3A-H16A- | APASSSTKKTQLQLEALLADLQMILNGINNYKNPKLTRMLTFKFYMPKKATEL |
| | L19A-E61Y-P65R- | KHLQCLEYELKRLEEVLNLAQSKNFHLRPRSLISNINVIVLELKGSETTFMCE |
| | D84S-C125S | YADETATIVEFLNRWITFSQSIISTLT |
| 178 | IL-2-T3A-H16A- | APASSSTKKTQLQLEALLLDLQDILNGINNYKNPKLTRMLTFKFYMPKKATEL |
| | M23D-E61R- | KHLQCLERELKRLEEVLNLAQSKNFHLRPRSLISNINVIVLQLKGSETTFMCE |
| | P65R-D84S-E95Q- | YADETATIVEFLNRWITFSQSIISTLT |
| | C125S | |
| 179 | IL-2-T3A-H16A- | APASSSTKKTQLQLEALLLDLQEILNGINNYKNPKLTRMLTFKFYMPKKATEL |
| | M23E-E61R-P65R- | KHLQCLERELKRLEEVLNLAQSKNFHLRPRSLISNINVIVLQLKGSETTFMCE |
| | D84S-E95Q-C125S | YADETATIVEFLNRWITFSQSIISTLT |
| 180 | IL-2-T3A-H16A- | APASSSTKKTQLQLEALLLDLQGILNGINNYKNPKLTRMLTFKFYMPKKATEL |
| | M23G-E61R- | KHLQCLERELKRLEEVLNLAQSKNFHLRPRSLISNINVIVLQLKGSETTFMCE |
| | P65R-D84S-E95Q- | YADETATIVEFLNRWITFSQSIISTLT |
| | C125S | |
| 181 | IL-2-T3A-H16A- | APASSSTKKTQLQLEALLLDLQIILNGINNYKNPKLTRMLTFKFYMPKKATEL |
| | M23I-E61R-P65R- | KHLQCLERELKRLEEVLNLAQSKNFHLRPRSLISNINVIVLQLKGSETTFMCE |
| | D84S-E95Q-C125S | YADETATIVEFLNRWITFSQSIISTLT |
| 182 | IL-2-T3A-H16A- | APASSSTKKTQLQLEALLLDLQKILNGINNYKNPKLTRMLTFKFYMPKKATEL |
| | M23K-E61R- | KHLQCLERELKRLEEVLNLAQSKNFHLRPRSLISNINVIVLQLKGSETTFMCE |
| | P65R-D84S-E95Q- | YADETATIVEFLNRWITFSQSIISTLT |
| | C125S | |
| 183 | IL-2-T3A-H16A- | APASSSTKKTQLQLEALLLDLQLILNGINNYKNPKLTRMLTFKFYMPKKATEL |
| | M23L-E61R-P65R- | KHLQCLERELKRLEEVLNLAQSKNFHLRPRSLISNINVIVLQLKGSETTFMCE |
| | D84S-E95Q-C125S | YADETATIVEFLNRWITFSQSIISTLT |
| 184 | IL-2-T3A-H16A- | APASSSTKKTQLQLEALLLDLQNILNGINNYKNPKLTRMLTFKFYMPKKATEL |
| | M23N-E61R- | KHLQCLERELKRLEEVLNLAQSKNFHLRPRSLISNINVIVLQLKGSETTFMCE |
| | P65R-D84S-E95Q- | YADETATIVEFLNRWITFSQSIISTLT |
| | C125S | |
| 185 | IL-2-T3A-H16A- | APASSSTKKTQLQLEALLLDLQPILNGINNYKNPKLTRMLTFKFYMPKKATEL |
| | M23P-E61R-P65R- | KHLQCLERELKRLEEVLNLAQSKNFHLRPRSLISNINVIVLQLKGSETTFMCE |
| | D84S-E95Q-C125S | YADETATIVEFLNRWITFSQSIISTLT |
| 186 | IL-2-T3A-H16A- | APASSSTKKTQLQLEALLLDLQQILNGINNYKNPKLTRMLTFKFYMPKKATEL |
| | M23Q-E61R- | KHLQCLERELKRLEEVLNLAQSKNFHLRPRSLISNINVIVLQLKGSETTFMCE |
| | P65R-D84S-E95Q- | YADETATIVEFLNRWITFSQSIISTLT |
| | C125S | |
| 187 | IL-2-T3A-H16A- | APASSSTKKTQLQLEALLLDLQRILNGINNYKNPKLTRMLTFKFYMPKKATEL |
| | M23R-E61R- | KHLQCLERELKRLEEVLNLAQSKNFHLRPRSLISNINVIVLQLKGSETTFMCE |
| | P65R-D84S-E95Q- | YADETATIVEFLNRWITFSQSIISTLT |
| | C125S | |
| 188 | IL-2-T3A-H16A- | APASSSTKKTQLQLEALLLDLQTILNGINNYKNPKLTRMLTFKFYMPKKATEL |
| | M23T-E61R-P65R- | KHLQCLERELKRLEEVLNLAQSKNFHLRPRSLISNINVIVLQLKGSETTFMCE |
| | D84S-E95Q-C125S | YADETATIVEFLNRWITFSQSIISTLT |
| 189 | IL-2-T3A-H16A- | APASSSTKKTQLQLEALLLDLQVILNGINNYKNPKLTRMLTFKFYMPKKATEL |
| | M23V-E61R- | KHLQCLERELKRLEEVLNLAQSKNFHLRPRSLISNINVIVLQLKGSETTFMCE |
| | P65R-D84S-E95Q- | YADETATIVEFLNRWITFSQSIISTLT |
| | C125S | |
| 190 | IL-2-T3A-H16A- | APASSSTKKTQLQLEALLLDLAMILNGINNYKNPKLTRMLTFKFYMPKKATEL |
| | Q22A-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 191 | IL-2-T3A-H16A- | APASSSTKKTQLQLEALLLDLDMILNGINNYKNPKLTRMLTFKFYMPKKATEL |
| | Q22D-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 192 | IL-2-T3A-H16A- | APASSSTKKTQLQLEALLLDLEMILNGINNYKNPKLTRMLTFKFYMPKKATEL |
| | Q22E-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 193 | IL-2-T3A-H16A- | APASSSTKKTQLQLEALLLDLGMILNGINNYKNPKLTRMLTFKFYMPKKATEL |
| | Q22G-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 194 | IL-2-T3A-H16A- | APASSSTKKTQLQLEALLLDLHMILNGINNYKNPKLTRMLTFKFYMPKKATEL |
| | Q22H-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 195 | IL-2-T3A-H16A- | APASSSTKKTQLQLEALLLDLKMILNGINNYKNPKLTRMLTFKFYMPKKATEL |
| | Q22K-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 196 | IL-2-T3A-H16A- | APASSSTKKTQLQLEALLLDLNMILNGINNYKNPKLTRMLTFKFYMPKKATEL |
| | Q22N-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 197 | IL-2-T3A-H16A- | APASSSTKKTQLQLEALLLDLPMILNGINNYKNPKLTRMLTFKFYMPKKATEL |
| | Q22P-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 198 | IL-2-T3A-H16A- | APASSSTKKTQLQLEALLLDLRMILNGINNYKNPKLTRMLTFKFYMPKKATEL |
| | Q22R-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 199 | IL-2-T3A-H16A- | APASSSTKKTQLQLEALLLDLSMILNGINNYKNPKLTRMLTFKFYMPKKATEL |
| | Q22S-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 200 | IL-2-T3A-H16A- | APASSSTKKTQLQLEALLLDLTMILNGINNYKNPKLTRMLTFKFYMPKKATEL |
| | Q22T-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 201 | IL-2-T3A-H16A- | APASSSTKKTQLQLEALLLDLVMILNGINNYKNPKLTRMLTFKFYMPKKATEL |
| | Q22V-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 202 | IL-2-T3A-H16A- | APASSSTKKTQLQLEALLLDLYMILNGINNYKNPKLTRMLTFKFYMPKKATEL |
| | Q22Y-E61R-P65R- | KHLQCLERELKRLEEVLNLAQSKNFHLRPRSLISNINVIVLELKGSETTFMCE |
| | D84S-C125S | YADETATIVEFLNRWITFSQSIISTLT |
| 203 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120A-C125S | YADETATIVEFLNAWITFSQSIISTLT |
| 204 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120D-C125S | YADETATIVEFLNDWITFSQSIISTLT |
| 205 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120E-C125S | YADETATIVEFLNEWITFSQSIISTLT |
| 206 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120F-C125S | YADETATIVEFLNEWITFSQSIISTLT |
| 207 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120G-C125S | YADETATIVEFLNGWITFSQSIISTLT |
| 208 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120H-C125S | YADETATIVEFLNHWITFSQSIISTLT |
| 209 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120K-C125S | YADETATIVEFLNRWITFSQSIISTLT |
| 210 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120N-C125S | YADETATIVEFLNNWITFSQSIISTLT |
| 211 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120P-C125S | YADETATIVEFLNPWITFSQSIISTLT |
| 212 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120Q-C125S | YADETATIVEFLNQWITFSQSIISTLT |
| 213 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120S-C125S | YADETATIVEFLNSWITFSQSIISTLT |
| 214 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120T-C125S | YADETATIVEFLNTWITFSQSIISTLT |
| 215 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120V-C125S | YADETATIVEFLNVWITFSQSIISTLT |
| 216 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | R120Y-C125S | YADETATIVEFLNYWITFSQSIISTLT |
| 217 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | T123D-C125S | YADETATIVEFLNRWIDFSQSIISTLT |
| 218 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | T123E-C125S | YADETATIVEFLNRWIEFSQSIISTLT |
| 219 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | T123H-C125S | YADETATIVEFLNRWIHFSQSIISTLT |
| 220 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | T123K-C125S | YADETATIVEFLNRWIKFSQSIISTLT |
| 221 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | T123N-C125S | YADETATIVEFLNRWINFSQSIISTLT |
| 222 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | T123Q-C125S | YADETATIVEFLNRWIQFSQSIISTLT |
| 223 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | T123R-C125S | YADETATIVEFLNRWIRFSQSIISTLT |
| 224 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S130D | YADETATIVEFLNRWITFSQSIIDTLT |
| 225 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S130E | YADETATIVEFLNRWITFSQSIIETLT |
| 226 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S130K | YADETATIVEFLNRWITFSQSIIKTLT |
| 227 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S130N | YADETATIVEFLNRWITFSQSIINTLT |
| 228 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S130Q | YADETATIVEFLNRWITFSQSIIQTLT |
| 229 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S130R | YADETATIVEFLNRWITFSQSIIRTLT |
| 230 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S127D | YADETATIVEFLNRWITFSQDIISTLT |
| 231 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S127E | YADETATIVEFLNRWITFSQSIISTLT |
| 232 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S127H | YADETATIVEFLNRWITFSQHIISTLT |
| 233 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S127P | YADETATIVEFLNRWITFSQPIISTLT |
| 234 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S127Q | YADETATIVEFLNRWITFSQQIISTLT |
| 235 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-S127R | YADETATIVEFLNRWITFSQRIISTLT |
| 236 | IL-2-T3A-H16A- | APASSSTKKTQLQLEALLADLAMILNGINNYKNPKLTAMLTFKFYMPKKATEL |
| | L19A-Q22A- | KHLQCLEEELKRLEEVLNLAQSKNFHLRPRSLISNINVIVLELKGSETTFMCE |
| | R38A-P65R-D84S- | YADETATIVEFLNRWITFSQSIISTLT |
| | C125S | |
| 237 | IL-2-T3A-H16A- | APASSSTKKTQLQLEALLADLQMILNGINNYKNPKLTAMLTFKFYMPKKATEL |
| | L19A-R38A-P65R- | KHLQCLEEELKRLEEVLNLAQSKNFHLRPRSLISNINVIVLELKGSETTFMCE |
| | D84S-R120A- | YADETATIVEFLNAWITFSQSIISTLT |
| | C125S | |
| 238 | IL-2-T3A-H16A- | APASSSTKKTQLQLEALLADLAMILNGINNYKNPKLTAMLTFKFYMPKKATEL |
| | L19A-Q22A- | KHLQCLEEELKRLEEVLNLAQSKNFHLRPRSLISNINVIVLELKGSETTFMCE |
| | R38A-P65R-D84S- | YADETATIVEFLNAWITFSQSIISTLT |
| | R120A-C125S | |
| 239 | IL-2-T3A-H16A- | APASSSTKKTQLQLEALLLDLAAILNGINNYKNPKLTAMLTFKFYMPKKATEL |
| | Q22A-M23A- | KHLQCLEEELKRLEEVLNLAQSKNFHLRPRSLISNINVIVLQLKGSETTFMCE |
| | R38A-P65R-D84S- | YADETATIVEFLNRWITFSQSIISTLT |
| | E95Q-C125S | |
| 240 | IL-2-T3A-H16A- | APASSSTKKTQLQLEALLLDLQAILNGINNYKNPKLTAMLTFKFYMPKKATEL |
| | M23A-R38A- | KHLQCLEEELKRLEEVLNLAQSKNFHLRPRSLISNINVIVLQLKGSETTFMCE |
| | P65R-D84S-E95Q- | YADETATIVEFLNAWITFSQSIISTLT |
| | R120A-C125S | |
| 241 | IL-2-T3A-H16A- | APASSSTKKTQLQLEALLLDLAAILNGINNYKNPKLTAMLTFKFYMPKKATEL |
| | Q22A-M23A- | KHLQCLEEELKRLEEVLNLAQSKNFHLRPRSLISNINVIVLQLKGSETTFMCE |
| | R38A-P65R-D84S- | YADETATIVEFLNAWITFSQSIISTLT |
| | E95Q-R120A- | |
| | C125S | |
| 242 | IL-2-T3A-H16A- | APASSSTKKTQLQLEALLNDLAMILNGINNYKNPKLTAMLTFKFYMPKKATEL |
| | L19N-Q22A- | KHLQCLEEELKRLEEVLNLAQSKNFHLRPRSLISNINVIVLELKGSETTFMCE |
| | R38A-P65R-D84S- | YADETATIVEFLNRWITFSQSIISTLT |
| | C125S | |
| 243 | IL-2-T3A-H16A- | APASSSTKKTQLQLEALLNDLQMILNGINNYKNPKLTAMLTFKFYMPKKATEL |
| | L19N-R38A-P65R- | KHLQCLEEELKRLEEVLNLAQSKNFHLRPRSLISNINVIVLELKGSETTFMCE |
| | D84S-R120A- | YADETATIVEFLNAWITFSQSIISTLT |
| | C125S | |
| 244 | IL-2-T3A-H16A- | APASSSTKKTQLQLEALLNDLAMILNGINNYKNPKLTAMLTFKFYMPKKATEL |
| | L19N-Q22A- | KHLQCLEEELKRLEEVLNLAQSKNFHLRPRSLISNINVIVLELKGSETTFMCE |
| | R38A-P65R-D84S- | YADETATIVEFLNAWITFSQSIISTLT |
| | R120A-C125S | |
| 245 | IL-2-A1G-P2G- | GGSGGSTKKTQLQLEALLADLQMILNGINNYKNPKLTAMLTFKFYMPKKATEL |
| | T3S-S4G-S5G- | KHLQCLEEELKRLEEVLNLAQSKNFHLRPRSLISNINVIVLELKGSETTFMCE |
| | H16A-L19A- | YADETATIVEFLNRWITFSQSIISTLT |
| | R38A-P65R-D84S- | |
| | C125S | |
| 246 | IL-2-del1-5-H16A- | STKKTQLQLEALLADLQMILNGINNYKNPKLTAMLTFKFYMPKKATELKHLQC |
| | L19A-R38A-P65R- | LEEELKRLEEVLNLAQSKNFHLRPRSLISNINVIVLELKGSETTFMCEYADET |
| | D84S-C125S | ATIVEFLNRWITFSQSIISTLT |
| 247 | IL-2-del1-5-H16A- | STKKTQLQLEALLADLQMILNGINNYKNPKLTAMLTFKFYMPKKATELKHLQC |
| | L19A-R38A-P65R- | LEEELKRLEEVLNLAQSKNFHLRPRSLISNINVIVLELKGSETTFMCEYADET |
| | D84S-C125V | ATIVEFLNRWITFVQSIISTLT |
| 248 | IL-2-T3A-L19N- | APASSSTKKTQLQLEHLLNDLQMILNGINNYKNPKLTAMLTFKFYMPKKATEL |
| | R38A-P65R-D84S- | KHLQCLEEELKRLEEVLNLAQSKNFHLRPRSLISNINVIVLELKGSETTFMCE |
| | C125S | YADETATIVEFLNRWITFSQSIISTLT |
| 249 | IL-2-T3A-H16A- | APASSSTKKTQLQLEALLNDLQMILNGINNYKNPKLTAMLTFKFYMPKKATEL |
| | L19N-R38A-P65R- | KHLQCLEEELKRLEEVLNLAQSKNFHLRPRDLISNINVIVLELKGSETTFMCE |
| | C125S | YADETATIVEFLNRWITFSQSIISTLT |
| 250 | IL-2-T3A-L19N- | APASSSTKKTQLQLEHLLNDLAMILNGINNYKNPKLTAMLTFKFYMPKKATEL |
| | Q22A-R38A- | KHLQCLEEELKRLEEVLNLAQSKNFHLRPRSLISNINVIVLELKGSETTFMCE |
| | P65R-D84S-C125S | YADETATIVEFLNRWITFSQSIISTLT |
| 251 | IL-2-T3A-H16A- | APASSSTKKTQLQLEALLNDLAMILNGINNYKNPKLTAMLTFKFYMPKKATEL |
| | L19N-Q22A- | KHLQCLEEELKRLEEVLNLAQSKNFHLRPRDLISNINVIVLELKGSETTFMCE |
| | R38A-P65R-C125S | YADETATIVEFLNRWITFSQSIISTLT |
| 252 | IL-2-T3A-L19N- | APASSSTKKTQLQLEHLLNDLAMILNGINNYKNPKLTAMLTFKFYMPKKATEL |
| | Q22A-R38A- | KHLQCLEEELKRLEEVLNLAQSKNFHLRPRSLISNINVIVLELKGSETTFMCE |
| | P65R-D84S- | YADETATIVEFLNAWITFSQSIISTLT |
| | R120A-C125S | |
| 253 | IL-2-T3A-H16A- | APASSSTKKTQLQLEALLNDLAMILNGINNYKNPKLTAMLTFKFYMPKKATEL |
| | L19N-Q22A- | KHLQCLEEELKRLEEVLNLAQSKNFHLRPRDLISNINVIVLELKGSETTFMCE |
| | R38A-P65R- | YADETATIVEFLNAWITFSQSIISTLT |
| | R120A-C125S | |
| 254 | IL-2-T3A-Q22A- | APASSSTKKTQLQLEHLLLDLAAILNGINNYKNPKLTAMLTFKFYMPKKATEL |
| | M23A-R38A- | KHLQCLEEELKRLEEVLNLAQSKNFHLRPRSLISNINVIVLQLKGSETTFMCE |
| | P65R-D84S-E95Q- | YADETATIVEFLNRWITFSQSIISTLT |
| | C125S | |
| 255 | IL-2-T3A-Q22A- | APASSSTKKTQLQLEALLLDLAAILNGINNYKNPKLTAMLTFKFYMPKKATEL |
| | M23A-R38A- | KHLQCLEEELKRLEEVLNLAQSKNFHLRPRDLISNINVIVLQLKGSETTFMCE |
| | P65R-E95Q-C125S | YADETATIVEFLNRWITFSQSIISTLT |
| 256 | IL-2-T3A-Q22A- | APASSSTKKTQLQLEHLLLDLAAILNGINNYKNPKLTAMLTFKFYMPKKATEL |
| | M23A-R38A- | KHLQCLEEELKRLEEVLNLAQSKNFHLRPRSLISNINVIVLQLKGSETTFMCE |
| | P65R-D84S-E95Q- | YADETATIVEFLNAWITFSQSIISTLT |
| | R120A-C125S | |
| 257 | IL-2-T3A-H16A- | APASSSTKKTQLQLEALLLDLAAILNGINNYKNPKLTAMLTFKFYMPKKATEL |
| | Q22A-M23A- | KHLQCLEEELKRLEEVLNLAQSKNFHLRPRDLISNINVIVLQLKGSETTFMCE |
| | R38A-P65R-E95Q- | YADETATIVEFLNAWITFSQSIISTLT |
| | R120A-C125S | |
| 258 | IL-2-T3A-L19A- | APASSSTKKTQLQLEHLLADLAMILNGINNYKNPKLTAMLTFKFYMPKKATEL |
| | Q22A-R38A- | KHLQCLEEELKRLEEVLNLAQSKNFHLRPRSLISNINVIVLELKGSETTFMCE |
| | P65R-D84S-C125S | YADETATIVEFLNRWITFSQSIISTLT |
| 259 | IL-2-T3A-H16A- | APASSSTKKTQLQLEALLADLAMILNGINNYKNPKLTAMLTFKFYMPKKATEL |
| | L19A-Q22A- | KHLQCLEEELKRLEEVLNLAQSKNFHLRPRDLISNINVIVLELKGSETTFMCE |
| | R38A-P65R-C125S | YADETATIVEFLNRWITFSQSIISTLT |
| 260 | IL-2-T3A-L19A- | APASSSTKKTQLQLEHLLADLAMILNGINNYKNPKLTAMLTFKFYMPKKATEL |
| | Q22A-R38A- | KHLQCLEEELKRLEEVLNLAQSKNFHLRPRSLISNINVIVLELKGSETTFMCE |
| | P65R-D84S- | YADETATIVEFLNAWITFSQSIISTLT |
| | R120A-C125S | |
| 261 | IL-2-T3A-H16A- | APASSSTKKTQLQLEALLADLAMILNGINNYKNPKLTAMLTFKFYMPKKATEL |
| | L19A-Q22A- | KHLQCLEEELKRLEEVLNLAQSKNFHLRPRDLISNINVIVLELKGSETTFMCE |
| | R38A-P65R- | YADETATIVEFLNAWITFSQSIISTLT |
| | R120A-C125S | |
| 262 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-I129A | YADETATIVEFLNRWITFSQSIASTLT |
| 263 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-I129H | YADETATIVEFLNRWITFSQSIHSTLT |
| 264 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-I129R | YADETATIVEFLNRWITFSQSIRSTLT |
| 265 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125S-I129S | YADETATIVEFLNRWITFSQSISSTLT |
| 266 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125C-Q126N | YADETATIVEFLNRWITFSNSIISTLT |
| 267 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125C-Q126Y | YADETATIVEFLNRWITFSYSIISTLT |
| 268 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125C-S127N | YADETATIVEFLNRWITFSQNIISTLT |
| 269 | IL-2-T3A-H16A- | APASSSTKKTQLQLEALLLDLQMILNGINNYKNPKLTRMLTFKFYMPKKATEL |
| | E61R-P65R-D84S- | KHLQCLERELKRLEEVLNLAQSKNFHLRPRSLISNINVIVLELKGSETTFMCE |
| | C125C-S130P | YADETATIVEFLNRWITFSQSIIPTLT |
| 270 | IL-2-T3A-H16A- | APASSSTKKTQLQLEALLLDLQMILNGINNYKNPKLTRMLTFKFYMPKKATEL |
| | E61R-P65R-D84Y- | KHLQCLERELKRLEEVLNLAQSKNFHLRPRYLISNINVIVLELKGSETTFMCE |
| | C125S | YADETATIVEFLNRWITFSQSIISTLT |
| 271 | IL-2-T3A-H16A- | APASSSTKKTQLQLEALLLDLQTILNGINNYKNPKLTRMLTFKFYMPKKATEL |
| | M23T-E61R-P65R- | KHLQCLERELKRLEEVLNLAQSKNFHLRPRYLISNINVIVLQLKGSETTFMCE |
| | D84Y-E95Q- | YADETATIVEFLNRWITFSQSIISTLT |
| | C125S | |
| 272 | IL-2-T3A-H16A- | APASSSTKKTQLQLEALLNDLQMILNGINNYKNPKLTRMLTFKFYMPKKATEL |
| | L19N-E61R-P65R- | KHLQCLERELKRLEEVLNLAQSKNFHLRPRYLISNINVIVLELKGSETTFMCE |
| | D84Y-C125S | YADETATIVEFLNRWITFSQSIISTLT |
| 273 | IL-2-T3A-H16A- | APASSSTKKTQLQLEALLNDLQTILNGINNYKNPKLTRMLTFKFYMPKKATEL |
| | L19N-M23T- | KHLQCLERELKRLEEVLNLAQSKNFHLRPRYLISNINVIVLQLKGSETTFMCE |
| | E61R-P65R-D84Y- | YADETATIVEFLNRWITFSQSIISTLT |
| | E95Q-C125S | |
| 274 | IL-2-T3A-H16A- | APASSSTKKTQLQLEALLLDLQMILNGINNYKNPKLTRMLTFKFYMPKKATEL |
| | E61R-P65R-D84Y- | KHLQCLERELKRLEEVLNLAQSKNFHLRPRYLISNINVIVLELKGSETTFMCE |
| | C125S-S127D | YADETATIVEFLNRWITFSQDIISTLT |
| 275 | IL-2-T3A-H16A- | APASSSTKKTQLQLEALLLDLQTILNGINNYKNPKLTRMLTFKFYMPKKATEL |
| | M23T-E61R-P65R- | KHLQCLERELKRLEEVLNLAQSKNFHLRPRYLISNINVIVLQLKGSETTFMCE |
| | D84Y-E95Q- | YADETATIVEFLNRWITFSQDIISTLT |
| | C125S-S127D | |
| 276 | IL-2-T3A-H16A- | APASSSTKKTQLQLEALLNDLQMILNGINNYKNPKLTRMLTFKFYMPKKATEL |
| | L19N-E61R-P65R- | KHLQCLERELKRLEEVLNLAQSKNFHLRPRYLISNINVIVLELKGSETTFMCE |
| | D84Y-C125S- | YADETATIVEFLNRWITFSQDIISTLT |
| | S127D | |
| 277 | IL-2-T3A-H16A- | APASSSTKKTQLQLEALLNDLQTILNGINNYKNPKLTRMLTFKFYMPKKATEL |
| | L19N-M23T- | KHLQCLERELKRLEEVLNLAQSKNFHLRPRYLISNINVIVLQLKGSETTFMCE |
| | E61R-P65R-D84Y- | YADETATIVEFLNRWITFSQDIISTLT |
| | E95Q-C125S- | |
| | S127D | |
| 278 | IL-2-T3A-H16A- | APASSSTKKTQLQLEALLLDLQMILNGINNYKNPKLTRMLT- |
| | ΔF42-P65R-D84S- | KFYMPKKATELKHLQCLEEELKRLEEVLNLAQSKNFHLRPRSLISNINVIVLE |
| | C125S | LKGSETTFMCEYADETATIVEFLNRWITFSQSIISTLT |
| 279 | IL-2-T3A-H16A- | APASSSTKKTQLQLEALLLDLQMILNGINNYKNPKLTRMLTFEFRMPKKATEL |
| | K43E-Y45R-P65R- | KHLQCLEEELKRLEEVLNLAQSKNFHLRPRSLISNINVIVLELKGSETTFMCE |
| | D84S-C125S | YADETATIVEFLNRWITFSQSIISTLT |
| 280 | IL-2-T3A-H16A- | APASSSTKKTQLQLEALLLDLQMILNGINNYKNPKLTRMLTFEFYMPKKATEL |
| | K43E-P65R-D84S- | KHLQCLEEELKRLEEVLNLAQSKNFHLRPRSLISNINVIVLELKGSETTFMCE |
| | C125S | YADETATIVEFLNRWITFSQSIISTLT |
| 281 | IL-2-T3A-H16A- | APASSSTKKTQLQLEALLLDLQMILNGINNYKNPKLTRMLTFKFYMPKKATEL |
| | P65R-D84S-I114F- | KHLQCLEEELKRLEEVLNLAQSKNFHLRPRSLISNINVIVLELKGSETTFMCE |
| | C125S | YADETATFVEFLNRWITFSQSIISTLT |
| 282 | IL-2-T3A-H16A- | APASSSTKKTQLQLEALLLDLQMILNGINNYKNPKLTRMLTRKFYMPKKATEL |
| | F42R-P65R-D84S- | KHLQCLEEELKRLEEVLNLAQSKNFHLRPRSLISNINVIVLELKGSETTFMCE |
| | C125S | YADETATIVEFLNRWITFSQSIISTLT |
| 283 | IL-2-T3A-K43E- | APASSSTKKTQLQLEHLLLDLQMILNGINNYKNPKLTRMLTFEFRMPKKATEL |
| | Y45R-C125S | KHLQCLEEELKPLEEVLNLAQSKNFHLRPRDLISNINVIVLELKGSETTFMCE |
| | | YADETATIVEFLNRWITFSQSIISTLT |
| 284 | IL-2-T3A-E61R- | APASSSTKKTQLQLEHLLLDLQMILNGINNYKNPKLTRMLTFKFYMPKKATEL |
| | C125S | KHLQCLERELKPLEEVLNLAQSKNFHLRPRDLISNINVIVLELKGSETTFMCE |
| | | YADETATIVEFLNRWITFSQSIISTLT |
| 285 | IL-2-T3A-I114F- | APASSSTKKTQLQLEHLLLDLQMILNGINNYKNPKLTRMLTFKFYMPKKATEL |
| | C125S | KHLQCLEEELKPLEEVLNLAQSKNFHLRPRDLISNINVIVLELKGSETTFMCE |
| | | YADETATFVEFLNRWITFSQSIISTLT |
| 286 | IL-2-T3A-K43E- | APASSSTKKTQLQLEHLLLDLQMILNGINNYKNPKLTRMLTFEFYMPKKATEL |
| | C125S | KHLQCLEEELKPLEEVLNLAQSKNFHLRPRDLISNINVIVLELKGSETTFMCE |
| | | YADETATIVEFLNRWITFSQSIISTLT |
| 287 | IL-2-T3A-P65R- | APASSSTKKTQLQLEHLLLDLQMILNGINNYKNPKLTRMLTFKFYMPKKATEL |
| | I114F-C125S | KHLQCLEEELKRLEEVLNLAQSKNFHLRPRDLISNINVIVLELKGSETTFMCE |
| | | YADETATFVEFLNRWITFSQSIISTLT |
| 288 | IL-2-T3A-K43E- | APASSSTKKTQLQLEHLLLDLQMILNGINNYKNPKLTRMLTFEFYMPKKATEL |
| | P65R-C125S | KHLQCLEEELKRLEEVLNLAQSKNFHLRPRDLISNINVIVLELKGSETTFMCE |
| | | YADETATIVEFLNRWITFSQSIISTLT |
| 289 | IL-2-T3A-K43E- | APASSSTKKTQLQLEHLLLDLQMILNGINNYKNPKLTRMLTFEFRMPKKATEL |
| | Y45R-P65R-C125S | KHLQCLEEELKRLEEVLNLAQSKNFHLRPRDLISNINVIVLELKGSETTFMCE |
| | | YADETATIVEFLNRWITFSQSIISTLT |
| 290 | IL-2-T3A-R38A- | APASSSTKKTQLQLEHLLLDLQMILNGINNYKNPKLTAMLTFKFYMPKKATEL |
| | C125S | KHLQCLEEELKPLEEVLNLAQSKNFHLRPRDLISNINVIVLELKGSETTFMCE |
| | | YADETATIVEFLNRWITFSQSIISTLT |
| 291 | IL-15 WT | MNWVNVISDLKKIEDLIQSMHIDATLYTESDVHPSCKVTAMKCFLLELQVISL |
| | | ESGDASIHDTVENLIILANNSLSSNGNVTESGCKECEELEEKNIKEFLQSFVH |
| | | IVQMFINTS |
| 292 | Fc region xELL | DKTHTCPPCPAPGGPSVFLFPPKPKDTLMISRTPEVTCVVVDVSHEDPEVKFN |
| | P329G knob | WYVDGVEVHNAKTKPREEQYNSTYRWSVLTVLHQDWLNGKEYKCKVSNKALG |
| | | APIEKTISKAKGQPREPQVYTLPPCRDELTKNQVSLWCLVKGFYPSDIAVEWE |
| | | SNGQPENNYKTTPPVLDSDGSFFLYSKLTVDKSRWQQGNVFSCSVMHEALHNH |
| | | YTQKSLSLSPG |
| 293 | Fc region xELL | DKTHTCPPCPAPGGPSVFLFPPKPKDTLMISRTPEVTCVVVDVSHEDPEVKFN |
| | P329G, H435R | WYVDGVEVHNAKTKPREEQYNSTYRWSVLTVLHQDWLNGKEYKCKVSNKALG |
| | hole | APIEKTISKAKGQPREPQVCTLPPSRDELTKNQVSLSCAVKGFYPSDIAVEWE |
| | | SNGQPENNYKTTPPVLDSDGSFFLVSKLTVDKSRWQQGNVFSCSVMHEALHNR |
| | | YTQKSLSLSPG |
| 294 | xELL-knob Fc - | DKTHTCPPCPAPGGPSVFLFPPKPKDTLMISRTPEVTCVVVDVSHEDPEVKFN |
| | IL-2-RAS-T3G, | WYVDGVEVHNAKTKPREEQYNSTYRVVSVLTVLHQDWLNGKEYKCKVSNKALP |
| | C125S | APIEKTISKAKGQPREPQVYTLPPCRDELTKNQVSLWCLVKGFYPSDIAVEWE |
| | | SNGQPENNYKTTPPVLDSDGSFFLYSKLTVDKSRWQQGNVFSCSVMHEALHNH |
| | | YTQKSLSLSPGGSGGSAPGSSSTKKTQLQLEALLLDLQMILNGINNYKNPKLT |
| | | RMLTFKFYMPKKATELKHLQCLEEELKRLEEVLNLAQSKNFHLRPRSLISNIN |
| | | VIVLELKGSETTFMCEYADETATIVEFLNRWITFSQSIISTLT |
| 295 | γδTCR 5C8 VHH- | EVQLVESGGGLVQAGGSLRLSCAASGRPFSNYAMGWFRQAPGKEREFVAAISW |
| | xELL- hole T366S, | SGGSTSYADSVKGRFTISRDNAKNTVYLQMNSPKPEDTAIYYCAAQFSGADYG |
| | L368A, Y407V, | FGRLGIRGYEYDYWGQGTQVTVKPGGGGDKTHTCPPCPAPGGPSVFLFPPKPK |
| | H435R Fc | DTLMISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVCTLPPS |
| | | RDELTKNQVSLSCAVKGFYPSDIAVEWESNGQPENNYKTTPPVLDSDGSFFLV |
| | | SKLTVDKSRWQQGNVFSCSVMHEALHNRYTQKSLSLSPGK |
| 296 | γδTCR 5C8 VHH- | EVQLVESGGGLVQAGGSLRLSCAASGRPFSNYAMGWFRQAPGKEREFVAAISW |
| | xELL-knob Fc-IL- | SGGSTSYADSVKGRFTISRDNAKNTVYLQMNSPKPEDTAIYYCAAQFSGADYG |
| | 2-T3A-H16A- | FGRLGIRGYEYDYWGQGTQVTVKPGGGGDKTHTCPPCPAPGGPSVFLFPPKPK |
| | E61R-P65R-D84Y- | DTLMISRTPEVTCVVVDVSHEDPEVKFNWYVDGVEVHNAKTKPREEQYNSTYR |
| | C125S | VVSVLTVLHQDWLNGKEYKCKVSNKALPAPIEKTISKAKGQPREPQVYTLPPC |
| | | RDELTKNQVSLWCLVKGFYPSDIAVEWESNGQPENNYKTTPPVLDSDGSFFLY |
| | | SKLTVDKSRWQQGNVFSCSVMHEALHNHYTQKSLSLSPGGSGGSAPASSSTKK |
| | | TQLQLEALLLDLQMILNGINNYKNPKLTRMLTFKFYMPKKATELKHLQCLERE |
| | | LKRLEEVLNLAQSKNFHLRPRYLISNINVIVLELKGSETTFMCEYADETATIV |
| | | EFLNRWITFSQSIISTLT |
| 298 | γδTCR 6C4 VHH- | EVQLVESGGGLVQAGGSLRLSCAVSVRTFSDYRMGWFRQAPGKEREFVSTISW |
| | xELL-knob Fc-IL- | SGGLTYYADSVKGRFTISRDNSKNTLYLQMNSLKPEDTAVYYCAAGGGYAGGT |
| | 2-T3A-H16A- | YYHPEEWGQGTQVTVKPGGGGDKTHTCPPCPAPGGPSVFLFPPKPKDTLMISR |
| | E61R-P65R-D84Y- | TPEVTCVVVDVSHEDPEVKFNWYVDGVEVHNAKTKPREEQYNSTYRVVSVLTV |
| | C125S | LHQDWLNGKEYKCKVSNKALPAPIEKTISKAKGQPREPQVYTLPPCRDELTKN |
| | | QVSLWCLVKGFYPSDIAVEWESNGQPENNYKTTPPVLDSDGSFFLYSKLTVDK |
| | | SRWQQGNVFSCSVMHEALHNHYTQKSLSLSPGGSGGSAPASSSTKKTQLQLEA |
| | | LLLDLQMILNGINNYKNPKLTRMLTFKFYMPKKATELKHLQCLERELKRLEEV |
| | | LNLAQSKNFHLRPRYLISNINVIVLELKGSETTFMCEYADETATIVEFLNRWI |
| | | TFSQSIISTLT |
| 299 | γδTCR 6C4 VHH- | EVQLVESGGGLVQAGGSLRLSCAVSVRTFSDYRMGWFRQAPGKEREFVSTISW |
| | xELL-hole T366S, | SGGLTYYADSVKGRFTISRDNSKNTLYLQMNSLKPEDTAVYYCAAGGGYAGGT |
| | L368A, Y407V | YYHPEEWGQGTQVTVKPGGGGDKTHTCPPCPAPGGPSVFLFPPKPKDTLMISR |
| | H435RFc | TPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVCTLPPSRDELTKN |
| | | QVSLSCAVKGFYPSDIAVEWESNGQPENNYKTTPPVLDSDGSFFLVSKLTVDK |
| | | SRWQQGNVFSCSVMHEALHNRYTQKSLSLSPGK |
| 300 | Untargeted VHH- | EVQLVESGGGEVQPGGSLRLSCAASGSINSINVMEWYRQAPGKERDLVAGITS |
| | xELL-hole T366S, | DGDTNYAESVKGRFTISRDNAKNTLYLQMSSLRAEDTAVYYCRARDWGSLTDY |
| | L368A, Y407V | WGQGTLVTVKPGGGGDKTHTCPPCPAPGGPSVFLFPPKPKDTLMISRTPEVTC |
| | H435RFc | VVVDVSHEDPEVKFNWYVDGVEVHNAKTKPREEQYNSTYRVVSVLTVLHQDWL |
| | | NGKEYKCKVSNKALPAPIEKTISKAKGQPREPQVCTLPPSRDELTKNQVSLSC |
| | | AVKGFYPSDIAVEWESNGQPENNYKTTPPVLDSDGSFFLVSKLTVDKSRWQQG |
| | | NVFSCSVMHEALHNRYTQKSLSLSPGK |
| 301 | Untargeted VHH- | EVQLVESGGGEVQPGGSLRLSCAASGSINSINVMEWYRQAPGKERDLVAGITS |
| | xELL-knob Fc- IL- | DGDTNYAESVKGRFTISRDNAKNTLYLQMSSLRAEDTAVYYCRARDWGSLTDY |
| | 2-T3A-H16A- | WGQGTLVTVKPGGGGDKTHTCPPCPAPGGPSVFLFPPKPKDTLMISRTPEVTC |
| | E61R-P65R-D84Y- | VVVDVSHEDPEVKFNWYVDGVEVHNAKTKPREEQYNSTYRVVSVLTVLHQDWL |
| | C125S | NGKEYKCKVSNKALPAPIEKTISKAKGQPREPQVYTLPPCRDELTKNQVSLWC |
| | | LVKGFYPSDIAVEWESNGQPENNYKTTPPVLDSDGSFFLYSKLTVDKSRWQQG |
| | | NVFSCSVMHEALHNHYTQKSLSLSPGGSGGSAPASSSTKKTQLQLEALLLDLQ |
| | | MILNGINNYKNPKLTRMLTFKFYMPKKATELKHLQCLERELKRLEEVLNLAQS |
| | | KNFHLRPRYLISNINVIVLELKGSETTFMCEYADETATIVEFLNRWITFSQSI |
| | | ISTLT |

In sequences that contain boxes or underlining, the boxes around individual letters indicate amino acid substitutions relative to a corresponding wild type or parental sequence; boxes around groups of letters indicate linker sequences. Underlined letters are linker sequences. Sequences that do not contain boxes or underlining may also contain amino acid substitutions and/or linker sequences.

What is claimed is:

1. A polypeptide comprising a modified IL-2, wherein the modified IL-2 comprises a D84Y substitution.

2. The polypeptide of claim 1, wherein the modified IL-2 has reduced affinity for CD122 compared to wild-type IL-2.

3. The polypeptide of any one of claims 1-2, wherein the modified IL-2 comprises at least one substitution at at least one amino acid position selected from H16, L19, M23, N88, and E95.

4. The polypeptide of claim 3, wherein the modified IL-2 comprises a substitution at amino acid position H16.

5. The polypeptide of claim 4, wherein the substitution is selected from H16A H16N, H16V, and H16T.

6. The polypeptide of any one of claims 1-5, wherein the modified IL-2 comprises a substitution at amino acid position L19.

7. The polypeptide of claim 6, wherein the substitution is selected from L19A, L19P, L19Q, L19Y, L19N, L19S, L19T, L19V.

8. The polypeptide of any one of claims 1-7, wherein the modified IL-2 comprises a substitution at amino acid position M23.

9. The polypeptide of claim 8, wherein the substitution is selected from M23A, M23G, M23S, M23T, M23V, M23D, M23E, M23I, M23K, M23L, M23N, M23Q, M23R, and M23Y.

10. The polypeptide of any one of claims 1-9, wherein the modified IL-2 comprises a substitution at amino acid position N88.

11. The polypeptide of claim 10, wherein the substitution is selected from N88T, N88A, and N88S.

12. The polypeptide of any one of claims 1-11, wherein the modified IL-2 comprises a substitution at amino acid position E95.

13. The polypeptide of claim 12, wherein the substitution is selected from E95Q, E95G, E95T, E95V, E95P, E95H, E95N, and E95Y.

14. The polypeptide of any one of claims 1-13, wherein the modified IL-2 comprises at least one substitution that reduces affinity for CD132 compared to wild-type IL-2.

15. The polypeptide of any one of claims 1-14, wherein the modified IL-2 comprises at least one substitution at at least one amino acid position selected from Q22, R120, T123, Q126, S127, I129, and S130.

16. The polypeptide of claim 15, wherein the modified IL-2 comprises a substitution at amino acid position Q22.

17. The polypeptide of claim 16, wherein the substitution is selected from Q22A, Q22D, Q22G, Q22H, Q22K, Q22N, Q22R, Q22S, Q22T, Q22V, and Q22Y.

18. The polypeptide of any one of claims 15-17, wherein the modified IL-2 comprises a substitution at amino acid position R120.

19. The polypeptide of claim 18, wherein the substitution is selected from R120A, R120D, R120E, R120F, R120G, R120H, R120K, R120N, R120Q, R120S, R120V, and R120Y.

20. The polypeptide of any one of claims 15-19, wherein the modified IL-2 comprises a substitution at amino acid position T123.

21. The polypeptide of claim 20, wherein the substitution is selected from T123D, T123E, T123H, T123K, T123N, T123Q, and T123R.

22. The polypeptide of any one of claims 15-21, wherein the modified IL-2 comprises a substitution at amino acid position Q126.

23. The polypeptide of claim 22, wherein the substitution is selected from Q126N, Q126A, and Q126Y.

24. The polypeptide of any one of claims 15-23, wherein the modified IL-2 comprises a substitution at amino acid position 5127.

25. The polypeptide of claim 24, wherein the substitution is selected from S127D, S127E, S127H, S127K, S127N, S127P, and S127R.

26. The polypeptide of any one of claims 15-25, wherein the modified IL-2 comprises a substitution at amino acid position I129.

27. The polypeptide of claim 26, wherein the substitution is selected from I129A, I129H, I129R, and I129S.

28. The polypeptide of any one of claims 15-27, wherein the modified IL-2 comprises a substitution at amino acid position S130.

29. The polypeptide of claim 28, wherein the substitution is selected from S130E, S130K, S130N, S130P, S130Q, and S130R.

30. A polypeptide comprising a modified IL-2, wherein the modified IL-2 comprises at least one substitution at at least one amino acid position selected from Q22, R120, T123, S127, and S130.

31. The polypeptide of claim 30, wherein the modified IL-2 has reduced affinity for CD132 compared to wild-type IL-2.

32. The polypeptide of any one of claims 30-31, wherein the modified IL-2 comprises a substitution at amino acid position Q22.

33. The polypeptide of claim 32, wherein the substitution is selected from Q22A, Q22D, Q22E, Q22G, Q22H, Q22K, Q22N, Q22P, Q22R, Q22S, Q22T, Q22V, and Q22Y.

34. The polypeptide of any one of claims 30-33, wherein the modified IL-2 comprises a substitution at amino acid position R120.

35. The polypeptide of claim 34, wherein the substitution is selected from R120A, R120D, R120E, R120F, R120G, R120H, R120K, R120N, R120P, R120Q, R120S, R120V, and R120Y.

36. The polypeptide of claim 30-35, wherein the modified IL-2 comprises a substitution at amino acid position T123.

37. The polypeptide of claim 36, wherein the substitution is selected from T123D, T123E, T123H, T123K, T123N, T123Q, and T123R.

38. The polypeptide of any one of claims 30-37, wherein the modified IL-2 comprises a substitution at amino acid position 5127.

39. The polypeptide of claim 38, wherein the substitution is selected from S127D, S127E, S127H, S127K, S127N, S127P, S127Q, and S127R.

40. The polypeptide of any one of claims 30-39, wherein the modified IL-2 comprises a substitution at amino acid position S130.

41. The polypeptide of claim 40, wherein the substitution is selected from S130D, S130E, S130H, S130K, S130N, S130P, S130Q, and S130R.

42. The polypeptide of any one of claims 30-41, wherein the modified IL-2 comprises at least one substitution that reduces affinity for CD122 compared to wild-type IL-2.

43. The polypeptide of claim 42, wherein the modified IL-2 comprises at least one substitution at at least one amino acid position selected from H16, L19, M23, D84, N88, and E95.

44. The polypeptide of claim 43, wherein the modified IL-2 comprises a substitution at amino acid position H16.

45. The polypeptide of claim 44, wherein the substitution is selected from H16A, H16T, H16V, and H16N.

46. The polypeptide of any one of claims 43-45, wherein the modified IL-2 comprises a substitution at amino acid position L19.

47. The polypeptide of claim 46, wherein the substitution is selected from L19A, L19P, L19Q, L19Y, L19N, L19S, L19T, L19V.

48. The polypeptide of any one of claims 43-47, wherein the modified IL-2 comprises a substitution at amino acid position M23.

49. The polypeptide of claim 48, wherein the substitution is selected from M23A, M23G, M23S, M23T, M23V, M23D, M23E, M23I, M23K, M23L, M23N, M23Q, M23R, and M23Y.

50. The polypeptide of any one of claims 43-49, wherein the modified IL-2 comprises a substitution at amino acid position D84.

51. The polypeptide of claim 50, wherein the substitution is selected from D84S, D84G, D84A, D84T, D84V, D84Y, and D84N

52. The polypeptide of any one of claims 43-51, wherein the modified IL-2 comprises a substitution at amino acid position N88.

53. The polypeptide of claim 52, wherein the substitution is selected from N88T, N88A, and N88S.

54. The polypeptide of any one of claims 43-53, wherein the modified IL-2 comprises a substitution at amino acid position E95.

55. The polypeptide of claim 54, wherein the substitution is selected from E95Q, E95G, E95T, E95V, E95P, E95H, E95N, and E95Y.

56. The polypeptide of any one of claims 1-55, wherein the modified IL-2 comprises at least one substitution that reduces affinity for CD25 compared to wild-type IL-2.

57. The polypeptide of any one of claims 1-56, wherein the modified IL-2 comprises at least one substitution at at least one amino acid position selected from K43, Y45, E61, I114, P65, F42, R38, and L72, and/or wherein the modified IL-2 comprises a deletion of amino acid F42.

58. The polypeptide of claim 57, wherein the modified IL-2 comprises a substitution at amino acid F42 or comprises a deletion of amino acid F42.

59. The polypeptide of claim 58, wherein the modified IL-2 comprise a substitution at amino acid position F42 selected from F42K, F42A, F42R, F42G, F42S, and F42T.

60. The polypeptide of claim 58, wherein the modified IL-2 comprise a deletion of amino acid F42.

61. The polypeptide of any one of claims 57-60, wherein the modified IL-2 comprises a substitution at amino acid position K43.

62. The polypeptide of claim 61, wherein the substitution is selected from K43E and K43D.

63. The polypeptide of any one of claims 57-62, wherein the modified IL-2 comprises a substitution at amino acid position Y45.

64. The polypeptide of claim 63, wherein the substitution is selected from Y45R and Y45K.

65. The polypeptide of any one of claims 57-64, wherein the modified IL-2 comprises a substitution at amino acid position E61.

66. The polypeptide of claim 65, wherein the substitution is selected from E61R, E61G, E61H, E61N, E61P, E61S, E61T, E61Y, E61A, E61Q, and E61K.

67. The polypeptide of any one of claims 57-66, wherein the modified IL-2 comprises a substitution at amino acid position I114.

68. The polypeptide of claim 67, wherein the substitution is I114F, I114Y, or I114W.

69. The polypeptide of any one of claims 57-68, wherein the modified IL-2 comprises a substitution at amino acid position P65.

70. The polypeptide of claim 69, wherein the substitution is selected from P65R, P65E, P65K, P65H, P65Y, P65Q, P65D, and P65N.

71. The polypeptide of any one of claims 57-70, wherein the modified IL-2 comprises a substitution at amino acid position R38.

72. The polypeptide of claim 71, wherein the substitution at R38 is selected from R38A and R38G.

73. The polypeptide of any one of claims 57-72, wherein the modified IL-2 comprises a substitution at amino acid position L72.

74. The polypeptide of claim 73, wherein the substitution at L72 is and L72G.

75. The polypeptide of any one of claims 1-74, wherein the modified IL-2 comprises substitution Q22A, or substitution R120A, or substitutions Q22A and R120A.

76. The polypeptide of any one of claims 1-75, wherein the modified IL-2 comprises substitutions P65R and R38A or substitution P65R and E61R.

77. The polypeptide of any one of claims 1-76, wherein the modified IL-2 comprises at least one substitution selected from H16A, L19A, L19N, M23A, D84S or D84Y, N88S, and E95Q.

78. The polypeptide of any one of claims 1-77, wherein the modified IL-2 comprises substitutions at amino acid positions P65, H16, and D84.

79. The polypeptide of claim 78, wherein the modified IL-2 comprises substitutions P65R, H16A, and D84S; or substitutions P65R, H16A, and D84Y.

80. The polypeptide of any one of claims 1-79, wherein the modified IL-2 comprises at least one substitution at at least one amino acid position selected from T3 and C125, and/or comprises a deletion of the first five amino acids of IL-2.

81. The polypeptide of claim 80, wherein the modified IL-2 comprises at least one substitution selected from T3A, C125A, C125V, and C125S.

82. The polypeptide of claim 81, wherein the modified IL-2 comprises T3A and C125S substitutions, or T3A and C125V substitutions.

83. The polypeptide of claim 81, wherein the modified IL-2 comprises a deletion of the first five amino acids of IL-2 and a C125S substitution or C125V substitution.

84. The polypeptide of any one of the preceding claims, wherein the modified IL-2 comprises a set of substitutions selected from [T3A, H16A, E61R, P65R, D84Y, C125S], [T3A, H16A, M23T, E61R, P65R, D84Y, E95Q, C125S], [T3A, H16A, L19N, E61R, P65R, D84Y, C125S], [T3A, H16A, L19N, M23T, E61R, P65R, D84Y, E95Q, C125S], [T3A, H16A, E61R, P65R, D84Y, C125S, S127D], [T3A, H16A, M23T, E61R, P65R, D84Y, E95Q, C125S, S127D], [T3A, H16A, L19N, E61R, P65R, D84Y, C125S, S127D], and [T3A, H16A, L19N, M23T, E61R, P65R, D84Y, E95Q, C125S, S127D].

85. A modified polypeptide comprising a modified IL-2, wherein the modified IL-2 comprises a set of substitutions selected from [T3A, H16A, E61R, P65R, D84Y, C125S], [T3A, H16A, M23T, E61R, P65R, D84Y, E95Q, C125S], [T3A, H16A, L19N, E61R, P65R, D84Y, C125S], [T3A, H16A, L19N, M23T, E61R, P65R, D84Y, E95Q, C125S], [T3A, H16A, E61R, P65R, D84Y, C125S, S127D], [T3A, H16A, M23T, E61R, P65R, D84Y, E95Q, C125S, S127D], [T3A, H16A, L19N, E61R, P65R, D84Y, C125S, S127D], and [T3A, H16A, L19N, M23T, E61R, P65R, D84Y, E95Q, C125S, S127D].

86. The polypeptide of any one of the preceding claims, wherein the modified IL-2 comprises the indicated substitutions, and does not comprise any additional substitutions.

87. The polypeptide of any one of the preceding claims, wherein the modified IL-2 is a modified human IL-2.

88. The polypeptide of any one of the preceding claims, wherein the amino acid positions correspond to the amino acid positions in SEQ ID NO: 1.

89. The polypeptide of any one of the preceding claims, wherein the modified IL-2 comprises an amino acid sequence at least 90%, 91%, 92%, 93%, 94%, 95%, 96%, 97%, 98%, or 99% identical to SEQ ID NO: 84 and comprising corresponding substitutions of an amino acid sequence selected from SEQ ID NOs: 105-290.

90. The polypeptide of any one of the preceding claims, wherein the modified IL-2 comprises an amino acid sequence at least 90%, 91%, 92%, 93%, 94%, 95%, 96%, 97%, 98%, 99%, or 100% identical to an amino acid sequence selected from SEQ ID NOs: 270-277 and comprises substitution D84Y.

91. The polypeptide of any one of the preceding claims, wherein the modified IL-2 comprises an amino acid sequence selected from SEQ ID NOs: 105-290.

92. The polypeptide of any one of the preceding claims, wherein the modified IL-2 comprises an amino acid sequence selected from SEQ ID NOs: 270-277.

93. The polypeptide of any one of the preceding claims, wherein the polypeptide comprises an Fc region.

94. The polypeptide of claim 93, wherein the modified IL-2 is fused to the N-terminus or the C-terminus of the Fc region.

95. The polypeptide of claim 93 or claim 94, wherein the Fc region comprises a substitution at Kabat amino acid position T366.

96. The polypeptide of claim 95, wherein the Fc region comprises a T366W substitution.

97. The polypeptide of claim 93 or claim 94, wherein the Fc region comprises at least one substitution at at least one Kabat amino acid position selected from T366, L368, and Y407.

98. The polypeptide of claim 108, wherein the Fc region comprises T366S, L368A, and Y407V mutations.

99. The polypeptide of any one of claims 93-98, wherein the Fc region comprises a substitution at a Kabat position selected from S354 and Y349.

100. The polypeptide of claim 99, wherein the Fc region comprises a S354C or a Y349C substitution.

101. The polypeptide of any one of claims 93-100, wherein the Fc region comprises a substitution at Kabat amino acid position H435.

102. The polypeptide of claim 101, wherein the Fc region comprises a substitution selected from H435R and H435K.

103. The polypeptide of any one of claims 93-102, wherein the Fc region comprises at least one substitution at at least one Kabat amino acid position selected from M252 and M428.

104. The polypeptide of claim 103, wherein the Fc region comprises M252Y and M428V substitutions.

105. The polypeptide of any one of claims 93-104, wherein the Fc region comprises a deletion of Kabat amino acids E233, L234, and L235.

106. The polypeptide of any one of claims 93-104, wherein the Fc region comprises at least one substitution at at least one amino acid position selected from L234, L235, and P329.

107. The polypeptide of claim 106, wherein the Fc region comprises L234A, L235A, and P329G substitutions.

108. The polypeptide of any one of claims 93-107, wherein the Fc region comprises an amino acid sequence at least 90%, 91%, 92%, 93%, 94%, 95%, 96%, 97%, 98%, 99%, or 100% identical to an amino acid sequence selected from SEQ ID NOs: 47-83, 292, and 293.

109. The polypeptide of any one of claims 93-108, wherein the Fc region is part of a heavy chain constant region.

110. The polypeptide of claim 109, wherein the heavy chain constant region is an IgG constant region.

111. The polypeptide of claim 110, wherein the heavy chain constant region is an IgG1, IgG2, IgG3, or IgG4 constant region.

112. The polypeptide of any one of claims 93-111, wherein the modified IL-2 is fused to the C-terminus of the Fc region or heavy chain constant region.

113. The polypeptide of claim 112, wherein the modified IL-2 is fused to the C-terminus of the Fc region or heavy chain constant region via a linker comprising 1-20 amino acids.

114. The polypeptide of claim 113, wherein the linker comprises glycine amino acids.

115. The polypeptide of claim 114, wherein the linker comprises glycine and serine amino acids.

116. The polypeptide of any one of claims 113-115, wherein a majority, or all, of the amino acids in the linker are glycine and serine.

117. The polypeptide of any one of claims 93-116, wherein the polypeptide comprises an amino acid sequence selected from SEQ ID NOs: 105-290 and an amino acid sequence selected from SEQ ID NOs: 48, 64, 292, and 293.

118. The polypeptide of any one of the preceding claims, wherein the polypeptide comprises at least one antigen binding domain.

119. The polypeptide of claim 118, wherein the polypeptide comprises two, three, or four antigen binding domains.

120. The polypeptide of claim 118 or claim 119, wherein at least one antigen binding domain specifically binds to a T-cell antigen or a natural killer cell antigen.

121. The polypeptide of any one of claims 118-120, wherein at least one antigen binding domain specifically binds to a CD4⁺ T-cell antigen or a CD8⁺ T-cell antigen.

122. The polypeptide of claim 121, wherein the at least one antigen binding domain specifically binds to an antigen on an activated CD4⁺ T-cell or an activated CD8⁺ T-cell.

123. The polypeptide of any one of claims 118-122, wherein at least one antigen binding domain is an agonist.

124. The polypeptide of any one of claims 118-122, wherein the antigen binding domain is an antagonist.

125. The polypeptide of any one of claims 118-124, wherein at least one antigen binding domain specifically binds to PD-1, CTLA-4, LAG3, TIM3, 4-1BB, OX40, GITR, CD8a, CD8b, CD4, NKp30, NKG2A, TIGIT, TGFβR1, TGFβR2, Fas, NKG2D, NKp46, PD-L1, CD107a, ICOS, TNFR2, CD16a, or γδTCR.

126. The polypeptide of any one of claims 118-124, wherein at least one antigen binding domain specifically binds to PD-1.

127. The polypeptide of any one of claims 118-126, wherein at least one antigen binding domain is a human or humanized antigen binding domain.

128. The polypeptide of claim 127, wherein each antigen binding domain is, independently, a human or humanized antigen binding domain.

129. The polypeptide of any one of claims 118-128, wherein at least one antigen binding domain comprises a VHH domain.

130. The polypeptide of claim 129, wherein each antigen binding domain comprises a VHH domain.

131. The polypeptide of any one of claims 118-128, wherein at least one antigen binding domain comprises a VH domain and a VL domain.

132. The polypeptide of claim 131, wherein at least one antigen binding domain comprises the VH domain and the VL domain of an antibody selected from pembrolizumab, nivolumab, AMP-514, TSR-042, STI-A1110, ipilimumab, tremelimumab, urelumab, utomilumab, atezolizumab, and durvalumab.

133. The polypeptide of claim 131 or 132, wherein the at least one antigen binding domain comprises a single chain Fv (scFv).

134. The polypeptide of claim 131 or 132, wherein the polypeptide comprises a heavy chain constant region, wherein the VH domain is fused to the heavy chain constant region, and wherein the VL domain is associated with the VH domain.

135. The polypeptide of claim 134, wherein the VL domain is fused to a light chain constant region.

136. The polypeptide of claim 135, wherein the light chain constant region is selected from kappa and lambda.

137. The polypeptide of any one of claims 118-136, wherein each of the antigen binding domains are the same.

138. The polypeptide of any one of claims 118-137, wherein each of the antigen binding domains specifically bind to the same antigen.

139. The polypeptide of any one of claims 118-136, wherein at least one of the antigen binding domains specifically binds to a different antigen than at least one of the other antigen binding domains.

140. The polypeptide of claim 139, wherein at least one antigen binding domain specifically binds to PD-1 and at least one other antigen binding domain specifically binds to a T-cell antigen or natural killer cell antigen other than PD-1.

141. The polypeptide of any one of claims 118-140, wherein at least one antigen binding domain binds to PD-1, CTLA-4, LAG3, TIM3, 4-1BB, OX40, GITR, CD8a, CD8b, CD4, NKp30, NKG2A, TIGIT, TGFβR1, TGFβR2, Fas, NKG2D, NKp46, PD-L1, CD107a, ICOS, TNFR2, CD16a, DNAM1, or γδTCR (Vγ9, Vγ2, Vδ1).

142. The polypeptide of any one of claims 93-141, wherein the polypeptide forms a homodimer under physiological conditions.

143. The polypeptide of any one of the preceding claims, wherein the modified IL-2 binds a human IL-2R with an affinity at least 2-fold, 3-fold, 4-fold, 5-fold, 6-fold, 7-fold, 8-fold, 9-fold, at least 10-fold, at least 20-fold, at least 30-fold, at least 50-fold, or at least 100-fold lower than the affinity of human wild type IL-2 for the IL-2R.

144. A complex comprising a first polypeptide and a second polypeptide, wherein the first polypeptide is the polypeptide of any one of the preceding claims.

145. The complex of claim 144, wherein the first polypeptide comprises a first Fc region and the second polypeptide comprises a second Fc region.

146. The complex of claim 144 or claim 145, wherein each Fc region is an isotype selected from human IgG1, IgG2, IgG3, an IgG4.

147. The complex of claim 146, wherein each Fc region is a human IgG1.

148. The complex of any one of claims 144-147, wherein each Fc region comprises a deletion of amino acids E233, L234, and L235.

149. The complex of any one of claims 144-148, wherein each Fc region comprises a H435R or H435K mutation.

150. The complex of any one of claims 155-160, wherein the Fc region comprises a mutations M252Y and M428L or mutations M252Y and M428V.

151. The complex of any one of claims 144-150, wherein the first Fc region or the second Fc region comprises a T366W mutation, and the other Fc region comprises mutations T366S, L368A, and Y407V.

152. The complex of claim 151, wherein the first Fc region or the second Fc region comprises a S354C mutation.

153. The complex of any one of claims 144-152, wherein each Fc region independently comprises an amino acid sequence at least 90%, 91%, 92%, 93%, 94%, 95%, 96%, 97%, 98%, 99%, or 100% identical to an amino acid sequence selected from SEQ ID NOs: 47-83, 292, and 293.

154. The complex of any one of claims 144-153, wherein the second polypeptide does not comprise a modified IL-2.

155. The complex of any one of claims 144-154, wherein the first polypeptide comprises at least one antigen binding domain.

156. The complex of any one of claims 144-155, wherein the second polypeptide comprises at least one antigen binding domain.

157. The complex of any one of claims 144-156, wherein the first polypeptide comprises a first antigen binding domain, an Fc region, and a modified IL-2.

158. The complex of claim 157, wherein the first antigen binding domain is fused to the N-terminus of the Fc region and the modified IL-2 is fused to the C-terminus of the Fc region.

159. The complex of claim 157 or claim 158, wherein the second polypeptide comprises a second antigen binding domain and an Fc region.

160. The complex of claim 159, wherein the first antigen binding domain and the second antigen binding domain are the same or different.

161. The complex of claim 160, wherein:
a) the first antigen binding domain and the second antigen binding domain both bind PD-1;
b) the first antigen binding domain binds PD-1, and the second antigen binding domain binds LAG3;
c) the first antigen binding domain binds PD-1, and the second antigen binding domain binds CTLA-4;
d) the first antigen binding domain binds PD-1, and the second antigen binding domain binds 4-1BB;
e) the first antigen binding domain binds PD-1, and the second antigen binding domain binds OX40;
f) the first antigen binding domain binds PD-1, and the second antigen binding domain binds GITR;
g) the first antigen binding domain binds PD-1, and the second antigen binding domain binds CD8a;
h) the first antigen binding domain binds PD-1, and the second antigen binding domain binds CD8b;
i) the first antigen binding domain binds PD-1, and the second antigen binding domain binds CD4;
j) the first antigen binding domain binds PD-1, and the second antigen binding domain binds NKp30;
k) the first antigen binding domain binds PD-1, and the second antigen binding domain binds NKG2A;
l. the first antigen binding domain binds PD-1, and the second antigen binding domain binds TIGIT;
m) the first antigen binding domain binds PD-1, and the second antigen binding domain binds NKG2D;
n) the first antigen binding domain binds PD-1, and the second antigen binding domain binds TGFBR2;
o) the first antigen binding domain binds PD-1, and the second antigen binding domain binds Fas;
p) the first antigen binding domain binds PD-1, and the second antigen binding domain binds CD107a;
q) the first antigen binding domain binds PD-1, and the second antigen binding domain binds NKp46;
r) the first antigen binding domain binds CD8a, and the second antigen binding domain binds TGFRβR2;
s) the first antigen binding domain binds CD8a, and the second antigen binding domain binds Fas;
t) the first antigen binding domain binds NKG2D, and the second antigen binding domain binds TGFRβR2;
u) the first antigen binding domain binds NKG2D, and the second antigen binding domain binds Fas;
v) the first antigen binding domain binds NKG2A, and the second antigen binding domain binds TGFRβR2;
w) the first antigen binding domain binds NKG2A, and the second antigen binding domain binds Fas;
x) the first antigen binding domain binds NKp46, and the second antigen binding domain binds TGFRβR2;
y) the first antigen binding domain binds NKp46, and the second antigen binding domain binds Fas;
z) the first antigen binding domain binds CTLA-4, and the second antigen binding domain binds LAGS;
aa) the first antigen binding domain binds CTLA-4, and the second antigen binding domain binds Tim3;
bb) the first antigen binding domain binds CTLA-4, and the second antigen binding domain binds OX40;
cc) the first antigen binding domain binds CTLA-4, and the second antigen binding domain binds GITR;
dd) the first antigen binding domain binds CTLA-4, and the second antigen binding domain binds CD107a;
ee) the first antigen binding domain binds CTLA-4, and the second antigen binding domain binds NKp46
ff) the first antigen binding domain binds ICOS, and the second antigen binding domain binds TNFR2;
gg) the first antigen binding domain binds γδTCR, and the second antigen binding domain binds NKG2D;
hh) the first antigen binding domain binds γδTCR, and the second antigen binding domain binds DNAM1;
ii) the first antigen binding domain binds γδTCR, and the second antigen binding domain binds TIGIT;
jj) the first antigen binding domain binds γδTCR, and the second antigen binding domain binds 4-1BB;
kk) the first antigen binding domain binds γδTCR, and the second antigen binding domain binds Fas;
ll) the first antigen binding domain binds γδTCR, and the second antigen binding domain binds NKG2A; or
mm) the first antigen binding domain binds γδTCR, and the second antigen binding domain binds CD16a.

162. The complex of any one of claims 144-161, wherein the modified IL-2 binds a human IL-2R with an affinity at least 2-fold, 3-fold, 4-fold, 5-fold, 6-fold, 7-fold, 8-fold, 9-fold, at least 10-fold, at least 20-fold, at least 30-fold, at least 50-fold, or at least 100-fold lower than the affinity of human wild type IL-2 for the IL-2R.

163. A pharmaceutical composition comprising a polypeptide of any one of claims 1-154 or the complex of any one of claims 144-162 and a pharmaceutically acceptable carrier.

164. An isolated nucleic acid the encodes a polypeptide of any one of claims 1-143 or the complex of any one of claims 144-162.

165. An expression vector comprising the nucleic acid of claim 164.

166. An isolated host cell comprising the nucleic acid of claim 164 or the expression vector of claim 165.

167. An isolated host cell that expresses the polypeptide of any one of claims 1-143 or the complex of any one of claims 144-162.

168. A method of producing the polypeptide of any one of claims 1-143 or the complex of any one of claims 144-162 comprising incubating the host cell of claim 166 or claim 167 under conditions suitable to express the polypeptide or complex.

169. The method of claim 168, further comprising isolating the polypeptide or complex.

170. A method of increasing CD4+ and/or CD8+ T cell proliferation comprising contacting T cells with the polypeptide of any one of claims 1-154 or the complex of any one of claims 144-162.

171. The method of claim 170, wherein the CD4+ and/or CD8+ T cells are in vitro.

172. The method of claim 170, wherein the CD4+ and/or CD8+ T cells are in vivo.

173. The method of any one of claims 170-172, wherein the increase is at least 1.5-fold, at least 2-fold, at least 3-fold, or by at least 5-fold.

174. A method of increasing NK cell proliferation comprising contacting NK cells with the polypeptide of any one of claims 1-143 or the complex of any one of claims 144-162.

175. The method of claim 174, wherein the increase is at least 1.5-fold, at least 2-fold, at least 3-fold, or by at least 5-fold.

176. A method of treating cancer comprising administering to a subject with cancer a pharmaceutically effective amount of the polypeptide of any one of claims 1-143 or the complex of any one of claims 144-162, or the pharmaceutical composition of claim 163.

177. The method of claim 176, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; and chronic myeloblastic leukemia.

178. The method of claim 176 or 177, further comprising administering an additional therapeutic agent.

179. The method of claim 178, wherein the additional therapeutic agent is an anti-cancer agent.

180. The method of claim 179, wherein the anti-cancer agent is selected from a chemotherapeutic agent, an anti-cancer biologic, radiation therapy, CAR-T therapy, and an oncolytic virus.

181. The method of claim 179 or claim 180, wherein the additional therapeutic agent is an anti-cancer biologic.

182. The method of claim 181, wherein the anti-cancer biologic is an agent that inhibits PD-1 and/or PD-L1.

183. The method of claim 181, wherein the anti-cancer biologic is an agent that inhibits VISTA, gpNMB, B7H3, B7H4, HHLA2, CTLA4, or TIGIT.

184. The method of any one of claims 179-183, wherein the anti-cancer agent is an antibody.

185. The method of claim 181, wherein the anti-cancer biologic is a cytokine.

186. The method of claim 179, wherein the anti-cancer agent is CAR-T therapy.

187. The method of claim 179, wherein the anti-cancer agent is an oncolytic virus.

188. The method of any one of claims 176-187, further comprising tumor resection and/or radiation therapy.
